(19) United States
(12) Patent Application Publication
(10) Pub. No.: US 2025/0263702 A1
(43) Pub. Date: Aug. 21, 2025

(54) RNAI AGENTS TARGETING CIDEB AND RELATED METHODS

(71) Applicant: FLAGSHIP PIONEERING INNOVATIONS VII, LLC, Cambridge, MA (US)

(72) Inventors: Scott Joseph HAYTON, Toronto (CA); Janno HARJES, Thurnau (DE); Kathrin Stefanie GERAUER, Prichsenstadt (DE); Monika KRAMPERT, Bamberg (DE); Jose Antonio Fidel CHAVEZ REATEGUI, Doylestown, PA (US); Luciano Henrique APPONI, Lynn, MA (US); David Charles BLAKEY, Prestbury (GB); Janeta Valentina POPOVICI-MULLER, Windham, NH (US)

(21) Appl. No.: 19/055,667

(22) Filed: Feb. 18, 2025

Related U.S. Application Data
(60) Provisional application No. 63/555,164, filed on Feb. 19, 2024.
(60) Provisional application No. 63/635,269, filed on Apr. 17, 2024.
(60) Provisional application No. 63/707,351, filed on Oct. 15, 2024.

(51) Int. Cl. C12N 15/113

(52) U.S. Cl. CPC C12N 15/113; C12N 2310/14; C12N 2310/351

(57) ABSTRACT

Provided herein are, inter alia, agents (e.g., RNAi agents, dsRNA agents) comprising a sense strand and an antisense strand targeting CIDEB (e.g., hCIDEB); and methods of manufacturing and pharmaceutical compositions comprising the same. Further provided herein are methods of utilizing the agents (e.g., RNAi agents, dsRNA agents) including, e.g., methods of inhibiting or decreasing CIDEB expression (e.g., mRNA expression), methods of treating CIDEB associated diseases, and methods of treating liver diseases (e.g., MASH).

### RELATED APPLICATIONS

This application claims priority to U.S. Ser. No.: 63/555,164, filed Feb. 19, 2024, U.S. Ser. No. 63/635,269, filed Apr. 17, 2024, and U.S. Ser. No. 63/707,351, filed Oct. 15, 2024, the entire contents of each of which is incorporated herein by reference.

### SEQUENCE LISTING

The instant application contains a Sequence Listing which has been submitted electronically in XML format and is hereby incorporated by reference in its entirety. Said XML copy, created on May 5, 2025, is named 62801.53US01 Sequence Listing.xml and is 6,883,805 bytes in size.

### 1. FIELD

This disclosure relates to RNAi agents (e.g., double stranded RNA (dsRNA) agents comprising a sense strand and an antisense strand) targeting cell death inducing DNA fragmentation factor alpha like effector B (CIDEB). The disclosure further relates to pharmaceutical compositions comprising the same; and methods of utilizing the same, including, e.g., methods of treating CIDEB associated diseases (e.g., liver diseases).

### 2. BACKGROUND

The CIDE (cell death inducing DNA fragmentation factor alpha like effector) protein family is a family of lipid droplet-associated proteins. There are three members of the CIDE protein family, CIDEA, CIDEB, and CIDEC. Each of the CIDE proteins comprises a common CIDE-N domain and a CIDE-C domain, which varies between each of the members. The tissue expression pattern of each CIDE protein differs but is correlative with their association with lipid droplets-CIDEA and CIDEC are primarily expressed in adipose tissue, while CIDEB is highly expressed in the liver. The association of CIDE proteins with lipid droplets can be a direct physical interaction with the surface of the lipid droplet, as well as an association with other lipid droplet proteins, such as perilipin.

### 3. SUMMARY

Provided herein are, inter alia, agents (e.g., RNAi agents, dsRNA agents) comprising a sense strand and an antisense strand targeting CIDEB (e.g., hCIDEB); and methods of manufacturing and pharmaceutical compositions comprising the same. Further provided herein are methods of utilizing the agents (e.g., RNAi agents, dsRNA agents) including, e.g., methods of inhibiting or decreasing CIDEB expression (e.g., mRNA expression), methods of treating CIDEB associated diseases, and methods of treating liver diseases (e.g., metabolic dysfunction-associated steatohepatitis (MASH)).

Accordingly, in one aspect provided herein are double stranded ribonucleic acid (dsRNA) agents for inhibiting expression of cell death inducing DNA fragmentation factor alpha like effector (CIDEB) (e.g., human CIDEB (hCIDEB)), wherein the dsRNA agent comprises a sense strand and an antisense strand forming a double stranded region, wherein the nucleotide sequence of the antisense strand differs by no more than 5 (e.g., 0, 1, 2, 3, 4, or 5) nucleotides from the nucleotide sequence of any one of the antisense strands set forth in Table 2 or Table 3 or set forth in any one of SEQ ID NOS: 187-369, 602-657, 893-1131, or 1188-1191.

In some embodiments, the nucleotide sequence of the sense strand differs by no more than 5 (e.g., 0, 1, 2, 3, 4, or 5) nucleotides from the nucleotide sequence of the corresponding sense strand set forth in Table 2 or Table 3 or the corresponding sense strand set forth in one of SEQ ID NOS: 4-186, 553-601, 658-892, or 1192.

In some embodiments, the nucleotide sequence of the antisense strand differs by no more than 3 (e.g., 0, 1, 2, or 3) nucleotides from the nucleotide sequence of any one of the antisense strands set forth in Table 2 or Table 3 or set forth in any one of SEQ ID NOS: 187-369, 602-657, 893-1131, or 1188-1191; and the nucleotide sequence of the sense strand differs by no more than 3 (e.g., 0, 1, 2, or 3) nucleotides from the nucleotide sequence of the corresponding sense strand set forth in Table 2 or Table 3 or the corresponding sense strand set forth in one of SEQ ID NOS: 4-186, 553-601, 658-892, or 1192.

In some embodiments, the nucleotide sequence of the antisense strand comprises the nucleotide sequence of any one of the antisense strands set forth in Table 2 or Table 3 or set forth in any one of SEQ ID NOS: 187-369, 602-657, 893-1131, or 1188-1191; and the nucleotide sequence of the sense strand comprises the nucleotide sequence of the corresponding sense strand set forth in Table 2 or Table 3 or the corresponding sense strand set forth in one of SEQ ID NOS: 4-186, 553-601, 658-892, or 1192.

In some embodiments, (a) the nucleotide sequence of the antisense strand differs by no more than 5 (e.g., 0, 1, 2, 3, 4, or 5) nucleotides from the nucleotide sequence set forth in SEQ ID NO: 355; and the nucleotide sequence of the sense strand differs by no more than 5 (e.g., 0, 1, 2, 3, 4, or 5) nucleotides from the nucleotide sequence set forth in SEQ ID NO: 172; (b) the nucleotide sequence of the antisense strand differs by no more than 5 (e.g., 0, 1, 2, 3, 4, or 5) nucleotides from the nucleotide sequence set forth in SEQ ID NO: 315; and the nucleotide sequence of the sense strand differs by no more than 5 (e.g., 0, 1, 2, 3, 4, or 5) nucleotides from the nucleotide sequence set forth in SEQ ID NO: 132; (c) the nucleotide sequence of the antisense strand differs by no more than 5 (e.g., 0, 1, 2, 3, 4, or 5) nucleotides from the nucleotide sequence set forth in SEQ ID NO: 356; and the nucleotide sequence of the sense strand differs by no more than 5 (e.g., 0, 1, 2, 3, 4, or 5) nucleotides from the nucleotide sequence set forth in SEQ ID NO: 173; or (d) the nucleotide sequence of the antisense strand differs by no more than 5 (e.g., 0, 1, 2, 3, 4, or 5) nucleotides from the nucleotide sequence set forth in SEQ ID NO: 321; and the nucleotide sequence of the sense strand differs by no more than 5 (e.g., 0, 1, 2, 3, 4, or 5) nucleotides from the nucleotide sequence set forth in SEQ ID NO: 138.

In some embodiments, (a) the nucleotide sequence of the antisense strand differs by no more than 3 (e.g., 0, 1, 2, or 3) nucleotides from the nucleotide sequence set forth in SEQ ID NO: 355; and the nucleotide sequence of the sense strand differs by no more than 3 (e.g., 0, 1, 2, or 3) nucleotides from the nucleotide sequence set forth in SEQ ID NO: 172; (b) the nucleotide sequence of the antisense strand differs by no more than 3 (e.g., 0, 1, 2, or 3) nucleotides from the nucleotide sequence set forth in SEQ ID NO: 315; and the nucleotide sequence of the sense strand differs by no more than 3 (e.g., 0, 1, 2, or 3) nucleotides from the nucleotide sequence set forth in SEQ ID NO: 132; (c) the nucleotide sequence of the antisense strand differs by no more than 3 (e.g., 0, 1, 2, or 3) nucleotides from the nucleotide sequence set forth in SEQ ID NO: 356; and the nucleotide sequence of the sense strand differs by no more than 3 (e.g., 0, 1, 2, or 3) nucleotides from the nucleotide sequence set forth in SEQ ID NO: 173; or (d) the nucleotide sequence of the antisense strand differs by no more than 3 (e.g., 0, 1, 2, or 3) nucleotides from the nucleotide sequence set forth in SEQ ID NO: 321; and the nucleotide sequence of the sense strand differs by no more than 3 (e.g., 0, 1, 2, or 3) nucleotides from the nucleotide sequence set forth in SEQ ID NO: 138.

In some embodiments, (a) the nucleotide sequence of the antisense strand comprises the nucleotide sequence set forth in SEQ ID NO: 355; and the nucleotide sequence of the sense strand comprises the nucleotide sequence set forth in SEQ ID NO: 172; (b) the nucleotide sequence of the antisense strand comprises the nucleotide sequence set forth in SEQ ID NO: 315; and the nucleotide sequence of the sense strand comprises the nucleotide sequence set forth in SEQ ID NO: 132; (c) the nucleotide sequence of the antisense strand comprises the nucleotide sequence set forth in SEQ ID NO: 356; and the nucleotide sequence of the sense strand comprises the nucleotide sequence set forth in SEQ ID NO: 173; or (d) the nucleotide sequence of the antisense strand comprises the nucleotide sequence set forth in SEQ ID NO: 321; and the nucleotide sequence of the sense strand comprises the nucleotide sequence set forth in SEQ ID NO: 138.

In some embodiments, (a) the nucleotide sequence of the antisense strand differs by no more than 5 (e.g., 0, 1, 2, 3, 4, or 5) nucleotides from the nucleotide sequence set forth in SEQ ID NO: 1191; and the nucleotide sequence of the sense strand differs by no more than 5 (e.g., 0, 1, 2, 3, 4, or 5) nucleotides from the nucleotide sequence set forth in SEQ ID NO: 1192; (b) the nucleotide sequence of the antisense strand differs by no more than 5 (e.g., 0, 1, 2, 3, 4, or 5) nucleotides from the nucleotide sequence set forth in SEQ ID NO: 1188; and the nucleotide sequence of the sense strand differs by no more than 5 (e.g., 0, 1, 2, 3, 4, or 5) nucleotides from the nucleotide sequence set forth in SEQ ID NO: 786; (c) the nucleotide sequence of the antisense strand differs by no more than 5 (e.g., 0, 1, 2, 3, 4, or 5) nucleotides from the nucleotide sequence set forth in SEQ ID NO: 1190; and the nucleotide sequence of the sense strand differs by no more than 5 (e.g., 0, 1, 2, 3, 4, or 5) nucleotides from the nucleotide sequence set forth in SEQ ID NO: 786; (d) the nucleotide sequence of the antisense strand differs by no more than 5 (e.g., 0, 1, 2, 3, 4, or 5) nucleotides from the nucleotide sequence set forth in SEQ ID NO: 1062; and the nucleotide sequence of the sense strand differs by no more than 5 (e.g., 0, 1, 2, 3, 4, or 5) nucleotides from the nucleotide sequence set forth in SEQ ID NO: 827; or (e) the nucleotide sequence of the antisense strand differs by no more than 5 (e.g., 0, 1, 2, 3, 4, or 5) nucleotides from the nucleotide sequence set forth in SEQ ID NO: 1189; and the nucleotide sequence of the sense strand differs by no more than 5 (e.g., 0, 1, 2, 3, 4, or 5) nucleotides from the nucleotide sequence set forth in SEQ ID NO: 792.

In some embodiments, (a) the nucleotide sequence of the antisense strand differs by no more than 3 (e.g., 0, 1, 2, or 3) nucleotides from the nucleotide sequence set forth in SEQ ID NO: 1191; and the nucleotide sequence of the sense strand differs by no more than 3 (e.g., 0, 1, 2, or 3) nucleotides from the nucleotide sequence set forth in SEQ ID NO: 1192; (b) the nucleotide sequence of the antisense strand differs by no more than 3 (e.g., 0, 1, 2, or 3) nucleotides from the nucleotide sequence set forth in SEQ ID NO: 1188; and the nucleotide sequence of the sense strand differs by no more than 3 (e.g., 0, 1, 2, or 3) nucleotides from the nucleotide sequence set forth in SEQ ID NO: 786; (c) the nucleotide sequence of the antisense strand differs by no more than 3 (e.g., 0, 1, 2, or 3) nucleotides from the nucleotide sequence set forth in SEQ ID NO: 1190; and the nucleotide sequence of the sense strand differs by no more than 3 (e.g., 0, 1, 2, or 3) nucleotides from the nucleotide sequence set forth in SEQ ID NO: 786; (d) the nucleotide sequence of the antisense strand differs by no more than 3 (e.g., 0, 1, 2, or 3) nucleotides from the nucleotide sequence set forth in SEQ ID NO: 1062; and the nucleotide sequence of the sense strand differs by no more than 3 (e.g., 0, 1, 2, or 3) nucleotides from the nucleotide sequence set forth in SEQ ID NO: 827; or (c) the nucleotide sequence of the antisense strand differs by no more than 3 (e.g., 0, 1, 2, or 3) nucleotides from the nucleotide sequence set forth in SEQ ID NO: 1189; and the nucleotide sequence of the sense strand differs by no more than 3 (e.g., 0, 1, 2, or 3) nucleotides from the nucleotide sequence set forth in SEQ ID NO: 792.

In some embodiments, (a) the nucleotide sequence of the antisense strand comprises the nucleotide sequence set forth in SEQ ID NO: 1191; and the nucleotide sequence of the sense strand comprises the nucleotide sequence set forth in SEQ ID NO: 1192; (b) the nucleotide sequence of the antisense strand comprises the nucleotide sequence set forth in SEQ ID NO: 1188; and the nucleotide sequence of the sense strand comprises the nucleotide sequence set forth in SEQ ID NO: 786; (c) the nucleotide sequence of the antisense strand comprises the nucleotide sequence set forth in SEQ ID NO: 1190; and the nucleotide sequence of the sense strand comprises the nucleotide sequence set forth in SEQ ID NO: 786; (d) the nucleotide sequence of the antisense strand comprises the nucleotide sequence set forth in SEQ ID NO: 1062; and the nucleotide sequence of the sense strand comprises the nucleotide sequence set forth in SEQ ID NO: 827; or (c) the nucleotide sequence of the antisense strand comprises the nucleotide sequence set forth in SEQ ID NO: 1189; and the nucleotide sequence of the sense strand comprises the nucleotide sequence set forth in SEQ ID NO: 792.

In one aspect, provided herein are dsRNA agents for inhibiting expression of CIDEB (e.g., hCIDEB), wherein the dsRNA agent comprises a sense strand and an antisense strand forming a double stranded region, and wherein the nucleotide sequence of the antisense strand differs by no more than 5 (e.g., 0, 1, 2, 3, 4, or 5) nucleotides from the nucleotide sequence of any one of the antisense strands of any one of dsRNA agents 1-482 set forth in Table 2 or Table 3; and the nucleotide sequence of the sense strand differs by no more than 5 (e.g., 0, 1, 2, 3, 4, or 5) nucleotides from the nucleotide sequence of the sense strand of the corresponding dsRNA agent set forth in Table 2 or Table 3.

In some embodiments, the nucleotide sequence of the antisense strand differs by no more than 3 (e.g., 0, 1, 2, or 3) nucleotides from the nucleotide sequence of any one of the antisense strands of any one of dsRNA agents 1-482 set forth in Table 2 or Table 3; and the nucleotide sequence of the sense strand differs by no more than 3 (e.g., 0, 1, 2, or 3) nucleotides from the nucleotide sequence of the sense strand of the corresponding dsRNA agent set forth in Table 2 or Table 3.

In some embodiments, (a) the nucleotide sequence of the antisense strand differs by no more than 5 (e.g., 0, 1, 2, 3, 4, or 5) nucleotides from the nucleotide sequence of the antisense strand of dsRNA agent 129; and the nucleotide sequence of the sense strand differs by no more than 5 (e.g., 0, 1, 2, 3, 4, or 5) nucleotides from the nucleotide sequence of dsRNA agent 129; (b) the nucleotide sequence of the antisense strand differs by no more than 5 (e.g., 0, 1, 2, 3, 4, or 5) nucleotides from the nucleotide sequence of the antisense strand of dsRNA agent 135; and the nucleotide sequence of the sense strand differs by no more than 5 (e.g., 0, 1, 2, 3, 4, or 5) nucleotides from the nucleotide sequence of dsRNA agent 135; (c) the nucleotide sequence of the antisense strand differs by no more than 5 (e.g., 0, 1, 2, 3, 4, or 5) nucleotides from the nucleotide sequence of the antisense strand of dsRNA agent 169; and the nucleotide sequence of the sense strand differs by no more than 5 (e.g., 0, 1, 2, 3, 4, or 5) nucleotides from the nucleotide sequence of dsRNA agent 169; or (d) the nucleotide sequence of the antisense strand differs by no more than 5 (e.g., 0, 1, 2, 3, 4, or 5) nucleotides from the nucleotide sequence of the antisense strand of dsRNA agent 170; and the nucleotide sequence of the sense strand differs by no more than 5 (e.g., 0, 1, 2, 3, 4, or 5) nucleotides from the nucleotide sequence of dsRNA agent 170.

In some embodiments, (a) the nucleotide sequence of the antisense strand differs by no more than 3 (e.g., 0, 1, 2, or 3) nucleotides from the nucleotide sequence of the antisense strand of dsRNA agent 129; and the nucleotide sequence of the sense strand differs by no more than 3 (e.g., 0, 1, 2, or 3) nucleotides from the nucleotide sequence of dsRNA agent 129; (b) the nucleotide sequence of the antisense strand differs by no more than 3 (e.g., 0, 1, 2, or 3) nucleotides from the nucleotide sequence of the antisense strand of dsRNA agent 135; and the nucleotide sequence of the sense strand differs by no more than 3 (e.g., 0, 1, 2, or 3) nucleotides from the nucleotide sequence of dsRNA agent 135; (c) the nucleotide sequence of the antisense strand differs by no more than 3 (e.g., 0, 1, 2, or 3) nucleotides from the nucleotide sequence of the antisense strand of dsRNA agent 169; and the nucleotide sequence of the sense strand differs by no more than 3 (e.g., 0, 1, 2, or 3) nucleotides from the nucleotide sequence of dsRNA agent 169; or (d) the nucleotide sequence of the antisense strand differs by no more than 3 (e.g., 0, 1, 2, or 3) nucleotides from the nucleotide sequence of the antisense strand of dsRNA agent 170; and the nucleotide sequence of the sense strand differs by no more than 3 (e.g., 0, 1, 2, or 3) nucleotides from the nucleotide sequence of dsRNA agent 170.

In some embodiments, (a) the nucleotide sequence of the antisense strand comprises the nucleotide sequence of the antisense strand of dsRNA agent 129; and the nucleotide sequence of the sense strand comprises of the sense strand of dsRNA agent 129; (b) the nucleotide sequence of the antisense strand comprises the nucleotide sequence of the antisense strand of dsRNA agent 135; and the nucleotide sequence of the sense strand comprises of the sense strand of dsRNA agent 135; (c) the nucleotide sequence of the antisense strand comprises the nucleotide sequence of the antisense strand of dsRNA agent 169; and the nucleotide sequence of the sense strand comprises of the sense strand of dsRNA agent 169; or (d) the nucleotide sequence of the antisense strand comprises the nucleotide sequence of the antisense strand of dsRNA agent 170; and the nucleotide sequence of the sense strand comprises of the sense strand of dsRNA agent 170.

In some embodiments, (a) the nucleotide sequence of the antisense strand differs by no more than 5 (e.g., 0, 1, 2, 3, 4, or 5) nucleotides from the nucleotide sequence of the antisense strand of dsRNA agent 479; and the nucleotide sequence of the sense strand differs by no more than 5 (e.g., 0, 1, 2, 3, 4, or 5) nucleotides from the nucleotide sequence of dsRNA agent 479; (b) the nucleotide sequence of the antisense strand differs by no more than 5 (e.g., 0, 1, 2, 3, 4, or 5) nucleotides from the nucleotide sequence of the antisense strand of dsRNA agent 481; and the nucleotide sequence of the sense strand differs by no more than 5 (e.g., 0, 1, 2, 3, 4, or 5) nucleotides from the nucleotide sequence of dsRNA agent 481; (c) the nucleotide sequence of the antisense strand differs by no more than 5 (e.g., 0, 1, 2, 3, 4, or 5) nucleotides from the nucleotide sequence of the antisense strand of dsRNA agent 482; and the nucleotide sequence of the sense strand differs by no more than 5 (e.g., 0, 1, 2, 3, 4, or 5) nucleotides from the nucleotide sequence of dsRNA agent 482; (d) the nucleotide sequence of the antisense strand differs by no more than 5 (e.g., 0, 1, 2, 3, 4, or 5) nucleotides from the nucleotide sequence of the antisense strand of dsRNA agent 480; and the nucleotide sequence of the sense strand differs by no more than 5 (e.g., 0, 1, 2, 3, 4, or 5) nucleotides from the nucleotide sequence of dsRNA agent 480; or (c) the nucleotide sequence of the antisense strand differs by no more than 5 (e.g., 0, 1, 2, 3, 4, or 5) nucleotides from the nucleotide sequence of the antisense strand of dsRNA agent 409; and the nucleotide sequence of the sense strand differs by no more than 5 (e.g., 0, 1, 2, 3, 4, or 5) nucleotides from the nucleotide sequence of dsRNA agent 409.

In some embodiments, (a) the nucleotide sequence of the antisense strand differs by no more than 3 (e.g., 0, 1, 2, or 3) nucleotides from the nucleotide sequence of the antisense strand of dsRNA agent 479; and the nucleotide sequence of the sense strand differs by no more than 3 (e.g., 0, 1, 2, or 3) nucleotides from the nucleotide sequence of dsRNA agent 479; (b) the nucleotide sequence of the antisense strand differs by no more than 3 (e.g., 0, 1, 2, or 3) nucleotides from the nucleotide sequence of the antisense strand of dsRNA agent 481; and the nucleotide sequence of the sense strand differs by no more than 3 (e.g., 0, 1, 2, or 3) nucleotides from the nucleotide sequence of dsRNA agent 481; (c) the nucleotide sequence of the antisense strand differs by no more than 3 (e.g., 0, 1, 2, or 3) nucleotides from the nucleotide sequence of the antisense strand of dsRNA agent 482; and the nucleotide sequence of the sense strand differs by no more than 3 (e.g., 0, 1, 2, or 3) nucleotides from the nucleotide sequence of dsRNA agent 482; (d) the nucleotide sequence of the antisense strand differs by no more than 3 (e.g., 0, 1, 2, or 3) nucleotides from the nucleotide sequence of the antisense strand of dsRNA agent 480; and the nucleotide sequence of the sense strand differs by no more than 3 (e.g., 0, 1, 2, or 3) nucleotides from the nucleotide sequence of dsRNA agent 480; or (c) the nucleotide sequence of the antisense strand differs by no more than 3 (e.g., 0, 1, 2, or 3) nucleotides from the nucleotide sequence of the antisense strand of dsRNA agent 409; and the nucleotide sequence of the sense strand differs by no more than 3 (e.g., 0, 1, 2, or 3) nucleotides from the nucleotide sequence of dsRNA agent 409.

In some embodiments, (a) the nucleotide sequence of the antisense strand comprises the nucleotide sequence of the antisense strand of dsRNA agent 479; and the nucleotide sequence of the sense strand comprises of the sense strand of dsRNA agent 479; (b) the nucleotide sequence of the antisense strand comprises the nucleotide sequence of the antisense strand of dsRNA agent 481; and the nucleotide sequence of the sense strand comprises of the sense strand of dsRNA agent 481; (c) the nucleotide sequence of the antisense strand comprises the nucleotide sequence of the antisense strand of dsRNA agent 482; and the nucleotide sequence of the sense strand comprises of the sense strand of dsRNA agent 482; (d) the nucleotide sequence of the antisense strand comprises the nucleotide sequence of the antisense strand of dsRNA agent 480; and the nucleotide sequence of the sense strand comprises of the sense strand of dsRNA agent 480; or (c) the nucleotide sequence of the antisense strand comprises the nucleotide sequence of the antisense strand of dsRNA agent 409; and the nucleotide sequence of the sense strand comprises of the sense strand of dsRNA agent 409.

In one aspect, provided herein are dsRNA agents for inhibiting expression of CIDEB (e.g., hCIDEB), wherein the dsRNA agent comprises a sense strand and an antisense strand forming a double stranded region, and wherein the sense strand comprises at least 15 (e.g., 16, 17, 18, 19, 20, 21) contiguous nucleotides of and differing by no more than 5 (e.g., 0, 1, 2, 3, 4, or 5 (e.g., 3 (e.g., 0, 1, 2, or 3))) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1230-1280, 1240-1290, 1250-1300, 1240-1270, 1240-1280, 1235-1270, 1245-1265, 1247-1267, 1252-1272, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 1920-1970, 1930-1970, 1930-1965, 1940-1970, 1940-1965, 1937-1957, 1942-1962, 1938-1958, 1943-1963, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; wherein the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5 (e.g., 2 or 3)) nucleotides from the nucleotide sequence of the corresponding complementary nucleotide sequence of SEQ ID NO: 2.

In some embodiments, the nucleotide sequence of the antisense strand differs by no more than 5 (e.g., 0, 1, 2, 3, 4, or 5 (e.g., 3 (e.g., 0, 1, 2, or 3))) nucleotides from the nucleotide sequence of any one of the antisense strands set forth in Table 2 or Table 3 or set forth in any one of SEQ ID NOS: 187-369, 602-657, 893-1131, or 1188-1191.

In some embodiments, the sense strand differs by no more than 3 (e.g., 0, 1, 2, or 3) nucleotides from the nucleotide sequence of any one of the sense strands set forth in Table 2 or Table 3 or set forth in any one of SEQ ID NOS: 4-186, 553-601, 658-892, or 1192.

In some embodiments, the sense strand comprises at least 15 (e.g., 16, 17, 18, 19, 20, 21) contiguous nucleotides of and differing by no more than 3 (e.g., 0, 1, 2, or 3) nucleotides from nucleotides 1220-1270, 1230-1280, 1240-1290, 1250-1300, 1240-1270, 1240-1280, 1235-1270, 1245-1265, 1247-1267, 1252-1272, 1915-1966, 1920-1970, 1930-1970, 1930-1965, 1940-1970, 1940-1965, 1937-1957, 1942-1962, 1938-1958, 1943-1963, of SEQ ID NO: 1.

In some embodiments, the sense strand comprises at least 15 (e.g., 16, 17, 18, 19, 20, 21) contiguous nucleotides of and differing by no more than 3 (e.g., 0, 1, 2, or 3) nucleotides from nucleotides 1220-1270, 1230-1280, 1240-1290, 1250-1300, 1240-1270, 1240-1280, 1235-1270, 1245-1265, 1247-1267, 1252-1272, and wherein the nucleotide sequence of the antisense strand comprises the nucleotide sequence set forth in any one of SEQ ID NOS: 315 or 321.

In some embodiments, the sense strand comprises at least 15 (e.g., 16, 17, 18, 19, 20, 21) contiguous nucleotides of and differing by no more than 3 (e.g., 0, 1, 2, or 3) nucleotides from nucleotides 1915-1966, 1920-1970, 1930-1970, 1930-1965, 1940-1970, 1940-1965, 1937-1957, 1942-1962, 1938-1958, 1943-1963, and wherein the nucleotide sequence of the antisense strand comprises the nucleotide sequence set forth in any one of SEQ ID NOS: 355 or 356.

In one aspect, provided herein are dsRNA agent for inhibiting expression of CIDEB (e.g., hCIDEB), wherein the dsRNA agent comprises a sense strand and an antisense strand forming a double stranded region, wherein the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of any one of the antisense strands set forth in Table 2 or Table 3 or set forth in any one of SEQ ID NOS: 187-369, 602-657, 893-1131, or 1188-1191.

In some embodiments, the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the corresponding sense strand set forth in Table 2 or Table 3 or the corresponding sense strand set forth in one of SEQ ID NOS: 4-186, 553-601, 658-892, or 1192.

In some embodiments, the nucleotide sequence of the antisense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of any one of the antisense strands set forth in Table 2 or Table 3 or set forth in any one of SEQ ID NOS: 187-369, 602-657, 893-1131, or 1188-1191; and wherein the nucleotide sequence of the sense strand comprises at least 15 (e.g., 16, 17, 18, 19, 20, 21) contiguous nucleotides of and differing by no more than 3 (e.g., 0, 1, 2, or 3) nucleotides from the nucleotide sequence of the corresponding sense strand set forth in Table 2 or Table 3 or the corresponding sense strand set forth in one of SEQ ID NOS: 4-186, 553-601, 658-892, or 1192.

In some embodiments, the nucleotide sequence of the antisense strand comprises at least 21 (e.g., 21, 22, or 23) contiguous nucleotides of the nucleotide sequence of any one of the antisense strands set forth in Table 2 or Table 3 or set forth in any one of SEQ ID NOS: 187-369, 602-657, 893-1131, or 1188-1191; and wherein the nucleotide sequence of the sense strand comprises at least 18 (e.g., 18, 19, 20, 21) contiguous nucleotides of the nucleotide sequence of the corresponding sense strand set forth in Table 2 or Table 3 or the corresponding sense strand set forth in one of SEQ ID NOS: 4-186, 553-601, 658-892, or 1192.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 355; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 172; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 315; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 132; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 356; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 173; or (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 321; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 138.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 1191; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1192; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 1188; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 786; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 1190; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 786; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 1062; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 827; or (e) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 1189; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 792.

In one aspect, provided herein are dsRNA agent for inhibiting expression of CIDEB (e.g., hCIDEB), wherein the dsRNA agent comprises a sense strand and an antisense strand forming a double stranded region, wherein the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of any one of the antisense strand of any one of dsRNA agents 129, 135, 169, 170, 409, 479, 480, 481, or 482.

In some embodiments, the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the corresponding sense strand of the dsRNA agent.

In some embodiments, the nucleotide sequence of the antisense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of any one of the antisense strand of any one of dsRNA agents 129, 135, 169, 170, 409, 479, 480, 481, or 482; and wherein the nucleotide sequence of the sense strand comprises at least 15 (e.g., 16, 17, 18, 19, 20, 21) contiguous nucleotides of and differing by no more than 3 (e.g., 0, 1, 2, or 3) nucleotides from the nucleotide sequence of the corresponding sense strand of the dsRNA agent.

In some embodiments, the nucleotide sequence of the antisense strand comprises at least 21 (e.g., 21, 22, or 23) contiguous nucleotides of the nucleotide sequence of any one of the antisense strands of any one of dsRNA agents 129, 135, 169, 170, 409, 479, 480, 481, or 482; and wherein the nucleotide sequence of the sense strand comprises at least 18 (e.g., 18, 19, 20, 21) contiguous nucleotides of the nucleotide sequence of the corresponding sense strand of the dsRNA agent.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 129; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 129; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 135; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 135; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 169; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 169; or (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 170; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 170.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 409; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 409; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 479; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 479; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 480; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 480; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 481; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 481; or (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 482; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 482.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand comprises the nucleotide sequence set forth in SEQ ID NO: 1191; and the nucleotide sequence of the sense strand comprises the nucleotide sequence set forth in SEQ ID NO: 1192; (b) the nucleotide sequence of the antisense strand comprises the nucleotide sequence set forth in SEQ ID NO: 1188; and the nucleotide sequence of the sense strand comprises the nucleotide sequence set forth in SEQ ID NO: 786; (c) the nucleotide sequence of the antisense strand comprises the nucleotide sequence set forth in SEQ ID NO: 1190; and the nucleotide sequence of the sense strand comprises the nucleotide sequence set forth in SEQ ID NO: 786; (d) the nucleotide sequence of the antisense strand comprises the nucleotide sequence set forth in SEQ ID NO: 1062; and the nucleotide sequence of the sense strand comprises the nucleotide sequence set forth in SEQ ID NO: 827; or (c) the nucleotide sequence of the antisense strand comprises the nucleotide sequence set forth in SEQ ID NO: 1189; and the nucleotide sequence of the sense strand comprises the nucleotide sequence set forth in SEQ ID NO: 792.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand differs by no more than 5 (e.g., 0, 1, 2, 3, 4, or 5) nucleotides from the nucleotide sequence set forth in SEQ ID NO: 1191; and the nucleotide sequence of the sense strand differs by no more than 5 (e.g., 0, 1, 2, 3, 4, or 5) nucleotides from the nucleotide sequence set forth in SEQ ID NO: 1192; (b) the nucleotide sequence of the antisense strand differs by no more than 5 (e.g., 0, 1, 2, 3, 4, or 5) nucleotides from the nucleotide sequence set forth in SEQ ID NO: 1188; and the nucleotide sequence of the sense strand differs by no more than 5 (e.g., 0, 1, 2, 3, 4, or 5) nucleotides from the nucleotide sequence set forth in SEQ ID NO: 786; (c) the nucleotide sequence of the antisense strand differs by no more than 5 (e.g., 0, 1, 2, 3, 4, or 5) nucleotides from the nucleotide sequence set forth in SEQ ID NO: 1190; and the nucleotide sequence of the sense strand differs by no more than 5 (e.g., 0, 1, 2, 3, 4, or 5) nucleotides from the nucleotide sequence set forth in SEQ ID NO: 786; (d) the nucleotide sequence of the antisense strand differs by no more than 5 (e.g., 0, 1, 2, 3, 4, or 5) nucleotides from the nucleotide sequence set forth in SEQ ID NO: 1062; and the nucleotide sequence of the sense strand differs by no more than 5 (e.g., 0, 1, 2, 3, 4, or 5) nucleotides from the nucleotide sequence set forth in SEQ ID NO: 827; or (e) the nucleotide sequence of the antisense strand differs by no more than 5 (e.g., 0, 1, 2, 3, 4, or 5) nucleotides from the nucleotide sequence set forth in SEQ ID NO: 1189; and the nucleotide sequence of the sense strand differs by no more than 5 (e.g., 0, 1, 2, 3, 4, or 5) nucleotides from the nucleotide sequence set forth in SEQ ID NO: 792.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand differs by no more than 3 (e.g., 0, 1, 2, or 3) nucleotides from the nucleotide sequence set forth in SEQ ID NO: 1191; and the nucleotide sequence of the sense strand differs by no more than 3 (e.g., 0, 1, 2, or 3) nucleotides from the nucleotide sequence set forth in SEQ ID NO: 1192; (b) the nucleotide sequence of the antisense strand differs by no more than 3 (e.g., 0, 1, 2, or 3) nucleotides from the nucleotide sequence set forth in SEQ ID NO: 1188; and the nucleotide sequence of the sense strand differs by no more than 3 (e.g., 0, 1, 2, or 3) nucleotides from the nucleotide sequence set forth in SEQ ID NO: 786; (c) the nucleotide sequence of the antisense strand differs by no more than 3 (e.g., 0, 1, 2, or 3) nucleotides from the nucleotide sequence set forth in SEQ ID NO: 1190; and the nucleotide sequence of the sense strand differs by no more than 3 (e.g., 0, 1, 2, or 3) nucleotides from the nucleotide sequence set forth in SEQ ID NO: 786; (d) the nucleotide sequence of the antisense strand differs by no more than 3 (e.g., 0, 1, 2, or 3) nucleotides from the nucleotide sequence set forth in SEQ ID NO: 1062; and the nucleotide sequence of the sense strand differs by no more than 3 (e.g., 0, 1, 2, or 3) nucleotides from the nucleotide sequence set forth in SEQ ID NO: 827; or (e) the nucleotide sequence of the antisense strand differs by no more than 3 (e.g., 0, 1, 2, or 3) nucleotides from the nucleotide sequence set forth in SEQ ID NO: 1189; and the nucleotide sequence of the sense strand differs by no more than 3 (e.g., 0, 1, 2, or 3) nucleotides from the nucleotide sequence set forth in SEQ ID NO: 792.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand differs by no more than 5 (e.g., 0, 1, 2, 3, 4, or 5) nucleotides from the nucleotide sequence set forth in SEQ ID NO: 355; and the nucleotide sequence of the sense strand differs by no more than 5 (e.g., 0, 1, 2, 3, 4, or 5) nucleotides from the nucleotide sequence set forth in SEQ ID NO: 172; (b) the nucleotide sequence of the antisense strand differs by no more than 5 (e.g., 0, 1, 2, 3, 4, or 5) nucleotides from the nucleotide sequence set forth in SEQ ID NO: 315; and the nucleotide sequence of the sense strand differs by no more than 5 (e.g., 0, 1, 2, 3, 4, or 5) nucleotides from the nucleotide sequence set forth in SEQ ID NO: 132; (c) the nucleotide sequence of the antisense strand differs by no more than 5 (e.g., 0, 1, 2, 3, 4, or 5) nucleotides from the nucleotide sequence set forth in SEQ ID NO: 356; and the nucleotide sequence of the sense strand differs by no more than 5 (e.g., 0, 1, 2, 3, 4, or 5) nucleotides from the nucleotide sequence set forth in SEQ ID NO: 173; or (d) the nucleotide sequence of the antisense strand differs by no more than 5 (e.g., 0, 1, 2, 3, 4, or 5) nucleotides from the nucleotide sequence set forth in SEQ ID NO: 321; and the nucleotide sequence of the sense strand differs by no more than 5 (e.g., 0, 1, 2, 3, 4, or 5) nucleotides from the nucleotide sequence set forth in SEQ ID NO: 138.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand differs by no more than 3 (e.g., 0, 1, 2, or 3) nucleotides from the nucleotide sequence set forth in SEQ ID NO: 355; and the nucleotide sequence of the sense strand differs by no more than 3 (e.g., 0, 1, 2, or 3) nucleotides from the nucleotide sequence set forth in SEQ ID NO: 172; (b) the nucleotide sequence of the antisense strand differs by no more than 3 (e.g., 0, 1, 2, or 3) nucleotides from the nucleotide sequence set forth in SEQ ID NO: 315; and the nucleotide sequence of the sense strand differs by no more than 3 (e.g., 0, 1, 2, or 3) nucleotides from the nucleotide sequence set forth in SEQ ID NO: 132; (c) the nucleotide sequence of the antisense strand differs by no more than 3 (e.g., 0, 1, 2, or 3) nucleotides from the nucleotide sequence set forth in SEQ ID NO: 356; and the nucleotide sequence of the sense strand differs by no more than 3 (e.g., 0, 1, 2, or 3) nucleotides from the nucleotide sequence set forth in SEQ ID NO: 173; or (d) the nucleotide sequence of the antisense strand differs by no more than 3 (e.g., 0, 1, 2, or 3) nucleotides from the nucleotide sequence set forth in SEQ ID NO: 321; and the nucleotide sequence of the sense strand differs by no more than 3 (e.g., 0, 1, 2, or 3) nucleotides from the nucleotide sequence set forth in SEQ ID NO: 138.

In one aspect, provided herein are dsRNA agents for inhibiting expression of CIDEB (e.g., hCIDEB), wherein the dsRNA agent comprises a sense strand and an antisense strand forming a double stranded region, and wherein (a) the nucleotide sequence of the antisense strand comprises the nucleotide sequence set forth in SEQ ID NO: 355; and the nucleotide sequence of the sense strand comprises the nucleotide sequence set forth in SEQ ID NO: 172; (b) the nucleotide sequence of the antisense strand comprises the nucleotide sequence set forth in SEQ ID NO: 315; and the nucleotide sequence of the sense strand comprises the nucleotide sequence set forth in SEQ ID NO: 132; (c) the nucleotide sequence of the antisense strand comprises the nucleotide sequence set forth in SEQ ID NO: 356; and the nucleotide sequence of the sense strand comprises the nucleotide sequence set forth in SEQ ID NO: 173; or (d) the nucleotide sequence of the antisense strand comprises the nucleotide sequence set forth in SEQ ID NO: 321; and the nucleotide sequence of the sense strand comprises the nucleotide sequence set forth in SEQ ID NO: 138.

For the sake of clarity, it is noted that the following embodiments are applicable to any of the foregoing aspects as if individually recited directly following each aspect.

In some embodiments, the sense strand comprises at least one modified nucleotide and/or the antisense strand comprises at least one modified nucleotide. In some embodiments, at least 25%, 30%, 35%, 40%, 45%, 50%, 55%, 60%, 65%, 70%, 75%, 80%, 85%, 90%, 95%, or 100% of the nucleotides of the sense strand and/or antisense strand are modified. In some embodiments, all of the nucleotides in the sense strand and/or antisense strand are modified. In some embodiments, all of the nucleotides in the sense strand and the antisense strand are modified.

In some embodiments, at least one of the modified nucleotides comprises a modified sugar (e.g., ribose moiety).

In some embodiments, (a) the sense strand comprises at least one 2′-O-methyl and/or the antisense strand comprises at least one 2′-O-methyl and/or (b) the sense strand comprises at least one 2′-deoxy-2′-fluoro and/or the antisense strand comprises at least one 2′-deoxy-2′-fluoro.

In some embodiments, (a) the sense strand comprises at least one 2′-O-methyl and at least one 2′-deoxy-2′-fluoro; and the antisense strand comprises at least one 2′-O-methyl and at least one 2′-deoxy-2′-fluoro.

In some embodiments, at least one of the modified nucleotides comprises a modified nucleobase.

In some embodiments, the sense strand comprises at least one vinyl-phosphonate and/or the antisense strand comprises at least one vinyl-phosphonate. In some embodiments, the antisense strand comprises a 5′ vinyl-phosphonate. In some embodiments, the sense strand comprises at least one vinyl-phosphonate-2′-O-methyl (e.g., vinyl-phosphonate-2′-O-methyluridine) and/or the antisense strand comprises at least one vinyl-phosphonate-2′-O-methyl (e.g., vinyl-phosphonate-2′-O-methyluridine) (e.g., wherein the antisense strand comprises at least one vinyl-phosphonate-2′-O-methyl (e.g., vinyl-phosphonate-2′-O-methyluridine). In some embodiments, the antisense strand comprises a 5′ vinyl-phosphonate-2′-O-methyl (e.g., vinyl-phosphonate-2′-O-methyluridine).

In some embodiments, the sense strand comprises at least one modified internucleoside linkage and/or the antisense strand comprises at least one modified internucleoside linkage.

In some embodiments, the sense strand comprises at least one phosphorothioate and/or the antisense strand comprises at least one phosphorothioate.

In some embodiments, the sense strand comprises at least one phosphorothioate and the antisense strand comprises at least one phosphorothioate.

In some embodiments, each of the antisense strand and the sense strand are not more than 30, 29, 28, 27, 26, 25, 24, 23, 22, 21, 20, 19, 18, 17, 16, or 15 nucleotides in length. In some embodiments, the antisense strand comprises from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides; and/or the sense strand comprises from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides. In some embodiments, antisense strand comprises from about 19-23 nucleotides; and/or the sense strand comprises from about 19-23 nucleotides. In some embodiments, antisense strand comprises or consists of about 23 nucleotides; and/or the sense strand comprises or consists of about 21 nucleotides.

In some embodiments, the sense strand and/or the antisense strand comprises a 3′ and/or 5′ overhang of 1, 2, or 3 nucleotides. In some embodiments, the antisense strand comprises a 3′ overhang of 1, 2, or 3 nucleotides (e.g., 2 nucleotides). In some embodiments, the antisense strand comprises a 3′ overhang of 2 nucleotides.

In some embodiments, the double stranded region is from about 19-30, 19-29, 19-28, 19-27, 19-26, 19-25, 19-24, 19-23, 19-22, 19-20, 19-21, 23-30, 23-29, 23-28, 23-27, 23-26, 23-25, 23-24, 21-30, 21-29, 21-28, 21-27, 21-26, 21-25, 21-24, 21-23, or 21-22 nucleotide pairs in length. In some embodiments, the double stranded region is from about 19-23 or 19-21 nucleotide pairs in length. In some embodiments, the double stranded region is about 21 nucleotide pairs in length.

In some embodiments, the sense strand and the antisense strand are part of a single nucleic acid molecule (e.g., wherein a hairpin loop is between the sense strand and the antisense strand of the single nucleic acid molecule.

In some embodiments, the sense strand and the antisense strand are separate nucleic acid molecules (i.e., connected only through the double stranded region).

In some embodiments, the nucleotide sequence of the antisense strand consists of 23 nucleotides; the nucleotide sequence of the sense strand consists of 21 nucleotides; the double stranded region is 21 nucleotide pairs in length; the antisense strand comprises a 3′ overhang of 2 nucleotides; the antisense strand comprises a 5′ vinyl-phosphonate-2′-O-methyl (e.g., vinyl-phosphonate-2′-O-methyluridine), at least one 2′-O-methyl, at least one 2′-deoxy-2′-fluoro and at least one phosphorothioate; and the sense strand comprises at least one 2′-O-methyl, at least one 2′-deoxy-2′-fluoro and at least one phosphorothioate.

In one aspect, provided herein are conjugates comprising a dsRNA agent described herein and a heterologous moiety.

In some embodiments, the heterologous moiety is a peptide, protein, carbohydrate, lipid, polymer, or small molecule.

In some embodiments, the heterologous moiety is carbohydrate. In some embodiments, the heterologous moiety comprises one or more GalNac. In some embodiments, the GalNac is triantennary GalNac.

In some embodiments, the heterologous moiety is a targeting moiety. In some embodiments, the targeting moiety specifically binds to a moiety expressed by hepatocytes (e.g., on the surface of the hepatocytes). In some embodiments, the targeting moiety comprises GalNac. In some embodiments, the GalNac is triantennary GalNac. In some embodiments, the targeting moiety directs the agent to hepatocytes through specific binding to the asialoglycoprotein receptor (e.g., expressed on the surface of hepatocytes).

In some embodiments, the heterologous moiety is attached to the dsRNA agent via a linker.

In some embodiments, the linker comprises triethylene glycol (TEG). In some embodiments, the heterologous moiety comprises GalNac and the linker is TEG. In some embodiments, the heterologous moiety comprises triantennary GalNac and the linker is TEG.

In some embodiments, the heterologous moiety and linker comprises Formula XXXIX below:

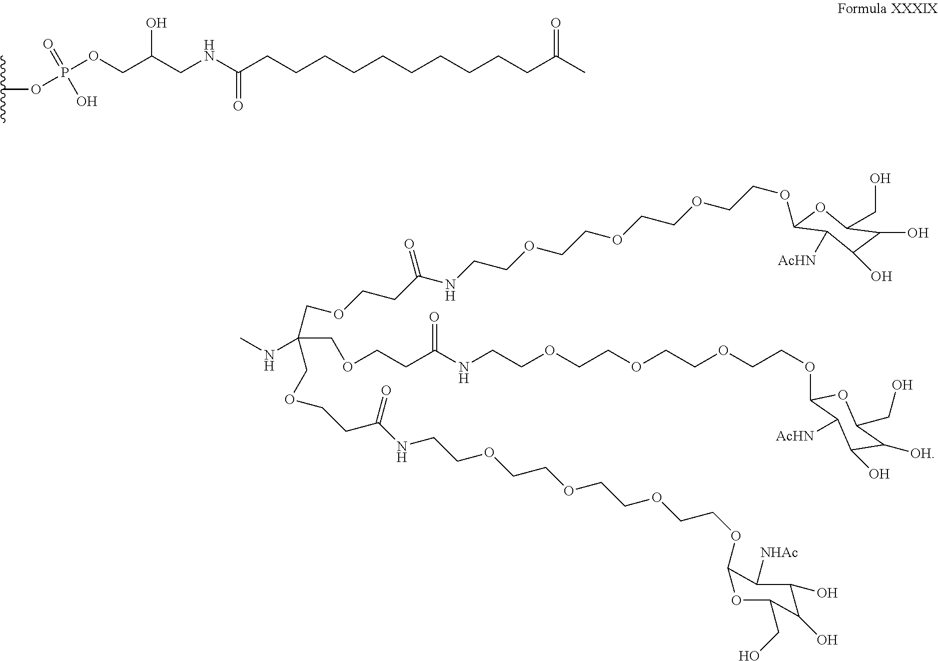

In some embodiments, the heterologous moiety attached to the 3′ end of the sense and/or antisense strand and/or the 5′ end of the sense and/or antisense strand, and/or at an internal site of the sense and/or antisense strand. In some embodiments, the heterologous moiety attached to the 3′ end of the sense strand.

In some embodiments, the conjugate is set forth in Table 11.

In some embodiments, (a) the nucleotide sequence of the antisense strand differs by no more than 5 (e.g., 0, 1, 2, 3, 4, or 5) nucleotides from the nucleotide sequence set forth in SEQ ID NO: 1188; and the nucleotide sequence of the sense strand differs by no more than 5 (e.g., 0, 1, 2, 3, 4, or 5) nucleotides from the nucleotide sequence set forth in SEQ ID NO: 1193; (b) the nucleotide sequence of the antisense strand differs by no more than 5 (e.g., 0, 1, 2, 3, 4, or 5) nucleotides from the nucleotide sequence set forth in SEQ ID NO: 1190; and the nucleotide sequence of the sense strand differs by no more than 5 (e.g., 0, 1, 2, 3, 4, or 5) nucleotides from the nucleotide sequence set forth in SEQ ID NO: 1193; (c) the nucleotide sequence of the antisense strand differs by no more than 5 (e.g., 0, 1, 2, 3, 4, or 5) nucleotides from the nucleotide sequence set forth in SEQ ID NO: 1191; and the nucleotide sequence of the sense strand differs by no more than 5 (e.g., 0, 1, 2, 3, 4, or 5) nucleotides from the nucleotide sequence set forth in SEQ ID NO: 1194; (d) the nucleotide sequence of the antisense strand differs by no more than 5 (e.g., 0, 1, 2, 3, 4, or 5) nucleotides from the nucleotide sequence set forth in SEQ ID NO: 1189; and the nucleotide sequence of the sense strand differs by no more than 5 (e.g., 0, 1, 2, 3, 4, or 5) nucleotides from the nucleotide sequence set forth in SEQ ID NO: 1195; or (e) the nucleotide sequence of the antisense strand differs by no more than 5 (e.g., 0, 1, 2, 3, 4, or 5) nucleotides from the nucleotide sequence set forth in SEQ ID NO: 1062; and the nucleotide sequence of the sense strand differs by no more than 5 (e.g., 0, 1, 2, 3, 4, or 5) nucleotides from the nucleotide sequence set forth in SEQ ID NO: 1196.

In some embodiments, (a) the nucleotide sequence of the antisense strand differs by no more than 3 (e.g., 0, 1, 2, or 3) nucleotides from the nucleotide sequence set forth in SEQ ID NO: 1188; and the nucleotide sequence of the sense strand differs by no more than 3 (e.g., 0, 1, 2, or 3) nucleotides from the nucleotide sequence set forth in SEQ ID NO: 1193; (b) the nucleotide sequence of the antisense strand differs by no more than 3 (e.g., 0, 1, 2, or 3) nucleotides from the nucleotide sequence set forth in SEQ ID NO: 1190; and the nucleotide sequence of the sense strand differs by no more than 3 (e.g., 0, 1, 2, or 3) nucleotides from the nucleotide sequence set forth in SEQ ID NO: 1193; (c) the nucleotide sequence of the antisense strand differs by no more than 3 (e.g., 0, 1, 2, or 3) nucleotides from the nucleotide sequence set forth in SEQ ID NO: 1191; and the nucleotide sequence of the sense strand differs by no more than 3 (e.g., 0, 1, 2, or 3) nucleotides from the nucleotide sequence set forth in SEQ ID NO: 1194; (d) the nucleotide sequence of the antisense strand differs by no more than 3 (e.g., 0, 1, 2, or 3) nucleotides from the nucleotide sequence set forth in SEQ ID NO: 1189; and the nucleotide sequence of the sense strand differs by no more than 3 (e.g., 0, 1, 2, or 3) nucleotides from the nucleotide sequence set forth in SEQ ID NO: 1195; or (c) the nucleotide sequence of the antisense strand differs by no more than 3 (e.g., 0, 1, 2, or 3) nucleotides from the nucleotide sequence set forth in SEQ ID NO: 1062; and the nucleotide sequence of the sense strand differs by no more than 3 (e.g., 0, 1, 2, or 3) nucleotides from the nucleotide sequence set forth in SEQ ID NO: 1196.

In some embodiments, (a) the nucleotide sequence of the antisense strand comprises the nucleotide sequence set forth in SEQ ID NO: 1188; and the nucleotide sequence of the sense strand comprises the nucleotide sequence set forth in SEQ ID NO: 1193; (b) the nucleotide sequence of the antisense strand comprises the nucleotide sequence set forth in SEQ ID NO: 1190; and the nucleotide sequence of the sense strand comprises the nucleotide sequence set forth in SEQ ID NO: 1193; (c) the nucleotide sequence of the antisense strand comprises the nucleotide sequence set forth in SEQ ID NO: 1191; and the nucleotide sequence of the sense strand comprises the nucleotide sequence set forth in SEQ ID NO: 1194; (d) the nucleotide sequence of the antisense strand comprises the nucleotide sequence set forth in SEQ ID NO: 1189; and the nucleotide sequence of the sense strand comprises the nucleotide sequence set forth in SEQ ID NO: 1195; or (c) the nucleotide sequence of the antisense strand comprises the nucleotide sequence set forth in SEQ ID NO: 1062; and the nucleotide sequence of the sense strand comprises the nucleotide sequence set forth in SEQ ID NO: 1196.

In some embodiments, the conjugate comprises any one of dsRNA agents 483-487.

In some embodiments, (a) the nucleotide sequence of the antisense strand differs by no more than 5 (e.g., 0, 1, 2, 3, 4, or 5) nucleotides from the nucleotide sequence of the antisense strand of dsRNA agent 483; and the nucleotide sequence of the sense strand differs by no more than 5 (e.g., 0, 1, 2, 3, 4, or 5) nucleotides from the nucleotide sequence of dsRNA agent 483; (b) the nucleotide sequence of the antisense strand differs by no more than 5 (e.g., 0, 1, 2, 3, 4, or 5) nucleotides from the nucleotide sequence of the antisense strand of dsRNA agent 484; and the nucleotide sequence of the sense strand differs by no more than 5 (e.g., 0, 1, 2, 3, 4, or 5) nucleotides from the nucleotide sequence of dsRNA agent 484; (c) the nucleotide sequence of the antisense strand differs by no more than 5 (e.g., 0, 1, 2, 3, 4, or 5) nucleotides from the nucleotide sequence of the antisense strand of dsRNA agent 485; and the nucleotide sequence of the sense strand differs by no more than 5 (e.g., 0, 1, 2, 3, 4, or 5) nucleotides from the nucleotide sequence of dsRNA agent 485; (d) the nucleotide sequence of the antisense strand differs by no more than 5 (e.g., 0, 1, 2, 3, 4, or 5) nucleotides from the nucleotide sequence of the antisense strand of dsRNA agent 486; and the nucleotide sequence of the sense strand differs by no more than 5 (e.g., 0, 1, 2, 3, 4, or 5) nucleotides from the nucleotide sequence of dsRNA agent 486; or (e) the nucleotide sequence of the antisense strand differs by no more than 5 (e.g., 0, 1, 2, 3, 4, or 5) nucleotides from the nucleotide sequence of the antisense strand of dsRNA agent 487; and the nucleotide sequence of the sense strand differs by no more than 5 (e.g., 0, 1, 2, 3, 4, or 5) nucleotides from the nucleotide sequence of dsRNA agent 487.

In some embodiments, (a) the nucleotide sequence of the antisense strand differs by no more than 3 (e.g., 0, 1, 2, or 3) nucleotides from the nucleotide sequence of the antisense strand of dsRNA agent 483; and the nucleotide sequence of the sense strand differs by no more than 3 (e.g., 0, 1, 2, or 3) nucleotides from the nucleotide sequence of dsRNA agent 483; (b) the nucleotide sequence of the antisense strand differs by no more than 3 (e.g., 0, 1, 2, or 3) nucleotides from the nucleotide sequence of the antisense strand of dsRNA agent 484; and the nucleotide sequence of the sense strand differs by no more than 3 (e.g., 0, 1, 2, or 3) nucleotides from the nucleotide sequence of dsRNA agent 484; (c) the nucleotide sequence of the antisense strand differs by no more than 3 (e.g., 0, 1, 2, or 3) nucleotides from the nucleotide sequence of the antisense strand of dsRNA agent 485; and the nucleotide sequence of the sense strand differs by no more than 3 (e.g., 0, 1, 2, or 3) nucleotides from the nucleotide sequence of dsRNA agent 485; (d) the nucleotide sequence of the antisense strand differs by no more than 3 (e.g., 0, 1, 2, or 3) nucleotides from the nucleotide sequence of the antisense strand of dsRNA agent 486; and the nucleotide sequence of the sense strand differs by no more than 3 (e.g., 0, 1, 2, or 3) nucleotides from the nucleotide sequence of dsRNA agent 486; or (e) the nucleotide sequence of the antisense strand differs by no more than 3 (e.g., 0, 1, 2, or 3) nucleotides from the nucleotide sequence of the antisense strand of dsRNA agent 487; and the nucleotide sequence of the sense strand differs by no more than 3 (e.g., 0, 1, 2, or 3) nucleotides from the nucleotide sequence of dsRNA agent 487.

In some embodiments, (a) the nucleotide sequence of the antisense strand comprises the nucleotide sequence of the antisense strand of dsRNA agent 483; and the nucleotide sequence of the sense strand comprises of the sense strand of dsRNA agent 483; (b) the nucleotide sequence of the antisense strand comprises the nucleotide sequence of the antisense strand of dsRNA agent 484; and the nucleotide sequence of the sense strand comprises of the sense strand of dsRNA agent 484; (c) the nucleotide sequence of the antisense strand comprises the nucleotide sequence of the antisense strand of dsRNA agent 485; and the nucleotide sequence of the sense strand comprises of the sense strand of dsRNA agent 485; (d) the nucleotide sequence of the antisense strand comprises the nucleotide sequence of the antisense strand of dsRNA agent 486; and the nucleotide sequence of the sense strand comprises of the sense strand of dsRNA agent 486; or (c) the nucleotide sequence of the antisense strand comprises the nucleotide sequence of the antisense strand of dsRNA agent 487; and the nucleotide sequence of the sense strand comprises of the sense strand of dsRNA agent 487.

In one aspect, provided herein are vectors (e.g., a viral vector, a non-viral vector) encoding an antisense strand described herein, a sense strand described herein, or both an antisense described herein and a sense strand described herein.

In one aspect, provided herein are carriers comprising a dsRNA agent described herein, a conjugate described herein, or a vector described herein.

In some embodiments, the carrier comprises a nanoparticle, a polymer, a lipid-based delivery system, as dendrimer, a cationic delivery system, or a hydrogel. In some embodiments the lipid-based delivery system is a lipid nanoparticle (LNP), liposome, lipoplex, nanoliposome, an exosome, or a micelle.

In one aspect, provided herein are cells (or population of cells) comprising a dsRNA agent described herein, a conjugate described herein, a vector described herein, or a carrier described herein.

In one aspect, provided herein are pharmaceutical compositions comprising a dsRNA agent described herein, a conjugate described herein, a vector described herein, a carrier described herein, or a cell (or population of cells) described herein, and a pharmaceutically acceptable excipient.

In one aspect, provided herein are kits comprising a dsRNA agent described herein, a conjugate described herein, a vector described herein, a carrier described herein, a cell (or population of cells) described herein, or a pharmaceutical composition described herein.

In one aspect, provided herein are methods of delivering a dsRNA, conjugate, vector, carrier, or pharmaceutical composition to a cell, the method comprising introducing into a cell a dsRNA agent described herein, a conjugate described herein, a vector described herein, a carrier described herein, a cell (or population of cells) described herein, or a pharmaceutical composition described herein, to thereby deliver the dsRNA, conjugate, vector, carrier, or pharmaceutical composition into the cell. In some embodiments, the cell is in vitro, ex vivo, or in vivo. In some embodiments the cell is a subject (e.g., a human subject).

In one aspect, provided herein are methods of delivering a dsRNA, conjugate, vector, carrier, cell, or pharmaceutical composition to a subject, the method comprising administering to the subject a dsRNA agent described herein, a conjugate described herein, a vector described herein, a carrier described herein, a cell (or population of cells) described herein, or a pharmaceutical composition described herein, to thereby deliver the dsRNA, conjugate, vector, carrier, cell, or pharmaceutical composition to the subject.

In one aspect, provided herein are methods of reducing or inhibiting expression of CIDEB (e.g., hCIDEB) in a cell, the method comprising delivering into the cell a dsRNA agent described herein, a conjugate described herein, a vector described herein, a carrier described herein, a cell (or population of cells) described herein, or a pharmaceutical composition described herein, to thereby reduce or inhibit expression of CIDEB (e.g., hCIDEB) in the cell.

In one aspect, provided herein are methods of reducing or inhibiting expression of CIDEB (e.g., hCIDEB) in a cell in a subject, the method comprising administering to the subject a dsRNA agent described herein, a conjugate described herein, a vector described herein, a carrier described herein, a cell (or population of cells) described herein, or a pharmaceutical composition described herein, to thereby reduce or inhibit expression of CIDEB (e.g., hCIDEB) in the cell in the subject.

In one aspect, provided herein are methods of treating, ameliorating, or preventing a CIDEB (e.g., hCIDEB) associated disease in a subject, the method comprising administering to the subject a dsRNA agent described herein, a conjugate described herein, a vector described herein, a carrier described herein, a cell (or population of cells) described herein, or a pharmaceutical composition described herein, to thereby treat, ameliorate, or prevent the CIDEB (e.g., hCIDEB) associated disease in the subject.

In some embodiments, the treating, ameliorating, or preventing of the CIDEB (e.g., hCIDEB) associated disease is mediated (at least in part) through the by reducing or inhibiting expression of CIDEB (e.g., hCIDEB) in the subject (e.g., a population of cells within the subject).

In some embodiments, the CIDEB (e.g., hCIDEB) associated disease is fatty liver disease, steatotic liver disease, liver inflammation, NASH, NAFLD, MASLD, MASH, MAFLD, MetALD, SLD, or cryptogenic SLD.

In some embodiments, the CIDEB (e.g., hCIDEB) associated disease is liver fibrosis, cirrhosis, liver failure, jaundice, AFLD, obesity-induced metabolic syndrome, insulin insensitivity, or type-2 diabetes.

In one aspect, provided herein are methods of treating, ameliorate, or preventing a liver disease in a subject, the method comprising administering to the subject a dsRNA agent described herein, a conjugate described herein, a vector described herein, a carrier described herein, a cell (or population of cells) described herein, or a pharmaceutical composition described herein, to thereby treat, ameliorate, or prevent the liver disease in the subject.

In some embodiments, the treating, ameliorating, or preventing of the liver disease is mediated (at least in part) through the by reducing or inhibiting expression of CIDEB (e.g., hCIDEB) in the subject (e.g., a population of cells within the subject).

In some embodiments, the liver disease is fatty liver disease, steatotic liver disease, liver inflammation, NASH, NAFLD, MASLD, MASH, MAFLD, MetALD, SLD, or cryptogenic SLD. In some embodiments, the liver disease is liver fibrosis, cirrhosis, liver failure, jaundice, AFLD, obesity-induced metabolic syndrome, insulin insensitivity, or type-2 diabetes.

In one aspect, provided herein are methods of diagnosing a CIDEB (e.g., hCIDEB) associated disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the one or more somatic mutation indicates that the subject has a CIDEB (e.g., hCIDEB) associated disease.

In some embodiments, the CIDEB (e.g., hCIDEB) associated disease is a liver disease. In some embodiments, the CIDEB (e.g., hCIDEB) associated disease is fatty liver disease, steatotic liver disease, liver inflammation, NASH, NAFLD, MASLD, MASH, MAFLD, MetALD, SLD, or cryptogenic SLD. In some embodiments, the CIDEB (e.g., hCIDEB) associated disease is liver fibrosis, cirrhosis, liver failure, jaundice, AFLD, obesity-induced metabolic syndrome, insulin insensitivity, or type-2 diabetes.

In one aspect, provided herein are methods of diagnosing a liver disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the one or more somatic mutation indicates that the subject has a liver disease.

In some embodiments, the liver disease is fatty liver disease, steatotic liver disease, liver inflammation, NASH, NAFLD, MASLD, MASH, MAFLD, MetALD, SLD, or cryptogenic SLD. In some embodiments, the liver disease is liver fibrosis, cirrhosis, liver failure, jaundice, AFLD, obesity-induced metabolic syndrome, insulin insensitivity, or type-2 diabetes.

In some embodiments, (a) comprises isolating and purifying DNA, or having DNA isolated and purified, from a sample obtained from the subject; and (b) comprises detecting, or having detected, the presence or absence of the one or more somatic CIDEB mutation in the DNA.

In some embodiments, the method further comprises administering to the subject an inhibitory nucleic acid molecule that inhibits expression of CIDEB if a CIDEB somatic mutation is detected in the DNA, RNA, or protein. In some embodiments, the inhibitory nucleic acid molecule comprises a dsRNA agent described herein, a conjugate described herein, a vector described herein, a carrier described herein, a cell (or population of cells) described herein, or a pharmaceutical composition described herein.

In one aspect, provided herein are methods of selecting a subject for administration of an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the subject is selected for administration of the inhibitory nucleic acid molecule if the one or more somatic mutation is present.

In some embodiments, (a) comprises isolating and purifying DNA, or having DNA isolated and purified, from a sample obtained from the subject; and (b) comprises detecting, or having detected, the presence or absence of the one or more somatic CIDEB mutation in the DNA.

In some embodiments, the method further comprises administering to the subject the inhibitory nucleic acid molecule if the subject is selected.

In some embodiments, the inhibitory nucleic acid molecule comprises a dsRNA agent described herein, a conjugate described herein, a vector described herein, a carrier described herein, a cell (or population of cells) described herein, or a pharmaceutical composition described herein.

In one aspect, provided herein are methods of treating, ameliorating, or preventing a CIDEB (e.g., hCIDEB) associated disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, and (c) administering to the subject an inhibitory nucleic acid that inhibits expression of CIDEB if the one or more CIDEB somatic mutation is detected in the DNA, RNA, or protein.

In some embodiments, the CIDEB (e.g., hCIDEB) associated disease is a liver disease. In some embodiments, the liver disease is fatty liver disease, steatotic liver disease, liver inflammation, NASH, NAFLD, MASLD, MASH, MAFLD, MetALD, SLD, or cryptogenic SLD.

In some embodiments, the liver disease is liver fibrosis, cirrhosis, liver failure, jaundice, AFLD, obesity-induced metabolic syndrome, insulin insensitivity, or type-2 diabetes.

In some embodiments, the treating, ameliorating, or preventing of the CIDEB (e.g., hCIDEB) associated disease is mediated (at least in part) through the by reducing or inhibiting expression of CIDEB (e.g., hCIDEB) in the subject (e.g., a population of cells within the subject).

In one aspect, provided herein are methods of treating, ameliorating, or preventing a liver disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, and (c) administering to the subject an inhibitory nucleic acid that inhibits expression of CIDEB if the one or more CIDEB somatic mutation is detected in the DNA, RNA, or protein.

In some embodiments, the liver disease is fatty liver, liver inflammation, NASH, NAFLD, obesity-induced metabolic syndrome, insulin insensitivity, obesity, type-2 diabetes, AFLD, cirrhosis, MASLD, MASH, MetALD, SLD, or cryptogenic SLD. In some embodiments, the liver disease is liver fibrosis, cirrhosis, liver failure, jaundice, AFLD, obesity-induced metabolic syndrome, insulin insensitivity, or type-2 diabetes.

In some embodiments, (a) comprises isolating and purifying DNA, or having DNA isolated and purified, from a sample obtained from the subject; and (b) comprises detecting, or having detected, the presence or absence of the one or more somatic CIDEB mutation in the DNA.

In some embodiments, the inhibitory nucleic acid molecule comprises a dsRNA agent described herein, a conjugate described herein, a vector described herein, a carrier described herein, a cell (or population of cells) described herein, or a pharmaceutical composition described herein.

In some embodiments, the treating, ameliorating, or preventing of the liver disease is mediated (at least in part) through the by reducing or inhibiting expression of CIDEB (e.g., hCIDEB) in the subject (e.g., a population of cells within the subject).

In one aspect, provided herein are in vitro methods of screening a sample from a subject for one or more somatic CIDEB mutation, the method comprising (a) isolating and purifying DNA, RNA, or protein from a sample obtained from the subject; and (b) detecting the presence or absence of one or more somatic CIDEB mutation in the DNA, RNA, or protein.

In some embodiments, the sample is a blood, tissue, or cell sample. In some embodiments, the sample is biopsy. In some embodiments, the sample is a liver biopsy.

In some embodiments, at least one of the one or more somatic mutation is a loss of function mutation. In some embodiments, at least one of the one or more somatic mutation is a gain of function mutation.

In some embodiments, the subject is a human.

In one aspect, provided herein are dsRNA agents described herein, conjugates described herein, vectors described herein, carriers described herein, cells (or populations of cells) described herein, or pharmaceutical compositions described herein for use in a method of treating, ameliorating, or preventing a CIDEB associated disease (e.g., a CIDEB associated disease described herein) in a subject.

In one aspect, provided herein are dsRNA agents described herein, conjugates described herein, vectors described herein, carriers described herein, cells (or populations of cells) described herein, or pharmaceutical compositions described herein for use in a method of treating, ameliorating, or preventing a liver disease (e.g., a liver disease described herein) in a subject.

In one aspect, provided herein are uses of a dsRNA agent described herein, a conjugate described herein, a vector described herein, a carrier described herein, a cell (or population of cells) described herein, or a pharmaceutical composition described herein for the manufacture of a medicament for the treatment, amelioration, or prevention of a CIDEB associated disease (e.g., a CIDEB associated disease described herein) in a subject.

In one aspect, provided herein are uses of a dsRNA agent described herein, a conjugate described herein, a vector described herein, a carrier described herein, a cell (or population of cells) described herein, or a pharmaceutical composition described herein for the manufacture of a medicament for the treatment, amelioration, or prevention of a liver disease (e.g., a liver disease described herein) in a subject.

### 4. DETAILED DESCRIPTION

The inventors have further discovered, inter alia, RNAi agents that inhibit expression of CIDEB (e.g., hCIDEB). As such, the RNAi agents described herein are useful for the treatment of CIDEB mediated diseases such as liver diseases (e.g., fatty liver disease, liver inflammation, MASH). As such, the current disclosure provides RNAi agents (e.g., dsRNAi agents comprising a sense strand and an antisense strand) capable of inhibiting CIDEB expression (e.g., in a cell, in a cell in a subject); and their use in, inter alia, pharmaceutical compositions, and methods of treating diseases (e.g., liver diseases).

TABLE OF CONTENTS

| | |
|---|---|
| 4.1 | Definitions |
| 4.2 | RNAi Agents |
| 4.2.1 | Antisense Strand |
| 4.2.1.1 | Targeting Region |
| 4.2.1.2 | Overall Length |
| 4.2.1.3 | Exemplary Antisense Strands |
| 4.2.2 | Sense Strand |
| 4.2.2.1 | Antisense Strand Complementarity |
| 4.2.2.2 | Overall Length |
| 4.2.2.3 | Exemplary Sense Strands |
| 4.2.3 | dsRNA Agents |
| 4.2.3.1 | Single & Multiple Nucleic Acid Molecules |
| 4.2.3.2. | Length of Double Stranded Region |
| 4.2.3.3 | Nucleotide Overhangs & Blunt Ends |
| 4.2.3.4 | Exemplary Structural Combinations of |
| | Sense & Antisense Strands |
| 4.2.3.5 | Exemplary Antisense Strands & Sense Strands |
| 4.2.3.6 | Exemplary dsRNA Agents |
| 4.2.4 | Nature of Nucleotide Modifications |
| 4.2.4.1 | Modified Nucleosides |
| 4.2.4.1(i) | Sugar Modifications |
| 4.2.4.1(i)(a) | Non-Bicyclic Sugar Modifications |
| 4.2.4.1(i)(b) | Bicyclic Sugar Modifications |
| 4.2.4.1(ii) | Nucleobase Modifications |
| 4.2.4.2 | Internucleoside Linkage Modifications |
| 4.2.4.2(i) | Modified Phosphorous Containing |
| | Internucleoside Linkages |
| 4.2.4.2(ii) | Modified Non-Phosphorous Containing |
| | Internucleoside Linkages |
| 4.2.4.3 | Additional Exemplary Nucleotide Modifications |
| 4.2.5 | Extent of Modified Nucleotides |
| 4.3 | Conjugates |
| 4.3.1 | Heterologous Moieties |
| 4.3.1.1 | Targeting Moieties |
| 4.3.1.1(i) | Hepatocyte Targeting Moieties |
| 4.3.2 | Linkers |
| 4.3.2.1 | Cleavable Linkers |
| 4.3.3 | Orientation |
| 4.3.4 | Exemplary Conjugates |
| 4.4 | Activity of RNAi Agents & Conjugates Thereof |
| 4.5 | Methods of Making RNAi Agents & |
| | Conjugates Thereof |
| 4.6 | Vectors |
| 4.7 | Carriers |
| 4.7.1 | Lipid Based Carriers/Lipid Nanoformulations |
| 4.7.1.1 | Cationic Lipids (Positively Charged) |
| | and Ionizable Lipids |
| 4.7.1.2 | Non-Cationic Lipids (e.g., Phospholipids) |
| 4.7.1.3 | Structural Lipids |
| 4.7.1.4 | Polymers and Polyethylene Glycol (PEG)-Lipids |
| 4.7.1.5 | Percentages of Lipid Nanoformulation Components |
| 4.8 | Host Cells |
| 4.9 | Pharmaceutical Compositions |
| 4.10 | Methods of Use |
| 4.10.1 | Methods of Delivery |
| 4.10.2 | Methods of Reducing or Inhibiting CIDEB Expression |
| 4.10.3 | Methods of Treating, Ameliorating, or |
| | Preventing a CIDEB Associated |
| | Disease |
| 4.10.4 | Methods of Treating, Ameliorating, |
| | or Preventing a Liver Disease |
| 4.10.5 | Methods of Diagnosing and/or |
| | Prognosticating a Liver Disease |
| 4.10.6 | Methods of Screening, Identifying, |
| | and Selecting a Subject for Treatment |
| | with a CIDEB Inhibitory Agent |
| 4.10.7 | In Vitro Methods of Screening |
| | Samples for Somatic CIDEB Mutations |
| 4.11 | Kits |

### 4.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

As used herein, the term “administering” refers to the physical introduction of an agent, e.g., a therapeutic agent (or a precursor of the therapeutic agent that is metabolized or altered within the body of the subject to produce the therapeutic agent in vivo) to a subject, using any of the various methods and delivery systems known to those skilled in the art. Administering can also be performed, for example, once, a plurality of times, and/or over one or more extended periods. Administration includes administration to a subject by a third party; as well as self-administration by the subject.

As used herein, the term “agent” is used generically to describe any macro or micro molecule. Exemplary moieties include, but are not limited polypeptides, proteins, peptides, polynucleotides (e.g., DNA, RNA), small molecules, carbohydrates, lipids, synthetic polymers (e.g., polymers of PEG).

As used herein, the term “antisense strand” refers to an RNA molecule (e.g., part of an RNAi agent (e.g., described herein), part of a dsRNA agent (e.g., described herein)) that comprises a region of complementarity comprising a nucleotide sequence that is at least partially (e.g., substantially, fully) complementary to a target nucleic acid sequence (e.g., a target mRNA (e.g., a CIDEB mRNA), a portion of a target mRNA (e.g., a CIDEB mRNA)).

As used herein, the term “bicyclic sugar” refers to a modified sugar (e.g., ribose) moiety comprising two rings, wherein the second ring is formed via a bridge connecting two of the atoms in the first ring thereby forming a bicyclic structure. In some embodiments, the first ring of the bicyclic sugar moiety is a furanosyl moiety. In some embodiments, the furanosyl sugar moiety is a ribosyl moiety.

As used herein, the term “bicyclic nucleoside” (“BNA”) is a nucleoside comprising a bicyclic sugar.

As used herein, the term “blunt end” refers to a dsRNA molecule that does not contain any unpaired nucleotides at the end (e.g., 3′ terminus, 5′ terminus) of the dsRNA molecule (i.e., no nucleotide overhang(s)). The dsRNA molecule can have, for example, a blunt end at the 3′ end, 5′ end, or both the 3′ and 5′ end of the molecule.

As used herein, the term “CIDEB” or “cell death inducing DNA fragmentation factor alpha like effector” refers to the lipid droplet-associated protein primarily expressed in the liver and functions, e.g., to promote unilocular lipid droplet formation by mediating lipid droplet fusion. The mRNA sequence of a reference hCIDEB gene is set forth in SEQ ID NO: 1 (NCBI Ref.: NM_014430.4). The amino acid sequence of a reference hCIDEB protein is set forth in SEQ ID NO: 3 (NCBI Ref.: NP_055245). The term CIDEB includes naturally occurring variants of CIDEB. CIDEB gene and mRNA sequences of e.g., human, mouse, rat, non-human primate (e.g., rhesus macaque, Macaca fascicularis (cynomolgus monkey)), are readily available through publicly available databases, including, e.g., GenBank, UniProt, OMIM, and the Macaca genome project web site.

As used herein, the term “complementary” in reference to a first nucleotide sequence (e.g., a sense strand or a target mRNA) in relation to a second nucleotide sequence (e.g., an antisense strand), refers to the ability of a nucleic acid molecule comprising the first nucleotide sequence to hybridize to a nucleic acid molecule comprising the second nucleotide sequence and form a double stranded region (through base pair hydrogen bonds) under suitable in vivo or vitro conditions (e.g., under certain standard conditions, under mammalian (e.g., human) physiological conditions). A person of ordinary skill in the art would be able to select the set of conditions most appropriate for a hybridization test. Complementary sequences include, e.g., Watson-Crick base pairs. For example, complementary nucleobase pairs include adenine (A) and thymine (T); adenine (A) and uracil (U); and cytosine (C) and guanine (G). Complementary nucleobase pairs include natural and modified nucleotides, and nucleotide mimics, at least to the extent that the above hybridization requirements are fulfilled. As such, determinations of complementarity (as described herein) are independent of nucleotide chemical modifications (e.g., as described herein). For example, (C) and 5-methyl cytosine (mC) are both complementary to (G).

As used herein, the term “conjugation” refers to chemical conjugation of an agent (e.g., a nucleic acid molecule) with a moiety (e.g., carbohydrate, small molecule, polypeptide, polynucleotide, lipid, synthetic polymer (e.g., polymers of polyethylene glycol (PEG)), etc.). The moiety can be directly connected to the agent (e.g., nucleic acid molecule) or indirectly connected through a linker, e.g., as described herein. Chemical conjugation methods are well known in the art, as are commercially available conjugation reagents and kits, with detailed instructions for their use readily available from the commercial suppliers.

As used herein, the term “differing by no more than X nucleotides” in reference to a nucleotide sequence means that the nucleotide sequence comprises no more than X (wherein X is a specified number (e.g., 3, 2, 1, 0)) nucleotide variations relative to a reference sequence. For example, the phrase “wherein the nucleotide sequence of the antisense strand differs by no more than 3 nucleotides from the nucleotide sequence of SEQ ID NO: X” means that the nucleotide sequence comprises no more than 3 nucleotide variations relative to the nucleotide sequence set forth in the cited SEQ ID NO: X.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology. The term disease includes infection (e.g., a viral, bacterial, fungal, protozoal infection). The term disease includes other conditions caused by an infection.

As used herein, the term “double stranded RNA agent” or “dsRNA agent” refers to a complex of two RNA molecules comprising a double stranded region comprising two anti-parallel and at least partially (e.g., substantially, fully) complementary nucleic acid sequences that form the double stranded region. For example, in some embodiments, the dsRNA agent comprises a sense strand and an antisense strand.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein, the term “fully complementary” means that in a hybridized pair of a first nucleic acid molecule and a second nucleic acid molecule, 100% (all), of the bases in a contiguous sequence of the first nucleic acid molecule will hybridize with the same number of bases in a contiguous sequence of the second nucleic acid molecule. The contiguous sequence may comprise all or a part of the first and/or second nucleic acid molecule.

As used herein, the term “heterologous,” when used to describe a first element in reference to a second element means that the first element and second element do not exist in nature disposed as described. For example, a nucleic acid molecule comprising a “heterologous moiety” means a nucleic acid molecule that is joined to a moiety (e.g., carbohydrate, small molecule, polypeptide, polynucleotide, lipid, synthetic polymer (e.g., polymers of PEG), etc.) that is not joined to the nucleic acid molecule in nature.

As used herein, the term “isolated” with reference to a polypeptide, protein, or polynucleotide refers to a polypeptide, protein, or polynucleotide that is substantially free of other cellular components with which it is associated in the natural state.

As used herein, the term “nucleotide variation,” “variant nucleotide,” or use of the term “variation” and the like in reference to a nucleotide or nucleic acid sequence refers to a nucleic acid molecule that comprises at least one substitution, addition, deletion, or inversion of one or more nucleotide compared to a reference nucleic acid molecule. As used herein, the term “variant” or “variation” with reference to a peptide or protein refers to a peptide or protein that comprises at least one substitution, addition, deletion, or inversion of an amino acid residue compared to a reference peptide or protein.

As used herein, the term “modified agent” refers to any agent (or any component thereof (e.g., any nucleic acid molecule thereof)) (e.g., described herein, e.g., an antisense strand, a sense strand, a dsRNA agent, RNAi agent, etc.) that comprises one or more modified nucleotide (as defined herein).

As used herein, the term “modified nucleotide,” “nucleotide modification,” or use of the term “modification” and the like in reference to a nucleotide or nucleic acid sequence refers to a nucleotide comprising a chemical modification, e.g., a modified sugar moiety, a modified nucleobase, and/or a modified internucleoside linkage, or any combination thereof. Exemplary modifications are provided herein, see, e.g., §§ 4.3, 4.3.1. In certain embodiments of the instant disclosure, inclusion of a deoxynucleotide-which is acknowledged as a naturally occurring form of nucleotide-if present within an RNAi agent or component thereof (e.g., described herein, e.g., a sense strand, an antisense strand, a dsRNA agent) is considered to constitute a modified nucleotide.

As used herein, the term “moiety” is used generically to describe any macro or micro molecule that can be operably connected to a protein described herein. Exemplary moieties include, but are not limited small molecules, polypeptides, polynucleotides (e.g., DNA, RNA), carbohydrates, lipids, synthetic polymers (e.g., polymers of PEG).

As used herein, the term “nucleotide overhang” refers to at least one unpaired nucleotide that extends from the double stranded region of a nucleic acid molecule (e.g., a dsRNA molecule (e.g., a dsRNA molecule described herein)). For example, when a 3′-end of one strand of a dsRNA extends beyond the 5′-end of the other strand, or vice versa, there is a nucleotide overhang.

As used herein, the term, “non-complementary nucleotide mismatch” refers to a nucleotide within a region of complementarity (as described herein) that is not complementary to the corresponding nucleotide in the target nucleic acid molecule.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

As used herein, “partially complementary” means that in a hybridized pair of a first nucleic acid molecule and a second nucleic acid molecule, at least 70%, but not all, of the bases in a contiguous sequence of the first nucleic acid molecule will hybridize with the same number of bases in a contiguous sequence of the second nucleic acid molecule. The contiguous sequence may comprise all or a part of a first or second nucleic acid molecule.

The determination of “percent identity” between two sequences (e.g., protein (amino acid sequences) or polynucleotide (nucleic acid sequences)) can be accomplished using a mathematical algorithm. Determinations of identity (as described herein) are independent of nucleotide chemical modifications (e.g., as described herein). For example, (mC) is identical to (C) for the purposes of determining identity. A specific, non-limiting example of a mathematical algorithm utilized for the comparison of two sequences is the algorithm of Karlin S & Altschul SF (1990) PNAS 87:2264-2268, modified as in Karlin S & Altschul SF (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleoside linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “plurality” means 2 or more (e.g., 3 or more, 4 or more, 5 or more, 6 or more, 7 or more, 9 or more, or 10 or more).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where proteins are contemplated herein, it should be understood that proteins folded into their three-dimensional structure are also provided herein.

As used herein, the term “region of complementarity” refers to a portion of a first nucleic acid molecule comprising a nucleotide sequence that is at least partially complementary to the nucleotide sequence of at least a portion of a second nucleic acid molecule.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleoside linkages, such as a phosphoroamidate linkage or a phosphorothioate linkage, instead of the phosphodiester found between the nucleotides of an unmodified nucleic acid molecule.

As used herein, the term “RNAi agent” refers to an agent that contains one or more RNA molecules which can mediate the targeted cleavage of an RNA molecule (e.g., an mRNA molecule) via an RNA-induced silencing complex (RISC) pathway. The RNAi agent, is thereby capable of e.g., modulating, e.g., inhibiting, the expression of a target gene (e.g., CIDEB) in a cell, e.g., a cell within a subject, such as a mammalian subject. In some embodiments, the RNAi agent is a dsRNA agent comprising a sense strand and an antisense strand that form a double stranded region, wherein optionally the sense strand and the antisense strand each independently comprise or consist of from about 19-23 nucleotides.

As used herein, the term “sense strand” refers to an RNA molecule (e.g., part of an RNAi agent (e.g., described herein), part of a dsRNA agent (e.g., described herein)) that comprises a region that is at least partially (e.g., substantially, fully) complementary to a region of the antisense strand (as defined herein). The sense strand is often referred to as such with reference to the orientation of the sequence of the sense strand being the same with respect to a target RNA (e.g., mRNA sequence).

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Palcaognathac (e.g., ostriches, emus), Columbiformes (e.g., pigeons, doves), or Psittaciformes (e.g., parrots).

As used herein, “substantially complementary” means that in a hybridized pair of a first nucleic acid molecule and a second nucleic acid molecule, at least 85%, but not all, of the bases in a contiguous sequence of the first nucleic acid molecule will hybridize with the same number of bases in a contiguous sequence of the second nucleic acid molecule. The contiguous sequence may comprise all or a part of a first or second nucleic acid molecule.

In some embodiments, the term “substantially all” means at least 95%, 96%, 97%, 98% or 99%, e.g., of the subject of said sentence. The term “substantially all” preferably excludes 100%. For example, in some embodiments, the term “substantially all of the nucleotides in the sense strand and/or antisense strand are modified” means that at least 95%, 96%, 97%, 98% or 99% of said nucleotides are modified. For example, in some embodiments, the term “substantially all of the nucleotides of the agent are modified” means that at least 95%, 96%, 97%, 98% or 99% of said nucleotides are modified. For example, in some embodiments, the term “substantially all of the nucleotides of the agent are unmodified” means that at least 95%, 96%, 97%, 98% or 99% of said nucleotides are unmodified. For example, in some embodiments the term “wherein the dsRNA agent is in the sodium salt form, sodium ions are present in the composition comprising the dsRNA agent as counterions for substantially all of the phosphodiester or phosphorothioate groups present in the dsRNA agent” means that wherein the dsRNA agent is in the sodium salt form, sodium ions are present in the composition comprising the dsRNA agent as counterions for at least 95%, 96%, 97%, 98% or 99% of the phosphodiester or phosphorothioate groups present in the dsRNA agent.

As used herein, the term “target nucleic acid sequence” refers to a contiguous portion of the nucleotide sequence of a nucleic acid sequence (e.g., an mRNA molecule formed during the transcription of a target gene (e.g., CIDEB)). In some embodiments, the target nucleic acid sequence is an mRNA molecule formed during the transcription of a target gene (e.g., CIDEB)). In some embodiments, the target nucleic acid molecule comprises an mRNA that is a product of RNA processing of a primary transcription product. The target portion of the sequence (e.g., mRNA) will be at least long enough to serve as a substrate for RNAi-directed cleavage at or near that portion of the nucleotide sequence of an mRNA molecule formed during the transcription of a CIDEB gene. In one embodiment, the target sequence is within the protein coding region of CIDEB.

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

### 4.2 RNAi Agents

Provided herein are, inter alia, agents (e.g., RNAi agents, dsRNA agents), useful in, inter alia, inhibiting expression of cell death inducing DFFA like effector B (CIDEB) (e.g., human CIDEB (hCIDEB)) (e.g., within a cell, e.g., within a cell in a subject, e.g., a mammalian subject, e.g., a human subject) (e.g., through the degradation of CIDEB (e.g., hCIDEB) mRNA).

CIDEB is a lipid droplet-associated protein primarily expressed in the liver and functions, e.g., to promote unilocular lipid droplet formation by mediating lipid droplet fusion. The mRNA sequence of a reference hCIDEB gene is set forth in SEQ ID NO: 1 (NCBI Ref.: NM_014430.4). The reverse complement sequence of the hCIDEB mRNA is set forth in SEQ ID NO: 2. The amino acid sequence of a reference hCIDEB protein is set forth in SEQ ID NO: 3 (NCBI Ref.: NP_055245). See Table 1, herein.

TABLE 1
The mRNA and Amino Acid Sequence of a Reference hCIDEB Protein.

| | | SEQ ID |
|---|---|---|
| Description | Amino Acid Sequence | NO |
| hCIDEB mRNA | CCCUUCCGGUGGAGCCAGCGCUGCGACCGCCUGCAGAAGGUUGACUGC | 1 |
| NCBI Ref.: | GUGGUAGGGGGCCCAGAGCAAGCCGAAGGCAAGCACGAUGGCGCUCAC | |
| NM_014430.4 | CAGCCGGCCCACCCGCGCCCCGUGCCGCCCGGAGCCCCAGCGGGCGCC | |
| | CCGCAGCCGUGCCAGCGUCACGCUGUAGCAGCCGAGCAUCAGCCCGAA | |
| | AGGAAGCACGAAAGCGGUCAGAGUCUCCAGGCUCAGGUGGGCGGCGGC | |
| | GUGGACCGGCGACGGGUGGCACAGCUGGCAUACGCGGUCCCUCCACAG | |
| | GUGGCGGUAGACGGCGGCCGGGACGGCGAGCAACAGGGCGGCCAGCCA | |
| | GACCGCCAGCAGCAGGCGGCGGGCCAGGGCCGGGCUGCGCAGCCGAGG | |
| | CGCCAGGAAGGGGCGGGUGACUGCGAGGCAGCGCUGCAGGCUGAGCAG | |
| | GCCGGUGAGCAGCACGCUGGCGUACAUGCUGAGCGCGCACACGUAGUA | |
| | CACCGCCUUGCAGCCCGCCUGGCCCAGCGGCCAGGCCUGCCGGGUCAG | |
| | GAAGGCCACAAAGAGCGGCGUGAGCAGCAGCACCGCGCCGUCGGCCAG | |
| | CGCCAGGUGCAGCACAAGCGUGGCCGCCAGCGGUCGCCCCCGUGCAGG | |
| | CCGCCAGCCCGCCAAGCUCCACACCACGAAGCCGUUGCCAGGCAGCCC | |
| | CAGCAGCGCCGCCAGCAGCAGGAAGGCUGUGCCUGUGGCCCGCGAAGU | |
| | CUUCCAGCUCAGCAGUGUCUCGUUCCCUGGGGGACGGUAGCAGACCGA | |
| | CAUCCUUCUGGGCCUACAGGACACAGAAAAAAAGUGGGGAAGCUGGGG | |
| | GACCCUACAAGGAUCCUUGGCAGGAAAGCAGGGAUUGUGUUCAUUUGA | |
| | GGGUUUCACUGUCAGUGAGAGUCUCAGCUUCCAUGCAACUGUCCAUCA | |
| | CGGCUGCAACUGAAAUCAGAGCUGGGACACAGCGCACCAGAAGCUAAA | |
| | GUCUUGAUGCCAUCAAAGGACAUCCCUGCCCCAUUCACAUCUCUGUCA | |
| | CGUCCACUAAUCGGCAAAAGGAGAAAAGUGAGAGAAGAUGACCUAAGU | |
| | GUGACUGCAGCAGGCAGCUCUGGAAAAUGAAGCCAGAGCAGUGAGCCA | |
| | GCCCCUCCUCCGACCAAGGAGGAAGGAAAGAGCAGCCCCAGCACAGGA | |
| | GAGAACCACCCAGCCCAGAAGUUCCAGGGAAGGAACUCUCCGGUCCAC | |
| | CAUGGAGUACCUCUCAGCUCUGAACCCCAGUGACUUACUCAGGUCAGU | |
| | AUCUAAUAUAAGCUCGGAGUUUGGACGGAGGGUCUGGACCUCAGCUCC | |
| | ACCACCCCAGCGACCUUUCCGUGUCUGUGAUCACAAGCGGACCAUCCG | |
| | GAAAGGCCUGACAGCUGCCACCCGCCAGGAGCUGCUAGCCAAAGCAUU | |
| | GGAGACCCUACUGCUGAAUGGAGUGCUAACCCUGGUGCUAGAGGAGGA | |
| | UGGAACUGCAGUGGACAGUGAGGACUUCUUCCAGCUGCUGGAGGAUGA | |
| | CACGUGCCUGAUGGUGUUGCAGUCUGGUCAGAGCUGGAGCCCUACAAG | |
| | GAGUGGAGUGCUGUCAUAUGGCCUGGGACGGGAGAGGCCCAAGCACAG | |
| | CAAGGACAUCGCCCGAUUCACCUUUGACGUGUACAAGCAAAACCCUCG | |
| | AGACCUCUUUGGCAGCCUGAAUGUCAAAGCCACAUUCUACGGGCUCUA | |
| | CUCUAUGAGUUGUGACUUUCAAGGACUUGGCCCAAAGAAAGUACUCAG | |
| | GGAGCUCCUUCGUUGGACCUCCACACUGCUGCAAGGCCUGGGCCAUAU | |
| | GUUGCUGGGAAUUUCCUCCACCCUUCGUCAUGCAGUGGAGGGGGCUGA | |
| | GCAGUGGCAGCAGAAGGGCCGCCUCCAUUCCUACUAAGGGGCUCUGAG | |
| | CUUCUGCCCCCAGAAUCAUUCCAACCGACCCACUGCAAAGACUAUGAC | |
| | AGCAUCAAAUUUCAGGACCUGCAGACAGUACAGGCUAGAUAACCCACC | |
| | CAAUUUCCCCACUGUCCUCUGAUCCCCUCGUGACAGAACCUUUCAGCA | |
| | UAACGCCUCACAUCCCAAGUCUAUACCCUUACCUGAAGAAUGCUGUUC | |
| | UUUCCUAGCCACCUUUCUGGCCUCCCACUUGCCCUGAAAGGCCAAGAU | |
| | CAAGAUGUCCCCCAGGCAUCUUGAUCCCAGCCUGACUGCUGCUACAUC | |
| | UAAUCCCCUACCAAUGCCUCCUGUCCCUAAACUCCCCAGCAUACUGAU | |
| | GACAGCCCUCUCUGACUUUACCUUGAGAUCUGUCUUCAUACCCUUCCC | |
| | CUCAAACUAACAAAAACAUUUCCAAUAAAAAUAUCAAAUAUUUACCAC | |
| | UAA | |
| Reverse | UUAGUGGUAAAUAUUUGAUAUUUUUAUUGGAAAUGUUUUUGUUAGUUU | 2 |
| Complement | GAGGGGAAGGGUAUGAAGACAGAUCUCAAGGUAAAGUCAGAGAGGGCU | |
| of hCIDEB | GUCAUCAGUAUGCUGGGGAGUUUAGGGACAGGAGGCAUUGGUAGGGGA | |
| mRNA | UUAGAUGUAGCAGCAGUCAGGCUGGGAUCAAGAUGCCUGGGGGACAUC | |
| | UUGAUCUUGGCCUUUCAGGGCAAGUGGGAGGCCAGAAAGGUGGCUAGG | |
| | AAAGAACAGCAUUCUUCAGGUAAGGGUAUAGACUUGGGAUGUGAGGCG | |
| | UUAUGCUGAAAGGUUCUGUCACGAGGGGAUCAGAGGACAGUGGGGAAA | |
| | UUGGGUGGGUUAUCUAGCCUGUACUGUCUGCAGGUCCUGAAAUUUGAU | |
| | GCUGUCAUAGUCUUUGCAGUGGGUCGGUUGGAAUGAUUCUGGGGGCAG | |
| | AAGCUCAGAGCCCCUUAGUAGGAAUGGAGGCGGCCCUUCUGCUGCCAC | |
| | UGCUCAGCCCCCUCCACUGCAUGACGAAGGGUGGAGGAAAUUCCCAGC | |
| | AACAUAUGGCCCAGGCCUUGCAGCAGUGUGGAGGUCCAACGAAGGAGC | |
| | UCCCUGAGUACUUUCUUUGGGCCAAGUCCUUGAAAGUCACAACUCAUA | |
| | GAGUAGAGCCCGUAGAAUGUGGCUUUGACAUUCAGGCUGCCAAAGAGG | |
| | UCUCGAGGGUUUUGCUUGUACACGUCAAAGGUGAAUCGGGCGAUGUCC | |
| | UUGCUGUGCUUGGGCCUCUCCCGUCCCAGGCCAUAUGACAGCACUCCA | |
| | CUCCUUGUAGGGCUCCAGCUCUGACCAGACUGCAACACCAUCAGGCAC | |
| | GUGUCAUCCUCCAGCAGCUGGAAGAAGUCCUCACUGUCCACUGCAGUU | |
| | CCAUCCUCCUCUAGCACCAGGGUUAGCACUCCAUUCAGCAGUAGGGUC | |
| | UCCAAUGCUUUGGCUAGCAGCUCCUGGCGGGUGGCAGCUGUCAGGCCU | |
| | UUCCGGAUGGUCCGCUUGUGAUCACAGACACGGAAAGGUCGCUGGGGU | |
| | GGUGGAGCUGAGGUCCAGACCCUCCGUCCAAACUCCGAGCUUAUAUUA | |
| | GAUACUGACCUGAGUAAGUCACUGGGGUUCAGAGCUGAGAGGUACUCC | |
| | AUGGUGGACCGGAGAGUUCCUUCCCUGGAACUUCUGGGCUGGGUGGUU | |
| | CUCUCCUGUGCUGGGGCUGCUCUUUCCUUCCUCCUUGGUCGGAGGAGG | |
| | GGCUGGCUCACUGCUCUGGCUUCAUUUUCCAGAGCUGCCUGCUGCAGU | |
| | CACACUUAGGUCAUCUUCUCUCACUUUUCUCCUUUUGCCGAUUAGUGG | |
| | ACGUGACAGAGAUGUGAAUGGGGCAGGGAUGUCCUUUGAUGGCAUCAA | |
| | GACUUUAGCUUCUGGUGCGCUGUGUCCCAGCUCUGAUUUCAGUUGCAG | |
| | CCGUGAUGGACAGUUGCAUGGAAGCUGAGACUCUCACUGACAGUGAAA | |
| | CCCUCAAAUGAACACAAUCCCUGCUUUCCUGCCAAGGAUCCUUGUAGG | |
| | GUCCCCCAGCUUCCCCACUUUUUUUCUGUGUCCUGUAGGCCCAGAAGG | |
| | AUGUCGGUCUGCUACCGUCCCCCAGGGAACGAGACACUGCUGAGCUGG | |
| | AAGACUUCGCGGGCCACAGGCACAGCCUUCCUGCUGCUGGCGGCGCUG | |
| | CUGGGGCUGCCUGGCAACGGCUUCGUGGUGUGGAGCUUGGCGGGCUGG | |
| | CGGCCUGCACGGGGGCGACCGCUGGCGGCCACGCUUGUGCUGCACCUG | |
| | GCGCUGGCCGACGGCGCGGUGCUGCUGCUCACGCCGCUCUUUGUGGCC | |
| | UUCCUGACCCGGCAGGCCUGGCCGCUGGGCCAGGCGGGCUGCAAGGCG | |
| | GUGUACUACGUGUGCGCGCUCAGCAUGUACGCCAGCGUGCUGCUCACC | |
| | GGCCUGCUCAGCCUGCAGCGCUGCCUCGCAGUCACCCGCCCCUUCCUG | |
| | GCGCCUCGGCUGCGCAGCCCGGCCCUGGCCCGCCGCCUGCUGCUGGCG | |
| | GUCUGGCUGGCCGCCCUGUUGCUCGCCGUCCCGGCCGCCGUCUACCGC | |
| | CACCUGUGGAGGGACCGCGUAUGCCAGCUGUGCCACCCGUCGCCGGUC | |
| | CACGCCGCCGCCCACCUGAGCCUGGAGACUCUGACCGCUUUCGUGCUU | |
| | CCUUUCGGGCUGAUGCUCGGCUGCUACAGCGUGACGCUGGCACGGCUG | |
| | CGGGGCGCCCGCUGGGGCUCCGGGCGGCACGGGGCGCGGGUGGGCCGG | |
| | CUGGUGAGCGCCAUCGUGCUUGCCUUCGGCUUGCUCUGGGCCCCCUAC | |
| | CACGCAGUCAACCUUCUGCAGGCGGUCGCAGCGCUGGCUCCACCGGAA | |
| | GGG | |
| hCIDEB | MEYLSALNPSDLLRSVSNISSEFGRRVWTSAPPPQRPFRVCDHKRTIR | 3 |
| Protein | KGLTAATRQELLAKALETLLLNGVLTLVLEEDGTAVDSEDFFQLLEDD | |
| NCBI Ref.: | TCLMVLQSGQSWSPTRSGVLSYGLGRERPKHSKDIARFTFDVYKQNPR | |
| NP_055245 | DLFGSLNVKATFYGLYSMSCDFQGLGPKKVLRELLRWTSTLLQGLGHM | |
| | LLGISSTLRHAVEGAEQWQQKGRLHSY | |

In some embodiments, the agent (e.g., RNAi agent, dsRNA agent) comprises one or more RNA molecule. In some embodiments, the agent (e.g., RNAi agent, dsRNA agent) comprises an antisense strand. In some embodiments, the agent (e.g., RNAi agent, dsRNA agent) comprises a sense strand. In some embodiments, the agent comprises one or more single stranded RNA (ssRNA) molecules. In some embodiments, the agent (e.g., RNAi agent, dsRNA agent) comprises a dsRNA agent.

In some embodiments, the agent (e.g., RNAi agent) comprises a dsRNA agent comprising a sense strand and an antisense strand. In some embodiments, the agent (e.g., RNAi agent) comprises a dsRNA agent comprising a sense strand and an antisense strand that form a double stranded region. In some embodiments, the agent (e.g., RNAi agent) comprises a dsRNA agent comprising a sense strand and an antisense strand that hybridize to form a double stranded region. In some embodiments, the sense strand and the antisense strand are part of a single nucleic acid molecule (e.g., a single nucleic acid molecule comprising a hairpin loop). In some embodiments, the sense strand and the antisense strand are separate nucleic acid molecules.

### 4.2.1 Antisense Strand

### 4.2.1.1 Targeting Region

As described above, antisense strands (e.g., described herein) comprise a region of complementarity that comprises a nucleotide sequence that is at least partially (e.g., substantially, fully) complementary to the nucleotide sequence of a target nucleic acid molecule (e.g., a target mRNA (e.g., a CIDEB mRNA), a portion of a target mRNA (e.g., a CIDEB mRNA)). In some embodiments, the nucleotide sequence of the region of complementarity is at least substantially complementary to the nucleotide sequence of the target nucleic acid molecule (e.g., a target mRNA (e.g., a CIDEB mRNA), a portion of a target mRNA (e.g., a CIDEB mRNA)). In some embodiments, the nucleotide sequence of the region of complementarity is fully complementary to the nucleotide sequence of the target nucleic acid molecule (e.g., a target mRNA (e.g., a CIDEB mRNA), a portion of a target mRNA (e.g., a CIDEB mRNA)).

In some embodiments, the nucleotide sequence of the region of complementarity is at least 70%, 75%, 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a CIDEB mRNA), a portion of a target mRNA (e.g., a CIDEB mRNA)). For example, the nucleotide sequence of the region of complementarity may be at least 70% complementary to the nucleotide sequence of the target nucleic acid molecule (e.g., a target mRNA (e.g., a CIDEB mRNA), a portion of a target mRNA (e.g., a CIDEB mRNA)). The nucleotide sequence of the region of complementarity may be at least 75% complementary to the nucleotide sequence of the target nucleic acid molecule (e.g., a target mRNA (e.g., a CIDEB mRNA), a portion of a target mRNA (e.g., a CIDEB mRNA)). The nucleotide sequence of the region of complementarity may be at least 80% complementary to the nucleotide sequence of the target nucleic acid molecule (e.g., a target mRNA (e.g., a CIDEB mRNA), a portion of a target mRNA (e.g., a CIDEB mRNA)). The nucleotide sequence of the region of complementarity may be at least 85% complementary to the nucleotide sequence of the target nucleic acid molecule (e.g., a target mRNA (e.g., a CIDEB mRNA), a portion of a target mRNA (e.g., a CIDEB mRNA)). The nucleotide sequence of the region of complementarity may be at least 90% complementary to the nucleotide sequence of the target nucleic acid molecule (e.g., a target mRNA (e.g., a CIDEB mRNA), a portion of a target mRNA (e.g., a CIDEB mRNA)). The nucleotide sequence of the region of complementarity may be at least 95% complementary to the nucleotide sequence of the target nucleic acid molecule (e.g., a target mRNA (e.g., a CIDEB mRNA), a portion of a target mRNA (e.g., a CIDEB mRNA)). In some embodiments, the nucleotide sequence of the region of complementarity is at least 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a CIDEB mRNA), a portion of a target mRNA (e.g., a CIDEB mRNA)). In some embodiments, the nucleotide sequence of the region of complementarity is at least 95%, 96%, 97%, 98%, 99%, or 100% (e.g., in some embodiments, preferably at least 95%, more preferably at least 98%) complementary to the nucleotide sequence of the target nucleic acid molecule (e.g., a target mRNA (e.g., a CIDEB mRNA), a portion of a target mRNA (e.g., a CIDEB mRNA)). In some embodiments, the nucleotide sequence of the region of complementarity is 100% complementary to the nucleotide sequence of the target nucleic acid molecule (e.g., a target mRNA (e.g., a CIDEB mRNA), a portion of a target mRNA (e.g., a CIDEB mRNA)).

In some embodiments, the nucleotide sequence of the region of complementarity comprises or consists of one or more non-complementary nucleotide mismatches relative to the nucleotide sequence of the target nucleic acid molecule (e.g., a target mRNA (e.g., a CIDEB mRNA), a portion of a target mRNA (e.g., a CIDEB mRNA)). In some embodiments, the nucleotide sequence of the region of complementarity comprises or consists of no more than 5 (e.g., 4, 3, 2, 1, or 0) non-complementary nucleotide mismatches relative to the nucleotide sequence of the target nucleic acid molecule. In some embodiments, the nucleotide sequence of the region of complementarity comprises or consists of no more than 3 (e.g., 2, 1, or 0) non-complementary nucleotide mismatches relative to the nucleotide sequence of the target nucleic acid molecule. In some embodiments, the nucleotide sequence of the region of complementarity comprises or consists of no more than 2 (e.g., 1 or 0) non-complementary nucleotide mismatches relative to the nucleotide sequence of the target nucleic acid molecule. In some embodiments, the nucleotide sequence of the region of complementarity comprises or consists of no more than 1 (e.g., 0) non-complementary nucleotide mismatch relative to the nucleotide sequence of the target nucleic acid molecule. In some embodiments, the nucleotide sequence of the region of complementarity comprises 0 non-complementary nucleotide mismatches relative to the nucleotide sequence of the target nucleic acid molecule. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. Methods known in the art and described herein can be utilized to evaluate the effect of any non-complementary mismatches between an antisense strand and a target nucleic acid molecule on functional properties (e.g., inhibition of expression of the target nucleic acid molecule (e.g., a target mRNA (e.g., a CIDEB mRNA), a portion of a target mRNA (e.g., a CIDEB mRNA))).

In some embodiments, the region of complementarity comprises or consists of from about 15-30 nucleotides, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides. In some embodiments, the region of complementarity comprises from about 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides. In some embodiments, the region of complementarity comprises from about 19-21 (e.g., 19-20) nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 19, 20, 21, 22, or 23 nucleotides. In some embodiments, the region of complementarity comprises or consists of about 19 nucleotides. In some embodiments, the region of complementarity comprises or consists of about 20 nucleotides. In some embodiments, the region of complementarity comprises or consists of about 21 nucleotides. In some embodiments, the region of complementarity comprises or consists of about 22 nucleotides. In some embodiments, the region of complementarity comprises or consists of about 23 nucleotides. Ranges and lengths intermediate to the above recited ranges and lengths are also contemplated to be part of the disclosure.

In some embodiments, the target nucleic acid molecule is part (e.g., a contiguous portion) of a larger nucleic acid molecule. For example, in some embodiments, the target nucleic acid molecule is a portion (e.g., a contiguous portion) of a target mRNA (e.g., a CIDEB mRNA). In some embodiments, the target nucleic acid molecule is a contiguous nucleotide sequence of a target mRNA (e.g., a CIDEB mRNA) of sufficient length to allow it to be a substrate for cleavage directed by an RNAi agent (e.g., an RNAi agent described herein, e.g., a dsRNA agent (e.g., described herein)) (i.e., cleavage through a RISC pathway).

In some embodiments, the target nucleic acid molecule is a target mRNA (e.g., a CIDEB mRNA). In some embodiments, the target nucleic acid molecule is at least a portion (e.g., a portion) of a target mRNA (e.g., a CIDEB mRNA). In some embodiments, the target nucleic acid molecule is at least a portion (e.g., a portion) of an mRNA (e.g., a CIDEB mRNA) formed in the expression of a target gene (e.g., a mammalian, primate, human, non-human primate, mouse, and/or rat gene) (e.g., a CIDEB gene). In some embodiments, the target nucleic acid molecule is at least a portion (e.g., a portion) of a CIDEB (e.g., hCIDEB) mRNA. In some embodiments, the target nucleic acid molecule is at least a portion (e.g., a portion) of an mRNA formed in the expression of a CIDEB (e.g., hCIDEB) gene. In some embodiments, the target nucleic acid molecule comprises at least a portion (e.g., a portion) of the nucleotide sequence set forth in SEQ ID NO: 1 (or a variant or fragment thereof). In some embodiments, the target nucleic acid molecule comprises at least a portion (e.g., a portion) of an mRNA encoding a target protein. In some embodiments, the target nucleic acid molecule comprises at least a portion (e.g., a portion) of an mRNA encoding a CIDEB (e.g., hCIDEB) protein. In some embodiments, the target nucleic acid molecule comprises at least a portion (e.g., a portion) of an mRNA sequence encoding a protein comprising the amino acid sequence set forth in SEQ ID NO: 3 (or a variant or fragment thereof).

In some embodiments, the target nucleic acid molecule comprises or consists of from about 19-30 nucleotides, e.g., 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, 21-22, 22-30, 22-29, 22-28, 22-27, 22-26, 22-25, 22-24, 22-23, 23-30, 23-29, 23-28, 23-27, 23-26, 23-27, 23-26, 23-25, or 23-24 nucleotides. In some embodiments, the target nucleic acid molecule comprises or consists of from about 19-25 nucleotides. In some embodiments, the target nucleic acid molecule comprises or consists of from about 19-23 nucleotides. In some embodiments, the target nucleic acid molecule comprises or consists of from about 21-25 nucleotides. In some embodiments, the target nucleic acid molecule comprises or consists of from about 21-23 nucleotides. In some embodiments, the target nucleic acid molecule comprises or consists of about 19, 18, 20, 21, 22, 23, 24, 25, 26, 27, 28, 29, or 30 nucleotides. In some embodiments, the target nucleic acid molecule comprises or consists of about 19 nucleotides. In some embodiments, the target nucleic acid molecule comprises or consists of about 20 nucleotides. In some embodiments, the target nucleic acid molecule comprises or consists of about 21 nucleotides. In some embodiments, the target nucleic acid molecule comprises or consists of about 23 nucleotides. Ranges and lengths intermediate to the above recited ranges and lengths are also contemplated to be part of the disclosure.

### 4.2.1.2 Overall Length

In some embodiments, the antisense strand comprises or consists of from about 15-30 nucleotides (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides). In some embodiments, the antisense strand comprises or consists of from about 18-25 nucleotides (e.g., 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides). In some embodiments, the antisense strand comprises or consists of from about 19-25 nucleotide (e.g., 19-20, 19-21, 19-22, 19-23, 19-24, 19-25, 20-21, 20-22, 20-23, 20-24, 20-25, 21-22, 21-23, 21-24, 21-25, 22-23, 22-24, 22-25, 23-24, 23-25, 24-25 nucleotides). In some embodiments, the antisense strand comprises or consists of from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides.

In some embodiments, the antisense strand comprises or consists of not more than about 15, 16, 17, 18, 19, 20, 21, 22, 23, 24, 25, 26, 27, 28, 29, or 30 nucleotides. In some embodiments, the antisense strand comprises or consists of about 15, 16, 17, 18, 19, 20, 21, 22, 23, 24, 25, 26, 27, 28, 29, or 30 nucleotides. In some embodiments, the antisense strand comprises or consists of about 21 nucleotides. In some embodiments, the antisense strand comprises or consists of about 23 nucleotides. Ranges and lengths intermediate to the above recited ranges and lengths are also contemplated to be part of the disclosure.

### 4.2.1.3 Exemplary Antisense Strands

In some embodiments, the nucleotide sequence of the antisense strand differs by no more than 5 (e.g., 0, 1, 2, 3, 4, or 5) nucleotides from the nucleotide sequence of any one of the antisense strands set forth in Table 2 or Table 3. In some embodiments, the nucleotide sequence of the antisense strand differs by no more than 3 (e.g., 0, 1, 2, or 3) nucleotides from the nucleotide sequence of any one of the antisense strands set forth in Table 2 or Table 3. In some embodiments, the nucleotide sequence of the antisense strand differs by no more than 2 (e.g., 0, 1, or 2) nucleotides from the nucleotide sequence of any one of the antisense strands set forth in Table 2 or Table 3. In some embodiments, the nucleotide sequence of the antisense strand differs by no more than 1 (e.g., 0 or 1) nucleotide from the nucleotide sequence of any one of the antisense strands set forth in Table 2 or Table 3.

In some embodiments, the nucleotide sequence of the antisense strand comprises the nucleotide sequence of any one of the antisense strands set forth in Table 2 or Table 3 differing by no more than 5 (e.g., 0, 1, 2, 3, 4, or 5) nucleotides from the antisense strand set forth in Table 2 or Table 3. In some embodiments, the nucleotide sequence of the antisense strand comprises the nucleotide sequence of any one of the antisense strands set forth in Table 2 or Table 3 differing by no more than 3 (e.g., 0, 1, 2, or 3) nucleotides from the antisense strand set forth in Table 2 or Table 3. In some embodiments, the nucleotide sequence of the antisense strand comprises the nucleotide sequence of any one of the antisense strands set forth in Table 2 or Table 3 differing by no more than 2 (e.g., 0, 1, or 2) nucleotides from the antisense strand set forth in Table 2 or Table 3. In some embodiments, the nucleotide sequence of the antisense strand comprises the nucleotide sequence of any one of the antisense strands set forth in Table 2 or Table 3 differing by no more than 1 (e.g., 0 or 1) nucleotide from the antisense strand set forth in Table 2 or Table 3.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands set forth in Table 2 or Table 3. In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands set forth in Table 2 or Table 3. In some embodiments, the nucleotide sequence of the antisense strand is at least 95%, 96%, 97%, 98%, 99% or 100% identical to the nucleotide sequence of any one of the antisense strands set forth in Table 2 or Table 3. In some embodiments, the nucleotide sequence of the antisense strand is at least 96%, 97%, 98%, 99% or 100% identical to the nucleotide sequence of any one of the antisense strands set forth in Table 2 or Table 3. In some embodiments, the nucleotide sequence of the antisense strand is at least 97%, identical to the nucleotide sequence of any one of the antisense strands set forth in Table 2 or Table 3. In some embodiments, the nucleotide sequence of the antisense strand is at least 98% identical to the nucleotide sequence of any one of the antisense strands set forth in Table 2 or Table 3. In some embodiments, the nucleotide sequence of the antisense strand is at least 99% identical to the nucleotide sequence of any one of the antisense strands set forth in Table 2 or Table 3. In some embodiments, the nucleotide sequence of the antisense strand is 100% identical to the nucleotide sequence of any one of the antisense strands set forth in Table 2 or Table 3.

In some embodiments, the nucleotide sequence of the antisense strand comprises the nucleotide sequence of any one of the antisense strands set forth in Table 2 or Table 3. In some embodiments, the nucleotide sequence of the antisense strand consists of the nucleotide sequence of any one of the antisense strands set forth in Table 2 or Table 3.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence of any one of the antisense strands set forth in Table 2 or Table 3 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the antisense strands set forth in Table 2 or Table 3.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in Table 2 or Table 3.

In some embodiments, the nucleotide sequence of the antisense strand comprises the nucleotide sequence of any one of the antisense strands set forth in Table 2 or Table 3 differing by no more than 3 (e.g., 0, 1, 2, or 3) nucleotides from the antisense strand set forth in Table 2 or Table 3. In some embodiments, the nucleotide sequence of the antisense strand comprises the nucleotide sequence of any one of the antisense strands set forth in Table 2 or Table 3.

Table 2 or Table 3 further identifies the target nucleic acid molecule within the cited reference CIDEB mRNA transcript (SEQ ID NO: 1) targeted by each of the antisense strands. As such, the disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in any one of SEQ ID NOS: 187-369, 602-657, 893-1131, or 1188-1191. In some embodiments, the nucleotide sequence of the antisense strand differs by no more than 3 (e.g., 0, 1, 2, or 3) nucleotides from the nucleotide sequence set forth in any one of SEQ ID NOS: 187-369, 602-657, 893-1131, or 1188-1191. In some embodiments, the nucleotide sequence of the antisense strand differs by no more than 2 (e.g., 0, 1, or 2) nucleotides from the nucleotide sequence set forth in any one of SEQ ID NOS: 187-369, 602-657, 893-1131, or 1188-1191. In some embodiments, the nucleotide sequence of the antisense strand differs by no more than 1 (e.g., 0 or 1) nucleotide from the nucleotide sequence set forth in any one of SEQ ID NOS: 187-369, 602-657, 893-1131, or 1188-1191.

In some embodiments, the nucleotide sequence of the antisense strand comprises the nucleotide sequence set forth in any one of SEQ ID NOS: 187-369, 602-657, 893-1131, or 1188-1191 differing by no more than 5 (e.g., 0, 1, 2, 3, 4, or 5) nucleotides from the nucleotide sequence set forth in any one of SEQ ID NOS: 187-369, 602-657, 893-1131, or 1188-1191. In some embodiments, the nucleotide sequence of the antisense strand comprises the nucleotide sequence set forth in any one of SEQ ID NOS: 187-369, 602-657, 893-1131, or 1188-1191 differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the nucleotide sequence of the antisense strand comprises the nucleotide sequence set forth in any one of SEQ ID NOS: 187-369, 602-657, 893-1131, or 1188-1191 differing by no more than 2 (e.g., 0, 1, or 2) nucleotides from the nucleotide sequence set forth in any one of SEQ ID NOS: 187-369, 602-657, 893-1131, or 1188-1191. In some embodiments, the nucleotide sequence of the antisense strand comprises the nucleotide sequence set forth in any one of SEQ ID NOS: 187-369, 602-657, 893-1131, or 1188-1191 differing by no more than 1 (e.g., 0 or 1) nucleotide from the nucleotide sequence set forth in any one of SEQ ID NOS: 187-369, 602-657, 893-1131, or 1188-1191.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the antisense strand is at least 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the antisense strand is at least 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the antisense strand is at least 97%, identical to the nucleotide sequence set forth in any one of SEQ ID NOS: 187-369, 602-657, 893-1131, or 1188-1191. In some embodiments, the nucleotide sequence of the antisense strand is at least 98% identical to the nucleotide sequence set forth in any one of SEQ ID NOS: 187-369, 602-657, 893-1131, or 1188-1191. In some embodiments, the nucleotide sequence of the antisense strand is at least 99% identical to the nucleotide sequence set forth in any one of SEQ ID NOS: 187-369, 602-657, 893-1131, or 1188-1191. In some embodiments, the nucleotide sequence of the antisense strand is 100% identical to the nucleotide sequence set forth in any one of SEQ ID NOS: 187-369, 602-657, 893-1131, or 1188-1191.

In some embodiments, the nucleotide sequence of the antisense strand comprises the nucleotide sequence set forth in any one of SEQ ID NOS: 187-369, 602-657, 893-1131, or 1188-1191. In some embodiments, the nucleotide sequence of the antisense strand consists of the nucleotide sequence set forth in any one of SEQ ID NOS: 187-369, 602-657, 893-1131, or 1188-1191.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 187-369, 602-657, 893-1131, or 1188-1191 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191.

In some embodiments, the nucleotide sequence of the antisense strand comprises the nucleotide sequence set forth in any one of SEQ ID NOS: 187-369, 602-657, 893-1131, or 1188-1191 differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the nucleotide sequence of the antisense strand comprises the nucleotide sequence set forth in any one of SEQ ID NOS: 187-369, 602-657, 893-1131, or 1188-1191.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 187-369, 602-657, 893-1131, or 1188-1191 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in any one of SEQ ID NOS: 315, 321, 355, or 356. In some embodiments, the nucleotide sequence of the antisense strand differs by no more than 3 (e.g., 0, 1, 2, or 3) nucleotides from the nucleotide sequence set forth in any one of SEQ ID NOS: 315, 321, 355, or 356. In some embodiments, the nucleotide sequence of the antisense strand differs by no more than 2 (e.g., 0, 1, or 2) nucleotides from the nucleotide sequence set forth in any one of SEQ ID NOS: 315, 321, 355, or 356. In some embodiments, the nucleotide sequence of the antisense strand differs by no more than 1 (e.g., 0 or 1) nucleotide from the nucleotide sequence set forth in any one of SEQ ID NOS: 315, 321, 355, or 356.

In some embodiments, the nucleotide sequence of the antisense strand comprises the nucleotide sequence set forth in any one of SEQ ID NOS: 315, 321, 355, or 356 differing by no more than 5 (e.g., 0, 1, 2, 3, 4, or 5) nucleotides from the nucleotide sequence set forth in any one of SEQ ID NOS: 315, 321, 355, 356. In some embodiments, the nucleotide sequence of the antisense strand comprises the nucleotide sequence set forth in any one of SEQ ID NOS: 315, 321, 355, or 356 differing by no more than 3 (e.g., 0, 1, 2, or 3) nucleotides from the nucleotide sequence set forth in any one of SEQ ID NOS: 315, 321, 355, 356. In some embodiments, the nucleotide sequence of the antisense strand comprises the nucleotide sequence set forth in any one of SEQ ID NOS: 315, 321, 355, or 356 differing by no more than 2 (e.g., 0, 1, or 2) nucleotides from the nucleotide sequence set forth in any one of SEQ ID NOS: 315, 321, 355, 356. In some embodiments, the nucleotide sequence of the antisense strand comprises the nucleotide sequence set forth in any one of SEQ ID NOS: 315, 321, 355, or 356 differing by no more than 1 (e.g., 0 or 1) nucleotide from the nucleotide sequence set forth in any one of SEQ ID NOS: 315, 321, 355, 356.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 315, 321, 355, or 356. In some embodiments, the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 315, 321, 355, or 356. In some embodiments, the nucleotide sequence of the antisense strand is at least 95%, 96%, 97%, 98%, 99% or 100% identical to the nucleotide sequence set forth in any one of SEQ ID NOS: 315, 321, 355, or 356. In some embodiments, the nucleotide sequence of the antisense strand is at least 96%, 97%, 98%, 99% or 100% identical to the nucleotide sequence set forth in any one of SEQ ID NOS: 315, 321, 355, or 356. In some embodiments, the nucleotide sequence of the antisense strand is at least 97%, identical to the nucleotide sequence set forth in any one of SEQ ID NOS: 315, 321, 355, or 356. In some embodiments, the nucleotide sequence of the antisense strand is at least 98% identical to the nucleotide sequence set forth in any one of SEQ ID NOS: 315, 321, 355, or 356. In some embodiments, the nucleotide sequence of the antisense strand is at least 99% identical to the nucleotide sequence set forth in any one of SEQ ID NOS: 315, 321, 355, or 356. In some embodiments, the nucleotide sequence of the antisense strand is 100% identical to the nucleotide sequence set forth in any one of SEQ ID NOS: 315, 321, 355, or 356.

In some embodiments, the nucleotide sequence of the antisense strand comprises the nucleotide sequence set forth in any one of SEQ ID NOS: 315, 321, 355, or 356. In some embodiments, the nucleotide sequence of the antisense strand consists of the nucleotide sequence set forth in any one of SEQ ID NOS: 315, 321, 355, or 356.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 315, 321, 355, or 356 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in any one of SEQ ID NOS: 1062, 1188, 1189, 1190, or 1191. In some embodiments, the nucleotide sequence of the antisense strand differs by no more than 3 (e.g., 0, 1, 2, or 3) nucleotides from the nucleotide sequence set forth in any one of SEQ ID NOS: 1062, 1188, 1189, 1190, or 1191. In some embodiments, the nucleotide sequence of the antisense strand differs by no more than 2 (e.g., 0, 1, or 2) nucleotides from the nucleotide sequence set forth in any one of SEQ ID NOS: 1062, 1188, 1189, 1190, or 1191. In some embodiments, the nucleotide sequence of the antisense strand differs by no more than 1 (e.g., 0 or 1) nucleotide from the nucleotide sequence set forth in any one of SEQ ID NOS: 1062, 1188, 1189, 1190, or 1191.

In some embodiments, the nucleotide sequence of the antisense strand comprises the nucleotide sequence set forth in any one of SEQ ID NOS: 1062, 1188, 1189, 1190, or 1191 differing by no more than 5 (e.g., 0, 1, 2, 3, 4, or 5) nucleotides from the nucleotide sequence set forth in any one of SEQ ID NOS: 315, 321, 355, 356. In some embodiments, the nucleotide sequence of the antisense strand comprises the nucleotide sequence set forth in any one of SEQ ID NOS: 1062, 1188, 1189, 1190, or 1191 differing by no more than 3 (e.g., 0, 1, 2, or 3) nucleotides from the nucleotide sequence set forth in any one of SEQ ID NOS: 1062, 1188, 1189, 1190, or 1191. In some embodiments, the nucleotide sequence of the antisense strand comprises the nucleotide sequence set forth in any one of SEQ ID NOS: 1062, 1188, 1189, 1190, or 1191 differing by no more than 2 (e.g., 0, 1, or 2) nucleotides from the nucleotide sequence set forth in any one of SEQ ID NOS: 1062, 1188, 1189, 1190, or 1191. In some embodiments, the nucleotide sequence of the antisense strand comprises the nucleotide sequence set forth in any one of SEQ ID NOS: 1062, 1188, 1189, 1190, or 1191 differing by no more than 1 (e.g., 0 or 1) nucleotide from the nucleotide sequence set forth in any one of SEQ ID NOS: 1062, 1188, 1189, 1190, or 1191.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 1062, 1188, 1189, 1190, or 1191. In some embodiments, the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 1062, 1188, 1189, 1190, or 1191. In some embodiments, the nucleotide sequence of the antisense strand is at least 95%, 96%, 97%, 98%, 99% or 100% identical to the nucleotide sequence set forth in any one of SEQ ID NOS: 1062, 1188, 1189, 1190, or 1191. In some embodiments, the nucleotide sequence of the antisense strand is at least 96%, 97%, 98%, 99% or 100% identical to the nucleotide sequence set forth in any one of SEQ ID NOS: 1062, 1188, 1189, 1190, or 1191. In some embodiments, the nucleotide sequence of the antisense strand is at least 97%, identical to the nucleotide sequence set forth in any one of SEQ ID NOS: 1062, 1188, 1189, 1190, or 1191. In some embodiments, the nucleotide sequence of the antisense strand is at least 98% identical to the nucleotide sequence set forth in any one of SEQ ID NOS: 1062, 1188, 1189, 1190, or 1191. In some embodiments, the nucleotide sequence of the antisense strand is at least 99% identical to the nucleotide sequence set forth in any one of SEQ ID NOS: 1062, 1188, 1189, 1190, or 1191. In some embodiments, the nucleotide sequence of the antisense strand is 100% identical to the nucleotide sequence set forth in any one of SEQ ID NOS: 1062, 1188, 1189, 1190, or 1191.

In some embodiments, the nucleotide sequence of the antisense strand comprises the nucleotide sequence set forth in any one of SEQ ID NOS: 1062, 1188, 1189, 1190, or 1191. In some embodiments, the nucleotide sequence of the antisense strand consists of the nucleotide sequence set forth in any one of SEQ ID NOS: 1062, 1188, 1189, 1190, or 1191.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 1062, 1188, 1189, 1190, or 1191 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 315. In some embodiments, the nucleotide sequence of the antisense strand differs by no more than 3 (e.g., 0, 1, 2, or 3) nucleotides from the nucleotide sequence set forth in SEQ ID NO: 315. In some embodiments, the nucleotide sequence of the antisense strand differs by no more than 2 (e.g., 0, 1, or 2) nucleotides from the nucleotide sequence set forth in SEQ ID NO: 315. In some embodiments, the nucleotide sequence of the antisense strand differs by no more than 1 (e.g., 0 or 1) nucleotide from the nucleotide sequence set forth in SEQ ID NO: 315.

In some embodiments, the nucleotide sequence of the antisense strand comprises the nucleotide sequence set forth in SEQ ID NO: 315 differing by no more than 5 (e.g., 0, 1, 2, 3, 4, or 5) nucleotides from the nucleotide sequence set forth in SEQ ID NO: 315. In some embodiments, the nucleotide sequence of the antisense strand comprises the nucleotide sequence set forth in SEQ ID NO: 315 differing by no more than 3 (e.g., 0, 1, 2, or 3) nucleotides from the nucleotide sequence set forth in SEQ ID NO: 315. In some embodiments, the nucleotide sequence of the antisense strand comprises the nucleotide sequence set forth in SEQ ID NO: 315 differing by no more than 2 (e.g., 0, 1, or 2) nucleotides from the nucleotide sequence set forth in SEQ ID NO: 315. In some embodiments, the nucleotide sequence of the antisense strand comprises the nucleotide sequence set forth in SEQ ID NO: 315 differing by no more than 1 (e.g., 0 or 1) nucleotide from nucleotide sequence set forth in SEQ ID NO: 315.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 315. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 315. In some embodiments, the nucleotide sequence of the antisense strand is at least 95%, 96%, 97%, 98%, 99% or 100% identical to the nucleotide sequence set forth in SEQ ID NO: 315. In some embodiments, the nucleotide sequence of the antisense strand is at least 96%, 97%, 98%, 99% or 100% identical to the nucleotide sequence set forth in SEQ ID NO: 315. In some embodiments, the nucleotide sequence of the antisense strand is at least 97%, identical to the nucleotide sequence set forth in SEQ ID NO: 315. In some embodiments, the nucleotide sequence of the antisense strand is at least 98% identical to the nucleotide sequence set forth in SEQ ID NO: 315. In some embodiments, the nucleotide sequence of the antisense strand is at least 99% identical to the nucleotide sequence set forth in SEQ ID NO: 315. In some embodiments, the nucleotide sequence of the antisense strand is 100% identical to the nucleotide sequence set forth in SEQ ID NO: 315.

In some embodiments, the nucleotide sequence of the antisense strand comprises the nucleotide sequence set forth in SEQ ID NO: 315. In some embodiments, the nucleotide sequence of the antisense strand consists of the nucleotide sequence set forth in SEQ ID NO: 315.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 315 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 315. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 315. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 315. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 315.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 315. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 315. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 315.

In some embodiments, the nucleotide sequence of the antisense strand comprises the nucleotide sequence set forth in SEQ ID NO: 315 differing by no more than 3 (e.g., 0, 1, 2, or 3) nucleotides from the nucleotide sequence set forth in any one of SEQ ID NOS: 315. In some embodiments, the nucleotide sequence of the antisense strand comprises the nucleotide sequence set forth in SEQ ID NO: 315.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 315 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 321. In some embodiments, the nucleotide sequence of the antisense strand differs by no more than 3 (e.g., 0, 1, 2, or 3) nucleotides from the nucleotide sequence set forth in SEQ ID NO: 321. In some embodiments, the nucleotide sequence of the antisense strand differs by no more than 2 (e.g., 0, 1, or 2) nucleotides from the nucleotide sequence set forth in SEQ ID NO: 321. In some embodiments, the nucleotide sequence of the antisense strand differs by no more than 1 (e.g., 0 or 1) nucleotide from the nucleotide sequence set forth in SEQ ID NO: 321.

In some embodiments, the nucleotide sequence of the antisense strand comprises the nucleotide sequence set forth in SEQ ID NO: 321 differing by no more than 5 (e.g., 0, 1, 2, 3, 4, or 5) nucleotides from the nucleotide sequence set forth in SEQ ID NO: 321. In some embodiments, the nucleotide sequence of the antisense strand comprises the nucleotide sequence set forth in SEQ ID NO: 321 differing by no more than 3 (e.g., 0, 1, 2, or 3) nucleotides from the nucleotide sequence set forth in SEQ ID NO: 321. In some embodiments, the nucleotide sequence of the antisense strand comprises the nucleotide sequence set forth in SEQ ID NO: 321 differing by no more than 2 (e.g., 0, 1, or 2) nucleotides from the nucleotide sequence set forth in SEQ ID NO: 321. In some embodiments, the nucleotide sequence of the antisense strand comprises the nucleotide sequence set forth in SEQ ID NO: 321 differing by no more than 1 (e.g., 0 or 1) nucleotide from nucleotide sequence set forth in SEQ ID NO: 321.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 321. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 321. In some embodiments, the nucleotide sequence of the antisense strand is at least 95%, 96%, 97%, 98%, 99% or 100% identical to the nucleotide sequence set forth in SEQ ID NO: 321. In some embodiments, the nucleotide sequence of the antisense strand is at least 96%, 97%, 98%, 99% or 100% identical to the nucleotide sequence set forth in SEQ ID NO: 321. In some embodiments, the nucleotide sequence of the antisense strand is at least 97%, identical to the nucleotide sequence set forth in SEQ ID NO: 321. In some embodiments, the nucleotide sequence of the antisense strand is at least 98% identical to the nucleotide sequence set forth in SEQ ID NO: 321. In some embodiments, the nucleotide sequence of the antisense strand is at least 99% identical to the nucleotide sequence set forth in SEQ ID NO: 321. In some embodiments, the nucleotide sequence of the antisense strand is 100% identical to the nucleotide sequence set forth in SEQ ID NO: 321.

In some embodiments, the nucleotide sequence of the antisense strand comprises the nucleotide sequence set forth in SEQ ID NO: 321. In some embodiments, the nucleotide sequence of the antisense strand consists of the nucleotide sequence set forth in SEQ ID NO: 321.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 321 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 321. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 321. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 321. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 321.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 321. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 321. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 321.

In some embodiments, the nucleotide sequence of the antisense strand comprises the nucleotide sequence set forth in SEQ ID NO: 321 differing by no more than 3 (e.g., 0, 1, 2, or 3) nucleotides from the nucleotide sequence set forth in any one of SEQ ID NOS: 321. In some embodiments, the nucleotide sequence of the antisense strand comprises the nucleotide sequence set forth in SEQ ID NO: 321.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 321 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 355. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 355. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 355. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 355.

In some embodiments, the nucleotide sequence of the antisense strand differs by no more than 5 (e.g., 0, 1, 2, 3, 4, or 5) nucleotides from the nucleotide sequence set forth in SEQ ID NO: 355. In some embodiments, the nucleotide sequence of the antisense strand differs by no more than 3 (e.g., 0, 1, 2, or 3) nucleotides from the nucleotide sequence set forth in SEQ ID NO: 355. In some embodiments, the nucleotide sequence of the antisense strand differs by no more than 2 (e.g., 0, 1, or 2) nucleotides from the nucleotide sequence set forth in SEQ ID NO: 355. In some embodiments, the nucleotide sequence of the antisense strand differs by no more than 1 (e.g., 0 or 1) nucleotide from the nucleotide sequence set forth in SEQ ID NO: 355.

In some embodiments, the nucleotide sequence of the antisense strand comprises the nucleotide sequence set forth in SEQ ID NO: 355 differing by no more than 5 (e.g., 0, 1, 2, 3, 4, or 5) nucleotides from the nucleotide sequence set forth in SEQ ID NO: 355. In some embodiments, the nucleotide sequence of the antisense strand comprises the nucleotide sequence set forth in SEQ ID NO: 355 differing by no more than 3 (e.g., 0, 1, 2, or 3) nucleotides from the nucleotide sequence set forth in SEQ ID NO: 355. In some embodiments, the nucleotide sequence of the antisense strand comprises the nucleotide sequence set forth in SEQ ID NO: 355 differing by no more than 2 (e.g., 0, 1, or 2) nucleotides from the nucleotide sequence set forth in SEQ ID NO: 355. In some embodiments, the nucleotide sequence of the antisense strand comprises the nucleotide sequence set forth in SEQ ID NO: 355 differing by no more than 1 (e.g., 0 or 1) nucleotide from nucleotide sequence set forth in SEQ ID NO: 355.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 355. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 355. In some embodiments, the nucleotide sequence of the antisense strand is at least 95%, 96%, 97%, 98%, 99% or 100% identical to the nucleotide sequence set forth in SEQ ID NO: 355. In some embodiments, the nucleotide sequence of the antisense strand is at least 96%, 97%, 98%, 99% or 100% identical to the nucleotide sequence set forth in SEQ ID NO: 355. In some embodiments, the nucleotide sequence of the antisense strand is at least 97%, identical to the nucleotide sequence set forth in SEQ ID NO: 355. In some embodiments, the nucleotide sequence of the antisense strand is at least 98% identical to the nucleotide sequence set forth in SEQ ID NO: 355. In some embodiments, the nucleotide sequence of the antisense strand is at least 99% identical to the nucleotide sequence set forth in SEQ ID NO: 355. In some embodiments, the nucleotide sequence of the antisense strand is 100% identical to the nucleotide sequence set forth in SEQ ID NO: 355.

In some embodiments, the nucleotide sequence of the antisense strand comprises the nucleotide sequence set forth in SEQ ID NO: 355. In some embodiments, the nucleotide sequence of the antisense strand consists of the nucleotide sequence set forth in SEQ ID NO: 355.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 355 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 355. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 355. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 355.

In some embodiments, the nucleotide sequence of the antisense strand comprises the nucleotide sequence set forth in SEQ ID NO: 355 differing by no more than 3 (e.g., 0, 1, 2, or 3) nucleotides from the nucleotide sequence set forth in any one of SEQ ID NOS: 355. In some embodiments, the nucleotide sequence of the antisense strand comprises the nucleotide sequence set forth in SEQ ID NO: 355.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 355 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 356. In some embodiments, the nucleotide sequence of the antisense strand differs by no more than 3 (e.g., 0, 1, 2, or 3) nucleotides from the nucleotide sequence set forth in SEQ ID NO: 356. In some embodiments, the nucleotide sequence of the antisense strand differs by no more than 2 (e.g., 0, 1, or 2) nucleotides from the nucleotide sequence set forth in SEQ ID NO: 356. In some embodiments, the nucleotide sequence of the antisense strand differs by no more than 1 (e.g., 0 or 1) nucleotide from the nucleotide sequence set forth in SEQ ID NO: 356.

In some embodiments, the nucleotide sequence of the antisense strand comprises the nucleotide sequence set forth in SEQ ID NO: 356 differing by no more than 5 (e.g., 0, 1, 2, 3, 4, or 5) nucleotides from the nucleotide sequence set forth in SEQ ID NO: 356. In some embodiments, the nucleotide sequence of the antisense strand comprises the nucleotide sequence set forth in SEQ ID NO: 356 differing by no more than 3 (e.g., 0, 1, 2, or 3) nucleotides from the nucleotide sequence set forth in SEQ ID NO: 356. In some embodiments, the nucleotide sequence of the antisense strand comprises the nucleotide sequence set forth in SEQ ID NO: 356 differing by no more than 2 (e.g., 0, 1, or 2) nucleotides from the nucleotide sequence set forth in SEQ ID NO: 356. In some embodiments, the nucleotide sequence of the antisense strand comprises the nucleotide sequence set forth in SEQ ID NO: 356 differing by no more than 1 (e.g., 0 or 1) nucleotide from nucleotide sequence set forth in SEQ ID NO: 356.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 356. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 356. In some embodiments, the nucleotide sequence of the antisense strand is at least 95%, 96%, 97%, 98%, 99% or 100% identical to the nucleotide sequence set forth in SEQ ID NO: 356. In some embodiments, the nucleotide sequence of the antisense strand is at least 96%, 97%, 98%, 99% or 100% identical to the nucleotide sequence set forth in SEQ ID NO: 356. In some embodiments, the nucleotide sequence of the antisense strand is at least 97%, identical to the nucleotide sequence set forth in SEQ ID NO: 356. In some embodiments, the nucleotide sequence of the antisense strand is at least 98% identical to the nucleotide sequence set forth in SEQ ID NO: 356. In some embodiments, the nucleotide sequence of the antisense strand is at least 99% identical to the nucleotide sequence set forth in SEQ ID NO: 356. In some embodiments, the nucleotide sequence of the antisense strand is 100% identical to the nucleotide sequence set forth in SEQ ID NO: 356.

In some embodiments, the nucleotide sequence of the antisense strand comprises the nucleotide sequence set forth in SEQ ID NO: 356. In some embodiments, the nucleotide sequence of the antisense strand consists of the nucleotide sequence set forth in SEQ ID NO: 356.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 356 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 356. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 356. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 356. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 356.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 356. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 356. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 356.

In some embodiments, the nucleotide sequence of the antisense strand comprises the nucleotide sequence set forth in SEQ ID NO: 356 differing by no more than 3 (e.g., 0, 1, 2, or 3) nucleotides from the nucleotide sequence set forth in any one of SEQ ID NOS: 356. In some embodiments, the nucleotide sequence of the antisense strand comprises the nucleotide sequence set forth in SEQ ID NO: 356.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 356 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 1188. In some embodiments, the nucleotide sequence of the antisense strand differs by no more than 3 (e.g., 0, 1, 2, or 3) nucleotides from the nucleotide sequence set forth in SEQ ID NO: 1188. In some embodiments, the nucleotide sequence of the antisense strand differs by no more than 2 (e.g., 0, 1, or 2) nucleotides from the nucleotide sequence set forth in SEQ ID NO: 1188. In some embodiments, the nucleotide sequence of the antisense strand differs by no more than 1 (e.g., 0 or 1) nucleotide from the nucleotide sequence set forth in SEQ ID NO: 1188.

In some embodiments, the nucleotide sequence of the antisense strand comprises the nucleotide sequence set forth in SEQ ID NO: 1188 differing by no more than 5 (e.g., 0, 1, 2, 3, 4, or 5) nucleotides from the nucleotide sequence set forth in SEQ ID NO: 1188. In some embodiments, the nucleotide sequence of the antisense strand comprises the nucleotide sequence set forth in SEQ ID NO: 1188 differing by no more than 3 (e.g., 0, 1, 2, or 3) nucleotides from the nucleotide sequence set forth in SEQ ID NO: 1188. In some embodiments, the nucleotide sequence of the antisense strand comprises the nucleotide sequence set forth in SEQ ID NO: 1188 differing by no more than 2 (e.g., 0, 1, or 2) nucleotides from the nucleotide sequence set forth in SEQ ID NO: 1188. In some embodiments, the nucleotide sequence of the antisense strand comprises the nucleotide sequence set forth in SEQ ID NO: 1188 differing by no more than 1 (e.g., 0 or 1) nucleotide from nucleotide sequence set forth in SEQ ID NO: 1188.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1188. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1188. In some embodiments, the nucleotide sequence of the antisense strand is at least 95%, 96%, 97%, 98%, 99% or 100% identical to the nucleotide sequence set forth in SEQ ID NO: 1188. In some embodiments, the nucleotide sequence of the antisense strand is at least 96%, 97%, 98%, 99% or 100% identical to the nucleotide sequence set forth in SEQ ID NO: 1188. In some embodiments, the nucleotide sequence of the antisense strand is at least 97%, identical to the nucleotide sequence set forth in SEQ ID NO: 1188. In some embodiments, the nucleotide sequence of the antisense strand is at least 98% identical to the nucleotide sequence set forth in SEQ ID NO: 1188. In some embodiments, the nucleotide sequence of the antisense strand is at least 99% identical to the nucleotide sequence set forth in SEQ ID NO: 1188. In some embodiments, the nucleotide sequence of the antisense strand is 100% identical to the nucleotide sequence set forth in SEQ ID NO: 1188.

In some embodiments, the nucleotide sequence of the antisense strand comprises the nucleotide sequence set forth in SEQ ID NO: 1188. In some embodiments, the nucleotide sequence of the antisense strand consists of the nucleotide sequence set forth in SEQ ID NO: 1188.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1188 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1188. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 1188. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1188. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1188.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1188. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1188. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1188.

In some embodiments, the nucleotide sequence of the antisense strand comprises the nucleotide sequence set forth in SEQ ID NO: 1188 differing by no more than 3 (e.g., 0, 1, 2, or 3) nucleotides from the nucleotide sequence set forth in any one of SEQ ID NOS: 1188. In some embodiments, the nucleotide sequence of the antisense strand comprises the nucleotide sequence set forth in SEQ ID NO: 1188.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1188 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 1190. In some embodiments, the nucleotide sequence of the antisense strand differs by no more than 3 (e.g., 0, 1, 2, or 3) nucleotides from the nucleotide sequence set forth in SEQ ID NO: 1190. In some embodiments, the nucleotide sequence of the antisense strand differs by no more than 2 (e.g., 0, 1, or 2) nucleotides from the nucleotide sequence set forth in SEQ ID NO: 1190. In some embodiments, the nucleotide sequence of the antisense strand differs by no more than 1 (e.g., 0 or 1) nucleotide from the nucleotide sequence set forth in SEQ ID NO: 1190.

In some embodiments, the nucleotide sequence of the antisense strand comprises the nucleotide sequence set forth in SEQ ID NO: 1190 differing by no more than 5 (e.g., 0, 1, 2, 3, 4, or 5) nucleotides from the nucleotide sequence set forth in SEQ ID NO: 1190. In some embodiments, the nucleotide sequence of the antisense strand comprises the nucleotide sequence set forth in SEQ ID NO: 1190 differing by no more than 3 (e.g., 0, 1, 2, or 3) nucleotides from the nucleotide sequence set forth in SEQ ID NO: 1190. In some embodiments, the nucleotide sequence of the antisense strand comprises the nucleotide sequence set forth in SEQ ID NO: 1190 differing by no more than 2 (e.g., 0, 1, or 2) nucleotides from the nucleotide sequence set forth in SEQ ID NO: 1190. In some embodiments, the nucleotide sequence of the antisense strand comprises the nucleotide sequence set forth in SEQ ID NO: 1190 differing by no more than 1 (e.g., 0 or 1) nucleotide from nucleotide sequence set forth in SEQ ID NO: 1190.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1190. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1190. In some embodiments, the nucleotide sequence of the antisense strand is at least 95%, 96%, 97%, 98%, 99% or 100% identical to the nucleotide sequence set forth in SEQ ID NO: 1190. In some embodiments, the nucleotide sequence of the antisense strand is at least 96%, 97%, 98%, 99% or 100% identical to the nucleotide sequence set forth in SEQ ID NO: 1190. In some embodiments, the nucleotide sequence of the antisense strand is at least 97%, identical to the nucleotide sequence set forth in SEQ ID NO: 1190. In some embodiments, the nucleotide sequence of the antisense strand is at least 98% identical to the nucleotide sequence set forth in SEQ ID NO: 1190. In some embodiments, the nucleotide sequence of the antisense strand is at least 99% identical to the nucleotide sequence set forth in SEQ ID NO: 1190. In some embodiments, the nucleotide sequence of the antisense strand is 100% identical to the nucleotide sequence set forth in SEQ ID NO: 1190.

In some embodiments, the nucleotide sequence of the antisense strand comprises the nucleotide sequence set forth in SEQ ID NO: 1190. In some embodiments, the nucleotide sequence of the antisense strand consists of the nucleotide sequence set forth in SEQ ID NO: 1190.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1190 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1190. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 1190. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1190. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1190.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1190. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1190. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1190.

In some embodiments, the nucleotide sequence of the antisense strand comprises the nucleotide sequence set forth in SEQ ID NO: 1190 differing by no more than 3 (e.g., 0, 1, 2, or 3) nucleotides from the nucleotide sequence set forth in any one of SEQ ID NOS: 1190. In some embodiments, the nucleotide sequence of the antisense strand comprises the nucleotide sequence set forth in SEQ ID NO: 1190.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1190 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 1191. In some embodiments, the nucleotide sequence of the antisense strand differs by no more than 3 (e.g., 0, 1, 2, or 3) nucleotides from the nucleotide sequence set forth in SEQ ID NO: 1191. In some embodiments, the nucleotide sequence of the antisense strand differs by no more than 2 (e.g., 0, 1, or 2) nucleotides from the nucleotide sequence set forth in SEQ ID NO: 1191. In some embodiments, the nucleotide sequence of the antisense strand differs by no more than 1 (e.g., 0 or 1) nucleotide from the nucleotide sequence set forth in SEQ ID NO: 1191.

In some embodiments, the nucleotide sequence of the antisense strand comprises the nucleotide sequence set forth in SEQ ID NO: 1191 differing by no more than 5 (e.g., 0, 1, 2, 3, 4, or 5) nucleotides from the nucleotide sequence set forth in SEQ ID NO: 1191. In some embodiments, the nucleotide sequence of the antisense strand comprises the nucleotide sequence set forth in SEQ ID NO: 1191 differing by no more than 3 (e.g., 0, 1, 2, or 3) nucleotides from the nucleotide sequence set forth in SEQ ID NO: 1191. In some embodiments, the nucleotide sequence of the antisense strand comprises the nucleotide sequence set forth in SEQ ID NO: 1191 differing by no more than 2 (e.g., 0, 1, or 2) nucleotides from the nucleotide sequence set forth in SEQ ID NO: 1191. In some embodiments, the nucleotide sequence of the antisense strand comprises the nucleotide sequence set forth in SEQ ID NO: 1191 differing by no more than 1 (e.g., 0 or 1) nucleotide from nucleotide sequence set forth in SEQ ID NO: 1191.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1191. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1191. In some embodiments, the nucleotide sequence of the antisense strand is at least 95%, 96%, 97%, 98%, 99% or 100% identical to the nucleotide sequence set forth in SEQ ID NO: 1191. In some embodiments, the nucleotide sequence of the antisense strand is at least 96%, 97%, 98%, 99% or 100% identical to the nucleotide sequence set forth in SEQ ID NO: 1191. In some embodiments, the nucleotide sequence of the antisense strand is at least 97%, identical to the nucleotide sequence set forth in SEQ ID NO: 1191. In some embodiments, the nucleotide sequence of the antisense strand is at least 98% identical to the nucleotide sequence set forth in SEQ ID NO: 1191. In some embodiments, the nucleotide sequence of the antisense strand is at least 99% identical to the nucleotide sequence set forth in SEQ ID NO: 1191. In some embodiments, the nucleotide sequence of the antisense strand is 100% identical to the nucleotide sequence set forth in SEQ ID NO: 1191.

In some embodiments, the nucleotide sequence of the antisense strand comprises the nucleotide sequence set forth in SEQ ID NO: 1191. In some embodiments, the nucleotide sequence of the antisense strand consists of the nucleotide sequence set forth in SEQ ID NO: 1191.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1191 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1191. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 1191. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1191. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1191.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1191. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1191. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1191.

In some embodiments, the nucleotide sequence of the antisense strand comprises the nucleotide sequence set forth in SEQ ID NO: 1191 differing by no more than 3 (e.g., 0, 1, 2, or 3) nucleotides from the nucleotide sequence set forth in any one of SEQ ID NOS: 1191. In some embodiments, the nucleotide sequence of the antisense strand comprises the nucleotide sequence set forth in SEQ ID NO: 1191.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1191 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 1189. In some embodiments, the nucleotide sequence of the antisense strand differs by no more than 3 (e.g., 0, 1, 2, or 3) nucleotides from the nucleotide sequence set forth in SEQ ID NO: 1189. In some embodiments, the nucleotide sequence of the antisense strand differs by no more than 2 (e.g., 0, 1, or 2) nucleotides from the nucleotide sequence set forth in SEQ ID NO: 1189. In some embodiments, the nucleotide sequence of the antisense strand differs by no more than 1 (e.g., 0 or 1) nucleotide from the nucleotide sequence set forth in SEQ ID NO: 1189.

In some embodiments, the nucleotide sequence of the antisense strand comprises the nucleotide sequence set forth in SEQ ID NO: 1189 differing by no more than 5 (e.g., 0, 1, 2, 3, 4, or 5) nucleotides from the nucleotide sequence set forth in SEQ ID NO: 1189. In some embodiments, the nucleotide sequence of the antisense strand comprises the nucleotide sequence set forth in SEQ ID NO: 1189 differing by no more than 3 (e.g., 0, 1, 2, or 3) nucleotides from the nucleotide sequence set forth in SEQ ID NO: 1189. In some embodiments, the nucleotide sequence of the antisense strand comprises the nucleotide sequence set forth in SEQ ID NO: 1189 differing by no more than 2 (e.g., 0, 1, or 2) nucleotides from the nucleotide sequence set forth in SEQ ID NO: 1189. In some embodiments, the nucleotide sequence of the antisense strand comprises the nucleotide sequence set forth in SEQ ID NO: 1189 differing by no more than 1 (e.g., 0 or 1) nucleotide from nucleotide sequence set forth in SEQ ID NO: 1189.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1189. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1189. In some embodiments, the nucleotide sequence of the antisense strand is at least 95%, 96%, 97%, 98%, 99% or 100% identical to the nucleotide sequence set forth in SEQ ID NO: 1189. In some embodiments, the nucleotide sequence of the antisense strand is at least 96%, 97%, 98%, 99% or 100% identical to the nucleotide sequence set forth in SEQ ID NO: 1189. In some embodiments, the nucleotide sequence of the antisense strand is at least 97%, identical to the nucleotide sequence set forth in SEQ ID NO: 1189. In some embodiments, the nucleotide sequence of the antisense strand is at least 98% identical to the nucleotide sequence set forth in SEQ ID NO: 1189. In some embodiments, the nucleotide sequence of the antisense strand is at least 99% identical to the nucleotide sequence set forth in SEQ ID NO: 1189. In some embodiments, the nucleotide sequence of the antisense strand is 100% identical to the nucleotide sequence set forth in SEQ ID NO: 1189.

In some embodiments, the nucleotide sequence of the antisense strand comprises the nucleotide sequence set forth in SEQ ID NO: 1189. In some embodiments, the nucleotide sequence of the antisense strand consists of the nucleotide sequence set forth in SEQ ID NO: 1189.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1189 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1189. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 1189. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1189. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1189.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1189. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1189. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1189.

In some embodiments, the nucleotide sequence of the antisense strand comprises the nucleotide sequence set forth in SEQ ID NO: 1189 differing by no more than 3 (e.g., 0, 1, 2, or 3) nucleotides from the nucleotide sequence set forth in any one of SEQ ID NOS: 1189. In some embodiments, the nucleotide sequence of the antisense strand comprises the nucleotide sequence set forth in SEQ ID NO: 1189.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1189 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in SEQ ID NO: 1062. In some embodiments, the nucleotide sequence of the antisense strand differs by no more than 3 (e.g., 0, 1, 2, or 3) nucleotides from the nucleotide sequence set forth in SEQ ID NO: 1062. In some embodiments, the nucleotide sequence of the antisense strand differs by no more than 2 (e.g., 0, 1, or 2) nucleotides from the nucleotide sequence set forth in SEQ ID NO: 1062. In some embodiments, the nucleotide sequence of the antisense strand differs by no more than 1 (e.g., 0 or 1) nucleotide from the nucleotide sequence set forth in SEQ ID NO: 1062.

In some embodiments, the nucleotide sequence of the antisense strand comprises the nucleotide sequence set forth in SEQ ID NO: 1062 differing by no more than 5 (e.g., 0, 1, 2, 3, 4, or 5) nucleotides from the nucleotide sequence set forth in SEQ ID NO: 1062. In some embodiments, the nucleotide sequence of the antisense strand comprises the nucleotide sequence set forth in SEQ ID NO: 1062 differing by no more than 3 (e.g., 0, 1, 2, or 3) nucleotides from the nucleotide sequence set forth in SEQ ID NO: 1062. In some embodiments, the nucleotide sequence of the antisense strand comprises the nucleotide sequence set forth in SEQ ID NO: 1062 differing by no more than 2 (e.g., 0, 1, or 2) nucleotides from the nucleotide sequence set forth in SEQ ID NO: 1062. In some embodiments, the nucleotide sequence of the antisense strand comprises the nucleotide sequence set forth in SEQ ID NO: 1062 differing by no more than 1 (e.g., 0 or 1) nucleotide from nucleotide sequence set forth in SEQ ID NO: 1062.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1062. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1062. In some embodiments, the nucleotide sequence of the antisense strand is at least 95%, 96%, 97%, 98%, 99% or 100% identical to the nucleotide sequence set forth in SEQ ID NO: 1062. In some embodiments, the nucleotide sequence of the antisense strand is at least 96%, 97%, 98%, 99% or 100% identical to the nucleotide sequence set forth in SEQ ID NO: 1062. In some embodiments, the nucleotide sequence of the antisense strand is at least 97%, identical to the nucleotide sequence set forth in SEQ ID NO: 1062. In some embodiments, the nucleotide sequence of the antisense strand is at least 98% identical to the nucleotide sequence set forth in SEQ ID NO: 1062. In some embodiments, the nucleotide sequence of the antisense strand is at least 99% identical to the nucleotide sequence set forth in SEQ ID NO: 1062. In some embodiments, the nucleotide sequence of the antisense strand is 100% identical to the nucleotide sequence set forth in SEQ ID NO: 1062.

In some embodiments, the nucleotide sequence of the antisense strand comprises the nucleotide sequence set forth in SEQ ID NO: 1062. In some embodiments, the nucleotide sequence of the antisense strand consists of the nucleotide sequence set forth in SEQ ID NO: 1062.

Table 2 or Table 3 further identifies the target nucleic acid molecule within the cited reference CIDEB mRNA transcript (SEQ ID NO: 1) targeted by each of the antisense strands. As such, the disclosure further provides an antisense strand wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1062 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1062. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NO: 1062. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1062. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1062.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1062. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1062. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1062.

In some embodiments, the nucleotide sequence of the antisense strand comprises the nucleotide sequence set forth in SEQ ID NO: 1062 differing by no more than 3 (e.g., 0, 1, 2, or 3) nucleotides from the nucleotide sequence set forth in any one of SEQ ID NOS: 1062. In some embodiments, the nucleotide sequence of the antisense strand comprises the nucleotide sequence set forth in SEQ ID NO: 1062.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1062 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence of any one of the antisense strands of any one of dsRNA agents 1-482. In some embodiments, the nucleotide sequence of the antisense strand differs by no more than 3 (e.g., 0, 1, 2, or 3) nucleotides from the nucleotide sequence of any one of the antisense strands of any one of dsRNA agents 1-482. In some embodiments, the nucleotide sequence of the antisense strand differs by no more than 2 (e.g., 0, 1, or 2) nucleotides from the nucleotide sequence of any one of the antisense strands of any one of dsRNA agents 1-482. In some embodiments, the nucleotide sequence of the antisense strand differs by no more than 1 (e.g., 0 or 1) nucleotide from the nucleotide sequence of any one of the antisense strands of any one of dsRNA agents 1-482.

In some embodiments, the nucleotide sequence of the antisense strand comprises the nucleotide sequence of any one of the antisense strands of any one of dsRNA agents 1-482 differing by no more than 5 (e.g., 0, 1, 2, 3, 4, or 5) nucleotides from the antisense strand of any one of dsRNA agents 1-482. In some embodiments, the nucleotide sequence of the antisense strand comprises the nucleotide sequence of any one of the antisense strands of any one of dsRNA agents 1-482 differing by no more than 3 (e.g., 0, 1, 2, or 3) nucleotides from the antisense strand of any one of dsRNA agents 1-482. In some embodiments, the nucleotide sequence of the antisense strand comprises the nucleotide sequence of any one of the antisense strands of any one of dsRNA agents 1-482 differing by no more than 2 (e.g., 0, 1, or 2) nucleotides from the antisense strand of any one of dsRNA agents 1-482. In some embodiments, the nucleotide sequence of the antisense strand comprises the nucleotide sequence of any one of the antisense strands of any one of dsRNA agents 1-482 differing by no more than 1 (e.g., 0 or 1) nucleotide from the antisense strand of any one of dsRNA agents 1-482.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands of any one of dsRNA agents 1-482. In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands of any one of dsRNA agents 1-482. In some embodiments, the nucleotide sequence of the antisense strand is at least 95%, 96%, 97%, 98%, 99% or 100% identical to the nucleotide sequence of any one of the antisense strands of any one of dsRNA agents 1-482. In some embodiments, the nucleotide sequence of the antisense strand is at least 96%, 97%, 98%, 99% or 100% identical to the nucleotide sequence of any one of the antisense strands of any one of dsRNA agents 1-482. In some embodiments, the nucleotide sequence of the antisense strand is at least 97%, identical to the nucleotide sequence of any one of the antisense strands of any one of dsRNA agents 1-482. In some embodiments, the nucleotide sequence of the antisense strand is at least 98% identical to the nucleotide sequence of any one of the antisense strands of any one of dsRNA agents 1-482. In some embodiments, the nucleotide sequence of the antisense strand is at least 99% identical to the nucleotide sequence of any one of the antisense strands of any one of dsRNA agents 1-482. In some embodiments, the nucleotide sequence of the antisense strand is 100% identical to the nucleotide sequence of any one of the antisense strands of any one of dsRNA agents 1-482.

In some embodiments, the nucleotide sequence of the antisense strand comprises the nucleotide sequence of any one of the antisense strands of any one of dsRNA agents 1-482. In some embodiments, the nucleotide sequence of the antisense strand consists of the nucleotide sequence of any one of the antisense strands of any one of dsRNA agents 1-482.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence of any one of the antisense strands of any one of dsRNA agents 1-482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-482. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-482. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-482. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the antisense strands of any one of dsRNA agents 1-482.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 1-482. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 1-482. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands of any one of dsRNA agents 1-482.

In some embodiments, the nucleotide sequence of the antisense strand comprises the nucleotide sequence of any one of the antisense strands of any one of dsRNA agents 1-482 differing by no more than 3 (e.g., 0, 1, 2, or 3) nucleotides from the antisense strand. In some embodiments, the nucleotide sequence of the antisense strand comprises the nucleotide sequence of any one of the antisense strands of any one of dsRNA agents 1-482.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence of any one of the antisense strands of any one of dsRNA agents 129, 135, 169, 170, 409, 479, 480, 481, or 482. In some embodiments, the nucleotide sequence of the antisense strand differs by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the nucleotide sequence of the antisense strand differs by no more than 2 (e.g., 0, 1, or 2) nucleotides from the nucleotide sequence of any one of the antisense strands of any one of dsRNA agents 129, 135, 169, 170, 409, 479, 480, 481, or 482. In some embodiments, the nucleotide sequence of the antisense strand differs by no more than 1 (e.g., 0 or 1) nucleotide from the nucleotide sequence of any one of the antisense strands of any one of dsRNA agents 129, 135, 169, 170, 409, 479, 480, 481, or 482.

In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 5 (e.g., 0, 1, 2, 3, 4, or 5) nucleotides from the antisense strand of any one of dsRNA agents 129, 135, 169, 170, 409, 479, 480, 481, or 482. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 3 (e.g., 0, 1, 2, or 3) nucleotides from the antisense strand of any one of dsRNA agents 129, 135, 169, 170, 409, 479, 480, 481, or 482. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 2 (e.g., 0, 1, or 2) nucleotides from the antisense strand of any one of dsRNA agents 129, 135, 169, 170, 409, 479, 480, 481, or 482. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 1 (e.g., 0 or 1) nucleotide from the antisense strand of any one of dsRNA agents 129, 135, 169, 170, 409, 479, 480, 481, or 482.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands of any one of dsRNA agents 129, 135, 169, 170, 409, 479, 480, 481, or 482. In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands of any one of dsRNA agents 129, 135, 169, 170, 409, 479, 480, 481, or 482. In some embodiments, the nucleotide sequence of the antisense strand is at least 95%, 96%, 97%, 98%, 99% or 100% identical to the nucleotide sequence of any one of the antisense strands of any one of dsRNA agents 129, 135, 169, 170, 409, 479, 480, 481, or 482. In some embodiments, the nucleotide sequence of the antisense strand is at least 96%, 97%, 98%, 99% or 100% identical to the nucleotide sequence of any one of the antisense strands of any one of dsRNA agents 129, 135, 169, 170, 409, 479, 480, 481, or 482. In some embodiments, the nucleotide sequence of the antisense strand is at least 97%, identical to the nucleotide sequence of any one of the antisense strands of any one of dsRNA agents 129, 135, 169, 170, 409, 479, 480, 481, or 482. In some embodiments, the nucleotide sequence of the antisense strand is at least 98% identical to the nucleotide sequence of any one of the antisense strands of any one of dsRNA agents 129, 135, 169, 170, 409, 479, 480, 481, or 482. In some embodiments, the nucleotide sequence of the antisense strand is at least 99% identical to the nucleotide sequence of any one of the antisense strands of any one of dsRNA agents 129, 135, 169, 170, 409, 479, 480, 481, or 482. In some embodiments, the nucleotide sequence of the antisense strand is 100% identical to the nucleotide sequence of any one of the antisense strands of any one of dsRNA agents 129, 135, 169, 170, 409, 479, 480, 481, or 482.

In some embodiments, the nucleotide sequence of the antisense strand comprises the nucleotide sequence of any one of the antisense strands of any one of dsRNA agents 129, 135, 169, 170, 409, 479, 480, 481, or 482. In some embodiments, the nucleotide sequence of the antisense strand consists of the nucleotide sequence of any one of the antisense strands of any one of dsRNA agents 129, 135, 169, 170, 409, 479, 480, 481, or 482.

Table 2 or Table 3 further identifies the target nucleic acid molecule within the cited reference CIDEB mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence of any one of the antisense strands of any one of dsRNA agents 129, 135, 169, 170, 409, 479, 480, 481, or 482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482.

In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 3 (e.g., 0, 1, 2, or 3) nucleotides from the antisense strand. In some embodiments, the nucleotide sequence of the antisense strand comprises the nucleotide sequence of any one of the antisense strands of any one of dsRNA agents 129, 135, 169, 170, 409, 479, 480, 481, or 482.

As described above, Table 2 or Table 3 further identifies the target nucleic acid molecule within the cited reference CIDEB mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 129, 135, 169, 170, 409, 479, 480, 481, or 482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select antisense strand.

It is to be understood, that although the antisense strands set forth in Table 2 or Table 3 are not described as being modified (e.g., comprising chemically modified nucleotides), conjugated, etc., the disclosure includes any antisense strand set forth in Table 2 or Table 3 that is unmodified, unconjugated, modified (e.g., as described herein), or conjugated (e.g., as described herein).

### 4.2.2 Sense Strand

### 4.2.2.1 Antisense Strand Complementarity

As described above, sense strands (e.g., described herein) comprise a region of complementarity that comprises a nucleotide sequence that is at least partially (e.g., substantially, fully) complementary to the nucleotide sequence of at least a portion of an antisense strand. As such, pairs of sense and antisense strands can hybridize to form a double stranded region (e.g., under conditions in which the pairs will be used).

In some embodiments, the nucleotide sequence of the region of complementarity is at least substantially complementary to the nucleotide sequence of at least a portion of an antisense strand. In some embodiments, the nucleotide sequence of the region of complementarity is fully complementary to the nucleotide sequence of at least a portion of an antisense strand.

In some embodiments, the nucleotide sequence of the region of complementarity is at least 70%, 75%, 80%, 85%, 90%, 91%, 92%, 93%, 94%, 95%, 96%, 97%, 98%, 99%, or 100% complementary to the nucleotide sequence of at least a portion of an antisense strand. For example, the nucleotide sequence of the region of complementarity may be at least 70% complementary to the nucleotide sequence of at least a portion of an antisense strand. The nucleotide sequence of the region of complementarity may be at least 75% complementary to the nucleotide sequence of at least a portion of an antisense strand. The nucleotide sequence of the region of complementarity may be at least 80% complementary to the nucleotide sequence of at least a portion of an antisense strand. The nucleotide sequence of the region of complementarity may be at least 85% complementary to the nucleotide sequence of at least a portion of an antisense strand. The nucleotide sequence of the region of complementarity may be at least 90% complementary to the nucleotide sequence of at least a portion of an antisense strand. The nucleotide sequence of the region of complementarity may be at least 95% complementary to the nucleotide sequence of at least a portion of an antisense strand. In some embodiments, the nucleotide sequence of the region of complementarity is at least 80%, 85%, 90%, 91%, 92%, 93%, 94%, 95%, 96%, 97%, 98%, 99%, or 100% complementary to the nucleotide sequence of at least a portion of an antisense strand. In some embodiments, the nucleotide sequence of the region of complementarity is at least 95%, 96%, 97%, 98%, 99%, or 100% complementary to the nucleotide sequence of at least a portion of an antisense strand.

In some embodiments, the nucleotide sequence of the region of complementarity comprises or consists of one or more non-complementary nucleotide mismatches relative to the nucleotide sequence of the at least a portion of an antisense strand. In some embodiments, the nucleotide sequence of the region of complementarity comprises or consists of no more than 5 (e.g., 4, 3, 2, 1, or 0) non-complementary nucleotide mismatches relative to the nucleotide sequence of the at least a portion of an antisense strand. In some embodiments, the nucleotide sequence of the region of complementarity comprises or consists of no more than 3 (e.g., 2, 1, or 0) non-complementary nucleotide mismatches relative to the nucleotide sequence of the at least a portion of an antisense strand. In some embodiments, the nucleotide sequence of the region of complementarity comprises or consists of no more than 2 (e.g., 1 or 0) non-complementary nucleotide mismatches relative to the nucleotide sequence of the at least a portion of an antisense strand. In some embodiments, the nucleotide sequence of the region of complementarity comprises or consists of no more than 1 (e.g., 0) non-complementary nucleotide mismatches relative to the nucleotide sequence of the at least a portion of an antisense strand. In some embodiments, the nucleotide sequence of the region of complementarity comprises 0 non-complementary nucleotide mismatches relative to the nucleotide sequence of the at least a portion of an antisense strand. In some embodiments, the region of complementarity comprises one or more (e.g., 2, 3, or more) non-complementary nucleotide mismatches relative to the nucleotide sequence of the at least a portion of an antisense strand, wherein the one or more non-complementary nucleotide mismatch is within the last 5 (e.g., 4, 3, 2, or 1) nucleotides from either the 5′- and/or 3′-end of the region of complementarity. In some embodiments, the region of complementarity comprises at least one but not more than 3 (e.g., 1, 2, or 3) non-complementary nucleotide mismatches relative to the nucleotide sequence of the at least a portion of an antisense strand, wherein the one or more non-complementary nucleotide mismatch is within the last 5 (e.g., 4, 3, 2, or 1) nucleotides from either the 5′- and/or 3′-end of the region of complementarity.

In some embodiments, the region of complementarity comprises from about 15-30 nucleotides, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides. In some embodiments, the region of complementarity comprises from about 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 nucleotides. In some embodiments, the region of complementarity comprises from about 19-21 (e.g., 19-20) nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 19, 20, or 21 nucleotides. In some embodiments, the region of complementarity comprises or consists of about 19 nucleotides. In some embodiments, the region of complementarity comprises or consists of about 20 nucleotides. In some embodiments, the region of complementarity comprises or consists of about 21 nucleotides. Ranges and lengths intermediate to the above recited ranges and lengths are also contemplated to be part of the disclosure.

### 4.2.2.2 Overall Length

In some embodiments, the sense strand comprises or consists of from about 15-30 nucleotides (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides). In some embodiments, the sense strand comprises or consists of from about 18-25 nucleotides (e.g., 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides). In some embodiments, the sense strand comprises or consists of from about 19-25 nucleotide (e.g., 19-20, 19-21, 19-22, 19-23, 19-24, 19-25, 20-21, 20-22, 20-23, 20-24, 20-25, 21-22, 21-23, 21-24, 21-25, 22-23, 22-24, 22-25, 23-24, 23-25, 24-25 nucleotides). In some embodiments, the sense strand comprises or consists of from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides.

In some embodiments, the sense strand comprises or consists of not more than about 15, 16, 17, 18, 19, 20, 21, 22, 23, 24, 25, 26, 27, 28, 29, or 30 nucleotides. In some embodiments, the sense strand comprises or consists of about 15, 16, 17, 18, 19, 20, 21, 22, 23, 24, 25, 26, 27, 28, 29, or 30 nucleotides. In some embodiments, the sense strand comprises or consists of about 19, 20, 21, 22, 23 nucleotides. In some embodiments, the sense strand comprises or consists of about 19, 20, 21 nucleotides. In some embodiments, the sense strand comprises or consists of about 20 nucleotides. In some embodiments, the sense strand comprises or consists of about 21 nucleotides. In some embodiments, the sense strand comprises or consists of about 21 nucleotides. In some embodiments, the sense strand comprises or consists of about 23 nucleotides. Ranges and lengths intermediate to the above recited ranges and lengths are also contemplated to be part of the disclosure.

### 4.2.2.3 Exemplary Sense Strands

In some embodiments, the nucleotide sequence of the sense strand differs by no more than 5 (e.g., 0, 1, 2, 3, 4, or 5) nucleotides from the nucleotide sequence of any one of the sense strands set forth in Table 2 or Table 3. In some embodiments, the nucleotide sequence of the sense strand differs by no more than 3 (e.g., 0, 1, 2, or 3) nucleotides from the nucleotide sequence of any one of the sense strands set forth in Table 2 or Table 3. In some embodiments, the nucleotide sequence of the sense strand differs by no more than 2 (e.g., 0, 1, or 2) nucleotides from the nucleotide sequence of any one of the sense strands set forth in Table 2 or Table 3. In some embodiments, the nucleotide sequence of the sense strand differs by no more than 1 (e.g., 0 or 1) nucleotide from the nucleotide sequence of any one of the sense strands set forth in Table 2 or Table 3.

In some embodiments, the nucleotide sequence of the sense strand comprises the nucleotide sequence of any one of the sense strands set forth in Table 2 or Table 3 differing by no more than 5 (e.g., 0, 1, 2, 3, 4, or 5) nucleotides from the sense strand set forth in Table 2 or Table 3. In some embodiments, the nucleotide sequence of the sense strand comprises the nucleotide sequence of any one of the sense strands set forth in Table 2 or Table 3 differing by no more than 3 (e.g., 0, 1, 2, or 3) nucleotides from the sense strand set forth in Table 2 or Table 3. In some embodiments, the nucleotide sequence of the sense strand comprises the nucleotide sequence of any one of the sense strands set forth in Table 2 or Table 3 differing by no more than 2 (e.g., 0, 1, or 2) nucleotides from the sense strand set forth in Table 2 or Table 3. In some embodiments, the nucleotide sequence of the sense strand comprises the nucleotide sequence of any one of the sense strands set forth in Table 2 or Table 3 differing by no more than 1 (e.g., 0 or 1) nucleotide from the sense strand set forth in Table 2 or Table 3.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands set forth in Table 2 or Table 3. In some embodiments, the nucleotide sequence of the sense strand is at least 90%, 91%, 92%, 93%, 94%, 95%, 96%, 97%, 98%, 99% or 100% identical to the nucleotide sequence of any one of the sense strands set forth in Table 2 or Table 3. In some embodiments, the nucleotide sequence of the sense strand is at least 95%, 96%, 97%, 98%, 99% or 100% identical to the nucleotide sequence of any one of the sense strands set forth in Table 2 or Table 3. In some embodiments, the nucleotide sequence of the sense strand is at least 96%, 97%, 98%, 99% or 100% identical to the nucleotide sequence of any one of the sense strands set forth in Table 2 or Table 3. In some embodiments, the nucleotide sequence of the sense strand is at least 97%, identical to the nucleotide sequence of any one of the sense strands set forth in Table 2 or Table 3. In some embodiments, the nucleotide sequence of the sense strand is at least 98% identical to the nucleotide sequence of any one of the sense strands set forth in Table 2 or Table 3. In some embodiments, the nucleotide sequence of the sense strand is at least 99% identical to the nucleotide sequence of any one of the sense strands set forth in Table 2 or Table 3. In some embodiments, the nucleotide sequence of the sense strand is 100% identical to the nucleotide sequence of any one of the sense strands set forth in Table 2 or Table 3.

In some embodiments, the nucleotide sequence of the sense strand comprises the nucleotide sequence of any one of the sense strands set forth in Table 2 or Table 3. In some embodiments, the nucleotide sequence of the sense strand consists of the nucleotide sequence of any one of the sense strands set forth in Table 2 or Table 3.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence of any one of the sense strands set forth in Table 2 or Table 3 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in Table 2 or Table 3. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in Table 2 or Table 3. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the sense strands set forth in Table 2 or Table 3. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the sense strands set forth in Table 2 or Table 3.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in Table 2 or Table 3. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in Table 2 or Table 3. In some embodiments, the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in Table 2 or Table 3.

In some embodiments, the nucleotide sequence of the sense strand comprises the nucleotide sequence of any one of the sense strands set forth in Table 2 or Table 3 differing by no more than 3 (e.g., 0, 1, 2, or 3) nucleotides from the sense strand set forth in Table 2 or Table 3. In some embodiments, the nucleotide sequence of the sense strand comprises the nucleotide sequence of any one of the sense strands set forth in Table 2 or Table 3.

Table 2 or Table 3 further identifies the target nucleic acid molecule within the reference CIDEB mRNA transcript at least partially identical to each of the sense strands. As such, the disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in Table 2 or Table 3 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 4-186, 553-601, 658-892, or 1192. In some embodiments, the nucleotide sequence of the sense strand differs by no more than 3 (e.g., 0, 1, 2, or 3) nucleotides from the nucleotide sequence set forth in any one of SEQ ID NOS: 4-186, 553-601, 658-892, or 1192. In some embodiments, the nucleotide sequence of the sense strand differs by no more than 2 (e.g., 0, 1, or 2) nucleotides from the nucleotide sequence set forth in any one of SEQ ID NOS: 4-186, 553-601, 658-892, or 1192. In some embodiments, the nucleotide sequence of the sense strand differs by no more than 1 (e.g., 0 or 1) nucleotide from the nucleotide sequence set forth in any one of SEQ ID NOS: 4-186, 553-601, 658-892, or 1192.

In some embodiments, the nucleotide sequence of the sense strand comprises the nucleotide sequence set forth in any one of SEQ ID NOS: 4-186, 553-601, 658-892, or 1192 differing by no more than 5 (e.g., 0, 1, 2, 3, 4, or 5) nucleotides from the nucleotide sequence set forth in any one of SEQ ID NOS: 4-186, 553-601, 658-892, or 1192. In some embodiments, the nucleotide sequence of the sense strand comprises the nucleotide sequence set forth in any one of SEQ ID NOS: 4-186, 553-601, 658-892, or 1192 differing by no more than 3 (e.g., 0, 1, 2, or 3) nucleotides from the nucleotide sequence set forth in any one of SEQ ID NOS: 4-186, 553-601, 658-892, or 1192. In some embodiments, the nucleotide sequence of the sense strand comprises the nucleotide sequence set forth in any one of SEQ ID NOS: 4-186, 553-601, 658-892, or 1192 differing by no more than 2 (e.g., 0, 1, or 2) nucleotides from the nucleotide sequence set forth in any one of SEQ ID NOS: 4-186, 553-601, 658-892, or 1192. In some embodiments, the nucleotide sequence of the sense strand comprises the nucleotide sequence set forth in any one of SEQ ID NOS: 4-186, 553-601, 658-892, or 1192 differing by no more than 1 (e.g., 0 or 1) nucleotide from the nucleotide sequence set forth in any one of SEQ ID NOS: 4-186, 553-601, 658-892, or 1192.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 4-186, 553-601, 658-892, or 1192. In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 4-186, 553-601, 658-892, or 1192. In some embodiments, the nucleotide sequence of the sense strand is at least 95%, 96%, 97%, 98%, 99% or 100% identical to the nucleotide sequence set forth in any one of SEQ ID NOS: 4-186, 553-601, 658-892, or 1192. In some embodiments, the nucleotide sequence of the sense strand is at least 96%, 97%, 98%, 99% or 100% identical to the nucleotide sequence set forth in any one of SEQ ID NOS: 4-186, 553-601, 658-892, or 1192. In some embodiments, the nucleotide sequence of the sense strand is at least 97%, identical to the nucleotide sequence set forth in any one of SEQ ID NOS: 4-186, 553-601, 658-892, or 1192. In some embodiments, the nucleotide sequence of the sense strand is at least 98% identical to the nucleotide sequence set forth in any one of SEQ ID NOS: 4-186, 553-601, 658-892, or 1192. In some embodiments, the nucleotide sequence of the sense strand is at least 99% identical to the nucleotide sequence set forth in any one of SEQ ID NOS: 4-186, 553-601, 658-892, or 1192. In some embodiments, the nucleotide sequence of the sense strand is 100% identical to the nucleotide sequence set forth in any one of SEQ ID NOS: 4-186, 553-601, 658-892, or 1192.

In some embodiments, the nucleotide sequence of the sense strand comprises the nucleotide sequence set forth in any one of SEQ ID NOS: 4-186, 553-601, 658-892, or 1192. In some embodiments, the nucleotide sequence of the sense strand consists of the nucleotide sequence set forth in any one of SEQ ID NOS: 4-186, 553-601, 658-892, or 1192.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 4-186, 553-601, 658-892, or 1192 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 4-186, 553-601, 658-892, or 1192. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NOS: 4-186, 553-601, 658-892, or 1192. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in any one of SEQ ID NOS: 4-186, 553-601, 658-892, or 1192. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in any one of SEQ ID NOS: 4-186, 553-601, 658-892, or 1192.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 4-186, 553-601, 658-892, or 1192. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 4-186, 553-601, 658-892, or 1192. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in any one of SEQ ID NOS: 4-186, 553-601, 658-892, or 1192.

In some embodiments, the nucleotide sequence of the sense strand comprises the nucleotide sequence set forth in any one of SEQ ID NOS: 4-186, 553-601, 658-892, or 1192 differing by no more than 3 (e.g., 0, 1, 2, or 3) nucleotides from the nucleotide sequence set forth in any one of SEQ ID NOS: 4-186, 553-601, 658-892, or 1192. In some embodiments, the nucleotide sequence of the sense strand comprises the nucleotide sequence set forth in any one of SEQ ID NOS: 4-186, 553-601, 658-892, or 1192.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 4-186, 553-601, 658-892, or 1192 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 132, 138, 172, or 173. In some embodiments, the nucleotide sequence of the sense strand differs by no more than 3 (e.g., 0, 1, 2, or 3) nucleotides from the nucleotide sequence set forth in any one of SEQ ID NOS: 132, 138, 172, or 173. In some embodiments, the nucleotide sequence of the sense strand differs by no more than 2 (e.g., 0, 1, or 2) nucleotides from the nucleotide sequence set forth in any one of SEQ ID NOS: 132, 138, 172, or 173. In some embodiments, the nucleotide sequence of the sense strand differs by no more than 1 (e.g., 0 or 1) nucleotide from the nucleotide sequence set forth in any one of SEQ ID NOS: 132, 138, 172, or 173.

In some embodiments, the nucleotide sequence of the sense strand comprises the nucleotide sequence set forth in any one of SEQ ID NOS: 132, 138, 172, or 173 differing by no more than 5 (e.g., 0, 1, 2, 3, 4, or 5) nucleotides from the nucleotide sequence set forth in any one of SEQ ID NOS: 315, 321, 355, 356. In some embodiments, the nucleotide sequence of the sense strand comprises the nucleotide sequence set forth in any one of SEQ ID NOS: 132, 138, 172, or 173 differing by no more than 3 (e.g., 0, 1, 2, or 3) nucleotides from the nucleotide sequence set forth in any one of SEQ ID NOS: 132, 138, 172, or 173. In some embodiments, the nucleotide sequence of the sense strand comprises the nucleotide sequence set forth in any one of SEQ ID NOS: 132, 138, 172, or 173 differing by no more than 2 (e.g., 0, 1, or 2) nucleotides from the nucleotide sequence set forth in any one of SEQ ID NOS: 132, 138, 172, or 173. In some embodiments, the nucleotide sequence of the sense strand comprises the nucleotide sequence set forth in any one of SEQ ID NOS: 132, 138, 172, or 173 differing by no more than 1 (e.g., 0 or 1) nucleotide from the nucleotide sequence set forth in any one of SEQ ID NOS: 132, 138, 172, or 173.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 132, 138, 172, or 173. In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 132, 138, 172, or 173. In some embodiments, the nucleotide sequence of the sense strand is at least 95%, 96%, 97%, 98%, 99% or 100% identical to the nucleotide sequence set forth in any one of SEQ ID NOS: 132, 138, 172, or 173. In some embodiments, the nucleotide sequence of the sense strand is at least 96%, 97%, 98%, 99% or 100% identical to the nucleotide sequence set forth in any one of SEQ ID NOS: 132, 138, 172, or 173. In some embodiments, the nucleotide sequence of the sense strand is at least 97%, identical to the nucleotide sequence set forth in any one of SEQ ID NOS: 132, 138, 172, or 173. In some embodiments, the nucleotide sequence of the sense strand is at least 98% identical to the nucleotide sequence set forth in any one of SEQ ID NOS: 132, 138, 172, or 173. In some embodiments, the nucleotide sequence of the sense strand is at least 99% identical to the nucleotide sequence set forth in any one of SEQ ID NOS: 132, 138, 172, or 173. In some embodiments, the nucleotide sequence of the sense strand is 100% identical to the nucleotide sequence set forth in any one of SEQ ID NOS: 132, 138, 172, or 173.

In some embodiments, the nucleotide sequence of the sense strand comprises the nucleotide sequence set forth in any one of SEQ ID NOS: 132, 138, 172, or 173. In some embodiments, the nucleotide sequence of the sense strand consists of the nucleotide sequence set forth in any one of SEQ ID NOS: 132, 138, 172, or 173.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 132, 138, 172, or 173 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 786, 792, 827, or 1192. In some embodiments, the nucleotide sequence of the sense strand differs by no more than 3 (e.g., 0, 1, 2, or 3) nucleotides from the nucleotide sequence set forth in any one of SEQ ID NOS: 786, 792, 827, or 1192. In some embodiments, the nucleotide sequence of the sense strand differs by no more than 2 (e.g., 0, 1, or 2) nucleotides from the nucleotide sequence set forth in any one of SEQ ID NOS: 786, 792, 827, or 1192. In some embodiments, the nucleotide sequence of the sense strand differs by no more than 1 (e.g., 0 or 1) nucleotide from the nucleotide sequence set forth in any one of SEQ ID NOS: 786, 792, 827, or 1192.

In some embodiments, the nucleotide sequence of the sense strand comprises the nucleotide sequence set forth in any one of SEQ ID NOS: 786, 792, 827, or 1192 differing by no more than 5 (e.g., 0, 1, 2, 3, 4, or 5) nucleotides from the nucleotide sequence set forth in any one of SEQ ID NOS: 315, 321, 355, 356. In some embodiments, the nucleotide sequence of the sense strand comprises the nucleotide sequence set forth in any one of SEQ ID NOS: 786, 792, 827, or 1192 differing by no more than 3 (e.g., 0, 1, 2, or 3) nucleotides from the nucleotide sequence set forth in any one of SEQ ID NOS: 786, 792, 827, or 1192. In some embodiments, the nucleotide sequence of the sense strand comprises the nucleotide sequence set forth in any one of SEQ ID NOS: 786, 792, 827, or 1192 differing by no more than 2 (e.g., 0, 1, or 2) nucleotides from the nucleotide sequence set forth in any one of SEQ ID NOS: 786, 792, 827, or 1192. In some embodiments, the nucleotide sequence of the sense strand comprises the nucleotide sequence set forth in any one of SEQ ID NOS: 786, 792, 827, or 1192 differing by no more than 1 (e.g., 0 or 1) nucleotide from the nucleotide sequence set forth in any one of SEQ ID NOS: 786, 792, 827, or 1192.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 786, 792, 827, or 1192. In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 786, 792, 827, or 1192. In some embodiments, the nucleotide sequence of the sense strand is at least 95%, 96%, 97%, 98%, 99% or 100% identical to the nucleotide sequence set forth in any one of SEQ ID NOS: 786, 792, 827, or 1192. In some embodiments, the nucleotide sequence of the sense strand is at least 96%, 97%, 98%, 99% or 100% identical to the nucleotide sequence set forth in any one of SEQ ID NOS: 786, 792, 827, or 1192. In some embodiments, the nucleotide sequence of the sense strand is at least 97%, identical to the nucleotide sequence set forth in any one of SEQ ID NOS: 786, 792, 827, or 1192. In some embodiments, the nucleotide sequence of the sense strand is at least 98% identical to the nucleotide sequence set forth in any one of SEQ ID NOS: 786, 792, 827, or 1192. In some embodiments, the nucleotide sequence of the sense strand is at least 99% identical to the nucleotide sequence set forth in any one of SEQ ID NOS: 786, 792, 827, or 1192. In some embodiments, the nucleotide sequence of the sense strand is 100% identical to the nucleotide sequence set forth in any one of SEQ ID NOS: 786, 792, 827, or 1192.

In some embodiments, the nucleotide sequence of the sense strand comprises the nucleotide sequence set forth in any one of SEQ ID NOS: 786, 792, 827, or 1192. In some embodiments, the nucleotide sequence of the sense strand consists of the nucleotide sequence set forth in any one of SEQ ID NOS: 786, 792, 827, or 1192.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 786, 792, 827, or 1192 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 132. In some embodiments, the nucleotide sequence of the sense strand differs by no more than 3 (e.g., 0, 1, 2, or 3) nucleotides from the nucleotide sequence set forth in SEQ ID NO: 132. In some embodiments, the nucleotide sequence of the sense strand differs by no more than 2 (e.g., 0, 1, or 2) nucleotides from the nucleotide sequence set forth in SEQ ID NO: 132. In some embodiments, the nucleotide sequence of the sense strand differs by no more than 1 (e.g., 0 or 1) nucleotide from the nucleotide sequence set forth in SEQ ID NO: 132.

In some embodiments, the nucleotide sequence of the sense strand comprises the nucleotide sequence set forth in SEQ ID NO: 132 differing by no more than 5 (e.g., 0, 1, 2, 3, 4, or 5) nucleotides from the nucleotide sequence set forth in SEQ ID NO: 132. In some embodiments, the nucleotide sequence of the sense strand comprises the nucleotide sequence set forth in SEQ ID NO: 132 differing by no more than 3 (e.g., 0, 1, 2, or 3) nucleotides from the nucleotide sequence set forth in SEQ ID NO: 132. In some embodiments, the nucleotide sequence of the sense strand comprises the nucleotide sequence set forth in SEQ ID NO: 132 differing by no more than 2 (e.g., 0, 1, or 2) nucleotides from the nucleotide sequence set forth in SEQ ID NO: 132. In some embodiments, the nucleotide sequence of the sense strand comprises the nucleotide sequence set forth in SEQ ID NO: 132 differing by no more than 1 (e.g., 0 or 1) nucleotide from nucleotide sequence set forth in SEQ ID NO: 132.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 132. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 132. In some embodiments, the nucleotide sequence of the sense strand is at least 95%, 96%, 97%, 98%, 99% or 100% identical to the nucleotide sequence set forth in SEQ ID NO: 132. In some embodiments, the nucleotide sequence of the sense strand is at least 96%, 97%, 98%, 99% or 100% identical to the nucleotide sequence set forth in SEQ ID NO: 132. In some embodiments, the nucleotide sequence of the sense strand is at least 97%, identical to the nucleotide sequence set forth in SEQ ID NO: 132. In some embodiments, the nucleotide sequence of the sense strand is at least 98% identical to the nucleotide sequence set forth in SEQ ID NO: 132. In some embodiments, the nucleotide sequence of the sense strand is at least 99% identical to the nucleotide sequence set forth in SEQ ID NO: 132. In some embodiments, the nucleotide sequence of the sense strand is 100% identical to the nucleotide sequence set forth in SEQ ID NO: 132.

In some embodiments, the nucleotide sequence of the sense strand comprises the nucleotide sequence set forth in SEQ ID NO: 132. In some embodiments, the nucleotide sequence of the sense strand consists of the nucleotide sequence set forth in SEQ ID NO: 132.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 132 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 132. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 132. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 132. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 132.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 132. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 132. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 132.

In some embodiments, the nucleotide sequence of the sense strand comprises the nucleotide sequence set forth in SEQ ID NO: 132 differing by no more than 3 (e.g., 0, 1, 2, or 3) nucleotides from the nucleotide sequence set forth in SEQ ID NO: 132. In some embodiments, the nucleotide sequence of the sense strand comprises the nucleotide sequence set forth in SEQ ID NO: 132.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 132 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 138. In some embodiments, the nucleotide sequence of the sense strand differs by no more than 3 (e.g., 0, 1, 2, or 3) nucleotides from the nucleotide sequence set forth in SEQ ID NO: 138. In some embodiments, the nucleotide sequence of the sense strand differs by no more than 2 (e.g., 0, 1, or 2) nucleotides from the nucleotide sequence set forth in SEQ ID NO: 138. In some embodiments, the nucleotide sequence of the sense strand differs by no more than 1 (e.g., 0 or 1) nucleotide from the nucleotide sequence set forth in SEQ ID NO: 138.

In some embodiments, the nucleotide sequence of the sense strand comprises the nucleotide sequence set forth in SEQ ID NO: 138 differing by no more than 5 (e.g., 0, 1, 2, 3, 4, or 5) nucleotides from the nucleotide sequence set forth in SEQ ID NO: 138. In some embodiments, the nucleotide sequence of the sense strand comprises the nucleotide sequence set forth in SEQ ID NO: 138 differing by no more than 3 (e.g., 0, 1, 2, or 3) nucleotides from the nucleotide sequence set forth in SEQ ID NO: 138. In some embodiments, the nucleotide sequence of the sense strand comprises the nucleotide sequence set forth in SEQ ID NO: 138 differing by no more than 2 (e.g., 0, 1, or 2) nucleotides from the nucleotide sequence set forth in SEQ ID NO: 138. In some embodiments, the nucleotide sequence of the sense strand comprises the nucleotide sequence set forth in SEQ ID NO: 138 differing by no more than 1 (e.g., 0 or 1) nucleotide from nucleotide sequence set forth in SEQ ID NO: 138.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 138. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 138. In some embodiments, the nucleotide sequence of the sense strand is at least 95%, 96%, 97%, 98%, 99% or 100% identical to the nucleotide sequence set forth in SEQ ID NO: 138. In some embodiments, the nucleotide sequence of the sense strand is at least 96%, 97%, 98%, 99% or 100% identical to the nucleotide sequence set forth in SEQ ID NO: 138. In some embodiments, the nucleotide sequence of the sense strand is at least 97%, identical to the nucleotide sequence set forth in SEQ ID NO: 138. In some embodiments, the nucleotide sequence of the sense strand is at least 98% identical to the nucleotide sequence set forth in SEQ ID NO: 138. In some embodiments, the nucleotide sequence of the sense strand is at least 99% identical to the nucleotide sequence set forth in SEQ ID NO: 138. In some embodiments, the nucleotide sequence of the sense strand is 100% identical to the nucleotide sequence set forth in SEQ ID NO: 138.

In some embodiments, the nucleotide sequence of the sense strand comprises the nucleotide sequence set forth in SEQ ID NO: 138. In some embodiments, the nucleotide sequence of the sense strand consists of the nucleotide sequence set forth in SEQ ID NO: 138.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 138 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 138. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 138. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 138. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 138.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 138. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 138. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 138.

In some embodiments, the nucleotide sequence of the sense strand comprises the nucleotide sequence set forth in SEQ ID NO: 138 differing by no more than 3 (e.g., 0, 1, 2, or 3) nucleotides from the nucleotide sequence set forth in SEQ ID NO: 138. In some embodiments, the nucleotide sequence of the sense strand comprises the nucleotide sequence set forth in SEQ ID NO: 138.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 138 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 172. In some embodiments, the nucleotide sequence of the sense strand differs by no more than 3 (e.g., 0, 1, 2, or 3) nucleotides from the nucleotide sequence set forth in SEQ ID NO: 172. In some embodiments, the nucleotide sequence of the sense strand differs by no more than 2 (e.g., 0, 1, or 2) nucleotides from the nucleotide sequence set forth in SEQ ID NO: 172. In some embodiments, the nucleotide sequence of the sense strand differs by no more than 1 (e.g., 0 or 1) nucleotide from the nucleotide sequence set forth in SEQ ID NO: 172.

In some embodiments, the nucleotide sequence of the sense strand comprises the nucleotide sequence set forth in SEQ ID NO: 172 differing by no more than 5 (e.g., 0, 1, 2, 3, 4, or 5) nucleotides from the nucleotide sequence set forth in SEQ ID NO: 172. In some embodiments, the nucleotide sequence of the sense strand comprises the nucleotide sequence set forth in SEQ ID NO: 172 differing by no more than 3 (e.g., 0, 1, 2, or 3) nucleotides from the nucleotide sequence set forth in SEQ ID NO: 172. In some embodiments, the nucleotide sequence of the sense strand comprises the nucleotide sequence set forth in SEQ ID NO: 172 differing by no more than 2 (e.g., 0, 1, or 2) nucleotides from the nucleotide sequence set forth in SEQ ID NO: 172. In some embodiments, the nucleotide sequence of the sense strand comprises the nucleotide sequence set forth in SEQ ID NO: 172 differing by no more than 1 (e.g., 0 or 1) nucleotide from nucleotide sequence set forth in SEQ ID NO: 172.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 172. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 172. In some embodiments, the nucleotide sequence of the sense strand is at least 95%, 96%, 97%, 98%, 99% or 100% identical to the nucleotide sequence set forth in SEQ ID NO: 172. In some embodiments, the nucleotide sequence of the sense strand is at least 96%, 97%, 98%, 99% or 100% identical to the nucleotide sequence set forth in SEQ ID NO: 172. In some embodiments, the nucleotide sequence of the sense strand is at least 97%, identical to the nucleotide sequence set forth in SEQ ID NO: 172. In some embodiments, the nucleotide sequence of the sense strand is at least 98% identical to the nucleotide sequence set forth in SEQ ID NO: 172. In some embodiments, the nucleotide sequence of the sense strand is at least 99% identical to the nucleotide sequence set forth in SEQ ID NO: 172. In some embodiments, the nucleotide sequence of the sense strand is 100% identical to the nucleotide sequence set forth in SEQ ID NO: 172.

In some embodiments, the nucleotide sequence of the sense strand comprises the nucleotide sequence set forth in SEQ ID NO: 172. In some embodiments, the nucleotide sequence of the sense strand consists of the nucleotide sequence set forth in SEQ ID NO: 172.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 172 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 172. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 172. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 172. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 172.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 172. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 172. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 172.

In some embodiments, the nucleotide sequence of the sense strand comprises the nucleotide sequence set forth in SEQ ID NO: 172 differing by no more than 3 (e.g., 0, 1, 2, or 3) nucleotides from the nucleotide sequence set forth in SEQ ID NO: 172. In some embodiments, the nucleotide sequence of the sense strand comprises the nucleotide sequence set forth in SEQ ID NO: 172.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 172 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 173. In some embodiments, the nucleotide sequence of the sense strand differs by no more than 3 (e.g., 0, 1, 2, or 3) nucleotides from the nucleotide sequence set forth in SEQ ID NO: 173. In some embodiments, the nucleotide sequence of the sense strand differs by no more than 2 (e.g., 0, 1, or 2) nucleotides from the nucleotide sequence set forth in SEQ ID NO: 173. In some embodiments, the nucleotide sequence of the sense strand differs by no more than 1 (e.g., 0 or 1) nucleotide from the nucleotide sequence set forth in SEQ ID NO: 173.

In some embodiments, the nucleotide sequence of the sense strand comprises the nucleotide sequence set forth in SEQ ID NO: 173 differing by no more than 5 (e.g., 0, 1, 2, 3, 4, or 5) nucleotides from the nucleotide sequence set forth in SEQ ID NO: 173. In some embodiments, the nucleotide sequence of the sense strand comprises the nucleotide sequence set forth in SEQ ID NO: 173 differing by no more than 3 (e.g., 0, 1, 2, or 3) nucleotides from the nucleotide sequence set forth in SEQ ID NO: 173. In some embodiments, the nucleotide sequence of the sense strand comprises the nucleotide sequence set forth in SEQ ID NO: 173 differing by no more than 2 (e.g., 0, 1, or 2) nucleotides from the nucleotide sequence set forth in SEQ ID NO: 173. In some embodiments, the nucleotide sequence of the sense strand comprises the nucleotide sequence set forth in SEQ ID NO: 173 differing by no more than 1 (e.g., 0 or 1) nucleotide from nucleotide sequence set forth in SEQ ID NO: 173.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 173. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 173. In some embodiments, the nucleotide sequence of the sense strand is at least 95%, 96%, 97%, 98%, 99% or 100% identical to the nucleotide sequence set forth in SEQ ID NO: 173. In some embodiments, the nucleotide sequence of the sense strand is at least 96%, 97%, 98%, 99% or 100% identical to the nucleotide sequence set forth in SEQ ID NO: 173. In some embodiments, the nucleotide sequence of the sense strand is at least 97%, identical to the nucleotide sequence set forth in SEQ ID NO: 173. In some embodiments, the nucleotide sequence of the sense strand is at least 98% identical to the nucleotide sequence set forth in SEQ ID NO: 173. In some embodiments, the nucleotide sequence of the sense strand is at least 99% identical to the nucleotide sequence set forth in SEQ ID NO: 173. In some embodiments, the nucleotide sequence of the sense strand is 100% identical to the nucleotide sequence set forth in SEQ ID NO: 173.

In some embodiments, the nucleotide sequence of the sense strand comprises the nucleotide sequence set forth in SEQ ID NO: 173. In some embodiments, the nucleotide sequence of the sense strand consists of the nucleotide sequence set forth in SEQ ID NO: 173.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 173 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 173. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 173. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 173. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 173.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 173. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 173. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 173.

In some embodiments, the nucleotide sequence of the sense strand comprises the nucleotide sequence set forth in SEQ ID NO: 173 differing by no more than 3 (e.g., 0, 1, 2, or 3) nucleotides from the nucleotide sequence set forth in SEQ ID NO: 173. In some embodiments, the nucleotide sequence of the sense strand comprises the nucleotide sequence set forth in SEQ ID NO: 173.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 173 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 786. In some embodiments, the nucleotide sequence of the sense strand differs by no more than 3 (e.g., 0, 1, 2, or 3) nucleotides from the nucleotide sequence set forth in SEQ ID NO: 786. In some embodiments, the nucleotide sequence of the sense strand differs by no more than 2 (e.g., 0, 1, or 2) nucleotides from the nucleotide sequence set forth in SEQ ID NO: 786. In some embodiments, the nucleotide sequence of the sense strand differs by no more than 1 (e.g., 0 or 1) nucleotide from the nucleotide sequence set forth in SEQ ID NO: 786.

In some embodiments, the nucleotide sequence of the sense strand comprises the nucleotide sequence set forth in SEQ ID NO: 786 differing by no more than 5 (e.g., 0, 1, 2, 3, 4, or 5) nucleotides from the nucleotide sequence set forth in SEQ ID NO: 786. In some embodiments, the nucleotide sequence of the sense strand comprises the nucleotide sequence set forth in SEQ ID NO: 786 differing by no more than 3 (e.g., 0, 1, 2, or 3) nucleotides from the nucleotide sequence set forth in SEQ ID NO: 786. In some embodiments, the nucleotide sequence of the sense strand comprises the nucleotide sequence set forth in SEQ ID NO: 786 differing by no more than 2 (e.g., 0, 1, or 2) nucleotides from the nucleotide sequence set forth in SEQ ID NO: 786. In some embodiments, the nucleotide sequence of the sense strand comprises the nucleotide sequence set forth in SEQ ID NO: 786 differing by no more than 1 (e.g., 0 or 1) nucleotide from nucleotide sequence set forth in SEQ ID NO: 786.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 786. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 786. In some embodiments, the nucleotide sequence of the sense strand is at least 95%, 96%, 97%, 98%, 99% or 100% identical to the nucleotide sequence set forth in SEQ ID NO: 786. In some embodiments, the nucleotide sequence of the sense strand is at least 96%, 97%, 98%, 99% or 100% identical to the nucleotide sequence set forth in SEQ ID NO: 786. In some embodiments, the nucleotide sequence of the sense strand is at least 97%, identical to the nucleotide sequence set forth in SEQ ID NO: 786. In some embodiments, the nucleotide sequence of the sense strand is at least 98% identical to the nucleotide sequence set forth in SEQ ID NO: 786. In some embodiments, the nucleotide sequence of the sense strand is at least 99% identical to the nucleotide sequence set forth in SEQ ID NO: 786. In some embodiments, the nucleotide sequence of the sense strand is 100% identical to the nucleotide sequence set forth in SEQ ID NO: 786.

In some embodiments, the nucleotide sequence of the sense strand comprises the nucleotide sequence set forth in SEQ ID NO: 786. In some embodiments, the nucleotide sequence of the sense strand consists of the nucleotide sequence set forth in SEQ ID NO: 786.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 786 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 786. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 786. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 786. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 786.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 786. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 786. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 786.

In some embodiments, the nucleotide sequence of the sense strand comprises the nucleotide sequence set forth in SEQ ID NO: 786 differing by no more than 3 (e.g., 0, 1, 2, or 3) nucleotides from the nucleotide sequence set forth in SEQ ID NO: 786. In some embodiments, the nucleotide sequence of the sense strand comprises the nucleotide sequence set forth in SEQ ID NO: 786.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 786 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 1192. In some embodiments, the nucleotide sequence of the sense strand differs by no more than 3 (e.g., 0, 1, 2, or 3) nucleotides from the nucleotide sequence set forth in SEQ ID NO: 1192. In some embodiments, the nucleotide sequence of the sense strand differs by no more than 2 (e.g., 0, 1, or 2) nucleotides from the nucleotide sequence set forth in SEQ ID NO: 1192. In some embodiments, the nucleotide sequence of the sense strand differs by no more than 1 (e.g., 0 or 1) nucleotide from the nucleotide sequence set forth in SEQ ID NO: 1192.

In some embodiments, the nucleotide sequence of the sense strand comprises the nucleotide sequence set forth in SEQ ID NO: 1192 differing by no more than 5 (e.g., 0, 1, 2, 3, 4, or 5) nucleotides from the nucleotide sequence set forth in SEQ ID NO: 1192. In some embodiments, the nucleotide sequence of the sense strand comprises the nucleotide sequence set forth in SEQ ID NO: 1192 differing by no more than 3 (e.g., 0, 1, 2, or 3) nucleotides from the nucleotide sequence set forth in SEQ ID NO: 1192. In some embodiments, the nucleotide sequence of the sense strand comprises the nucleotide sequence set forth in SEQ ID NO: 1192 differing by no more than 2 (e.g., 0, 1, or 2) nucleotides from the nucleotide sequence set forth in SEQ ID NO: 1192. In some embodiments, the nucleotide sequence of the sense strand comprises the nucleotide sequence set forth in SEQ ID NO: 1192 differing by no more than 1 (e.g., 0 or 1) nucleotide from nucleotide sequence set forth in SEQ ID NO: 1192.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1192. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 1192. In some embodiments, the nucleotide sequence of the sense strand is at least 95%, 96%, 97%, 98%, 99% or 100% identical to the nucleotide sequence set forth in SEQ ID NO: 1192. In some embodiments, the nucleotide sequence of the sense strand is at least 96%, 97%, 98%, 99% or 100% identical to the nucleotide sequence set forth in SEQ ID NO: 1192. In some embodiments, the nucleotide sequence of the sense strand is at least 97%, identical to the nucleotide sequence set forth in SEQ ID NO: 1192. In some embodiments, the nucleotide sequence of the sense strand is at least 98% identical to the nucleotide sequence set forth in SEQ ID NO: 1192. In some embodiments, the nucleotide sequence of the sense strand is at least 99% identical to the nucleotide sequence set forth in SEQ ID NO: 1192. In some embodiments, the nucleotide sequence of the sense strand is 100% identical to the nucleotide sequence set forth in SEQ ID NO: 1192.

In some embodiments, the nucleotide sequence of the sense strand comprises the nucleotide sequence set forth in SEQ ID NO: 1192. In some embodiments, the nucleotide sequence of the sense strand consists of the nucleotide sequence set forth in SEQ ID NO: 1192.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 1192 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1192. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 1192. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 1192. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 1192.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1192. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 1192. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 1192.

In some embodiments, the nucleotide sequence of the sense strand comprises the nucleotide sequence set forth in SEQ ID NO: 1192 differing by no more than 3 (e.g., 0, 1, 2, or 3) nucleotides from the nucleotide sequence set forth in SEQ ID NO: 1192. In some embodiments, the nucleotide sequence of the sense strand comprises the nucleotide sequence set forth in SEQ ID NO: 1192.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 1192 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 792. In some embodiments, the nucleotide sequence of the sense strand differs by no more than 3 (e.g., 0, 1, 2, or 3) nucleotides from the nucleotide sequence set forth in SEQ ID NO: 792. In some embodiments, the nucleotide sequence of the sense strand differs by no more than 2 (e.g., 0, 1, or 2) nucleotides from the nucleotide sequence set forth in SEQ ID NO: 792. In some embodiments, the nucleotide sequence of the sense strand differs by no more than 1 (e.g., 0 or 1) nucleotide from the nucleotide sequence set forth in SEQ ID NO: 792.

In some embodiments, the nucleotide sequence of the sense strand comprises the nucleotide sequence set forth in SEQ ID NO: 792 differing by no more than 5 (e.g., 0, 1, 2, 3, 4, or 5) nucleotides from the nucleotide sequence set forth in SEQ ID NO: 792. In some embodiments, the nucleotide sequence of the sense strand comprises the nucleotide sequence set forth in SEQ ID NO: 792 differing by no more than 3 (e.g., 0, 1, 2, or 3) nucleotides from the nucleotide sequence set forth in SEQ ID NO: 792. In some embodiments, the nucleotide sequence of the sense strand comprises the nucleotide sequence set forth in SEQ ID NO: 792 differing by no more than 2 (e.g., 0, 1, or 2) nucleotides from the nucleotide sequence set forth in SEQ ID NO: 792. In some embodiments, the nucleotide sequence of the sense strand comprises the nucleotide sequence set forth in SEQ ID NO: 792 differing by no more than 1 (e.g., 0 or 1) nucleotide from nucleotide sequence set forth in SEQ ID NO: 792.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 792. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 792. In some embodiments, the nucleotide sequence of the sense strand is at least 95%, 96%, 97%, 98%, 99% or 100% identical to the nucleotide sequence set forth in SEQ ID NO: 792. In some embodiments, the nucleotide sequence of the sense strand is at least 96%, 97%, 98%, 99% or 100% identical to the nucleotide sequence set forth in SEQ ID NO: 792. In some embodiments, the nucleotide sequence of the sense strand is at least 97%, identical to the nucleotide sequence set forth in SEQ ID NO: 792. In some embodiments, the nucleotide sequence of the sense strand is at least 98% identical to the nucleotide sequence set forth in SEQ ID NO: 792. In some embodiments, the nucleotide sequence of the sense strand is at least 99% identical to the nucleotide sequence set forth in SEQ ID NO: 792. In some embodiments, the nucleotide sequence of the sense strand is 100% identical to the nucleotide sequence set forth in SEQ ID NO: 792.

In some embodiments, the nucleotide sequence of the sense strand comprises the nucleotide sequence set forth in SEQ ID NO: 792. In some embodiments, the nucleotide sequence of the sense strand consists of the nucleotide sequence set forth in SEQ ID NO: 792.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 792 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 792. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 792. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 792. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 792.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 792. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 792. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 792.

In some embodiments, the nucleotide sequence of the sense strand comprises the nucleotide sequence set forth in SEQ ID NO: 792 differing by no more than 3 (e.g., 0, 1, 2, or 3) nucleotides from the nucleotide sequence set forth in SEQ ID NO: 792. In some embodiments, the nucleotide sequence of the sense strand comprises the nucleotide sequence set forth in SEQ ID NO: 792.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 792 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in SEQ ID NO: 827. In some embodiments, the nucleotide sequence of the sense strand differs by no more than 3 (e.g., 0, 1, 2, or 3) nucleotides from the nucleotide sequence set forth in SEQ ID NO: 827. In some embodiments, the nucleotide sequence of the sense strand differs by no more than 2 (e.g., 0, 1, or 2) nucleotides from the nucleotide sequence set forth in SEQ ID NO: 827. In some embodiments, the nucleotide sequence of the sense strand differs by no more than 1 (e.g., 0 or 1) nucleotide from the nucleotide sequence set forth in SEQ ID NO: 827.

In some embodiments, the nucleotide sequence of the sense strand comprises the nucleotide sequence set forth in SEQ ID NO: 827 differing by no more than 5 (e.g., 0, 1, 2, 3, 4, or 5) nucleotides from the nucleotide sequence set forth in SEQ ID NO: 827. In some embodiments, the nucleotide sequence of the sense strand comprises the nucleotide sequence set forth in SEQ ID NO: 827 differing by no more than 3 (e.g., 0, 1, 2, or 3) nucleotides from the nucleotide sequence set forth in SEQ ID NO: 827. In some embodiments, the nucleotide sequence of the sense strand comprises the nucleotide sequence set forth in SEQ ID NO: 827 differing by no more than 2 (e.g., 0, 1, or 2) nucleotides from the nucleotide sequence set forth in SEQ ID NO: 827. In some embodiments, the nucleotide sequence of the sense strand comprises the nucleotide sequence set forth in SEQ ID NO: 827 differing by no more than 1 (e.g., 0 or 1) nucleotide from nucleotide sequence set forth in SEQ ID NO: 827.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 827. In some embodiments, the nucleotide sequence of the sense strand is at least 90%, 91%, 92%, 93%, 94%, 95%, 96%, 97%, 98%, 99% or 100% identical to the nucleotide sequence set forth in SEQ ID NO: 827. In some embodiments, the nucleotide sequence of the sense strand is at least 95%, 96%, 97%, 98%, 99% or 100% identical to the nucleotide sequence set forth in SEQ ID NO: 827. In some embodiments, the nucleotide sequence of the sense strand is at least 96%, 97%, 98%, 99% or 100% identical to the nucleotide sequence set forth in SEQ ID NO: 827. In some embodiments, the nucleotide sequence of the sense strand is at least 97%, identical to the nucleotide sequence set forth in SEQ ID NO: 827. In some embodiments, the nucleotide sequence of the sense strand is at least 98% identical to the nucleotide sequence set forth in SEQ ID NO: 827. In some embodiments, the nucleotide sequence of the sense strand is at least 99% identical to the nucleotide sequence set forth in SEQ ID NO: 827. In some embodiments, the nucleotide sequence of the sense strand is 100% identical to the nucleotide sequence set forth in SEQ ID NO: 827.

In some embodiments, the nucleotide sequence of the sense strand comprises the nucleotide sequence set forth in SEQ ID NO: 827. In some embodiments, the nucleotide sequence of the sense strand consists of the nucleotide sequence set forth in SEQ ID NO: 827.

Table 2 or Table 3 further identifies the target nucleic acid molecule within the cited reference CIDEB mRNA transcript (SEQ ID NO: 1) targeted by each of the sense strands. As such, the disclosure further provides an sense strand wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in SEQ ID NO: 827 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 827. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NO: 827. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in SEQ ID NO: 827. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in SEQ ID NO: 827.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 827. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in SEQ ID NO: 827. In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in SEQ ID NO: 827.

In some embodiments, the nucleotide sequence of the sense strand comprises the nucleotide sequence set forth in SEQ ID NO: 827 differing by no more than 3 (e.g., 0, 1, 2, or 3) nucleotides from the nucleotide sequence set forth in SEQ ID NO: 827. In some embodiments, the nucleotide sequence of the sense strand comprises the nucleotide sequence set forth in SEQ ID NO: 827.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in SEQ ID NO: 827 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the nucleotide sequence of the sense strand differs by no more than 5 (e.g., 0, 1, 2, 3, 4, or 5) nucleotides from the nucleotide sequence of any one of the sense strands of any one of dsRNA agents 1-482. In some embodiments, the nucleotide sequence of the sense strand differs by no more than 3 (e.g., 0, 1, 2, or 3) nucleotides from the nucleotide sequence of any one of the sense strands of any one of dsRNA agents 1-482. In some embodiments, the nucleotide sequence of the sense strand differs by no more than 2 (e.g., 0, 1, or 2) nucleotides from the nucleotide sequence of any one of the sense strands of any one of dsRNA agents 1-482. In some embodiments, the nucleotide sequence of the sense strand differs by no more than 1 (e.g., 0 or 1) nucleotide from the nucleotide sequence of any one of the sense strands of any one of dsRNA agents 1-482.

In some embodiments, the nucleotide sequence of the sense strand comprises the nucleotide sequence of any one of the sense strands of any one of dsRNA agents 1-482 differing by no more than 5 (e.g., 0, 1, 2, 3, 4, or 5) nucleotides from the sense strand of any one of dsRNA agents 1-482. In some embodiments, the nucleotide sequence of the sense strand comprises the nucleotide sequence of any one of the sense strands of any one of dsRNA agents 1-482 differing by no more than 3 (e.g., 0, 1, 2, or 3) nucleotides from the sense strand of any one of dsRNA agents 1-482. In some embodiments, the nucleotide sequence of the sense strand comprises the nucleotide sequence of any one of the sense strands of any one of dsRNA agents 1-482 differing by no more than 2 (e.g., 0, 1, or 2) nucleotides from the sense strand of any one of dsRNA agents 1-482. In some embodiments, the nucleotide sequence of the sense strand comprises the nucleotide sequence of any one of the sense strands of any one of dsRNA agents 1-482 differing by no more than 1 (e.g., 0 or 1) nucleotide from the sense strand of any one of dsRNA agents 1-482.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands of any one of dsRNA agents 1-482. In some embodiments, the nucleotide sequence of the sense strand is at least 90%, 91%, 92%, 93%, 94%, 95%, 96%, 97%, 98%, 99% or 100% identical to the nucleotide sequence of any one of the sense strands of any one of dsRNA agents 1-482. In some embodiments, the nucleotide sequence of the sense strand is at least 95%, 96%, 97%, 98%, 99% or 100% identical to the nucleotide sequence of any one of the sense strands of any one of dsRNA agents 1-482. In some embodiments, the nucleotide sequence of the sense strand is at least 96%, 97%, 98%, 99% or 100% identical to the nucleotide sequence of any one of the sense strands of any one of dsRNA agents 1-482. In some embodiments, the nucleotide sequence of the sense strand is at least 97%, identical to the nucleotide sequence of any one of the sense strands of any one of dsRNA agents 1-482. In some embodiments, the nucleotide sequence of the sense strand is at least 98% identical to the nucleotide sequence of any one of the sense strands of any one of dsRNA agents 1-482. In some embodiments, the nucleotide sequence of the sense strand is at least 99% identical to the nucleotide sequence of any one of the sense strands of any one of dsRNA agents 1-482. In some embodiments, the nucleotide sequence of the sense strand is 100% identical to the nucleotide sequence of any one of the sense strands of any one of dsRNA agents 1-482.

In some embodiments, the nucleotide sequence of the sense strand comprises the nucleotide sequence of any one of the sense strands of any one of dsRNA agents 1-482. In some embodiments, the nucleotide sequence of the sense strand consists of the nucleotide sequence of any one of the sense strands of any one of dsRNA agents 1-482.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence of any one of the sense strands of any one of dsRNA agents 1-482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-482. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-482. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-482. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the sense strands of any one of dsRNA agents 1-482.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 1-482. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 1-482. In some embodiments, the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands of any one of dsRNA agents 1-482.

In some embodiments, the nucleotide sequence of the sense strand comprises the nucleotide sequence of any one of the sense strands of any one of dsRNA agents 1-482 differing by no more than 3 (e.g., 0, 1, 2, or 3) nucleotides from the sense strand. In some embodiments, the nucleotide sequence of the sense strand comprises the nucleotide sequence of any one of the sense strands of any one of dsRNA agents 1-482.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-482 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the sense strands of any one of dsRNA agents 1-482.

In some embodiments, the nucleotide sequence of the sense strand differs by no more than 5 (e.g., 0, 1, 2, 3, 4, or 5) nucleotides from the nucleotide sequence of any one of the sense strands of any one of dsRNA agents 129, 135, 169, 170, 409, 479, 480, 481, or 482. In some embodiments, the nucleotide sequence of the sense strand differs by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the nucleotide sequence of the sense strand differs by no more than 2 (e.g., 0, 1, or 2) nucleotides from the nucleotide sequence of any one of the sense strands of any one of dsRNA agents 129, 135, 169, 170, 409, 479, 480, 481, or 482. In some embodiments, the nucleotide sequence of the sense strand differs by no more than 1 (e.g., 0 or 1) nucleotide from the nucleotide sequence of any one of the sense strands of any one of dsRNA agents 129, 135, 169, 170, 409, 479, 480, 481, or 482.

In some embodiments, the nucleotide sequence of the sense strand comprises the nucleotide sequence of any one of the sense strands of any one of dsRNA agents 129, 135, 169, 170, 409, 479, 480, 481, or 482 differing by no more than 5 (e.g., 0, 1, 2, 3, 4, or 5) nucleotides from the sense strand of any one of dsRNA agents 129, 135, 169, 170, 409, 479, 480, 481, or 482. In some embodiments, the nucleotide sequence of the sense strand comprises the nucleotide sequence of any one of the sense strands of any one of dsRNA agents 129, 135, 169, 170, 409, 479, 480, 481, or 482 differing by no more than 3 (e.g., 0, 1, 2, or 3) nucleotides from the sense strand of any one of dsRNA agents 129, 135, 169, 170, 409, 479, 480, 481, or 482. In some embodiments, the nucleotide sequence of the sense strand comprises the nucleotide sequence of any one of the sense strands of any one of dsRNA agents 129, 135, 169, 170, 409, 479, 480, 481, or 482 differing by no more than 2 (e.g., 0, 1, or 2) nucleotides from the sense strand of any one of dsRNA agents 129, 135, 169, 170, 409, 479, 480, 481, or 482. In some embodiments, the nucleotide sequence of the sense strand comprises the nucleotide sequence of any one of the sense strands of any one of dsRNA agents 129, 135, 169, 170, 409, 479, 480, 481, or 482 differing by no more than 1 (e.g., 0 or 1) nucleotide from the sense strand of any one of dsRNA agents 129, 135, 169, 170, 409, 479, 480, 481, or 482.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands of any one of dsRNA agents 129, 135, 169, 170, 409, 479, 480, 481, or 482. In some embodiments, the nucleotide sequence of the sense strand is at least 90%, 91%, 92%, 93%, 94%, 95%, 96%, 97%, 98%, 99% or 100% identical to the nucleotide sequence of any one of the sense strands of any one of dsRNA agents 129, 135, 169, 170, 409, 479, 480, 481, or 482. In some embodiments, the nucleotide sequence of the sense strand is at least 95%, 96%, 97%, 98%, 99% or 100% identical to the nucleotide sequence of any one of the sense strands of any one of dsRNA agents 129, 135, 169, 170, 409, 479, 480, 481, or 482. In some embodiments, the nucleotide sequence of the sense strand is at least 96%, 97%, 98%, 99% or 100% identical to the nucleotide sequence of any one of the sense strands of any one of dsRNA agents 129, 135, 169, 170, 409, 479, 480, 481, or 482. In some embodiments, the nucleotide sequence of the sense strand is at least 97%, identical to the nucleotide sequence of any one of the sense strands of any one of dsRNA agents 129, 135, 169, 170, 409, 479, 480, 481, or 482. In some embodiments, the nucleotide sequence of the sense strand is at least 98% identical to the nucleotide sequence of any one of the sense strands of any one of dsRNA agents 129, 135, 169, 170, 409, 479, 480, 481, or 482. In some embodiments, the nucleotide sequence of the sense strand is at least 99% identical to the nucleotide sequence of any one of the sense strands of any one of dsRNA agents 129, 135, 169, 170, 409, 479, 480, 481, or 482. In some embodiments, the nucleotide sequence of the sense strand is 100% identical to the nucleotide sequence of any one of the sense strands of any one of dsRNA agents 129, 135, 169, 170, 409, 479, 480, 481, or 482.

In some embodiments, the nucleotide sequence of the sense strand comprises the nucleotide sequence of any one of the sense strands of any one of dsRNA agents 129, 135, 169, 170, 409, 479, 480, 481, or 482. In some embodiments, the nucleotide sequence of the sense strand consists of the nucleotide sequence of any one of the sense strands of any one of dsRNA agents 129, 135, 169, 170, 409, 479, 480, 481, or 482.

Table 2 or Table 3 further identifies the target nucleic acid molecule within the cited reference CIDEB mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence of any one of the sense strands of any one of dsRNA agents 129, 135, 169, 170, 409, 479, 480, 481, or 482 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the CIDEB mRNA transcript targeted by the select sense strand.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482. In some embodiments, the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482.

In some embodiments, the nucleotide sequence of the sense strand comprises the nucleotide sequence of any one of the sense strands of any one of dsRNA agents 129, 135, 169, 170, 409, 479, 480, 481, or 482 differing by no more than 3 (e.g., 0, 1, 2, or 3) nucleotides from the sense strand. In some embodiments, the nucleotide sequence of the sense strand comprises the nucleotide sequence of any one of the sense strands of any one of dsRNA 129, 135, 169, 170, 409, 479, 480, 481, or 482.

As described above, Table 2 or Table 3 further identifies the target nucleic acid molecule within the reference CIDEB mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 129, 135, 169, 170, 409, 479, 480, 481, or 482 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the sense strand comprises at least 15 (e.g., 16, 17, 18, 19, 20, 21) contiguous nucleotides differing by no more than 3 (e.g., 0, 1, 2, or 3) nucleotides from 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1. In some embodiments, the sense strand comprises at least 19 (e.g., 20, 21) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1. In some embodiments, the sense strand comprises at least 21 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740,1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1. In some embodiments, the sense strand comprises from about 21-23 (e.g., 21-22, 22-23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1.

As described above, Table 2 or Table 3 further identifies the target nucleic acid molecule within the reference CIDEB mRNA transcript at least partially identical to each of the sense strands. As such, the disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

In some embodiments, the sense strand comprises at least 15 (e.g., 16, 17, 18, 19, 20, 21) contiguous nucleotides differing by no more than 3 (e.g., 0, 1, 2, or 3) nucleotides from 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1. In some embodiments, the sense strand comprises at least 19 (e.g., 20, 21) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1. In some embodiments, the sense strand comprises at least 21 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1. In some embodiments, the sense strand comprises from about 21-23 (e.g., 21-22, 22-23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1.

As described above, Table 2 or Table 3 further identifies the target nucleic acid molecule within the reference CIDEB mRNA transcript at least partially identical to each of the sense strands. As such, the disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1 and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the CIDEB mRNA transcript identical to the select sense strand.

It is to be understood, that although the sense strands are not described as being modified (e.g., comprising chemically modified nucleotides), conjugated, etc., the disclosure includes any sense strand that is unmodified, unconjugated, modified (e.g., as described herein), or conjugated (e.g., as described herein).

4.2.3 dsRNA Agents

In some embodiments, the agent (e.g., RNAi agent) comprises a dsRNA agent comprising an antisense strand (e.g., described herein, e.g., described in § 4.2.1) and a sense strand (e.g., described herein, e.g., described in § 4.2.2) that hybridize to form a double stranded region (e.g., under conditions in which the dsRNA will be used (e.g., under physiological (e.g., mammalian, e.g., human) conditions within a cell)).

As described above, antisense strands (e.g., described herein) comprise a region of complementarity that comprises a nucleotide sequence that is at least partially (e.g., substantially, fully) complementary to the nucleotide sequence of a target nucleic acid molecule (e.g., a target mRNA (e.g., a CIDEB mRNA), a portion of a target mRNA (e.g., a CIDEB mRNA)); and the sense strands comprise a region of complementarity that comprises a nucleotide sequence that is at least partially (e.g., substantially, fully) complementary to the nucleotide sequence of at least a portion of an antisense strand.

### 4.2.3.1 Single & Multiple Nucleic Acid Molecules

As described herein, and known in the art, the sense strand and the antisense strand can be part of a single larger nucleic acid molecule (connected as a single stranded nucleic acid molecule) or separate nucleic acid molecules (only connected through the double stranded region). In some embodiments, the sense strand and the antisense strand are separate nucleic acid molecules. In some embodiments, sense strand and the antisense strand are part of a single larger nucleic acid molecule.

In embodiments wherein the sense and antisense strands are part of a single nucleic acid molecule, the nucleic acid molecule may comprise a hairpin loop between the antisense strand and the sense strand to allow for formation of the double stranded region. In some embodiments, the hairpin loop comprises at least 1 (e.g., at least 2, 3, 4, 5, 6, 7, 8, 9, 10, 20, 23, 25 or more) unpaired nucleotides (non-complementary nucleotide mismatches). In some embodiments, the hairpin loop comprises at least one but less than 25, 23, 20, 10, 9, 8, 7, 6, 5, 4, 3, or 2 unpaired nucleotides (non-complementary nucleotide mismatches). In some embodiments, the hairpin loop comprises about 25, 23, 20, 9, 8, 7, 6, 5, 4, 3, or 1 unpaired nucleotide (non-complementary nucleotide mismatch).

Without wishing to be bound by theory, in embodiments wherein the sense strand and the antisense strand are part of a single nucleic acid molecule, after introduction into a suitable cell (e.g., a mammalian cell, e.g., a human cell), the nucleic acid molecule may be cleaved into a dsRNA molecule wherein the two strands of the dsRNA molecule are no longer part of the same nucleic acid molecule e.g., by a Type III endonuclease (e.g., Dicer) (see, e.g., Sharp et al. (2001) Genes Dev. 15:485, the entire contents of which are incorporated by herein by reference for all purposes).

### 4.2.3.2 Length of Double Stranded Region

In some embodiments, the double stranded region is about 15-30 base pairs in length (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length). In some embodiments, the double stranded region is about 18-25 base pairs in length (e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length (e.g., 19-21 base pairs in length)). In some embodiments, the double stranded region is about 15-30, 15-29, 15-28, 15-27, 15-26, 15-25, 15-24, 15-23, 15-22, 15-21, 15-20, 15-19, 15-18, 15-17, 15-16, 19-30, 19-29, 19-28, 19-27, 19-26, 19-25, 19-24, 19-23, 19-22, 19-20, 19-21, 23-30, 23-29, 23-28, 23-27, 23-26, 23-25, 23-24, 21-30, 21-29, 21-28, 21-27, 21-26, 21-25, 21-24, 21-23, or 21-22 base pairs in length. In some embodiments, the double stranded region is about 19-21 (e.g., 19-20) base pairs in length.

In some embodiments, the double stranded region is not more than about 15, 16, 17, 18, 19, 20, 21, 22, 23, 24, 25, 26, 27, 28, 29, or 30 base pairs in length. In some embodiments, the double stranded region is about 15, 16, 17, 18, 19, 20, 21, 22, 23, 24, 25, 26, 27, 28, 29, or 30 base pairs in length. In some embodiments, the double stranded region is about 19, 20, or 21 base pairs in length. In some embodiments, the double stranded region is about 19 base pairs in length. In some embodiments, the double stranded region is about 20 base pairs in length. In some embodiments, the double stranded region is about 21 base pairs in length. In some embodiments, the double stranded region is about 23 base pairs in length. Ranges and lengths intermediate to the above recited ranges and lengths are also contemplated to be part of the disclosure.

### 4.2.3.3 Nucleotide Overhangs & Blunt Ends

In some embodiments, the dsRNA agent comprises one or more (e.g., 1 or 2) nucleotide overhang. As is clear from the disclosure, but for the sake of clarity, the nucleotides of a nucleotide overhang can include one or more a modified (e.g., chemically modified) nucleotide (e.g., described herein, e.g., described in §§ 4.3, 4.3.1).

In some embodiments, the nucleotide overhang comprises from about 1-5 nucleotides, e.g., 1-4, 1-3, 1-2, 2-5, 2-4, 2-3, 3-5, 3-4, 4-5 nucleotides. In some embodiments, the nucleotide overhang comprises or consists of about 1, 2, 3, 4, or 5 nucleotides. In some embodiments, the nucleotide overhang comprises or consists of about 1 nucleotide. In some embodiments, the nucleotide overhang comprises or consists of about 2 nucleotides.

The nucleotide overhang(s) can be on the sense strand, the antisense strand, or both the sense strand and the antisense strand. In some embodiments, the sense strand comprises a nucleotide overhang. In some embodiments, the antisense strand comprises a nucleotide overhang. In some embodiments, the sense strand and the antisense strand both comprise a nucleotide overhang.

Furthermore, the nucleotide(s) of an overhang can be present on the 5′-end, 3′-end, or both the 5′-end, 3′-end of an antisense or sense strand. In some embodiments, the sense strand comprises a nucleotide overhang at the 5′-end. In some embodiments, the sense strand comprises a nucleotide overhang at the 3′-end. In some embodiments, the sense strand comprises a nucleotide overhang at the 5′-end and the 3′-end. In some embodiments, the antisense strand comprises a nucleotide overhang at the 5′-end. In some embodiments, the antisense strand comprises a nucleotide overhang at the 3′-end. In some embodiments, the antisense strand comprises a nucleotide overhang at the 5′-end and the 3′-end. In some embodiments, the antisense strand comprises a nucleotide overhang at the 3′-end; and the sense strand comprises a nucleotide overhang at the 3′-end. In some embodiments, the antisense strand comprises a nucleotide overhang at the 5′-end; and the sense strand comprises a nucleotide overhang at the 5′-end.

In some embodiments, the dsRNA agent comprises one or more blunt end. In some embodiments, the dsRNA agent comprises a blunt end at the end of the agent comprising the 3′ end of the sense strand and the 5′ end of the antisense strand. In some embodiments, the dsRNA agent comprises a blunt end at the end of the agent comprising the 5′ end of the sense strand and the 3′ end of the antisense strand. In some embodiments, both ends of the dsRNA agent are blunt ends.

### 4.2.3.4 Exemplary Structural Combinations of Sense & Antisense Strands

In some embodiments, the antisense strand and the sense strand contain the same number of nucleotides. In some embodiments, the antisense strand and the sense strand contain different numbers of nucleotides. In some embodiments, the nucleotide sequence of the sense strand is from about 1-5, 1-3, or 1-2 nucleotides shorter than the nucleotide sequence of the antisense strand. In some embodiments, the nucleotide sequence of the sense strand is about 1, 2, 3, 4, or 5 nucleotides shorter than the nucleotide sequence of the antisense strand. In some embodiments, the nucleotide sequence of the sense strand is about 2 nucleotides shorter than the nucleotide sequence of the antisense strand. In some embodiments, the nucleotide sequence of the antisense strand is from about 1-5, 1-3, or 1-2 nucleotides shorter than the nucleotide sequence of the sense strand. In some embodiments, the nucleotide sequence of the antisense strand is about 1, 2, 3, 4, or 5 nucleotides shorter than the nucleotide sequence of the sense strand. In some embodiments, the nucleotide sequence of the antisense strand is about 2 nucleotides shorter than the nucleotide sequence of the sense strand.

In some embodiments, the sense strand comprises or consists of 21 nucleotides. In some embodiments, the antisense strand comprises or consists of 23 nucleotides. In some embodiments, the sense strand comprises or consists of 21 nucleotides; and the antisense strand comprises or consists of 23 nucleotides. In some embodiments, the double stranded region comprises or consists of 21 nucleotides. In some embodiments, the antisense strand comprises a 2-nucleotide overhang at the 3′ end. In some embodiments, the 5′ end of the antisense strand and 3′ end of the sense strand form a blunt end. In some embodiments, the sense strand comprises or consists of 21 nucleotides; the antisense strand comprises or consists of 23 nucleotides; the double stranded region comprises or consists of 21 nucleotides; the antisense strand comprises a 2-nucleotide overhang at the 3′ end; and the 5′ end of the antisense strand and 3′ end of the sense strand form a blunt end.

In some embodiments, the sense strand comprises or consists of 19 nucleotides. In some embodiments, the antisense strand comprises or consists of 21 nucleotides. In some embodiments, the sense strand comprises or consists of 19 nucleotides; and the antisense strand comprises or consists of 21 nucleotides. In some embodiments, the double stranded region comprises or consists of 19 nucleotides. In some embodiments, the antisense strand comprises a 2-nucleotide overhang at the 3′ end. In some embodiments, the 5′ end of the antisense strand and 3′ end of the sense strand form a blunt end. In some embodiments, the sense strand comprises or consists of 19 nucleotides; the antisense strand comprises or consists of 21 nucleotides; the double stranded region comprises or consists of 19 nucleotides; the antisense strand comprises a 2-nucleotide overhang at the 3′ end; and the 5′ end of the antisense strand and 3′ end of the sense strand form a blunt end.

In some embodiments, the sense strand comprises or consists of 21 nucleotides. In some embodiments, the antisense strand comprises or consists of 21 nucleotides. In some embodiments, the sense strand comprises or consists of 21 nucleotides; and the antisense strand comprises or consists of 21 nucleotides. In some embodiments, the double stranded region comprises or consists of 19 nucleotides. In some embodiments, the antisense strand comprises a 2-nucleotide overhang at the 3′ end. In some embodiments, the sense strand comprises a 2-nucleotide overhang at the 3′ end. In some embodiments, the sense strand comprises or consists of 21 nucleotides; the antisense strand comprises or consists of 21 nucleotides; the double stranded region comprises or consists of 19 nucleotides; the antisense strand comprises a 2-nucleotide overhang at the 3′ end; and the sense strand comprises a 2-nucleotide overhang at the 3′ end.

In some embodiments, the sense strand comprises or consists of 20 nucleotides. In some embodiments, the antisense strand comprises or consists of 19 nucleotides. In some embodiments, the sense strand comprises or consists of 20 nucleotides; and the antisense strand comprises or consists of 19 nucleotides. In some embodiments, the double stranded region comprises or consists of 20 nucleotides. In some embodiments, the sense strand comprises a 1-nucleotide overhang at the 5′ end. In some embodiments, the 5′ end of the antisense strand and 3′ end of the sense strand form a blunt end. In some embodiments, the sense strand comprises or consists of 20 nucleotides; the antisense strand comprises or consists of 19 nucleotides; the double stranded region comprises or consists of 20 nucleotides; the sense strand comprises a 1-nucleotide overhang at the 5′ end; and the 5′ end of the antisense strand and 3′ end of the sense strand form a blunt end.

In some embodiments, the sense strand comprises or consists of 21 nucleotides. In some embodiments, the antisense strand comprises or consists of 19 nucleotides. In some embodiments, the sense strand comprises or consists of 21 nucleotides; and the antisense strand comprises or consists of 19 nucleotides. In some embodiments, the double stranded region comprises or consists of 19 nucleotides. In some embodiments, the sense strand comprises a 1-nucleotide overhang at the 3′ end. In some embodiments, the sense strand comprises a 1-nucleotide overhang at the 5′ end. In some embodiments, the sense strand comprises or consists of 21 nucleotides; the antisense strand comprises or consists of 19 nucleotides; the double stranded region comprises or consists of 19 nucleotides; the sense strand comprises a 1-nucleotide overhang at the 3′ end; and the sense strand comprises a 1-nucleotide overhang at the 5′ end.

In some embodiments, the sense strand comprises or consists of 24 nucleotides. In some embodiments, the antisense strand comprises or consists of 23 nucleotides. In some embodiments, the sense strand comprises or consists of 24 nucleotides; and the antisense strand comprises or consists of 23 nucleotides. In some embodiments, the double stranded region comprises or consists of 21 nucleotides. In some embodiments, the antisense strand comprises a 2-nucleotide overhang at the 3′ end. In some embodiments, the sense strand comprises a 3-nucleotide overhang at the 3′ end. In some embodiments, the sense strand comprises or consists of 24 nucleotides; the antisense strand comprises or consists of 23 nucleotides; the double stranded region comprises or consists of 21 nucleotides; the antisense strand comprises a 2-nucleotide overhang at the 3′ end; and the sense strand comprises a 3-nucleotide overhang at the 3′ end.

In some embodiments, the sense strand comprises or consists of 19 nucleotides. In some embodiments, the antisense strand comprises or consists of 19 nucleotides. In some embodiments, the sense strand comprises or consists of 19 nucleotides; and the antisense strand comprises or consists of 19 nucleotides. In some embodiments, the double stranded region comprises or consists of 19 nucleotides. In some embodiments, the 5′ end of the antisense strand (and 3′ end of the sense strand) form a blunt end. In some embodiments, the 3′ end of the antisense strand (and 5′ end of the sense strand) form a blunt end. In some embodiments, the sense strand comprises or consists of 19 nucleotides; the antisense strand comprises or consists of 19 nucleotides; the double stranded region comprises or consists of 19 nucleotides; the 5′ end of the antisense strand (and 3′ end of the sense strand) form a blunt end; and the 3′ end of the antisense strand (and 5′ end of the sense strand) form a blunt end

In some embodiments, the antisense strand and the sense strand are part of the same larger nucleic acid molecule, wherein the nucleic acid molecule comprises or consists of 44 nucleotides, the antisense portion comprises or consists of 21 nucleotides, the sense strand portion of the nucleic acid molecule comprises 19 nucleotides, the double stranded region comprises or consists of 19 nucleotides, the antisense strand comprises a 2-nucleotide overhang at the 3′ end, and the intervening nucleotide sequence between the antisense strand and the sense strand comprises or consists of 4 unpaired nucleotides that create a hairpin loop.

### 4.2.3.5 Exemplary Antisense Strands & Sense Strands

In some embodiments, the antisense strand is an antisense strand described herein. In some embodiments, the sense strand is a sense strand described herein. In some embodiments, the antisense strand is an antisense strand described in § 4.2.1. In some embodiments, the sense strand is a sense strand described in § 4.2.2. In some embodiments, the antisense strand is an antisense strand described in § 4.2.1; and the sense strand is a sense strand described in § 4.2.2. It is to be understood that any sense strand described herein (e.g., in § 4.2.2); and be utilized in combination with any antisense strand in a dsRNA agent described herein (e.g., in § 4.2.1). For the sake of clarity, the entire contents of in §§ 4.2.1 and § 4.2.2, are incorporated by reference into the instant section in § 4.2.3.5.

In some embodiments, the nucleotide sequence of the sense strand differs by no more than 5 (e.g., 0, 1, 2, 3, 4, or 5) nucleotides from the nucleotide sequence of any one of the sense strands set forth in Table 2 or Table 3; and the nucleotide sequence of the antisense strand differs by no more than 5 (e.g., 0, 1, 2, 3, 4, or 5) nucleotides from the nucleotide sequence of any one of the antisense strands set forth in Table 2 or Table 3. In some embodiments, the nucleotide sequence of the sense strand differs by no more than 3 (e.g., 0, 1, 2, or 3) nucleotides from the nucleotide sequence of any one of the sense strands set forth in Table 2 or Table 3; and the nucleotide sequence of the antisense strand differs by no more than 3 (e.g., 0, 1, 2, or 3) nucleotides from the nucleotide sequence of any one of the antisense strands set forth in Table 2 or Table 3. In some embodiments, the nucleotide sequence of the sense strand differs by no more than 2 (e.g., 0, 1, or 2) nucleotides from the nucleotide sequence of any one of the sense strands set forth in Table 2 or Table 3; and the nucleotide sequence of the antisense strand differs by no more than 2 (e.g., 0, 1, or 2) nucleotides from the nucleotide sequence of any one of the antisense strands set forth in Table 2 or Table 3. In some embodiments, the nucleotide sequence of the sense strand differs by no more than 1 (e.g., 0 or 1) nucleotide from the nucleotide sequence of any one of the sense strands set forth in Table 2 or Table 3; and the nucleotide sequence of the antisense strand differs by no more than 1 (e.g., 0 or 1) nucleotide from the nucleotide sequence of any one of the antisense strands set forth in Table 2 or Table 3.

In some embodiments, the nucleotide sequence of the sense strand comprises the nucleotide sequence of any one of the sense strands set forth in Table 2 or Table 3 differing by no more than 5 (e.g., 0, 1, 2, 3, 4, or 5) nucleotides from the sense strand set forth in Table 2 or Table 3; and the nucleotide sequence of the antisense strand comprises the nucleotide sequence of any one of the antisense strands set forth in Table 2 or Table 3 differing by no more than 5 (e.g., 0, 1, 2, 3, 4, or 5) nucleotides from the antisense strand set forth in Table 2 or Table 3. In some embodiments, the nucleotide sequence of the sense strand comprises the nucleotide sequence of any one of the sense strands set forth in Table 2 or Table 3 differing by no more than 3 (e.g., 0, 1, 2, or 3) nucleotides from the sense strand set forth in Table 2 or Table 3; and the nucleotide sequence of the antisense strand comprises the nucleotide sequence of any one of the antisense strands set forth in Table 2 or Table 3 differing by no more than 3 (e.g., 0, 1, 2, or 3) nucleotides from the antisense strand set forth in Table 2 or Table 3. In some embodiments, the nucleotide sequence of the sense strand comprises the nucleotide sequence of any one of the sense strands set forth in Table 2 or Table 3 differing by no more than 2 (e.g., 0, 1, or 2) nucleotides from the sense strand set forth in Table 2 or Table 3; and the nucleotide sequence of the antisense strand comprises the nucleotide sequence of any one of the antisense strands set forth in Table 2 or Table 3 differing by no more than 2 (e.g., 0, 1, or 2) nucleotides from the antisense strand set forth in Table 2 or Table 3. In some embodiments, the nucleotide sequence of the sense strand comprises the nucleotide sequence of any one of the sense strands set forth in Table 2 or Table 3 differing by no more than 1 (e.g., 0 or 1) nucleotide from the sense strand set forth in Table 2 or Table 3; and the nucleotide sequence of the antisense strand comprises the nucleotide sequence of any one of the antisense strands set forth in Table 2 or Table 3 differing by no more than 1 (e.g., 0 or 1) nucleotide from the antisense strand set forth in Table 2 or Table 3.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands set forth in Table 2 or Table 3; and the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands set forth in Table 2 or Table 3. In some embodiments, the nucleotide sequence of the sense strand is at least 90%, 91%, 92%, 93%, 94%, 95%, 96%, 97%, 98%, 99% or 100% identical to the nucleotide sequence of any one of the sense strands set forth in Table 2 or Table 3; and the nucleotide sequence of the antisense strand is at least 90%, 91%, 92%, 93%, 94%, 95%, 96%, 97%, 98%, 99% or 100% identical to the nucleotide sequence of any one of the antisense strands set forth in Table 2 or Table 3. In some embodiments, the nucleotide sequence of the sense strand is at least 95%, 96%, 97%, 98%, 99% or 100% identical to the nucleotide sequence of any one of the sense strands set forth in Table 2 or Table 3; and the nucleotide sequence of the antisense strand is at least 95%, 96%, 97%, 98%, 99% or 100% identical to the nucleotide sequence of any one of the antisense strands set forth in Table 2 or Table 3. In some embodiments, the nucleotide sequence of the sense strand is at least 96%, 97%, 98%, 99% or 100% identical to the nucleotide sequence of any one of the sense strands set forth in Table 2 or Table 3; and the nucleotide sequence of the antisense strand is at least 96%, 97%, 98%, 99% or 100% identical to the nucleotide sequence of any one of the antisense strands set forth in Table 2 or Table 3. In some embodiments, the nucleotide sequence of the sense strand is at least 97%, identical to the nucleotide sequence of any one of the sense strands set forth in Table 2 or Table 3; and the nucleotide sequence of the antisense strand is at least 97%, identical to the nucleotide sequence of any one of the antisense strands set forth in Table 2 or Table 3. In some embodiments, the nucleotide sequence of the sense strand is at least 98% identical to the nucleotide sequence of any one of the sense strands set forth in Table 2 or Table 3; and the nucleotide sequence of the antisense strand is at least 98% identical to the nucleotide sequence of any one of the antisense strands set forth in Table 2 or Table 3. In some embodiments, the nucleotide sequence of the sense strand is at least 99% identical to the nucleotide sequence of any one of the sense strands set forth in Table 2 or Table 3; and the nucleotide sequence of the antisense strand is at least 99% identical to the nucleotide sequence of any one of the antisense strands set forth in Table 2 or Table 3. In some embodiments, the nucleotide sequence of the sense strand is 100% identical to the nucleotide sequence of any one of the sense strands set forth in Table 2 or Table 3; and the nucleotide sequence of the antisense strand is 100% identical to the nucleotide sequence of any one of the antisense strands set forth in Table 2 or Table 3.

In some embodiments, the nucleotide sequence of the sense strand comprises the nucleotide sequence of any one of the sense strands set forth in Table 2 or Table 3; and the nucleotide sequence of the antisense strand comprises the nucleotide sequence of any one of the antisense strands set forth in Table 2 or Table 3. In some embodiments, the nucleotide sequence of the sense strand consists of the nucleotide sequence of any one of the sense strands set forth in Table 2 or Table 3; and the nucleotide sequence of the antisense strand consists of the nucleotide sequence of any one of the antisense strands set forth in Table 2 or Table 3.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in Table 2 or Table 3.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in Table 2 or Table 3.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 23 contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 21 contiguous nucleotides differing by no more than 3 (e.g., 0, 1, 2, or 3) nucleotides from the nucleotide sequence of any one of the sense strands set forth in Table 2 or Table 3. In some embodiments, the antisense strand comprises or consists of about 21 contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 19 contiguous nucleotides differing by no more than 3 (e.g., 0, 1, 2, or 3) nucleotides from the nucleotide sequence of any one of the sense strands set forth in Table 2 or Table 3.

In some embodiments, the nucleotide sequence of the antisense strand comprises or consists of the nucleotide sequence of any one of the antisense strands set forth in Table 2 or Table 3 differing by no more than 3 (e.g., 0, 1, 2, or 3) nucleotides from the antisense strand set forth in Table 2 or Table 3; and the nucleotide sequence of the sense strand comprises or consists of the nucleotide sequence of any one of the sense strands set forth in Table 2 or Table 3 differing by no more than 3 (e.g., 0, 1, 2, or 3) nucleotides from the sense strand set forth in Table 2 or Table 3.

In some embodiments, the nucleotide sequence of the antisense strand comprises or consists of the nucleotide sequence of any one of any one of the antisense strands set forth in Table 2 or Table 3; and the nucleotide sequence of the sense strand comprises or consists of the nucleotide sequence of any one of the sense strands set forth in Table 2 or Table 3.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in Table 2 or Table 3; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand set forth in Table 2 or Table 3.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in Table 2 or Table 3; and the sense strand comprises from about 21-23 (e.g., 21-22, 22-23) contiguous nucleotides differing by no more than 3 (e.g., 0, 1, 2, or 3) nucleotides from the nucleotide sequence of the corresponding sense strand set forth in Table 2 or Table 3.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 23 contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 21 contiguous nucleotides differing by no more than 3 (e.g., 0, 1, 2, or 3) nucleotides from the nucleotide sequence of the corresponding sense strand set forth in Table 2 or Table 3. In some embodiments, the antisense strand comprises or consists of about 21 contiguous nucleotides differing by no more than 3 (e.g., 0, 1, 2, or 3) nucleotides from the nucleotide sequence of any one of the antisense strands set forth in Table 2 or Table 3; and the sense strand comprises or consists of about 19 contiguous nucleotides differing by no more than 3 (e.g., 0, 1, 2, or 3) nucleotides from the nucleotide sequence of the corresponding sense strand set forth in Table 2 or Table 3.

In some embodiments, the nucleotide sequence of the antisense strand comprises or consists of the nucleotide sequence of any one of the antisense strands set forth in Table 2 or Table 3 differing by no more than 3 (e.g., 0, 1, 2, or 3) nucleotides from the antisense strand set forth in Table 2 or Table 3; and the nucleotide sequence of the sense strand comprises or consists of the nucleotide sequence of the corresponding sense strand set forth in Table 2 or Table 3 differing by no more than 3 (e.g., 0, 1, 2, or 3) nucleotides from the corresponding sense strand set forth in Table 2 or Table 3.

In some embodiments, the nucleotide sequence of the antisense strand comprises or consists of the nucleotide sequence of any one of any one of the antisense strands set forth in Table 2 or Table 3; and the nucleotide sequence of the sense strand comprises or consists of the nucleotide sequence of the corresponding sense strand set forth in Table 2 or Table 3.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 4-186, 553-601, 658-892, or 1192; and the nucleotide sequence of the antisense strand comprises the nucleotide sequence set forth in any one of SEQ ID NOS: 187-369, 602-657, 893-1131, or 1188-1191 differing by no more than 5 (e.g., 0, 1, 2, 3, 4, or 5) nucleotides from the nucleotide sequence set forth in any one of SEQ ID NOS: 187-369, 602-657, 893-1131, or 1188-1191. In some embodiments, the nucleotide sequence of the sense strand differs by no more than 3 (e.g., 0, 1, 2, or 3) nucleotides from the nucleotide sequence set forth in any one of SEQ ID NOS: 4-186, 553-601, 658-892, or 1192; and the nucleotide sequence of the antisense strand comprises the nucleotide sequence set forth in any one of SEQ ID NOS: 187-369, 602-657, 893-1131, or 1188-1191 differing by no more than 3 (e.g., 0, 1, 2, or 3) nucleotides from the nucleotide sequence set forth in any one of SEQ ID NOS: 187-369, 602-657, 893-1131, or 1188-1191. In some embodiments, the nucleotide sequence of the sense strand differs by no more than 2 (e.g., 0, 1, or 2) nucleotides from the nucleotide sequence set forth in any one of SEQ ID NOS: 4-186, 553-601, 658-892, or 1192; and the nucleotide sequence of the antisense strand comprises the nucleotide sequence set forth in any one of SEQ ID NOS: 187-369, 602-657, 893-1131, or 1188-1191 differing by no more than 2 (e.g., 0, 1, or 2) nucleotides from the nucleotide sequence set forth in any one of SEQ ID NOS: 187-369, 602-657, 893-1131, or 1188-1191. In some embodiments, the nucleotide sequence of the sense strand differs by no more than 1 (e.g., 0 or 1) nucleotide from the nucleotide sequence set forth in any one of SEQ ID NOS: 4-186, 553-601, 658-892, or 1192; and the nucleotide sequence of the antisense strand comprises the nucleotide sequence set forth in any one of SEQ ID NOS: 187-369, 602-657, 893-1131, or 1188-1191 differing by no more than 1 (e.g., 0 or 1) nucleotide from the nucleotide sequence set forth in any one of SEQ ID NOS: 187-369, 602-657, 893-1131, or 1188-1191.

In some embodiments, the nucleotide sequence of the sense strand comprises the nucleotide sequence set forth in any one of SEQ ID NOS: 4-186, 553-601, 658-892, or 1192 differing by no more than 5 (e.g., 0, 1, 2, 3, 4, or 5) nucleotides from the nucleotide sequence set forth in any one of SEQ ID NOS: 4-186, 553-601, 658-892, or 1192; and the nucleotide sequence of the antisense strand differs by no more than 5 (e.g., 0, 1, 2, 3, 4, or 5) nucleotides from the nucleotide sequence set forth in any one of SEQ ID NOS: 187-369, 602-657, 893-1131, or 1188-1191. In some embodiments, the nucleotide sequence of the sense strand comprises the nucleotide sequence set forth in any one of SEQ ID NOS: 4-186, 553-601, 658-892, or 1192 differing by no more than 3 (e.g., 0, 1, 2, or 3) nucleotides from the nucleotide sequence set forth in any one of SEQ ID NOS: 4-186, 553-601, 658-892, or 1192; and the nucleotide sequence of the antisense strand differs by no more than 3 (e.g., 0, 1, 2, or 3) nucleotides from the nucleotide sequence set forth in any one of SEQ ID NOS: 187-369, 602-657, 893-1131, or 1188-1191. In some embodiments, the nucleotide sequence of the sense strand comprises the nucleotide sequence set forth in any one of SEQ ID NOS: 4-186, 553-601, 658-892, or 1192 differing by no more than 2 (e.g., 0, 1, or 2) nucleotides from the nucleotide sequence set forth in any one of SEQ ID NOS: 4-186, 553-601, 658-892, or 1192; and the nucleotide sequence of the antisense strand differs by no more than 2 (e.g., 0, 1, or 2) nucleotides from the nucleotide sequence set forth in any one of SEQ ID NOS: 187-369, 602-657, 893-1131, or 1188-1191. In some embodiments, the nucleotide sequence of the sense strand comprises the nucleotide sequence set forth in any one of SEQ ID NOS: 4-186, 553-601, 658-892, or 1192 differing by no more than 1 (e.g., 0 or 1) nucleotide from the nucleotide sequence set forth in any one of SEQ ID NOS: 4-186, 553-601, 658-892, or 1192; and the nucleotide sequence of the antisense strand differs by no more than 1 (e.g., 0 or 1) nucleotide from the nucleotide sequence set forth in any one of SEQ ID NOS: 187-369, 602-657, 893-1131, or 1188-1191.

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 4-186, 553-601, 658-892, or 1192; and the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 4-186, 553-601, 658-892, or 1192; and the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the sense strand is at least 95%, 96%, 97%, 98%, 99% or 100% identical to the nucleotide sequence set forth in any one of SEQ ID NOS: 4-186, 553-601, 658-892, or 1192; and the nucleotide sequence of the antisense strand is at least 95%, 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the sense strand is at least 96%, 97%, 98%, 99% or 100% identical to the nucleotide sequence set forth in any one of SEQ ID NOS: 4-186, 553-601, 658-892, or 1192; and the nucleotide sequence of the antisense strand is at least 96%, 97%, 98%, 99% or 100% identical to the nucleotide sequence set forth in any one of SEQ ID NOS: 187-369, 602-657, 893-1131, or 1188-1191. In some embodiments, the nucleotide sequence of the sense strand is at least 97%, identical to the nucleotide sequence set forth in any one of SEQ ID NOS: 4-186, 553-601, 658-892, or 1192; and the nucleotide sequence of the antisense strand is at least 97%, identical to the nucleotide sequence set forth in any one of SEQ ID NOS: 187-369, 602-657, 893-1131, or 1188-1191. In some embodiments, the nucleotide sequence of the sense strand is at least 98% identical to the nucleotide sequence set forth in any one of SEQ ID NOS: 4-186, 553-601, 658-892, or 1192; and the nucleotide sequence of the antisense strand is at least 98% identical to the nucleotide sequence set forth in any one of SEQ ID NOS: 187-369, 602-657, 893-1131, or 1188-1191. In some embodiments, the nucleotide sequence of the sense strand is at least 99% identical to the nucleotide sequence set forth in any one of SEQ ID NOS: 4-186, 553-601, 658-892, or 1192; and the nucleotide sequence of the antisense strand is at least 99% identical to the nucleotide sequence set forth in any one of SEQ ID NOS: 187-369, 602-657, 893-1131, or 1188-1191. In some embodiments, the nucleotide sequence of the sense strand is 100% identical to the nucleotide sequence set forth in any one of SEQ ID NOS: 4-186, 553-601, 658-892, or 1192; and the nucleotide sequence of the antisense strand is 100% identical to the nucleotide sequence set forth in any one of SEQ ID NOS: 187-369, 602-657, 893-1131, or 1188-1191.

In some embodiments, the nucleotide sequence of the sense strand comprises the nucleotide sequence set forth in any one of SEQ ID NOS: 4-186, 553-601, 658-892, or 1192; and the nucleotide sequence of the antisense strand comprises the nucleotide sequence set forth in any one of SEQ ID NOS: 187-369, 602-657, 893-1131, or 1188-1191. In some embodiments, the nucleotide sequence of the sense strand consists of the nucleotide sequence set forth in any one of SEQ ID NOS: 4-186, 553-601, 658-892, or 1192; and the nucleotide sequence of the antisense strand consists of the nucleotide sequence set forth in any one of SEQ ID NOS: 187-369, 602-657, 893-1131, or 1188-1191.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 4-186, 553-601, 658-892, or 1192. In some embodiments, the nucleotide sequence of the sense strand differs by no more than 3 (e.g., 0, 1, 2, or 3) nucleotides from the nucleotide sequence set forth in any one of SEQ ID NOS: 4-186, 553-601, 658-892, or 1192. In some embodiments, the nucleotide sequence of the sense strand differs by no more than 2 (e.g., 0, 1, or 2) nucleotides from the nucleotide sequence set forth in any one of SEQ ID NOS: 4-186, 553-601, 658-892, or 1192. In some embodiments, the nucleotide sequence of the sense strand differs by no more than 1 (e.g., 0 or 1) nucleotide from the nucleotide sequence set forth in any one of SEQ ID NOS: 4-186, 553-601, 658-892, or 1192.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 21 contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192. In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 19 contiguous nucleotides differing by no more than 3 (e.g., 0, 1, 2, or 3) nucleotides from the nucleotide sequence of any one of the sense strands set forth in any one of SEQ ID NOS: 4-186, 553-601, 658-892, or 1192.

In some embodiments, the nucleotide sequence of the antisense strand comprises or consists of the nucleotide sequence of any one of the antisense strands set forth in any one of SEQ ID NOS: 187-369, 602-657, 893-1131, or 1188-1191 differing by no more than 3 (e.g., 0, 1, 2, or 3) nucleotides from the antisense strand set forth in any one of SEQ ID NOS: 187-369, 602-657, 893-1131, or 1188-1191; and the nucleotide sequence of the sense strand comprises or consists of the nucleotide sequence of any one of the sense strands set forth in any one of SEQ ID NOS: 4-186, 553-601, 658-892, or 1192 differing by no more than 3 (e.g., 0, 1, 2, or 3) nucleotides from the sense strand set forth in any one of SEQ ID NOS: 4-186, 553-601, 658-892, or 1192.

In some embodiments, the nucleotide sequence of the antisense strand comprises or consists of the nucleotide sequence of any one of any one of the antisense strands set forth in any one of SEQ ID NOS: 187-369, 602-657, 893-1131, or 1188-1191; and the nucleotide sequence of the sense strand comprises or consists of the nucleotide sequence of any one of the sense strands set forth in any one of SEQ ID NOS: 4-186, 553-601, 658-892, or 1192.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises from about 21-23 (e.g., 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192).

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 21 contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192). In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of SEQ ID NOS: 187-369, 602-657, 893-1131, or 1188-1191; and the sense strand comprises or consists of about 19 contiguous nucleotides differing by no more than 3 (e.g., 0, 1, 2, or 3) nucleotides from the nucleotide sequence of the corresponding sense strand (e.g., as set forth in one of SEQ ID NOS: 4-186, 553-601, 658-892, or 1192).

In some embodiments, the nucleotide sequence of the antisense strand comprises or consists of the nucleotide sequence of any one of the antisense strands set forth in any one of SEQ ID NOS: 187-369, 602-657, 893-1131, or 1188-1191 differing by no more than 3 (e.g., 0, 1, 2, or 3) nucleotides from the antisense strand set forth in any one of SEQ ID NOS: 187-369, 602-657, 893-1131, or 1188-1191; and the nucleotide sequence of the sense strand comprises or consists of the nucleotide sequence of the corresponding sense strand (e.g., as set forth in one of SEQ ID NOS: 4-186, 553-601, 658-892, or 1192) differing by no more than 3 (e.g., 0, 1, 2, or 3) nucleotides from the sense strand set forth in any one of SEQ ID NOS: 4-186, 553-601, 658-892, or 1192.

In some embodiments, the nucleotide sequence of the antisense strand comprises or consists of the nucleotide sequence of any one of any one of the antisense strands set forth in any one of SEQ ID NOS: 187-369, 602-657, 893-1131, or 1188-1191; and the nucleotide sequence of the sense strand comprises or consists of the nucleotide sequence of the corresponding sense strand (e.g., as set forth in one of SEQ ID NOS: 4-186, 553-601, 658-892, or 1192).

In some embodiments, the nucleotide sequence of the antisense strand differs by no more than 5 (e.g., 0, 1, 2, 3, 4, or 5) nucleotides from the nucleotide sequence set forth in SEQ ID NO: 355; and the nucleotide sequence of the sense strand differs by no more than 5 (e.g., 0, 1, 2, 3, 4, or 5) nucleotides from the nucleotide sequence set forth in SEQ ID NO: 172. In some embodiments, the nucleotide sequence of the antisense strand differs by no more than 3 (e.g., 0, 1, 2, or 3) nucleotides from the nucleotide sequence set forth in SEQ ID NO: 355; and the nucleotide sequence of the sense strand differs by no more than 3 (e.g., 0, 1, 2, or 3) nucleotides from the nucleotide sequence set forth in SEQ ID NO: 172. In some embodiments, the nucleotide sequence of the antisense strand differs by no more than 2 (e.g., 0, 1, or 2) nucleotides from the nucleotide sequence set forth in SEQ ID NO: 355; and the nucleotide sequence of the sense strand differs by no more than 2 (e.g., 0, 1, or 2) nucleotides from the nucleotide sequence set forth in SEQ ID NO: 172. In some embodiments, the nucleotide sequence of the antisense strand differs by no more than 1 (e.g., 0 or 1) nucleotide from the nucleotide sequence set forth in SEQ ID NO: 355; and the nucleotide sequence of the sense strand differs by no more than 1 (e.g., 0 or 1) nucleotide from the nucleotide sequence set forth in SEQ ID NO: 172.

In some embodiments, the nucleotide sequence of the antisense strand comprises the nucleotide sequence set forth in SEQ ID NO: 355 differing by no more than 5 (e.g., 0, 1, 2, 3, 4, or 5) nucleotides from the nucleotide sequence set forth in SEQ ID NO: 355; and the nucleotide sequence of the sense strand comprises the nucleotide sequence set forth in SEQ ID NO: 355 differing by no more than 5 (e.g., 0, 1, 2, 3, 4, or 5) nucleotides from the nucleotide sequence set forth in SEQ ID NO: 172. In some embodiments, the nucleotide sequence of the antisense strand comprises the nucleotide sequence set forth in SEQ ID NO: 355 differing by no more than 3 (e.g., 0, 1, 2, or 3) nucleotides from the nucleotide sequence set forth in SEQ ID NO: 355; and the nucleotide sequence of the sense strand comprises the nucleotide sequence set forth in SEQ ID NO: 172 differing by no more than 3 (e.g., 0, 1, 2, or 3) nucleotides from the nucleotide sequence set forth in SEQ ID NO: 172. In some embodiments, the nucleotide sequence of the antisense strand comprises the nucleotide sequence set forth in SEQ ID NO: 355 differing by no more than 2 (e.g., 0, 1, or 2) nucleotides from the nucleotide sequence set forth in SEQ ID NO: 355; and the nucleotide sequence of the sense strand comprises the nucleotide sequence set forth in SEQ ID NO: 172 differing by no more than 2 (e.g., 0, 1, or 2) nucleotides from the nucleotide sequence set forth in SEQ ID NO: 172. In some embodiments, the nucleotide sequence of the antisense strand comprises the nucleotide sequence set forth in SEQ ID NO: 355 differing by no more than 1 (e.g., 0 or 1) nucleotide from nucleotide sequence set forth in SEQ ID NO: 355; and the nucleotide sequence of the sense strand comprises the nucleotide sequence set forth in SEQ ID NO: 172 differing by no more than 1 (e.g., 0 or 1) nucleotide from nucleotide sequence set forth in SEQ ID NO: 172.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 355; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 172. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 355; and the nucleotide sequence of the sense strand is at least 90%, 91%, 92%, 93%, 94%, 95%, 96%, 97%, 98%, 99% or 100% identical to the nucleotide sequence set forth in SEQ ID NO: 172. In some embodiments, the nucleotide sequence of the antisense strand is at least 95%, 96%, 97%, 98%, 99% or 100% identical to the nucleotide sequence set forth in SEQ ID NO: 355; and the nucleotide sequence of the sense strand is at least 95%, 96%, 97%, 98%, 99% or 100% identical to the nucleotide sequence set forth in SEQ ID NO: 172. In some embodiments, the nucleotide sequence of the antisense strand is at least 96%, 97%, 98%, 99% or 100% identical to the nucleotide sequence set forth in SEQ ID NO: 355; and the nucleotide sequence of the sense strand is at least 96%, 97%, 98%, 99% or 100% identical to the nucleotide sequence set forth in SEQ ID NO: 172. In some embodiments, the nucleotide sequence of the antisense strand is at least 97%, identical to the nucleotide sequence set forth in SEQ ID NO: 355; and the nucleotide sequence of the sense strand is at least 97%, identical to the nucleotide sequence set forth in SEQ ID NO: 172. In some embodiments, the nucleotide sequence of the antisense strand is at least 98% identical to the nucleotide sequence set forth in SEQ ID NO: 355; and the nucleotide sequence of the sense strand is at least 98% identical to the nucleotide sequence set forth in SEQ ID NO: 172. In some embodiments, the nucleotide sequence of the antisense strand is at least 99% identical to the nucleotide sequence set forth in SEQ ID NO: 355; and the nucleotide sequence of the sense strand is at least 99% identical to the nucleotide sequence set forth in SEQ ID NO: 172. In some embodiments, the nucleotide sequence of the antisense strand is 100% identical to the nucleotide sequence set forth in SEQ ID NO: 355; and the nucleotide sequence of the sense strand is 100% identical to the nucleotide sequence set forth in SEQ ID NO: 172.

In some embodiments, the nucleotide sequence of the antisense strand comprises the nucleotide sequence set forth in SEQ ID NO: 355; and the nucleotide sequence of the sense strand comprises the nucleotide sequence set forth in SEQ ID NO: 172. In some embodiments, the nucleotide sequence of the antisense strand consists of the nucleotide sequence set forth in SEQ ID NO: 355; and the nucleotide sequence of the sense strand consists of the nucleotide sequence set forth in SEQ ID NO: 172.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 355; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 355; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 355; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 355; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 172.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 355; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 355; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 355; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 355; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 172.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 355; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 355; and the sense strand comprises about 21 contiguous nucleotides differing by no more than 3 (e.g., 0, 1, 2, or 3) nucleotides from the nucleotide sequence of the sense strand set forth in SEQ ID NO: 172. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 355; and the sense strand comprises about 19 contiguous nucleotides differing by no more than 3 (e.g., 0, 1, 2, or 3) nucleotides from the nucleotide sequence of the sense strand set forth in SEQ ID NO: 172.

In some embodiments, the nucleotide sequence of the antisense strand comprises the nucleotide sequence of the antisense strand set forth in SEQ ID NO: 355 differing by no more than 3 (e.g., 0, 1, 2, or 3) nucleotides from the nucleotide sequence of the antisense strand set forth in SEQ ID NO: 355; and the nucleotide sequence of the sense strand comprises the nucleotide sequence of the sense strand set forth in SEQ ID NO: 172 differing by no more than 3 (e.g., 0, 1, 2, or 3) nucleotides from the sense strand set forth in SEQ ID NO: 172.

In some embodiments, the nucleotide sequence of the antisense strand comprises the nucleotide sequence of antisense strand set forth in SEQ ID NO: 355; and the nucleotide sequence of the sense strand comprises the nucleotide sequence of the sense strand set forth in SEQ ID NO: 172.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 355; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 355; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 355; and the sense strand consists of about 21 contiguous nucleotides differing by no more than 3 (e.g., 0, 1, 2, or 3) nucleotides from the nucleotide sequence of the sense strand set forth in SEQ ID NO: 172. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 355; and the sense strand consists of about 19 contiguous nucleotides differing by no more than 3 (e.g., 0, 1, 2, or 3) nucleotides from the nucleotide sequence of the sense strand set forth in SEQ ID NO: 172.

In some embodiments, the nucleotide sequence of the antisense strand consists of the nucleotide sequence of the antisense strand set forth in SEQ ID NO: 355 differing by no more than 3 (e.g., 0, 1, 2, or 3) nucleotides from the nucleotide sequence of the antisense strand set forth in SEQ ID NO: 355; and the nucleotide sequence of the sense strand consists of the nucleotide sequence of the sense strand set forth in SEQ ID NO: 172 differing by no more than 3 (e.g., 0, 1, 2, or 3) nucleotides from the sense strand set forth in SEQ ID NO: 172.

In some embodiments, the nucleotide sequence of the antisense strand consists of the nucleotide sequence of antisense strand set forth in SEQ ID NO: 355; and the nucleotide sequence of the sense strand consists of the nucleotide sequence of the sense strand set forth in SEQ ID NO: 172.

In some embodiments, the nucleotide sequence of the antisense strand differs by no more than 5 (e.g., 0, 1, 2, 3, 4, or 5) nucleotides from the nucleotide sequence set forth in SEQ ID NO: 315; and the nucleotide sequence of the sense strand differs by no more than 5 (e.g., 0, 1, 2, 3, 4, or 5) nucleotides from the nucleotide sequence set forth in SEQ ID NO: 132. In some embodiments, the nucleotide sequence of the antisense strand differs by no more than 3 (e.g., 0, 1, 2, or 3) nucleotides from the nucleotide sequence set forth in SEQ ID NO: 315; and the nucleotide sequence of the sense strand differs by no more than 3 (e.g., 0, 1, 2, or 3) nucleotides from the nucleotide sequence set forth in SEQ ID NO: 132. In some embodiments, the nucleotide sequence of the antisense strand differs by no more than 2 (e.g., 0, 1, or 2) nucleotides from the nucleotide sequence set forth in SEQ ID NO: 315; and the nucleotide sequence of the sense strand differs by no more than 2 (e.g., 0, 1, or 2) nucleotides from the nucleotide sequence set forth in SEQ ID NO: 132. In some embodiments, the nucleotide sequence of the antisense strand differs by no more than 1 (e.g., 0 or 1) nucleotide from the nucleotide sequence set forth in SEQ ID NO: 315; and the nucleotide sequence of the sense strand differs by no more than 1 (e.g., 0 or 1) nucleotide from the nucleotide sequence set forth in SEQ ID NO: 132.

In some embodiments, the nucleotide sequence of the antisense strand comprises the nucleotide sequence set forth in SEQ ID NO: 315 differing by no more than 5 (e.g., 0, 1, 2, 3, 4, or 5) nucleotides from the nucleotide sequence set forth in SEQ ID NO: 315; and the nucleotide sequence of the sense strand comprises the nucleotide sequence set forth in SEQ ID NO: 315 differing by no more than 5 (e.g., 0, 1, 2, 3, 4, or 5) nucleotides from the nucleotide sequence set forth in SEQ ID NO: 132. In some embodiments, the nucleotide sequence of the antisense strand comprises the nucleotide sequence set forth in SEQ ID NO: 315 differing by no more than 3 (e.g., 0, 1, 2, or 3) nucleotides from the nucleotide sequence set forth in SEQ ID NO: 315; and the nucleotide sequence of the sense strand comprises the nucleotide sequence set forth in SEQ ID NO: 132 differing by no more than 3 (e.g., 0, 1, 2, or 3) nucleotides from the nucleotide sequence set forth in SEQ ID NO: 132. In some embodiments, the nucleotide sequence of the antisense strand comprises the nucleotide sequence set forth in SEQ ID NO: 315 differing by no more than 2 (e.g., 0, 1, or 2) nucleotides from the nucleotide sequence set forth in SEQ ID NO: 315; and the nucleotide sequence of the sense strand comprises the nucleotide sequence set forth in SEQ ID NO: 132 differing by no more than 2 (e.g., 0, 1, or 2) nucleotides from the nucleotide sequence set forth in SEQ ID NO: 132. In some embodiments, the nucleotide sequence of the antisense strand comprises the nucleotide sequence set forth in SEQ ID NO: 315 differing by no more than 1 (e.g., 0 or 1) nucleotide from nucleotide sequence set forth in SEQ ID NO: 315; and the nucleotide sequence of the sense strand comprises the nucleotide sequence set forth in SEQ ID NO: 132 differing by no more than 1 (e.g., 0 or 1) nucleotide from nucleotide sequence set forth in SEQ ID NO: 132.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 315; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 132. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 315; and the nucleotide sequence of the sense strand is at least 90%, 91%, 92%, 93%, 94%, 95%, 96%, 97%, 98%, 99% or 100% identical to the nucleotide sequence set forth in SEQ ID NO: 132. In some embodiments, the nucleotide sequence of the antisense strand is at least 95%, 96%, 97%, 98%, 99% or 100% identical to the nucleotide sequence set forth in SEQ ID NO: 315; and the nucleotide sequence of the sense strand is at least 95%, 96%, 97%, 98%, 99% or 100% identical to the nucleotide sequence set forth in SEQ ID NO: 132. In some embodiments, the nucleotide sequence of the antisense strand is at least 96%, 97%, 98%, 99% or 100% identical to the nucleotide sequence set forth in SEQ ID NO: 315; and the nucleotide sequence of the sense strand is at least 96%, 97%, 98%, 99% or 100% identical to the nucleotide sequence set forth in SEQ ID NO: 132. In some embodiments, the nucleotide sequence of the antisense strand is at least 97%, identical to the nucleotide sequence set forth in SEQ ID NO: 315; and the nucleotide sequence of the sense strand is at least 97%, identical to the nucleotide sequence set forth in SEQ ID NO: 132. In some embodiments, the nucleotide sequence of the antisense strand is at least 98% identical to the nucleotide sequence set forth in SEQ ID NO: 315; and the nucleotide sequence of the sense strand is at least 98% identical to the nucleotide sequence set forth in SEQ ID NO: 132. In some embodiments, the nucleotide sequence of the antisense strand is at least 99% identical to the nucleotide sequence set forth in SEQ ID NO: 315; and the nucleotide sequence of the sense strand is at least 99% identical to the nucleotide sequence set forth in SEQ ID NO: 132. In some embodiments, the nucleotide sequence of the antisense strand is 100% identical to the nucleotide sequence set forth in SEQ ID NO: 315; and the nucleotide sequence of the sense strand is 100% identical to the nucleotide sequence set forth in SEQ ID NO: 132.

In some embodiments, the nucleotide sequence of the antisense strand comprises the nucleotide sequence set forth in SEQ ID NO: 315; and the nucleotide sequence of the sense strand comprises the nucleotide sequence set forth in SEQ ID NO: 132. In some embodiments, the nucleotide sequence of the antisense strand consists of the nucleotide sequence set forth in SEQ ID NO: 315; and the nucleotide sequence of the sense strand consists of the nucleotide sequence set forth in SEQ ID NO: 132.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 315; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 315; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 315; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 315; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 132.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 315; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 315; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 315; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 315; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 132.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 315; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 315; and the sense strand comprises about 21 contiguous nucleotides differing by no more than 3 (e.g., 0, 1, 2, or 3) nucleotides from the nucleotide sequence of the sense strand set forth in SEQ ID NO: 132. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 315; and the sense strand comprises about 19 contiguous nucleotides differing by no more than 3 (e.g., 0, 1, 2, or 3) nucleotides from the nucleotide sequence of the sense strand set forth in SEQ ID NO: 132.

In some embodiments, the nucleotide sequence of the antisense strand comprises the nucleotide sequence of the antisense strand set forth in SEQ ID NO: 315 differing by no more than 3 (e.g., 0, 1, 2, or 3) nucleotides from the nucleotide sequence of the antisense strand set forth in SEQ ID NO: 315; and the nucleotide sequence of the sense strand comprises the nucleotide sequence of the sense strand set forth in SEQ ID NO: 132 differing by no more than 3 (e.g., 0, 1, 2, or 3) nucleotides from the sense strand set forth in SEQ ID NO: 132.

In some embodiments, the nucleotide sequence of the antisense strand comprises the nucleotide sequence of antisense strand set forth in SEQ ID NO: 315; and the nucleotide sequence of the sense strand comprises the nucleotide sequence of the sense strand set forth in SEQ ID NO: 132.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 315; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 315; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 315; and the sense strand consists of about 21 contiguous nucleotides differing by no more than 3 (e.g., 0, 1, 2, or 3) nucleotides from the nucleotide sequence of the sense strand set forth in SEQ ID NO: 132. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 315; and the sense strand consists of about 19 contiguous nucleotides differing by no more than 3 (e.g., 0, 1, 2, or 3) nucleotides from the nucleotide sequence of the sense strand set forth in SEQ ID NO: 132.

In some embodiments, the nucleotide sequence of the antisense strand consists of the nucleotide sequence of the antisense strand set forth in SEQ ID NO: 315 differing by no more than 3 (e.g., 0, 1, 2, or 3) nucleotides from the nucleotide sequence of the antisense strand set forth in SEQ ID NO: 315; and the nucleotide sequence of the sense strand consists of the nucleotide sequence of the sense strand set forth in SEQ ID NO: 132 differing by no more than 3 (e.g., 0, 1, 2, or 3) nucleotides from the sense strand set forth in SEQ ID NO: 132.

In some embodiments, the nucleotide sequence of the antisense strand consists of the nucleotide sequence of antisense strand set forth in SEQ ID NO: 315; and the nucleotide sequence of the sense strand consists of the nucleotide sequence of the sense strand set forth in SEQ ID NO: 132.

In some embodiments, the nucleotide sequence of the antisense strand differs by no more than 5 (e.g., 0, 1, 2, 3, 4, or 5) nucleotides from the nucleotide sequence set forth in SEQ ID NO: 356; and the nucleotide sequence of the sense strand differs by no more than 5 (e.g., 0, 1, 2, 3, 4, or 5) nucleotides from the nucleotide sequence set forth in SEQ ID NO: 173. In some embodiments, the nucleotide sequence of the antisense strand differs by no more than 3 (e.g., 0, 1, 2, or 3) nucleotides from the nucleotide sequence set forth in SEQ ID NO: 356; and the nucleotide sequence of the sense strand differs by no more than 3 (e.g., 0, 1, 2, or 3) nucleotides from the nucleotide sequence set forth in SEQ ID NO: 173. In some embodiments, the nucleotide sequence of the antisense strand differs by no more than 2 (e.g., 0, 1, or 2) nucleotides from the nucleotide sequence set forth in SEQ ID NO: 356; and the nucleotide sequence of the sense strand differs by no more than 2 (e.g., 0, 1, or 2) nucleotides from the nucleotide sequence set forth in SEQ ID NO: 173. In some embodiments, the nucleotide sequence of the antisense strand differs by no more than 1 (e.g., 0 or 1) nucleotide from the nucleotide sequence set forth in SEQ ID NO: 356; and the nucleotide sequence of the sense strand differs by no more than 1 (e.g., 0 or 1) nucleotide from the nucleotide sequence set forth in SEQ ID NO: 173.

In some embodiments, the nucleotide sequence of the antisense strand comprises the nucleotide sequence set forth in SEQ ID NO: 356 differing by no more than 5 (e.g., 0, 1, 2, 3, 4, or 5) nucleotides from the nucleotide sequence set forth in SEQ ID NO: 356; and the nucleotide sequence of the sense strand comprises the nucleotide sequence set forth in SEQ ID NO: 356 differing by no more than 5 (e.g., 0, 1, 2, 3, 4, or 5) nucleotides from the nucleotide sequence set forth in SEQ ID NO: 173. In some embodiments, the nucleotide sequence of the antisense strand comprises the nucleotide sequence set forth in SEQ ID NO: 356 differing by no more than 3 (e.g., 0, 1, 2, or 3) nucleotides from the nucleotide sequence set forth in SEQ ID NO: 356; and the nucleotide sequence of the sense strand comprises the nucleotide sequence set forth in SEQ ID NO: 173 differing by no more than 3 (e.g., 0, 1, 2, or 3) nucleotides from the nucleotide sequence set forth in SEQ ID NO: 173. In some embodiments, the nucleotide sequence of the antisense strand comprises the nucleotide sequence set forth in SEQ ID NO: 356 differing by no more than 2 (e.g., 0, 1, or 2) nucleotides from the nucleotide sequence set forth in SEQ ID NO: 356; and the nucleotide sequence of the sense strand comprises the nucleotide sequence set forth in SEQ ID NO: 173 differing by no more than 2 (e.g., 0, 1, or 2) nucleotides from the nucleotide sequence set forth in SEQ ID NO: 173. In some embodiments, the nucleotide sequence of the antisense strand comprises the nucleotide sequence set forth in SEQ ID NO: 356 differing by no more than 1 (e.g., 0 or 1) nucleotide from nucleotide sequence set forth in SEQ ID NO: 356; and the nucleotide sequence of the sense strand comprises the nucleotide sequence set forth in SEQ ID NO: 173 differing by no more than 1 (e.g., 0 or 1) nucleotide from nucleotide sequence set forth in SEQ ID NO: 173.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 356; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 173. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 356; and the nucleotide sequence of the sense strand is at least 90%, 91%, 92%, 93%, 94%, 95%, 96%, 97%, 98%, 99% or 100% identical to the nucleotide sequence set forth in SEQ ID NO: 173. In some embodiments, the nucleotide sequence of the antisense strand is at least 95%, 96%, 97%, 98%, 99% or 100% identical to the nucleotide sequence set forth in SEQ ID NO: 356; and the nucleotide sequence of the sense strand is at least 95%, 96%, 97%, 98%, 99% or 100% identical to the nucleotide sequence set forth in SEQ ID NO: 173. In some embodiments, the nucleotide sequence of the antisense strand is at least 96%, 97%, 98%, 99% or 100% identical to the nucleotide sequence set forth in SEQ ID NO: 356; and the nucleotide sequence of the sense strand is at least 96%, 97%, 98%, 99% or 100% identical to the nucleotide sequence set forth in SEQ ID NO: 173. In some embodiments, the nucleotide sequence of the antisense strand is at least 97%, identical to the nucleotide sequence set forth in SEQ ID NO: 356; and the nucleotide sequence of the sense strand is at least 97%, identical to the nucleotide sequence set forth in SEQ ID NO: 173. In some embodiments, the nucleotide sequence of the antisense strand is at least 98% identical to the nucleotide sequence set forth in SEQ ID NO: 356; and the nucleotide sequence of the sense strand is at least 98% identical to the nucleotide sequence set forth in SEQ ID NO: 173. In some embodiments, the nucleotide sequence of the antisense strand is at least 99% identical to the nucleotide sequence set forth in SEQ ID NO: 356; and the nucleotide sequence of the sense strand is at least 99% identical to the nucleotide sequence set forth in SEQ ID NO: 173. In some embodiments, the nucleotide sequence of the antisense strand is 100% identical to the nucleotide sequence set forth in SEQ ID NO: 356; and the nucleotide sequence of the sense strand is 100% identical to the nucleotide sequence set forth in SEQ ID NO: 173.

In some embodiments, the nucleotide sequence of the antisense strand comprises the nucleotide sequence set forth in SEQ ID NO: 356; and the nucleotide sequence of the sense strand comprises the nucleotide sequence set forth in SEQ ID NO: 173. In some embodiments, the nucleotide sequence of the antisense strand consists of the nucleotide sequence set forth in SEQ ID NO: 356; and the nucleotide sequence of the sense strand consists of the nucleotide sequence set forth in SEQ ID NO: 173.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 356; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 356; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 356; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 356; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 173.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 356; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 356; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 356; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 356; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 173.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 356; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 356; and the sense strand comprises about 21 contiguous nucleotides differing by no more than 3 (e.g., 0, 1, 2, or 3) nucleotides from the nucleotide sequence of the sense strand set forth in SEQ ID NO: 173. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 356; and the sense strand comprises about 19 contiguous nucleotides differing by no more than 3 (e.g., 0, 1, 2, or 3) nucleotides from the nucleotide sequence of the sense strand set forth in SEQ ID NO: 173.

In some embodiments, the nucleotide sequence of the antisense strand comprises the nucleotide sequence of the antisense strand set forth in SEQ ID NO: 356 differing by no more than 3 (e.g., 0, 1, 2, or 3) nucleotides from the nucleotide sequence of the antisense strand set forth in SEQ ID NO: 356; and the nucleotide sequence of the sense strand comprises the nucleotide sequence of the sense strand set forth in SEQ ID NO: 173 differing by no more than 3 (e.g., 0, 1, 2, or 3) nucleotides from the sense strand set forth in SEQ ID NO: 173.

In some embodiments, the nucleotide sequence of the antisense strand comprises the nucleotide sequence of antisense strand set forth in SEQ ID NO: 356; and the nucleotide sequence of the sense strand comprises the nucleotide sequence of the sense strand set forth in SEQ ID NO: 173.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 356; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 356; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 356; and the sense strand consists of about 21 contiguous nucleotides differing by no more than 3 (e.g., 0, 1, 2, or 3) nucleotides from the nucleotide sequence of the sense strand set forth in SEQ ID NO: 173. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 356; and the sense strand consists of about 19 contiguous nucleotides differing by no more than 3 (e.g., 0, 1, 2, or 3) nucleotides from the nucleotide sequence of the sense strand set forth in SEQ ID NO: 173.

In some embodiments, the nucleotide sequence of the antisense strand consists of the nucleotide sequence of the antisense strand set forth in SEQ ID NO: 356 differing by no more than 3 (e.g., 0, 1, 2, or 3) nucleotides from the nucleotide sequence of the antisense strand set forth in SEQ ID NO: 356; and the nucleotide sequence of the sense strand consists of the nucleotide sequence of the sense strand set forth in SEQ ID NO: 173 differing by no more than 3 (e.g., 0, 1, 2, or 3) nucleotides from the sense strand set forth in SEQ ID NO: 173.

In some embodiments, the nucleotide sequence of the antisense strand consists of the nucleotide sequence of antisense strand set forth in SEQ ID NO: 356; and the nucleotide sequence of the sense strand consists of the nucleotide sequence of the sense strand set forth in SEQ ID NO: 173.

In some embodiments, the nucleotide sequence of the antisense strand differs by no more than 5 (e.g., 0, 1, 2, 3, 4, or 5) nucleotides from the nucleotide sequence set forth in SEQ ID NO: 321; and the nucleotide sequence of the sense strand differs by no more than 5 (e.g., 0, 1, 2, 3, 4, or 5) nucleotides from the nucleotide sequence set forth in SEQ ID NO: 138. In some embodiments, the nucleotide sequence of the antisense strand differs by no more than 3 (e.g., 0, 1, 2, or 3) nucleotides from the nucleotide sequence set forth in SEQ ID NO: 321; and the nucleotide sequence of the sense strand differs by no more than 3 (e.g., 0, 1, 2, or 3) nucleotides from the nucleotide sequence set forth in SEQ ID NO: 138. In some embodiments, the nucleotide sequence of the antisense strand differs by no more than 2 (e.g., 0, 1, or 2) nucleotides from the nucleotide sequence set forth in SEQ ID NO: 321; and the nucleotide sequence of the sense strand differs by no more than 2 (e.g., 0, 1, or 2) nucleotides from the nucleotide sequence set forth in SEQ ID NO: 138. In some embodiments, the nucleotide sequence of the antisense strand differs by no more than 1 (e.g., 0 or 1) nucleotide from the nucleotide sequence set forth in SEQ ID NO: 321; and the nucleotide sequence of the sense strand differs by no more than 1 (e.g., 0 or 1) nucleotide from the nucleotide sequence set forth in SEQ ID NO: 138.

In some embodiments, the nucleotide sequence of the antisense strand comprises the nucleotide sequence set forth in SEQ ID NO: 321 differing by no more than 5 (e.g., 0, 1, 2, 3, 4, or 5) nucleotides from the nucleotide sequence set forth in SEQ ID NO: 321; and the nucleotide sequence of the sense strand comprises the nucleotide sequence set forth in SEQ ID NO: 321 differing by no more than 5 (e.g., 0, 1, 2, 3, 4, or 5) nucleotides from the nucleotide sequence set forth in SEQ ID NO: 138. In some embodiments, the nucleotide sequence of the antisense strand comprises the nucleotide sequence set forth in SEQ ID NO: 321 differing by no more than 3 (e.g., 0, 1, 2, or 3) nucleotides from the nucleotide sequence set forth in SEQ ID NO: 321; and the nucleotide sequence of the sense strand comprises the nucleotide sequence set forth in SEQ ID NO: 138 differing by no more than 3 (e.g., 0, 1, 2, or 3) nucleotides from the nucleotide sequence set forth in SEQ ID NO: 138. In some embodiments, the nucleotide sequence of the antisense strand comprises the nucleotide sequence set forth in SEQ ID NO: 321 differing by no more than 2 (e.g., 0, 1, or 2) nucleotides from the nucleotide sequence set forth in SEQ ID NO: 321; and the nucleotide sequence of the sense strand comprises the nucleotide sequence set forth in SEQ ID NO: 138 differing by no more than 2 (e.g., 0, 1, or 2) nucleotides from the nucleotide sequence set forth in SEQ ID NO: 138. In some embodiments, the nucleotide sequence of the antisense strand comprises the nucleotide sequence set forth in SEQ ID NO: 321 differing by no more than 1 (e.g., 0 or 1) nucleotide from nucleotide sequence set forth in SEQ ID NO: 321; and the nucleotide sequence of the sense strand comprises the nucleotide sequence set forth in SEQ ID NO: 138 differing by no more than 1 (e.g., 0 or 1) nucleotide from nucleotide sequence set forth in SEQ ID NO: 138.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 321; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 138. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 321; and the nucleotide sequence of the sense strand is at least 90%, 91%, 92%, 93%, 94%, 95%, 96%, 97%, 98%, 99% or 100% identical to the nucleotide sequence set forth in SEQ ID NO: 138. In some embodiments, the nucleotide sequence of the antisense strand is at least 95%, 96%, 97%, 98%, 99% or 100% identical to the nucleotide sequence set forth in SEQ ID NO: 321; and the nucleotide sequence of the sense strand is at least 95%, 96%, 97%, 98%, 99% or 100% identical to the nucleotide sequence set forth in SEQ ID NO: 138. In some embodiments, the nucleotide sequence of the antisense strand is at least 96%, 97%, 98%, 99% or 100% identical to the nucleotide sequence set forth in SEQ ID NO: 321; and the nucleotide sequence of the sense strand is at least 96%, 97%, 98%, 99% or 100% identical to the nucleotide sequence set forth in SEQ ID NO: 138. In some embodiments, the nucleotide sequence of the antisense strand is at least 97%, identical to the nucleotide sequence set forth in SEQ ID NO: 321; and the nucleotide sequence of the sense strand is at least 97%, identical to the nucleotide sequence set forth in SEQ ID NO: 138. In some embodiments, the nucleotide sequence of the antisense strand is at least 98% identical to the nucleotide sequence set forth in SEQ ID NO: 321; and the nucleotide sequence of the sense strand is at least 98% identical to the nucleotide sequence set forth in SEQ ID NO: 138. In some embodiments, the nucleotide sequence of the antisense strand is at least 99% identical to the nucleotide sequence set forth in SEQ ID NO: 321; and the nucleotide sequence of the sense strand is at least 99% identical to the nucleotide sequence set forth in SEQ ID NO: 138. In some embodiments, the nucleotide sequence of the antisense strand is 100% identical to the nucleotide sequence set forth in SEQ ID NO: 321; and the nucleotide sequence of the sense strand is 100% identical to the nucleotide sequence set forth in SEQ ID NO: 138.

In some embodiments, the nucleotide sequence of the antisense strand comprises the nucleotide sequence set forth in SEQ ID NO: 321; and the nucleotide sequence of the sense strand comprises the nucleotide sequence set forth in SEQ ID NO: 138. In some embodiments, the nucleotide sequence of the antisense strand consists of the nucleotide sequence set forth in SEQ ID NO: 321; and the nucleotide sequence of the sense strand consists of the nucleotide sequence set forth in SEQ ID NO: 138.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 321; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 321; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 321; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 321; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 138.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 321; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 321; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 321; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 321; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 138.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 321; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 321; and the sense strand comprises about 21 contiguous nucleotides differing by no more than 3 (e.g., 0, 1, 2, or 3) nucleotides from the nucleotide sequence of the sense strand set forth in SEQ ID NO: 138. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 321; and the sense strand comprises about 19 contiguous nucleotides differing by no more than 3 (e.g., 0, 1, 2, or 3) nucleotides from the nucleotide sequence of the sense strand set forth in SEQ ID NO: 138.

In some embodiments, the nucleotide sequence of the antisense strand comprises the nucleotide sequence of the antisense strand set forth in SEQ ID NO: 321 differing by no more than 3 (e.g., 0, 1, 2, or 3) nucleotides from the nucleotide sequence of the antisense strand set forth in SEQ ID NO: 321; and the nucleotide sequence of the sense strand comprises the nucleotide sequence of the sense strand set forth in SEQ ID NO: 138 differing by no more than 3 (e.g., 0, 1, 2, or 3) nucleotides from the sense strand set forth in SEQ ID NO: 138.

In some embodiments, the nucleotide sequence of the antisense strand comprises the nucleotide sequence of antisense strand set forth in SEQ ID NO: 321; and the nucleotide sequence of the sense strand comprises the nucleotide sequence of the sense strand set forth in SEQ ID NO: 138.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 321; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 321; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 321; and the sense strand consists of about 21 contiguous nucleotides differing by no more than 3 (e.g., 0, 1, 2, or 3) nucleotides from the nucleotide sequence of the sense strand set forth in SEQ ID NO: 138. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 321; and the sense strand consists of about 19 contiguous nucleotides differing by no more than 3 (e.g., 0, 1, 2, or 3) nucleotides from the nucleotide sequence of the sense strand set forth in SEQ ID NO: 138.

In some embodiments, the nucleotide sequence of the antisense strand consists of the nucleotide sequence of the antisense strand set forth in SEQ ID NO: 321 differing by no more than 3 (e.g., 0, 1, 2, or 3) nucleotides from the nucleotide sequence of the antisense strand set forth in SEQ ID NO: 321; and the nucleotide sequence of the sense strand consists of the nucleotide sequence of the sense strand set forth in SEQ ID NO: 138 differing by no more than 3 (e.g., 0, 1, 2, or 3) nucleotides from the sense strand set forth in SEQ ID NO: 138.

In some embodiments, the nucleotide sequence of the antisense strand consists of the nucleotide sequence of antisense strand set forth in SEQ ID NO: 321; and the nucleotide sequence of the sense strand consists of the nucleotide sequence of the sense strand set forth in SEQ ID NO: 138.

In some embodiments, the nucleotide sequence of the antisense strand differs by no more than 5 (e.g., 0, 1, 2, 3, 4, or 5) nucleotides from the nucleotide sequence set forth in SEQ ID NO: 1062; and the nucleotide sequence of the sense strand differs by no more than 5 (e.g., 0, 1, 2, 3, 4, or 5) nucleotides from the nucleotide sequence set forth in SEQ ID NO: 827. In some embodiments, the nucleotide sequence of the antisense strand differs by no more than 3 (e.g., 0, 1, 2, or 3) nucleotides from the nucleotide sequence set forth in SEQ ID NO: 1062; and the nucleotide sequence of the sense strand differs by no more than 3 (e.g., 0, 1, 2, or 3) nucleotides from the nucleotide sequence set forth in SEQ ID NO: 827. In some embodiments, the nucleotide sequence of the antisense strand differs by no more than 2 (e.g., 0, 1, or 2) nucleotides from the nucleotide sequence set forth in SEQ ID NO: 1062; and the nucleotide sequence of the sense strand differs by no more than 2 (e.g., 0, 1, or 2) nucleotides from the nucleotide sequence set forth in SEQ ID NO: 827. In some embodiments, the nucleotide sequence of the antisense strand differs by no more than 1 (e.g., 0 or 1) nucleotide from the nucleotide sequence set forth in SEQ ID NO: 1062; and the nucleotide sequence of the sense strand differs by no more than 1 (e.g., 0 or 1) nucleotide from the nucleotide sequence set forth in SEQ ID NO: 827.

In some embodiments, the nucleotide sequence of the antisense strand comprises the nucleotide sequence set forth in SEQ ID NO: 1062 differing by no more than 5 (e.g., 0, 1, 2, 3, 4, or 5) nucleotides from the nucleotide sequence set forth in SEQ ID NO: 1062; and the nucleotide sequence of the sense strand comprises the nucleotide sequence set forth in SEQ ID NO: 1062 differing by no more than 5 (e.g., 0, 1, 2, 3, 4, or 5) nucleotides from the nucleotide sequence set forth in SEQ ID NO: 827. In some embodiments, the nucleotide sequence of the antisense strand comprises the nucleotide sequence set forth in SEQ ID NO: 1062 differing by no more than 3 (e.g., 0, 1, 2, or 3) nucleotides from the nucleotide sequence set forth in SEQ ID NO: 1062; and the nucleotide sequence of the sense strand comprises the nucleotide sequence set forth in SEQ ID NO: 827 differing by no more than 3 (e.g., 0, 1, 2, or 3) nucleotides from the nucleotide sequence set forth in SEQ ID NO: 827. In some embodiments, the nucleotide sequence of the antisense strand comprises the nucleotide sequence set forth in SEQ ID NO: 1062 differing by no more than 2 (e.g., 0, 1, or 2) nucleotides from the nucleotide sequence set forth in SEQ ID NO: 1062; and the nucleotide sequence of the sense strand comprises the nucleotide sequence set forth in SEQ ID NO: 827 differing by no more than 2 (e.g., 0, 1, or 2) nucleotides from the nucleotide sequence set forth in SEQ ID NO: 827. In some embodiments, the nucleotide sequence of the antisense strand comprises the nucleotide sequence set forth in SEQ ID NO: 1062 differing by no more than 1 (e.g., 0 or 1) nucleotide from nucleotide sequence set forth in SEQ ID NO: 1062; and the nucleotide sequence of the sense strand comprises the nucleotide sequence set forth in SEQ ID NO: 827 differing by no more than 1 (e.g., 0 or 1) nucleotide from nucleotide sequence set forth in SEQ ID NO: 827.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1062; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 827. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1062; and the nucleotide sequence of the sense strand is at least 90%, 91%, 92%, 93%, 94%, 95%, 96%, 97%, 98%, 99% or 100% identical to the nucleotide sequence set forth in SEQ ID NO: 827. In some embodiments, the nucleotide sequence of the antisense strand is at least 95%, 96%, 97%, 98%, 99% or 100% identical to the nucleotide sequence set forth in SEQ ID NO: 1062; and the nucleotide sequence of the sense strand is at least 95%, 96%, 97%, 98%, 99% or 100% identical to the nucleotide sequence set forth in SEQ ID NO: 827. In some embodiments, the nucleotide sequence of the antisense strand is at least 96%, 97%, 98%, 99% or 100% identical to the nucleotide sequence set forth in SEQ ID NO: 1062; and the nucleotide sequence of the sense strand is at least 96%, 97%, 98%, 99% or 100% identical to the nucleotide sequence set forth in SEQ ID NO: 827. In some embodiments, the nucleotide sequence of the antisense strand is at least 97%, identical to the nucleotide sequence set forth in SEQ ID NO: 1062; and the nucleotide sequence of the sense strand is at least 97%, identical to the nucleotide sequence set forth in SEQ ID NO: 827. In some embodiments, the nucleotide sequence of the antisense strand is at least 98% identical to the nucleotide sequence set forth in SEQ ID NO: 1062; and the nucleotide sequence of the sense strand is at least 98% identical to the nucleotide sequence set forth in SEQ ID NO: 827. In some embodiments, the nucleotide sequence of the antisense strand is at least 99% identical to the nucleotide sequence set forth in SEQ ID NO: 1062; and the nucleotide sequence of the sense strand is at least 99% identical to the nucleotide sequence set forth in SEQ ID NO: 827. In some embodiments, the nucleotide sequence of the antisense strand is 100% identical to the nucleotide sequence set forth in SEQ ID NO: 1062; and the nucleotide sequence of the sense strand is 100% identical to the nucleotide sequence set forth in SEQ ID NO: 827.

In some embodiments, the nucleotide sequence of the antisense strand comprises the nucleotide sequence set forth in SEQ ID NO: 1062; and the nucleotide sequence of the sense strand comprises the nucleotide sequence set forth in SEQ ID NO: 827. In some embodiments, the nucleotide sequence of the antisense strand consists of the nucleotide sequence set forth in SEQ ID NO: 1062; and the nucleotide sequence of the sense strand consists of the nucleotide sequence set forth in SEQ ID NO: 827.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1062; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1062; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1062; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1062; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 827.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1062; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1062; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1062; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1062; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 827.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1062; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 1062; and the sense strand comprises about 21 contiguous nucleotides differing by no more than 3 (e.g., 0, 1, 2, or 3) nucleotides from the nucleotide sequence of the sense strand set forth in SEQ ID NO: 827. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 1062; and the sense strand comprises about 19 contiguous nucleotides differing by no more than 3 (e.g., 0, 1, 2, or 3) nucleotides from the nucleotide sequence of the sense strand set forth in SEQ ID NO: 827.

In some embodiments, the nucleotide sequence of the antisense strand comprises the nucleotide sequence of the antisense strand set forth in SEQ ID NO: 1062 differing by no more than 3 (e.g., 0, 1, 2, or 3) nucleotides from the nucleotide sequence of the antisense strand set forth in SEQ ID NO: 1062; and the nucleotide sequence of the sense strand comprises the nucleotide sequence of the sense strand set forth in SEQ ID NO: 827 differing by no more than 3 (e.g., 0, 1, 2, or 3) nucleotides from the sense strand set forth in SEQ ID NO: 827.

In some embodiments, the nucleotide sequence of the antisense strand comprises the nucleotide sequence of antisense strand set forth in SEQ ID NO: 1062; and the nucleotide sequence of the sense strand comprises the nucleotide sequence of the sense strand set forth in SEQ ID NO: 827.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1062; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1062; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 1062; and the sense strand consists of about 21 contiguous nucleotides differing by no more than 3 (e.g., 0, 1, 2, or 3) nucleotides from the nucleotide sequence of the sense strand set forth in SEQ ID NO: 827. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 1062; and the sense strand consists of about 19 contiguous nucleotides differing by no more than 3 (e.g., 0, 1, 2, or 3) nucleotides from the nucleotide sequence of the sense strand set forth in SEQ ID NO: 827.

In some embodiments, the nucleotide sequence of the antisense strand consists of the nucleotide sequence of the antisense strand set forth in SEQ ID NO: 1062 differing by no more than 3 (e.g., 0, 1, 2, or 3) nucleotides from the nucleotide sequence of the antisense strand set forth in SEQ ID NO: 1062; and the nucleotide sequence of the sense strand consists of the nucleotide sequence of the sense strand set forth in SEQ ID NO: 827 differing by no more than 3 (e.g., 0, 1, 2, or 3) nucleotides from the sense strand set forth in SEQ ID NO: 827.

In some embodiments, the nucleotide sequence of the antisense strand consists of the nucleotide sequence of antisense strand set forth in SEQ ID NO: 1062; and the nucleotide sequence of the sense strand consists of the nucleotide sequence of the sense strand set forth in SEQ ID NO: 827.

In some embodiments, the nucleotide sequence of the antisense strand differs by no more than 5 (e.g., 0, 1, 2, 3, 4, or 5) nucleotides from the nucleotide sequence set forth in SEQ ID NO: 1189; and the nucleotide sequence of the sense strand differs by no more than 5 (e.g., 0, 1, 2, 3, 4, or 5) nucleotides from the nucleotide sequence set forth in SEQ ID NO: 792. In some embodiments, the nucleotide sequence of the antisense strand differs by no more than 3 (e.g., 0, 1, 2, or 3) nucleotides from the nucleotide sequence set forth in SEQ ID NO: 1189; and the nucleotide sequence of the sense strand differs by no more than 3 (e.g., 0, 1, 2, or 3) nucleotides from the nucleotide sequence set forth in SEQ ID NO: 792. In some embodiments, the nucleotide sequence of the antisense strand differs by no more than 2 (e.g., 0, 1, or 2) nucleotides from the nucleotide sequence set forth in SEQ ID NO: 1189; and the nucleotide sequence of the sense strand differs by no more than 2 (e.g., 0, 1, or 2) nucleotides from the nucleotide sequence set forth in SEQ ID NO: 792. In some embodiments, the nucleotide sequence of the antisense strand differs by no more than 1 (e.g., 0 or 1) nucleotide from the nucleotide sequence set forth in SEQ ID NO: 1189; and the nucleotide sequence of the sense strand differs by no more than 1 (e.g., 0 or 1) nucleotide from the nucleotide sequence set forth in SEQ ID NO: 792.

In some embodiments, the nucleotide sequence of the antisense strand comprises the nucleotide sequence set forth in SEQ ID NO: 1189 differing by no more than 5 (e.g., 0, 1, 2, 3, 4, or 5) nucleotides from the nucleotide sequence set forth in SEQ ID NO: 1189; and the nucleotide sequence of the sense strand comprises the nucleotide sequence set forth in SEQ ID NO: 1189 differing by no more than 5 (e.g., 0, 1, 2, 3, 4, or 5) nucleotides from the nucleotide sequence set forth in SEQ ID NO: 792. In some embodiments, the nucleotide sequence of the antisense strand comprises the nucleotide sequence set forth in SEQ ID NO: 1189 differing by no more than 3 (e.g., 0, 1, 2, or 3) nucleotides from the nucleotide sequence set forth in SEQ ID NO: 1189; and the nucleotide sequence of the sense strand comprises the nucleotide sequence set forth in SEQ ID NO: 792 differing by no more than 3 (e.g., 0, 1, 2, or 3) nucleotides from the nucleotide sequence set forth in SEQ ID NO: 792. In some embodiments, the nucleotide sequence of the antisense strand comprises the nucleotide sequence set forth in SEQ ID NO: 1189 differing by no more than 2 (e.g., 0, 1, or 2) nucleotides from the nucleotide sequence set forth in SEQ ID NO: 1189; and the nucleotide sequence of the sense strand comprises the nucleotide sequence set forth in SEQ ID NO: 792 differing by no more than 2 (e.g., 0, 1, or 2) nucleotides from the nucleotide sequence set forth in SEQ ID NO: 792. In some embodiments, the nucleotide sequence of the antisense strand comprises the nucleotide sequence set forth in SEQ ID NO: 1189 differing by no more than 1 (e.g., 0 or 1) nucleotide from nucleotide sequence set forth in SEQ ID NO: 1189; and the nucleotide sequence of the sense strand comprises the nucleotide sequence set forth in SEQ ID NO: 792 differing by no more than 1 (e.g., 0 or 1) nucleotide from nucleotide sequence set forth in SEQ ID NO: 792.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1189; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 792. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1189; and the nucleotide sequence of the sense strand is at least 90%, 91%, 92%, 93%, 94%, 95%, 96%, 97%, 98%, 99% or 100% identical to the nucleotide sequence set forth in SEQ ID NO: 792. In some embodiments, the nucleotide sequence of the antisense strand is at least 95%, 96%, 97%, 98%, 99% or 100% identical to the nucleotide sequence set forth in SEQ ID NO: 1189; and the nucleotide sequence of the sense strand is at least 95%, 96%, 97%, 98%, 99% or 100% identical to the nucleotide sequence set forth in SEQ ID NO: 792. In some embodiments, the nucleotide sequence of the antisense strand is at least 96%, 97%, 98%, 99% or 100% identical to the nucleotide sequence set forth in SEQ ID NO: 1189; and the nucleotide sequence of the sense strand is at least 96%, 97%, 98%, 99% or 100% identical to the nucleotide sequence set forth in SEQ ID NO: 792. In some embodiments, the nucleotide sequence of the antisense strand is at least 97%, identical to the nucleotide sequence set forth in SEQ ID NO: 1189; and the nucleotide sequence of the sense strand is at least 97%, identical to the nucleotide sequence set forth in SEQ ID NO: 792. In some embodiments, the nucleotide sequence of the antisense strand is at least 98% identical to the nucleotide sequence set forth in SEQ ID NO: 1189; and the nucleotide sequence of the sense strand is at least 98% identical to the nucleotide sequence set forth in SEQ ID NO: 792. In some embodiments, the nucleotide sequence of the antisense strand is at least 99% identical to the nucleotide sequence set forth in SEQ ID NO: 1189; and the nucleotide sequence of the sense strand is at least 99% identical to the nucleotide sequence set forth in SEQ ID NO: 792. In some embodiments, the nucleotide sequence of the antisense strand is 100% identical to the nucleotide sequence set forth in SEQ ID NO: 1189; and the nucleotide sequence of the sense strand is 100% identical to the nucleotide sequence set forth in SEQ ID NO: 792.

In some embodiments, the nucleotide sequence of the antisense strand comprises the nucleotide sequence set forth in SEQ ID NO: 1189; and the nucleotide sequence of the sense strand comprises the nucleotide sequence set forth in SEQ ID NO: 792. In some embodiments, the nucleotide sequence of the antisense strand consists of the nucleotide sequence set forth in SEQ ID NO: 1189; and the nucleotide sequence of the sense strand consists of the nucleotide sequence set forth in SEQ ID NO: 792.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1189; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1189; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1189; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1189; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 792.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1189; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1189; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1189; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1189; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 792.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1189; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 1189; and the sense strand comprises about 21 contiguous nucleotides differing by no more than 3 (e.g., 0, 1, 2, or 3) nucleotides from the nucleotide sequence of the sense strand set forth in SEQ ID NO: 792. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 1189; and the sense strand comprises about 19 contiguous nucleotides differing by no more than 3 (e.g., 0, 1, 2, or 3) nucleotides from the nucleotide sequence of the sense strand set forth in SEQ ID NO: 792.

In some embodiments, the nucleotide sequence of the antisense strand comprises the nucleotide sequence of the antisense strand set forth in SEQ ID NO: 1189 differing by no more than 3 (e.g., 0, 1, 2, or 3) nucleotides from the nucleotide sequence of the antisense strand set forth in SEQ ID NO: 1189; and the nucleotide sequence of the sense strand comprises the nucleotide sequence of the sense strand set forth in SEQ ID NO: 792 differing by no more than 3 (e.g., 0, 1, 2, or 3) nucleotides from the sense strand set forth in SEQ ID NO: 792.

In some embodiments, the nucleotide sequence of the antisense strand comprises the nucleotide sequence of antisense strand set forth in SEQ ID NO: 1189; and the nucleotide sequence of the sense strand comprises the nucleotide sequence of the sense strand set forth in SEQ ID NO: 792.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1189; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1189; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 1189; and the sense strand consists of about 21 contiguous nucleotides differing by no more than 3 (e.g., 0, 1, 2, or 3) nucleotides from the nucleotide sequence of the sense strand set forth in SEQ ID NO: 792. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 1189; and the sense strand consists of about 19 contiguous nucleotides differing by no more than 3 (e.g., 0, 1, 2, or 3) nucleotides from the nucleotide sequence of the sense strand set forth in SEQ ID NO: 792.

In some embodiments, the nucleotide sequence of the antisense strand consists of the nucleotide sequence of the antisense strand set forth in SEQ ID NO: 1189 differing by no more than 3 (e.g., 0, 1, 2, or 3) nucleotides from the nucleotide sequence of the antisense strand set forth in SEQ ID NO: 1189; and the nucleotide sequence of the sense strand consists of the nucleotide sequence of the sense strand set forth in SEQ ID NO: 792 differing by no more than 3 (e.g., 0, 1, 2, or 3) nucleotides from the sense strand set forth in SEQ ID NO: 792.

In some embodiments, the nucleotide sequence of the antisense strand consists of the nucleotide sequence of antisense strand set forth in SEQ ID NO: 1189; and the nucleotide sequence of the sense strand consists of the nucleotide sequence of the sense strand set forth in SEQ ID NO: 792.

In some embodiments, the nucleotide sequence of the antisense strand differs by no more than 5 (e.g., 0, 1, 2, 3, 4, or 5) nucleotides from the nucleotide sequence set forth in SEQ ID NO: 1191; and the nucleotide sequence of the sense strand differs by no more than 5 (e.g., 0, 1, 2, 3, 4, or 5) nucleotides from the nucleotide sequence set forth in SEQ ID NO: 1192. In some embodiments, the nucleotide sequence of the antisense strand differs by no more than 3 (e.g., 0, 1, 2, or 3) nucleotides from the nucleotide sequence set forth in SEQ ID NO: 1191; and the nucleotide sequence of the sense strand differs by no more than 3 (e.g., 0, 1, 2, or 3) nucleotides from the nucleotide sequence set forth in SEQ ID NO: 1192. In some embodiments, the nucleotide sequence of the antisense strand differs by no more than 2 (e.g., 0, 1, or 2) nucleotides from the nucleotide sequence set forth in SEQ ID NO: 1191; and the nucleotide sequence of the sense strand differs by no more than 2 (e.g., 0, 1, or 2) nucleotides from the nucleotide sequence set forth in SEQ ID NO: 1192. In some embodiments, the nucleotide sequence of the antisense strand differs by no more than 1 (e.g., 0 or 1) nucleotide from the nucleotide sequence set forth in SEQ ID NO: 1191; and the nucleotide sequence of the sense strand differs by no more than 1 (e.g., 0 or 1) nucleotide from the nucleotide sequence set forth in SEQ ID NO: 1192.

In some embodiments, the nucleotide sequence of the antisense strand comprises the nucleotide sequence set forth in SEQ ID NO: 1191 differing by no more than 5 (e.g., 0, 1, 2, 3, 4, or 5) nucleotides from the nucleotide sequence set forth in SEQ ID NO: 1191; and the nucleotide sequence of the sense strand comprises the nucleotide sequence set forth in SEQ ID NO: 1191 differing by no more than 5 (e.g., 0, 1, 2, 3, 4, or 5) nucleotides from the nucleotide sequence set forth in SEQ ID NO: 1192. In some embodiments, the nucleotide sequence of the antisense strand comprises the nucleotide sequence set forth in SEQ ID NO: 1191 differing by no more than 3 (e.g., 0, 1, 2, or 3) nucleotides from the nucleotide sequence set forth in SEQ ID NO: 1191; and the nucleotide sequence of the sense strand comprises the nucleotide sequence set forth in SEQ ID NO: 1192 differing by no more than 3 (e.g., 0, 1, 2, or 3) nucleotides from the nucleotide sequence set forth in SEQ ID NO: 1192. In some embodiments, the nucleotide sequence of the antisense strand comprises the nucleotide sequence set forth in SEQ ID NO: 1191 differing by no more than 2 (e.g., 0, 1, or 2) nucleotides from the nucleotide sequence set forth in SEQ ID NO: 1191; and the nucleotide sequence of the sense strand comprises the nucleotide sequence set forth in SEQ ID NO: 1192 differing by no more than 2 (e.g., 0, 1, or 2) nucleotides from the nucleotide sequence set forth in SEQ ID NO: 1192. In some embodiments, the nucleotide sequence of the antisense strand comprises the nucleotide sequence set forth in SEQ ID NO: 1191 differing by no more than 1 (e.g., 0 or 1) nucleotide from nucleotide sequence set forth in SEQ ID NO: 1191; and the nucleotide sequence of the sense strand comprises the nucleotide sequence set forth in SEQ ID NO: 1192 differing by no more than 1 (e.g., 0 or 1) nucleotide from nucleotide sequence set forth in SEQ ID NO: 1192.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1191; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1192. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1191; and the nucleotide sequence of the sense strand is at least 90%, 91%, 92%, 93%, 94%, 95%, 96%, 97%, 98%, 99% or 100% identical to the nucleotide sequence set forth in SEQ ID NO: 1192. In some embodiments, the nucleotide sequence of the antisense strand is at least 95%, 96%, 97%, 98%, 99% or 100% identical to the nucleotide sequence set forth in SEQ ID NO: 1191; and the nucleotide sequence of the sense strand is at least 95%, 96%, 97%, 98%, 99% or 100% identical to the nucleotide sequence set forth in SEQ ID NO: 1192. In some embodiments, the nucleotide sequence of the antisense strand is at least 96%, 97%, 98%, 99% or 100% identical to the nucleotide sequence set forth in SEQ ID NO: 1191; and the nucleotide sequence of the sense strand is at least 96%, 97%, 98%, 99% or 100% identical to the nucleotide sequence set forth in SEQ ID NO: 1192. In some embodiments, the nucleotide sequence of the antisense strand is at least 97%, identical to the nucleotide sequence set forth in SEQ ID NO: 1191; and the nucleotide sequence of the sense strand is at least 97%, identical to the nucleotide sequence set forth in SEQ ID NO: 1192. In some embodiments, the nucleotide sequence of the antisense strand is at least 98% identical to the nucleotide sequence set forth in SEQ ID NO: 1191; and the nucleotide sequence of the sense strand is at least 98% identical to the nucleotide sequence set forth in SEQ ID NO: 1192. In some embodiments, the nucleotide sequence of the antisense strand is at least 99% identical to the nucleotide sequence set forth in SEQ ID NO: 1191; and the nucleotide sequence of the sense strand is at least 99% identical to the nucleotide sequence set forth in SEQ ID NO: 1192. In some embodiments, the nucleotide sequence of the antisense strand is 100% identical to the nucleotide sequence set forth in SEQ ID NO: 1191; and the nucleotide sequence of the sense strand is 100% identical to the nucleotide sequence set forth in SEQ ID NO: 1192.

In some embodiments, the nucleotide sequence of the antisense strand comprises the nucleotide sequence set forth in SEQ ID NO: 1191; and the nucleotide sequence of the sense strand comprises the nucleotide sequence set forth in SEQ ID NO: 1192. In some embodiments, the nucleotide sequence of the antisense strand consists of the nucleotide sequence set forth in SEQ ID NO: 1191; and the nucleotide sequence of the sense strand consists of the nucleotide sequence set forth in SEQ ID NO: 1192.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1191; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1191; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 1192.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1191; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1191; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1191; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 1192.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1191; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 1191; and the sense strand comprises about 21 contiguous nucleotides differing by no more than 3 (e.g., 0, 1, 2, or 3) nucleotides from the nucleotide sequence of the sense strand set forth in SEQ ID NO: 1192. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 1191; and the sense strand comprises about 19 contiguous nucleotides differing by no more than 3 (e.g., 0, 1, 2, or 3) nucleotides from the nucleotide sequence of the sense strand set forth in SEQ ID NO: 1192.

In some embodiments, the nucleotide sequence of the antisense strand comprises the nucleotide sequence of the antisense strand set forth in SEQ ID NO: 1191 differing by no more than 3 (e.g., 0, 1, 2, or 3) nucleotides from the nucleotide sequence of the antisense strand set forth in SEQ ID NO: 1191; and the nucleotide sequence of the sense strand comprises the nucleotide sequence of the sense strand set forth in SEQ ID NO: 1192 differing by no more than 3 (e.g., 0, 1, 2, or 3) nucleotides from the sense strand set forth in SEQ ID NO: 1192.

In some embodiments, the nucleotide sequence of the antisense strand comprises the nucleotide sequence of antisense strand set forth in SEQ ID NO: 1191; and the nucleotide sequence of the sense strand comprises the nucleotide sequence of the sense strand set forth in SEQ ID NO: 1192.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1191; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1191; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 1191; and the sense strand consists of about 21 contiguous nucleotides differing by no more than 3 (e.g., 0, 1, 2, or 3) nucleotides from the nucleotide sequence of the sense strand set forth in SEQ ID NO: 1192. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 1191; and the sense strand consists of about 19 contiguous nucleotides differing by no more than 3 (e.g., 0, 1, 2, or 3) nucleotides from the nucleotide sequence of the sense strand set forth in SEQ ID NO: 1192.

In some embodiments, the nucleotide sequence of the antisense strand consists of the nucleotide sequence of the antisense strand set forth in SEQ ID NO: 1191 differing by no more than 3 (e.g., 0, 1, 2, or 3) nucleotides from the nucleotide sequence of the antisense strand set forth in SEQ ID NO: 1191; and the nucleotide sequence of the sense strand consists of the nucleotide sequence of the sense strand set forth in SEQ ID NO: 1192 differing by no more than 3 (e.g., 0, 1, 2, or 3) nucleotides from the sense strand set forth in SEQ ID NO: 1192.

In some embodiments, the nucleotide sequence of the antisense strand consists of the nucleotide sequence of antisense strand set forth in SEQ ID NO: 1191; and the nucleotide sequence of the sense strand consists of the nucleotide sequence of the sense strand set forth in SEQ ID NO: 1192.

In some embodiments, the nucleotide sequence of the antisense strand differs by no more than 5 (e.g., 0, 1, 2, 3, 4, or 5) nucleotides from the nucleotide sequence set forth in SEQ ID NO: 1190; and the nucleotide sequence of the sense strand differs by no more than 5 (e.g., 0, 1, 2, 3, 4, or 5) nucleotides from the nucleotide sequence set forth in SEQ ID NO: 786. In some embodiments, the nucleotide sequence of the antisense strand differs by no more than 3 (e.g., 0, 1, 2, or 3) nucleotides from the nucleotide sequence set forth in SEQ ID NO: 1190; and the nucleotide sequence of the sense strand differs by no more than 3 (e.g., 0, 1, 2, or 3) nucleotides from the nucleotide sequence set forth in SEQ ID NO: 786. In some embodiments, the nucleotide sequence of the antisense strand differs by no more than 2 (e.g., 0, 1, or 2) nucleotides from the nucleotide sequence set forth in SEQ ID NO: 1190; and the nucleotide sequence of the sense strand differs by no more than 2 (e.g., 0, 1, or 2) nucleotides from the nucleotide sequence set forth in SEQ ID NO: 786. In some embodiments, the nucleotide sequence of the antisense strand differs by no more than 1 (e.g., 0 or 1) nucleotide from the nucleotide sequence set forth in SEQ ID NO: 1190; and the nucleotide sequence of the sense strand differs by no more than 1 (e.g., 0 or 1) nucleotide from the nucleotide sequence set forth in SEQ ID NO: 786.

In some embodiments, the nucleotide sequence of the antisense strand comprises the nucleotide sequence set forth in SEQ ID NO: 1190 differing by no more than 5 (e.g., 0, 1, 2, 3, 4, or 5) nucleotides from the nucleotide sequence set forth in SEQ ID NO: 1190; and the nucleotide sequence of the sense strand comprises the nucleotide sequence set forth in SEQ ID NO: 1190 differing by no more than 5 (e.g., 0, 1, 2, 3, 4, or 5) nucleotides from the nucleotide sequence set forth in SEQ ID NO: 786. In some embodiments, the nucleotide sequence of the antisense strand comprises the nucleotide sequence set forth in SEQ ID NO: 1190 differing by no more than 3 (e.g., 0, 1, 2, or 3) nucleotides from the nucleotide sequence set forth in SEQ ID NO: 1190; and the nucleotide sequence of the sense strand comprises the nucleotide sequence set forth in SEQ ID NO: 786 differing by no more than 3 (e.g., 0, 1, 2, or 3) nucleotides from the nucleotide sequence set forth in SEQ ID NO: 786. In some embodiments, the nucleotide sequence of the antisense strand comprises the nucleotide sequence set forth in SEQ ID NO: 1190 differing by no more than 2 (e.g., 0, 1, or 2) nucleotides from the nucleotide sequence set forth in SEQ ID NO: 1190; and the nucleotide sequence of the sense strand comprises the nucleotide sequence set forth in SEQ ID NO: 786 differing by no more than 2 (e.g., 0, 1, or 2) nucleotides from the nucleotide sequence set forth in SEQ ID NO: 786. In some embodiments, the nucleotide sequence of the antisense strand comprises the nucleotide sequence set forth in SEQ ID NO: 1190 differing by no more than 1 (e.g., 0 or 1) nucleotide from nucleotide sequence set forth in SEQ ID NO: 1190; and the nucleotide sequence of the sense strand comprises the nucleotide sequence set forth in SEQ ID NO: 786 differing by no more than 1 (e.g., 0 or 1) nucleotide from nucleotide sequence set forth in SEQ ID NO: 786.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1190; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 786. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1190; and the nucleotide sequence of the sense strand is at least 90%, 91%, 92%, 93%, 94%, 95%, 96%, 97%, 98%, 99% or 100% identical to the nucleotide sequence set forth in SEQ ID NO: 786. In some embodiments, the nucleotide sequence of the antisense strand is at least 95%, 96%, 97%, 98%, 99% or 100% identical to the nucleotide sequence set forth in SEQ ID NO: 1190; and the nucleotide sequence of the sense strand is at least 95%, 96%, 97%, 98%, 99% or 100% identical to the nucleotide sequence set forth in SEQ ID NO: 786. In some embodiments, the nucleotide sequence of the antisense strand is at least 96%, 97%, 98%, 99% or 100% identical to the nucleotide sequence set forth in SEQ ID NO: 1190; and the nucleotide sequence of the sense strand is at least 96%, 97%, 98%, 99% or 100% identical to the nucleotide sequence set forth in SEQ ID NO: 786. In some embodiments, the nucleotide sequence of the antisense strand is at least 97%, identical to the nucleotide sequence set forth in SEQ ID NO: 1190; and the nucleotide sequence of the sense strand is at least 97%, identical to the nucleotide sequence set forth in SEQ ID NO: 786. In some embodiments, the nucleotide sequence of the antisense strand is at least 98% identical to the nucleotide sequence set forth in SEQ ID NO: 1190; and the nucleotide sequence of the sense strand is at least 98% identical to the nucleotide sequence set forth in SEQ ID NO: 786. In some embodiments, the nucleotide sequence of the antisense strand is at least 99% identical to the nucleotide sequence set forth in SEQ ID NO: 1190; and the nucleotide sequence of the sense strand is at least 99% identical to the nucleotide sequence set forth in SEQ ID NO: 786. In some embodiments, the nucleotide sequence of the antisense strand is 100% identical to the nucleotide sequence set forth in SEQ ID NO: 1190; and the nucleotide sequence of the sense strand is 100% identical to the nucleotide sequence set forth in SEQ ID NO: 786.

In some embodiments, the nucleotide sequence of the antisense strand comprises the nucleotide sequence set forth in SEQ ID NO: 1190; and the nucleotide sequence of the sense strand comprises the nucleotide sequence set forth in SEQ ID NO: 786. In some embodiments, the nucleotide sequence of the antisense strand consists of the nucleotide sequence set forth in SEQ ID NO: 1190; and the nucleotide sequence of the sense strand consists of the nucleotide sequence set forth in SEQ ID NO: 786.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1190; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1190; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1190; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1190; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1190; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1190; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1190; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1190; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 786.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1190; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 1190; and the sense strand comprises about 21 contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 1190; and the sense strand comprises about 19 contiguous nucleotides differing by no more than 3 (e.g., 0, 1, 2, or 3) nucleotides from the nucleotide sequence of the sense strand set forth in SEQ ID NO: 786.

In some embodiments, the nucleotide sequence of the antisense strand comprises the nucleotide sequence of the antisense strand set forth in SEQ ID NO: 1190 differing by no more than 3 (e.g., 0, 1, 2, or 3) nucleotides from the nucleotide sequence of the antisense strand set forth in SEQ ID NO: 1190; and the nucleotide sequence of the sense strand comprises the nucleotide sequence of the sense strand set forth in SEQ ID NO: 786 differing by no more than 3 (e.g., 0, 1, 2, or 3) nucleotides from the sense strand set forth in SEQ ID NO: 786.

In some embodiments, the nucleotide sequence of the antisense strand comprises the nucleotide sequence of antisense strand set forth in SEQ ID NO: 1190; and the nucleotide sequence of the sense strand comprises the nucleotide sequence of the sense strand set forth in SEQ ID NO: 786.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1190; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1190; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 1190; and the sense strand consists of about 21 contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 1190; and the sense strand consists of about 19 contiguous nucleotides differing by no more than 3 (e.g., 0, 1, 2, or 3) nucleotides from the nucleotide sequence of the sense strand set forth in SEQ ID NO: 786.

In some embodiments, the nucleotide sequence of the antisense strand consists of the nucleotide sequence of the antisense strand set forth in SEQ ID NO: 1190 differing by no more than 3 (e.g., 0, 1, 2, or 3) nucleotides from the nucleotide sequence of the antisense strand set forth in SEQ ID NO: 1190; and the nucleotide sequence of the sense strand consists of the nucleotide sequence of the sense strand set forth in SEQ ID NO: 786 differing by no more than 3 (e.g., 0, 1, 2, or 3) nucleotides from the sense strand set forth in SEQ ID NO: 786.

In some embodiments, the nucleotide sequence of the antisense strand consists of the nucleotide sequence of antisense strand set forth in SEQ ID NO: 1190; and the nucleotide sequence of the sense strand consists of the nucleotide sequence of the sense strand set forth in SEQ ID NO: 786.

In some embodiments, the nucleotide sequence of the antisense strand differs by no more than 5 (e.g., 0, 1, 2, 3, 4, or 5) nucleotides from the nucleotide sequence set forth in SEQ ID NO: 1188; and the nucleotide sequence of the sense strand differs by no more than 5 (e.g., 0, 1, 2, 3, 4, or 5) nucleotides from the nucleotide sequence set forth in SEQ ID NO: 786. In some embodiments, the nucleotide sequence of the antisense strand differs by no more than 3 (e.g., 0, 1, 2, or 3) nucleotides from the nucleotide sequence set forth in SEQ ID NO: 1188; and the nucleotide sequence of the sense strand differs by no more than 3 (e.g., 0, 1, 2, or 3) nucleotides from the nucleotide sequence set forth in SEQ ID NO: 786. In some embodiments, the nucleotide sequence of the antisense strand differs by no more than 2 (e.g., 0, 1, or 2) nucleotides from the nucleotide sequence set forth in SEQ ID NO: 1188; and the nucleotide sequence of the sense strand differs by no more than 2 (e.g., 0, 1, or 2) nucleotides from the nucleotide sequence set forth in SEQ ID NO: 786. In some embodiments, the nucleotide sequence of the antisense strand differs by no more than 1 (e.g., 0 or 1) nucleotide from the nucleotide sequence set forth in SEQ ID NO: 1188; and the nucleotide sequence of the sense strand differs by no more than 1 (e.g., 0 or 1) nucleotide from the nucleotide sequence set forth in SEQ ID NO: 786.

In some embodiments, the nucleotide sequence of the antisense strand comprises the nucleotide sequence set forth in SEQ ID NO: 1188 differing by no more than 5 (e.g., 0, 1, 2, 3, 4, or 5) nucleotides from the nucleotide sequence set forth in SEQ ID NO: 1188; and the nucleotide sequence of the sense strand comprises the nucleotide sequence set forth in SEQ ID NO: 1188 differing by no more than 5 (e.g., 0, 1, 2, 3, 4, or 5) nucleotides from the nucleotide sequence set forth in SEQ ID NO: 786. In some embodiments, the nucleotide sequence of the antisense strand comprises the nucleotide sequence set forth in SEQ ID NO: 1188 differing by no more than 3 (e.g., 0, 1, 2, or 3) nucleotides from the nucleotide sequence set forth in SEQ ID NO: 1188; and the nucleotide sequence of the sense strand comprises the nucleotide sequence set forth in SEQ ID NO: 786 differing by no more than 3 (e.g., 0, 1, 2, or 3) nucleotides from the nucleotide sequence set forth in SEQ ID NO: 786. In some embodiments, the nucleotide sequence of the antisense strand comprises the nucleotide sequence set forth in SEQ ID NO: 1188 differing by no more than 2 (e.g., 0, 1, or 2) nucleotides from the nucleotide sequence set forth in SEQ ID NO: 1188; and the nucleotide sequence of the sense strand comprises the nucleotide sequence set forth in SEQ ID NO: 786 differing by no more than 2 (e.g., 0, 1, or 2) nucleotides from the nucleotide sequence set forth in SEQ ID NO: 786. In some embodiments, the nucleotide sequence of the antisense strand comprises the nucleotide sequence set forth in SEQ ID NO: 1188 differing by no more than 1 (e.g., 0 or 1) nucleotide from nucleotide sequence set forth in SEQ ID NO: 1188; and the nucleotide sequence of the sense strand comprises the nucleotide sequence set forth in SEQ ID NO: 786 differing by no more than 1 (e.g., 0 or 1) nucleotide from nucleotide sequence set forth in SEQ ID NO: 786.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 1188; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 786. In some embodiments, the nucleotide sequence of the antisense strand is at least 90%, 91%, 92%, 93%, 94%, 95%, 96%, 97%, 98%, 99% or 100% identical to the nucleotide sequence set forth in SEQ ID NO: 1188; and the nucleotide sequence of the sense strand is at least 90%, 91%, 92%, 93%, 94%, 95%, 96%, 97%, 98%, 99% or 100% identical to the nucleotide sequence set forth in SEQ ID NO: 786. In some embodiments, the nucleotide sequence of the antisense strand is at least 95%, 96%, 97%, 98%, 99% or 100% identical to the nucleotide sequence set forth in SEQ ID NO: 1188; and the nucleotide sequence of the sense strand is at least 95%, 96%, 97%, 98%, 99% or 100% identical to the nucleotide sequence set forth in SEQ ID NO: 786. In some embodiments, the nucleotide sequence of the antisense strand is at least 96%, 97%, 98%, 99% or 100% identical to the nucleotide sequence set forth in SEQ ID NO: 1188; and the nucleotide sequence of the sense strand is at least 96%, 97%, 98%, 99% or 100% identical to the nucleotide sequence set forth in SEQ ID NO: 786. In some embodiments, the nucleotide sequence of the antisense strand is at least 97%, identical to the nucleotide sequence set forth in SEQ ID NO: 1188; and the nucleotide sequence of the sense strand is at least 97%, identical to the nucleotide sequence set forth in SEQ ID NO: 786. In some embodiments, the nucleotide sequence of the antisense strand is at least 98% identical to the nucleotide sequence set forth in SEQ ID NO: 1188; and the nucleotide sequence of the sense strand is at least 98% identical to the nucleotide sequence set forth in SEQ ID NO: 786. In some embodiments, the nucleotide sequence of the antisense strand is at least 99% identical to the nucleotide sequence set forth in SEQ ID NO: 1188; and the nucleotide sequence of the sense strand is at least 99% identical to the nucleotide sequence set forth in SEQ ID NO: 786. In some embodiments, the nucleotide sequence of the antisense strand is 100% identical to the nucleotide sequence set forth in SEQ ID NO: 1188; and the nucleotide sequence of the sense strand is 100% identical to the nucleotide sequence set forth in SEQ ID NO: 786.

In some embodiments, the nucleotide sequence of the antisense strand comprises the nucleotide sequence set forth in SEQ ID NO: 1188; and the nucleotide sequence of the sense strand comprises the nucleotide sequence set forth in SEQ ID NO: 786. In some embodiments, the nucleotide sequence of the antisense strand consists of the nucleotide sequence set forth in SEQ ID NO: 1188; and the nucleotide sequence of the sense strand consists of the nucleotide sequence set forth in SEQ ID NO: 786.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1188; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1188; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1188; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand set forth in SEQ ID NO: 1188; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand set forth in SEQ ID NO: 786.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1188; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand set forth in SEQ ID NO: 1188; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1188; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand set forth in SEQ ID NO: 1188; and the sense strand comprises from about 21-23 (e.g., 21-22, 22-23) contiguous nucleotides differing by no more than 3 (e.g., 0, 1, 2, or 3) nucleotides from the nucleotide sequence of the sense strand set forth in SEQ ID NO: 786.

In some embodiments, the antisense strand comprises about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand comprises about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand comprises 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1188; and the sense strand comprises about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 23 contiguous nucleotides differing by no more than 3 (e.g., 0, 1, 2, or 3) nucleotides from the nucleotide sequence of the antisense strand set forth in SEQ ID NO: 1188; and the sense strand comprises about 21 contiguous nucleotides differing by no more than 3 (e.g., 0, 1, 2, or 3) nucleotides from the nucleotide sequence of the sense strand set forth in SEQ ID NO: 786. In some embodiments, the antisense strand comprises about 21 contiguous nucleotides differing by no more than 3 (e.g., 0, 1, 2, or 3) nucleotides from the nucleotide sequence of the antisense strand set forth in SEQ ID NO: 1188; and the sense strand comprises about 19 contiguous nucleotides differing by no more than 3 (e.g., 0, 1, 2, or 3) nucleotides from the nucleotide sequence of the sense strand set forth in SEQ ID NO: 786.

In some embodiments, the nucleotide sequence of the antisense strand comprises the nucleotide sequence of the antisense strand set forth in SEQ ID NO: 1188 differing by no more than 3 (e.g., 0, 1, 2, or 3) nucleotides from the nucleotide sequence of the antisense strand set forth in SEQ ID NO: 1188; and the nucleotide sequence of the sense strand comprises the nucleotide sequence of the sense strand set forth in SEQ ID NO: 786 differing by no more than 3 (e.g., 0, 1, 2, or 3) nucleotides from the sense strand set forth in SEQ ID NO: 786.

In some embodiments, the nucleotide sequence of the antisense strand comprises the nucleotide sequence of antisense strand set forth in SEQ ID NO: 1188; and the nucleotide sequence of the sense strand comprises the nucleotide sequence of the sense strand set forth in SEQ ID NO: 786.

In some embodiments, the antisense strand consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand set forth in SEQ ID NO: 1188; and the sense strand consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand set forth in SEQ ID NO: 1188; and the sense strand consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 23 contiguous nucleotides differing by no more than 3 (e.g., 0, 1, 2, or 3) nucleotides from the nucleotide sequence of the antisense strand set forth in SEQ ID NO: 1188; and the sense strand consists of about 21 contiguous nucleotides differing by no more than 3 (e.g., 0, 1, 2, or 3) nucleotides from the nucleotide sequence of the sense strand set forth in SEQ ID NO: 786. In some embodiments, the antisense strand consists of about 21 contiguous nucleotides differing by no more than 3 (e.g., 0, 1, 2, or 3) nucleotides from the nucleotide sequence of the antisense strand set forth in SEQ ID NO: 1188; and the sense strand consists of about 19 contiguous nucleotides differing by no more than 3 (e.g., 0, 1, 2, or 3) nucleotides from the nucleotide sequence of the sense strand set forth in SEQ ID NO: 786.

In some embodiments, the nucleotide sequence of the antisense strand consists of the nucleotide sequence of the antisense strand set forth in SEQ ID NO: 1188 differing by no more than 3 (e.g., 0, 1, 2, or 3) nucleotides from the nucleotide sequence of the antisense strand set forth in SEQ ID NO: 1188; and the nucleotide sequence of the sense strand consists of the nucleotide sequence of the sense strand set forth in SEQ ID NO: 786 differing by no more than 3 (e.g., 0, 1, 2, or 3) nucleotides from the sense strand set forth in SEQ ID NO: 786.

In some embodiments, the nucleotide sequence of the antisense strand consists of the nucleotide sequence of antisense strand set forth in SEQ ID NO: 1188; and the nucleotide sequence of the sense strand consists of the nucleotide sequence of the sense strand set forth in SEQ ID NO: 786.

In some embodiments, the nucleotide sequence of the antisense strand differs by no more than 5 (e.g., 0, 1, 2, 3, 4, or 5) nucleotides from the nucleotide sequence of any one of the antisense strands of any one of dsRNA agents 1-482; and the nucleotide sequence of the sense strand differs by no more than 5 (e.g., 0, 1, 2, 3, 4, or 5) nucleotides from the nucleotide sequence of the sense strand of the corresponding (the same) dsRNA agent. In some embodiments, the nucleotide sequence of the antisense strand differs by no more than 3 (e.g., 0, 1, 2, or 3) nucleotides from the nucleotide sequence of any one of the antisense strands of any one of dsRNA agents 1-482; and the nucleotide sequence of the sense strand differs by no more than 3 (e.g., 0, 1, 2, or 3) nucleotides from the nucleotide sequence of the sense strand of the corresponding (the same) dsRNA agent. In some embodiments, the nucleotide sequence of the antisense strand differs by no more than 2 (e.g., 0, 1, or 2) nucleotides from the nucleotide sequence of any one of the antisense strands of any one of dsRNA agents 1-482; and the nucleotide sequence of the sense strand differs by no more than 2 (e.g., 0, 1, or 2) nucleotides from the nucleotide sequence the sense strand of the corresponding (the same) dsRNA agent. In some embodiments, the nucleotide sequence of the antisense strand differs by no more than 1 (e.g., 0 or 1) nucleotide from the nucleotide sequence of any one of the antisense strands of any one of dsRNA agents 1-482; and the nucleotide sequence of the sense strand differs by no more than 1 (e.g., 0 or 1) nucleotide from the nucleotide sequence of the sense strand of the corresponding (the same) dsRNA agent.

In some embodiments, the nucleotide sequence of the antisense strand comprises the nucleotide sequence of any one of the antisense strands of any one of dsRNA agents 1-482 differing by no more than 5 (e.g., 0, 1, 2, 3, 4, or 5) nucleotides from the antisense strand of any one of dsRNA agents 1-482; and the nucleotide sequence of the sense strand comprises the nucleotide sequence of the corresponding (the same) dsRNA agent differing by no more than 5 (e.g., 0, 1, 2, 3, 4, or 5) nucleotides from the sense strand of the corresponding (the same) dsRNA agent. In some embodiments, the nucleotide sequence of the antisense strand comprises the nucleotide sequence of any one of the antisense strands of any one of dsRNA agents 1-482 differing by no more than 3 (e.g., 0, 1, 2, or 3) nucleotides from the antisense strand of any one of dsRNA agents 1-482; and the nucleotide sequence of the sense strand comprises the nucleotide sequence of any one of the sense strand of the corresponding (the same) dsRNA agent differing by no more than 3 (e.g., 0, 1, 2, or 3) nucleotides from the sense strand of the corresponding (the same) dsRNA agent. In some embodiments, the nucleotide sequence of the antisense strand comprises the nucleotide sequence of any one of the antisense strands of any one of dsRNA agents 1-482 differing by no more than 2 (e.g., 0, 1, or 2) nucleotides from the antisense strand of any one of dsRNA agents 1-482; and the nucleotide sequence of the sense strand comprises the nucleotide sequence of any one of the sense strands of the corresponding (the same) dsRNA agent differing by no more than 2 (e.g., 0, 1, or 2) nucleotides from the sense strand of the corresponding (the same) dsRNA agent. In some embodiments, the nucleotide sequence of the antisense strand comprises the nucleotide sequence of any one of the antisense strands of any one of dsRNA agents 1-482 differing by no more than 1 (e.g., 0 or 1) nucleotide from the antisense strand of any one of dsRNA agents 1-482; and the nucleotide sequence of the sense strand comprises the nucleotide sequence of any one of the sense strand of the corresponding (the same) dsRNA agent differing by no more than 1 (e.g., 0 or 1) nucleotide from the sense strand of the corresponding (the same) dsRNA agent.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands of any one of dsRNA agents 1-482; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of the sense strand of the corresponding (the same) dsRNA agent. In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands of any one of dsRNA agents 1-482; and the nucleotide sequence of the sense strand is at least 90%, 91%, 92%, 93%, 94%, 95%, 96%, 97%, 98%, 99% or 100% identical to the nucleotide sequence of the sense strand of the corresponding (the same) dsRNA agent. In some embodiments, the nucleotide sequence of the antisense strand is at least 95%, 96%, 97%, 98%, 99% or 100% identical to the nucleotide sequence of any one of the antisense strands of any one of dsRNA agents 1-482; and the nucleotide sequence of the sense strand is at least 95%, 96%, 97%, 98%, 99% or 100% identical to the nucleotide sequence of the sense strand of the corresponding (the same) dsRNA agent. In some embodiments, the nucleotide sequence of the antisense strand is at least 96%, 97%, 98%, 99% or 100% identical to the nucleotide sequence of any one of the antisense strands of any one of dsRNA agents 1-482; and the nucleotide sequence of the sense strand is at least 96%, 97%, 98%, 99% or 100% identical to the nucleotide sequence of the sense strand of the corresponding (the same) dsRNA agent. In some embodiments, the nucleotide sequence of the antisense strand is at least 97%, identical to the nucleotide sequence of any one of the antisense strands of any one of dsRNA agents 1-482; and the nucleotide sequence of the sense strand is at least 97%, identical to the nucleotide sequence of the sense strand of the corresponding (the same) dsRNA agent. In some embodiments, the nucleotide sequence of the antisense strand is at least 98% identical to the nucleotide sequence of any one of the antisense strands of any one of dsRNA agents 1-482; and the nucleotide sequence of the sense strand is at least 98% identical to the nucleotide sequence of the sense strand of the corresponding (the same) dsRNA agent. In some embodiments, the nucleotide sequence of the antisense strand is at least 99% identical to the nucleotide sequence of any one of the antisense strands of any one of dsRNA agents 1-482; and the nucleotide sequence of the sense strand is at least 99% identical to the nucleotide sequence of the sense strand of the corresponding (the same) dsRNA agent. In some embodiments, the nucleotide sequence of the antisense strand is 100% identical to the nucleotide sequence of any one of the antisense strands of any one of dsRNA agents 1-482; and the nucleotide sequence of the sense strand is 100% identical to the nucleotide sequence of the sense strand of the corresponding (the same) dsRNA agent.

In some embodiments, the nucleotide sequence of the antisense strand comprises the nucleotide sequence of any one of the antisense strands of any one of dsRNA agents 1-482; and the nucleotide sequence of the sense strand comprises the nucleotide sequence of the sense strand of the corresponding (the same) dsRNA agent. In some embodiments, the nucleotide sequence of the antisense strand consists of the nucleotide sequence of any one of the antisense strands of any one of dsRNA agents 1-482; and the nucleotide sequence of the sense strand consists of the nucleotide sequence of the sense strand of the corresponding (the same) dsRNA agent.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-482; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-482; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-482; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-482; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand of any one of dsRNA agents 1-482; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 1-482; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 1-482; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 1-482; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 1-482; and the sense strand comprises from about 21-23 (e.g., 21-22, 22-23) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-482; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-482; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-482; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-482; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand of any one of dsRNA agents 1-482; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23 contiguous nucleotides differing by no more than 3 (e.g., 0, 1, 2, or 3) nucleotides from the nucleotide sequence of the antisense strand of any one of dsRNA agents 1-482; and the sense strand comprises or consists of about 21 contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21 contiguous nucleotides differing by no more than 3 (e.g., 0, 1, 2, or 3) nucleotides from the nucleotide sequence of the antisense strand of any one of dsRNA agents 1-482; and the sense strand comprises or consists of about 19 contiguous nucleotides differing by no more than 3 (e.g., 0, 1, 2, or 3) nucleotides from the nucleotide sequence of the sense strand of the corresponding (the same) dsRNA agent.

In some embodiments, the nucleotide sequence of the antisense strand comprises or consists of the nucleotide sequence of the antisense strand of any one of dsRNA agents 1-482 differing by no more than 3 (e.g., 0, 1, 2, or 3) nucleotides from the antisense strand set forth in any one of the dsRNA agents 1-482; and the nucleotide sequence of the sense strand comprises or consists of the nucleotide sequence of the sense strand of the corresponding (the same) dsRNA agent differing by no more than 3 (e.g., 0, 1, 2, or 3) nucleotides from the sense strand set forth in the corresponding (the same) dsRNA agent.

In some embodiments, the nucleotide sequence of the antisense strand comprises or consists of the nucleotide sequence the antisense strand of any one of dsRNA agents 1-482; and the nucleotide sequence of the sense strand comprises or consists of the nucleotide sequence of the sense strand of the corresponding (the same) dsRNA agent.

In some embodiments, the nucleotide sequence of the antisense strand differs by no more than 5 (e.g., 0, 1, 2, 3, 4, or 5) nucleotides from the nucleotide sequence of any one of the antisense strands of any one of dsRNA agents 129, 135, 169, 170, 409, 479, 480, 481, or 482; and the nucleotide sequence of the sense strand differs by no more than 5 (e.g., 0, 1, 2, 3, 4, or 5) nucleotides from the nucleotide sequence of the sense strand of the corresponding (the same) dsRNA agent. In some embodiments, the nucleotide sequence of the antisense strand differs by no more than 3 (e.g., 0, 1, 2, or 3) nucleotides from the nucleotide sequence of any one of the antisense strands of any one of dsRNA agents 129, 135, 169, 170, 409, 479, 480, 481, or 482; and the nucleotide sequence of the sense strand differs by no more than 3 (e.g., 0, 1, 2, or 3) nucleotides from the nucleotide sequence of the sense strand of the corresponding (the same) dsRNA agent. In some embodiments, the nucleotide sequence of the antisense strand differs by no more than 2 (e.g., 0, 1, or 2) nucleotides from the nucleotide sequence of any one of the antisense strands of any one of dsRNA agents 129, 135, 169, 170, 409, 479, 480, 481, or 482; and the nucleotide sequence of the sense strand differs by no more than 2 (e.g., 0, 1, or 2) nucleotides from the nucleotide sequence the sense strand of the corresponding (the same) dsRNA agent. In some embodiments, the nucleotide sequence of the antisense strand differs by no more than 1 (e.g., 0 or 1) nucleotide from the nucleotide sequence of any one of the antisense strands of any one of dsRNA agents 129, 135, 169, 170, 409, 479, 480, 481, or 482; and the nucleotide sequence of the sense strand differs by no more than 1 (e.g., 0 or 1) nucleotide from the nucleotide sequence of the sense strand of the corresponding (the same) dsRNA agent.

In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 5 (e.g., 0, 1, 2, 3, 4, or 5) nucleotides from the antisense strand of any one of dsRNA agents 129, 135, 169, 170, 409, 479, 480, 481, or 482; and the nucleotide sequence of the sense strand comprises the nucleotide sequence of the corresponding (the same) dsRNA agent differing by no more than 5 (e.g., 0, 1, 2, 3, 4, or 5) nucleotides from the sense strand of the corresponding (the same) dsRNA agent. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 3 (e.g., 0, 1, 2, or 3) nucleotides from the antisense strand of any one of dsRNA agents 129, 135, 169, 170, 409, 479, 480, 481, or 482; and the nucleotide sequence of the sense strand comprises the nucleotide sequence of any one of the sense strand of the corresponding (the same) dsRNA agent differing by no more than 3 (e.g., 0, 1, 2, or 3) nucleotides from the sense strand of the corresponding (the same) dsRNA agent. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 2 (e.g., 0, 1, or 2) nucleotides from the antisense strand of any one of dsRNA agents 129, 135, 169, 170, 409, 479, 480, 481, or 482; and the nucleotide sequence of the sense strand comprises the nucleotide sequence of any one of the sense strands of the corresponding (the same) dsRNA agent differing by no more than 2 (e.g., 0, 1, or 2) nucleotides from the sense strand of the corresponding (the same) dsRNA agent. In some embodiments, the nucleotide sequence of the antisense strand comprises the nucleotide sequence of any one of the antisense strands of any one of dsRNA agents 129, 135, 169, 170, 409, 479, 480, 481, or 482 differing by no more than 1 (e.g., 0 or 1) nucleotide from the antisense strand of any one of dsRNA agents 129, 135, 169, 170, 409, 479, 480, 481, or 482; and the nucleotide sequence of the sense strand comprises the nucleotide sequence of any one of the sense strand of the corresponding (the same) dsRNA agent differing by no more than 1 (e.g., 0 or 1) nucleotide from the sense strand of the corresponding (the same) dsRNA agent.

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands of any one of dsRNA agents 129, 135, 169, 170, 409, 479, 480, 481, or 482; and the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of the sense strand of the corresponding (the same) dsRNA agent. In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands of any one of dsRNA agents 129, 135, 169, 170, 409, 479, 480, 481, or 482; and the nucleotide sequence of the sense strand is at least 90%, 91%, 92%, 93%, 94%, 95%, 96%, 97%, 98%, 99% or 100% identical to the nucleotide sequence of the sense strand of the corresponding (the same) dsRNA agent. In some embodiments, the nucleotide sequence of the antisense strand is at least 95%, 96%, 97%, 98%, 99% or 100% identical to the nucleotide sequence of any one of the antisense strands of any one of dsRNA agents 129, 135, 169, 170, 409, 479, 480, 481, or 482; and the nucleotide sequence of the sense strand is at least 95%, 96%, 97%, 98%, 99% or 100% identical to the nucleotide sequence of the sense strand of the corresponding (the same) dsRNA agent. In some embodiments, the nucleotide sequence of the antisense strand is at least 96%, 97%, 98%, 99% or 100% identical to the nucleotide sequence of any one of the antisense strands of any one of dsRNA agents 129, 135, 169, 170, 409, 479, 480, 481, or 482; and the nucleotide sequence of the sense strand is at least 96%, 97%, 98%, 99% or 100% identical to the nucleotide sequence of the sense strand of the corresponding (the same) dsRNA agent. In some embodiments, the nucleotide sequence of the antisense strand is at least 97%, identical to the nucleotide sequence of any one of the antisense strands of any one of dsRNA agents 129, 135, 169, 170, 409, 479, 480, 481, or 482; and the nucleotide sequence of the sense strand is at least 97%, identical to the nucleotide sequence of the sense strand of the corresponding (the same) dsRNA agent. In some embodiments, the nucleotide sequence of the antisense strand is at least 98% identical to the nucleotide sequence of any one of the antisense strands of any one of dsRNA agents 129, 135, 169, 170, 409, 479, 480, 481, or 482; and the nucleotide sequence of the sense strand is at least 98% identical to the nucleotide sequence of the sense strand of the corresponding (the same) dsRNA agent. In some embodiments, the nucleotide sequence of the antisense strand is at least 99% identical to the nucleotide sequence of any one of the antisense strands of any one of dsRNA agents 129, 135, 169, 170, 409, 479, 480, 481, or 482; and the nucleotide sequence of the sense strand is at least 99% identical to the nucleotide sequence of the sense strand of the corresponding (the same) dsRNA agent. In some embodiments, the nucleotide sequence of the antisense strand is 100% identical to the nucleotide sequence of any one of the antisense strands of any one of dsRNA agents 129, 135, 169, 170, 409, 479, 480, 481, or 482; and the nucleotide sequence of the sense strand is 100% identical to the nucleotide sequence of the sense strand of the corresponding (the same) dsRNA agent.

In some embodiments, the nucleotide sequence of the antisense strand comprises the nucleotide sequence of any one of the antisense strands of any one of dsRNA agents 129, 135, 169, 170, 409, 479, 480, 481, or 482; and the nucleotide sequence of the sense strand comprises the nucleotide sequence of the sense strand of the corresponding (the same) dsRNA agent. In some embodiments, the nucleotide sequence of the antisense strand consists of the nucleotide sequence of any one of the antisense strands of any one of dsRNA agents 129, 135, 169, 170, 409, 479, 480, 481, or 482; and the nucleotide sequence of the sense strand consists of the nucleotide sequence of the sense strand of the corresponding (the same) dsRNA agent.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises from about 21-23 (e.g., 21-22, 22-23) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23 contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of about 21 contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21 contiguous nucleotides differing by no more than 3 (e.g., 0, 1, 2, or 3) nucleotides from the nucleotide sequence of the antisense strand of any one of dsRNA agents 129, 135, 169, 170, 409, 479, 480, 481, or 482; and the sense strand comprises or consists of about 19 contiguous nucleotides differing by no more than 3 (e.g., 0, 1, 2, or 3) nucleotides from the nucleotide sequence of the sense strand of the corresponding (the same) dsRNA agent.

In some embodiments, the nucleotide sequence of the antisense strand comprises or consists of the nucleotide sequence of the antisense strand of any one of dsRNA agents 129, 135, 169, 170, 409, 479, 480, 481, or 482 differing by no more than 3 (e.g., 0, 1, 2, or 3) nucleotides from the antisense strand set forth in any one of the dsRNA agents 169, 129, 170, 175, 134, 204, 218, 135, 238, 167, 137, 233, 148, 152, 130, 166, 136, 149, 151, or 172; and the nucleotide sequence of the sense strand comprises or consists of the nucleotide sequence of the sense strand of the corresponding (the same) dsRNA agent differing by no more than 3 (e.g., 0, 1, 2, or 3) nucleotides from the sense strand set forth in the corresponding (the same) dsRNA agent.

In some embodiments, the nucleotide sequence of the antisense strand comprises or consists of the nucleotide sequence the antisense strand of any one of dsRNA agents 129, 135, 169, 170, 409, 479, 480, 481, or 482; and the nucleotide sequence of the sense strand comprises or consists of the nucleotide sequence of the sense strand of the corresponding (the same) dsRNA agent.

In some embodiments, the dsRNA agent is any one of dsRNA agents 1-482. In specific embodiments the dsRNA agent is any one of dsRNA agents 129, 135, 169, 170, 409, 479, 480, 481, or 482. In specific embodiments the dsRNA agent is dsRNA agent 129. In specific embodiments the dsRNA agent is dsRNA agent 135. In specific embodiments the dsRNA agent is dsRNA agent 169. In specific embodiments the dsRNA agent is dsRNA agent 170. In specific embodiments the dsRNA agent is dsRNA agent 409. In specific embodiments the dsRNA agent is dsRNA agent 479. In specific embodiments the dsRNA agent is dsRNA agent 479. In specific embodiments the dsRNA agent is dsRNA agent 480. In specific embodiments the dsRNA agent is dsRNA agent 481. In specific embodiments the dsRNA agent is dsRNA agent 482.

In some embodiments, the sense strand comprises at least 15 (e.g., 16, 17, 18, 19, 20, 21) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises at least 15 (e.g., 16, 17, 18, 19, 20, 21, 22, or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 19 (e.g., 20, 21) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises at least 19 (e.g., 20, 21, 22, or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises at least 21 (e.g., 22 or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises at least 21 (e.g., 22 or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises at least 23 contiguous nucleotides differing by no more than 3 (e.g., 0, 1, 2, or 3) nucleotides from the corresponding complementary nucleotide sequence of SEQ ID NO: 2.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about from about 21-23 (e.g., 21-22, 22-23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about 21-23 (e.g., 21-22, 22-23) contiguous nucleotides differing by no more than 3 (e.g., 0, 1, 2, or 3) nucleotides from 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises from about 21-23 (e.g., 21-22, 22-23) contiguous nucleotides differing by no more than 3 (e.g., 0, 1, 2, or 3) nucleotides from the corresponding complementary nucleotide sequence of SEQ ID NO: 2.

In some embodiments, the sense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740,1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 23, 24, 25, 26, 27, 28, 29, 30 (e.g., 23) contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 23 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of about 21 contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21 contiguous nucleotides differing by no more than 3 (e.g., 0, 1, 2, or 3) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1294-1346, 1366-1400, 1370-1410, 1360-1400, 1470-1510, 1410-1460, 1520-1560, 1580-1620, 1640-1680, 1640-1690, 1670-1710, 1700-1740, 1700-1745, 1724-1753, 1750-1790, 1757-1812, 1770-1810, 1833-1864, 1880-1920, 1900-1940, 1900-1927, 1915-1966, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1; and the antisense strand comprises or consists of about 19 contiguous nucleotides differing by no more than 3 (e.g., 0, 1, 2, or 3) nucleotides from the corresponding complementary nucleotide sequence of SEQ ID NO: 2.

In some embodiments, the sense strand comprises at least 15 (e.g., 16, 17, 18, 19, 20, 21) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises at least 15 (e.g., 16, 17, 18, 19, 20, 21, 22, or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 19 (e.g., 20, 21) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises at least 19 (e.g., 20, 21, 22, or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises at least 21 (e.g., 22 or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises at least 21 (e.g., 22 or 23) contiguous nucleotides differing by no more than 3 (e.g., 0, 1, 2, or 3) nucleotides from the corresponding complementary nucleotide sequence of SEQ ID NO: 2. In some embodiments, the sense strand comprises at least 21 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises at least 23 contiguous nucleotides differing by no more than 3 (e.g., 0, 1, 2, or 3) nucleotides from the corresponding complementary nucleotide sequence of SEQ ID NO: 2.

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about from about 21-23 (e.g., 21-22, 22-23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the corresponding complementary nucleotide sequence of SEQ ID NO: 2. In some embodiments, the sense strand comprises from about 21-23 (e.g., 21-22, 22-23) contiguous nucleotides differing by no more than 3 (e.g., 0, 1, 2, or 3) nucleotides from 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises from about 21-23 (e.g., 21-22, 22-23) contiguous nucleotides differing by no more than 3 (e.g., 0, 1, 2, or 3) nucleotides from the corresponding complementary nucleotide sequence of SEQ ID NO: 2.

In some embodiments, the sense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 23, 24, 25, 26, 27, 28, 29, 30 (e.g., 23) contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 23 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of about 21 contiguous nucleotides differing by no more than 3 (e.g., 0, 1, 2, or 3) nucleotides from the corresponding complementary nucleotide sequence of SEQ ID NO: 2. In some embodiments, the sense strand comprises or consists of about 21 contiguous nucleotides differing by no more than 3 (e.g., 0, 1, 2, or 3) nucleotides from nucleotides 1225-1285, 1235-1275, 1240-1270, 1245-1265, 1227-1287, 1237-1277, 1242-1272, 1247-1267, 1231-1291, 1241-1261, 1246-1276, 1251-1271, 1235-1295, 1245-1265, 1250-1280, 1255-1275, 1353-1411, 1363-1381, 1368-1396, 1373-1391, 1762-1820, 1772-1810, 1757-1805, 1782-1800, 1912-1972, 1922-1942, 1927-1957, 1932-1952, 1922-1982, 1932-1972, 1937-1967, 1942-1962, 1923-1983, 1933-1973, 1938-1968, or 1943-1963 of SEQ ID NO: 1; and the antisense strand comprises or consists of about 19 contiguous nucleotides differing by no more than 3 (e.g., 0, 1, 2, or 3) nucleotides from the corresponding complementary nucleotide sequence of SEQ ID NO: 2.

4.2.3.6 Exemplary dsRNA Agents

The nucleotide sequence of exemplary unmodified dsRNA agents comprising a sense and antisense strand (e.g., suitable for targeting hCIDEB, suitable for inhibiting hCIDEB expression)) are set forth in Table 2. More specifically, Table 2 sets forth the nucleotide sequence of exemplary sense strands, antisense strands, and dsRNA agent pairs of sense and antisense strands. It is to be understood that while the sense and antisense strands are set forth in pairs in Table 2, the disclosure encompasses dsRNA agents comprising any sense strand and any antisense set forth in Table 2 (e.g., that are at least partially complementary (e.g., as could be determined by a person of ordinary skill in the art)). It is to be understood that while the nucleotide sequence of the sense strands and antisense strands in Table 2 are set forth as unmodified (not containing any modified nucleotides), the disclosure encompasses the sense and antisense sense strands set forth in Table 2 comprising one or more modified nucleotide (e.g., as described herein).

TABLE 2
Unmodified Sense and Antisense Strand Sequences of
CIDEB dsRNA Agents.

| dsRNA | Sense | SEQ | | Antisense | SEQ | | mRNA | SEQ |
|---|---|---|---|---|---|---|---|---|
| Agent | Sequence | ID | Range in | Sequence | ID | Range in | Target | ID |
| ID | 5′ to 3′ | NO | NM_014430.4 | 5′ to 3′ | NO | NM_014430.4 | Sequence | NO |
| 1 | AGAGCAAGC | 4 | 63-83 | UGCUUGCCUU | 187 | 61-83 | CCAGAGCAAG | 370 |
| | CGAAGGCAA | | | CGGCUUGCUC | | | CCGAAGGCAA | |
| | GCA | | | UGG | | | GCA | |
| 2 | AAGCCGAAG | 5 | 68-88 | UAUCGUGCUU | 188 | 66-88 | GCAAGCCGAA | 371 |
| | GCAAGCACG | | | GCCUUCGGCU | | | GGCAAGCACG | |
| | AUA | | | UGC | | | AUG | |
| 3 | AGCCGAAGG | 6 | 69-89 | UCAUCGUGCU | 189 | 67-89 | CAAGCCGAAG | 372 |
| | CAAGCACGA | | | UGCCUUCGGC | | | GCAAGCACGA | |
| | UGA | | | UUG | | | UGG | |
| 4 | CCGAAGGCA | 7 | 71-91 | UGCCAUCGUG | 190 | 69-91 | AGCCGAAGGC | 373 |
| | AGCACGAUG | | | CUUGCCUUCG | | | AAGCACGAUG | |
| | GCA | | | GCU | | | GCG | |
| 5 | CGAAGGCAA | 8 | 72-92 | UCGCCAUCGU | 191 | 70-92 | GCCGAAGGCA | 374 |
| | GCACGAUGG | | | GCUUGCCUUC | | | AGCACGAUGG | |
| | CGA | | | GGC | | | CGC | |
| 6 | GGCAAGCAC | 9 | 76-96 | UUGAGCGCCA | 192 | 74-96 | AAGGCAAGCA | 375 |
| | GAUGGCGCU | | | UCGUGCUUGC | | | CGAUGGCGCU | |
| | CAA | | | CUU | | | CAC | |
| 7 | GCAAGCACG | 10 | 77-97 | UGUGAGCGCC | 193 | 75-97 | AGGCAAGCAC | 376 |
| | AUGGCGCUC | | | AUCGUGCUUG | | | GAUGGCGCUC | |
| | ACA | | | CCU | | | ACC | |
| 8 | CAAGCACGA | 11 | 78-98 | UGGUGAGCGC | 194 | 76-98 | GGCAAGCACG | 377 |
| | UGGCGCUCA | | | CAUCGUGCUU | | | AUGGCGCUCA | |
| | CCA | | | GCC | | | CCA | |
| 9 | AAGCACGAU | 12 | 79-99 | UUGGUGAGCG | 195 | 77-99 | GCAAGCACGA | 378 |
| | GGCGCUCAC | | | CCAUCGUGCU | | | UGGCGCUCAC | |
| | CAA | | | UGC | | | CAG | |
| 10 | ACGAUGGCG | 13 | 83-103 | UCGGCUGGUG | 196 | 81-103 | GCACGAUGGC | 379 |
| | CUCACCAGC | | | AGCGCCAUCG | | | GCUCACCAGC | |
| | CGA | | | UGC | | | CGG | |
| 11 | CCCCGCAGC | 14 | 143-163 | UACGCUGGCA | 197 | 141-163 | CGCCCCGCAG | 380 |
| | CGUGCCAGC | | | CGGCUGCGGG | | | CCGUGCCAGC | |
| | GUA | | | GCG | | | GUC | |
| 12 | GCAGCCGUG | 15 | 147-167 | UCGUGACGCU | 198 | 145-167 | CCGCAGCCGU | 381 |
| | CCAGCGUCA | | | GGCACGGCUG | | | GCCAGCGUCA | |
| | CGA | | | CGG | | | CGC | |
| 13 | CAGCCGUGC | 16 | 148-168 | UGCGUGACGC | 199 | 146-168 | CGCAGCCGUG | 382 |
| | CAGCGUCAC | | | UGGCACGGCU | | | CCAGCGUCAC | |
| | GCA | | | GCG | | | GCU | |
| 14 | AGCCGUGCC | 17 | 149-169 | UAGCGUGACG | 200 | 147-169 | GCAGCCGUGC | 383 |
| | AGCGUCACG | | | CUGGCACGGC | | | CAGCGUCACG | |
| | CUA | | | UGC | | | CUG | |
| 15 | GCCGUGCCA | 18 | 150-170 | UCAGCGUGAC | 201 | 148-170 | CAGCCGUGCC | 384 |
| | GCGUCACGC | | | GCUGGCACGG | | | AGCGUCACGC | |
| | UGA | | | CUG | | | UGU | |
| 16 | CGUGCCAGC | 19 | 152-172 | UUACAGCGUG | 202 | 150-172 | GCCGUGCCAG | 385 |
| | GUCACGCUG | | | ACGCUGGCAC | | | CGUCACGCUG | |
| | UAA | | | GGC | | | UAG | |
| 17 | GCCAGCGUC | 20 | 155-175 | UUGCUACAGC | 203 | 153-175 | GUGCCAGCGU | 386 |
| | ACGCUGUAG | | | GUGACGCUGG | | | CACGCUGUAG | |
| | CAA | | | CAC | | | CAG | |
| 18 | CCAGCGUCA | 21 | 156-176 | UCUGCUACAG | 204 | 154-176 | UGCCAGCGUC | 387 |
| | CGCUGUAGC | | | CGUGACGCUG | | | ACGCUGUAGC | |
| | AGA | | | GCA | | | AGC | |
| 19 | CAGCGUCAC | 22 | 157-177 | UGCUGCUACA | 205 | 155-177 | GCCAGCGUCA | 388 |
| | GCUGUAGCA | | | GCGUGACGCU | | | CGCUGUAGCA | |
| | GCA | | | GGC | | | GCC | |
| 20 | GCGUCACGC | 23 | 159-179 | UCGGCUGCUA | 206 | 157-179 | CAGCGUCACG | 389 |
| | UGUAGCAGC | | | CAGCGUGACG | | | CUGUAGCAGC | |
| | CGA | | | CUG | | | CGA | |
| 21 | CGUCACGCU | 24 | 160-180 | UUCGGCUGCU | 207 | 158-180 | AGCGUCACGC | 390 |
| | GUAGCAGCC | | | ACAGCGUGAC | | | UGUAGCAGCC | |
| | GAA | | | GCU | | | GAG | |
| 22 | GUCACGCUG | 25 | 161-181 | UCUCGGCUGC | 208 | 159-181 | GCGUCACGCU | 391 |
| | UAGCAGCCG | | | UACAGCGUGA | | | GUAGCAGCCG | |
| | AGA | | | CGC | | | AGC | |
| 23 | CACGCUGUA | 26 | 163-183 | UUGCUCGGCU | 209 | 161-183 | GUCACGCUGU | 392 |
| | GCAGCCGAG | | | GCUACAGCGU | | | AGCAGCCGAG | |
| | CAA | | | GAC | | | CAU | |
| 24 | ACGCUGUAG | 27 | 164-184 | UAUGCUCGGC | 210 | 162-184 | UCACGCUGUA | 393 |
| | CAGCCGAGC | | | UGCUACAGCG | | | GCAGCCGAGC | |
| | AUA | | | UGA | | | AUC | |
| 25 | GCUGUAGCA | 28 | 166-186 | UUGAUGCUCG | 211 | 164-186 | ACGCUGUAGC | 394 |
| | GCCGAGCAU | | | GCUGCUACAG | | | AGCCGAGCAU | |
| | CAA | | | CGU | | | CAG | |
| 26 | AGCCGAGCA | 29 | 174-194 | UUUUCGGGCU | 212 | 172-194 | GCAGCCGAGC | 395 |
| | UCAGCCCGA | | | GAUGCUCGGC | | | AUCAGCCCGA | |
| | AAA | | | UGC | | | AAG | |
| 27 | CGAGCAUCA | 30 | 177-197 | UUCCUUUCGG | 213 | 175-197 | GCCGAGCAUC | 396 |
| | GCCCGAAAG | | | GCUGAUGCUC | | | AGCCCGAAAG | |
| | GAA | | | GGC | | | GAA | |
| 28 | CAGCCCGAA | 31 | 184-204 | UUCGUGCUUC | 214 | 182-204 | AUCAGCCCGA | 397 |
| | AGGAAGCAC | | | CUUUCGGGCU | | | AAGGAAGCAC | |
| | GAA | | | GAU | | | GAA | |
| 29 | AGCCCGAAA | 32 | 185-205 | UUUCGUGCUU | 215 | 183-205 | UCAGCCCGAA | 398 |
| | GGAAGCACG | | | CCUUUCGGGC | | | AGGAAGCACG | |
| | AAA | | | UGA | | | AAA | |
| 30 | GCCCGAAAG | 33 | 186-206 | UUUUCGUGCU | 216 | 184-206 | CAGCCCGAAA | 399 |
| | GAAGCACGA | | | UCCUUUCGGG | | | GGAAGCACGA | |
| | AAA | | | CUG | | | AAG | |
| 31 | CGGCGGCGU | 34 | 234-254 | UGUCGCCGGU | 217 | 232-254 | GGCGGCGGCG | 400 |
| | GGACCGGCG | | | CCACGCCGCC | | | UGGACCGGCG | |
| | ACA | | | GCC | | | ACG | |
| 32 | GGCGGCGUG | 35 | 235-255 | UCGUCGCCGG | 218 | 233-255 | GCGGCGGCGU | 401 |
| | GACCGGCGA | | | UCCACGCCGC | | | GGACCGGCGA | |
| | CGA | | | CGC | | | CGG | |
| 33 | GCGGCGUGG | 36 | 236-256 | UCCGUCGCCG | 219 | 234-256 | CGGCGGCGUG | 402 |
| | ACCGGCGAC | | | GUCCACGCCG | | | GACCGGCGAC | |
| | GGA | | | CCG | | | GGG | |
| 34 | GUGGACCGG | 37 | 241-261 | UGCCACCCGU | 220 | 239-261 | GCGUGGACCG | 403 |
| | CGACGGGUG | | | CGCCGGUCCA | | | GCGACGGGUG | |
| | GCA | | | CGC | | | GCA | |
| 35 | UGGACCGGC | 38 | 242-262 | UUGCCACCCG | 221 | 240-262 | CGUGGACCGG | 404 |
| | GACGGGUGG | | | UCGCCGGUCC | | | CGACGGGUGG | |
| | CAA | | | ACG | | | CAC | |
| 36 | GGACCGGCG | 39 | 243-263 | UGUGCCACCC | 222 | 241-263 | GUGGACCGGC | 405 |
| | ACGGGUGGC | | | GUCGCCGGUC | | | GACGGGUGGC | |
| | ACA | | | CAC | | | ACA | |
| 37 | GACCGGCGA | 40 | 244-264 | UUGUGCCACC | 223 | 242-264 | UGGACCGGCG | 406 |
| | CGGGUGGCA | | | CGUCGCCGGU | | | ACGGGUGGCA | |
| | CAA | | | CCA | | | CAG | |
| 38 | CGGCGACGG | 41 | 247-267 | UAGCUGUGCC | 224 | 245-267 | ACCGGCGACG | 407 |
| | GUGGCACAG | | | ACCCGUCGCC | | | GGUGGCACAG | |
| | CUA | | | GGU | | | CUG | |
| 39 | GCGACGGGU | 42 | 249-269 | UCCAGCUGUG | 225 | 247-269 | CGGCGACGGG | 408 |
| | GGCACAGCU | | | CCACCCGUCG | | | UGGCACAGCU | |
| | GGA | | | CCG | | | GGC | |
| 40 | GGUGGCACA | 43 | 255-275 | UCGUAUGCCA | 226 | 253-275 | CGGGUGGCAC | 409 |
| | GCUGGCAUA | | | GCUGUGCCAC | | | AGCUGGCAUA | |
| | CGA | | | CCG | | | CGC | |
| 41 | GUGGCACAG | 44 | 256-276 | UGCGUAUGCC | 227 | 254-276 | GGGUGGCACA | 410 |
| | CUGGCAUAC | | | AGCUGUGCCA | | | GCUGGCAUAC | |
| | GCA | | | CCC | | | GCG | |
| 42 | UGGCACAGC | 45 | 257-277 | UCGCGUAUGC | 228 | 255-277 | GGUGGCACAG | 411 |
| | UGGCAUACG | | | CAGCUGUGCC | | | CUGGCAUACG | |
| | CGA | | | ACC | | | CGG | |
| 43 | CCUCCACAG | 46 | 280-300 | UUCUACCGCC | 229 | 278-300 | UCCCUCCACA | 412 |
| | GUGGCGGUA | | | ACCUGUGGAG | | | GGUGGCGGUA | |
| | GAA | | | GGA | | | GAC | |
| 44 | UCCACAGGU | 47 | 282-302 | UCGUCUACCG | 230 | 280-302 | CCUCCACAGG | 413 |
| | GGCGGUAGA | | | CCACCUGUGG | | | UGGCGGUAGA | |
| | CGA | | | AGG | | | CGG | |
| 45 | CACAGGUGG | 48 | 284-304 | UGCCGUCUAC | 231 | 282-304 | UCCACAGGUG | 414 |
| | CGGUAGACG | | | CGCCACCUGU | | | GCGGUAGACG | |
| | GCA | | | GGA | | | GCG | |
| 46 | GUGGCGGUA | 49 | 289-309 | UCGGCCGCCG | 232 | 287-309 | AGGUGGCGGU | 415 |
| | GACGGCGGC | | | UCUACCGCCA | | | AGACGGCGGC | |
| | CGA | | | CCU | | | CGG | |
| 47 | CGGCCGGGA | 50 | 303-323 | UGUUGCUCGC | 233 | 301-323 | GGCGGCCGGG | 416 |
| | CGGCGAGCA | | | CGUCCCGGCC | | | ACGGCGAGCA | |
| | ACA | | | GCC | | | ACA | |
| 48 | GGCCGGGAC | 51 | 304-324 | UUGUUGCUCG | 234 | 302-324 | GCGGCCGGGA | 417 |
| | GGCGAGCAA | | | CCGUCCCGGC | | | CGGCGAGCAA | |
| | CAA | | | CGC | | | CAG | |
| 49 | CUGGCGUAC | 52 | 449-469 | UGCGCUCAGC | 235 | 447-469 | CGCUGGCGUA | 418 |
| | AUGCUGAGC | | | AUGUACGCCA | | | CAUGCUGAGC | |
| | GCA | | | GCG | | | GCG | |
| 50 | UGGCGUACA | 53 | 450-470 | UCGCGCUCAG | 236 | 448-470 | GCUGGCGUAC | 419 |
| | UGCUGAGCG | | | CAUGUACGCC | | | AUGCUGAGCG | |
| | CGA | | | AGC | | | CGC | |
| 51 | GGCGUACAU | 54 | 451-471 | UGCGCGCUCA | 237 | 449-471 | CUGGCGUACA | 420 |
| | GCUGAGCGC | | | GCAUGUACGC | | | UGCUGAGCGC | |
| | GCA | | | CAG | | | GCA | |
| 52 | GCGUACAUG | 55 | 452-472 | UUGCGCGCUC | 238 | 450-472 | UGGCGUACAU | 421 |
| | CUGAGCGCG | | | AGCAUGUACG | | | GCUGAGCGCG | |
| | CAA | | | CCA | | | CAC | |
| 53 | GCUGAGCGC | 56 | 460-480 | UACUACGUGU | 239 | 458-480 | AUGCUGAGCG | 422 |
| | GCACACGUA | | | GCGCGCUCAG | | | CGCACACGUA | |
| | GUA | | | CAU | | | GUA | |
| 54 | CUGAGCGCG | 57 | 461-481 | UUACUACGUG | 240 | 459-481 | UGCUGAGCGC | 423 |
| | CACACGUAG | | | UGCGCGCUCA | | | GCACACGUAG | |
| | UAA | | | GCA | | | UAC | |
| 55 | GAGCGCGCA | 58 | 463-483 | UUGUACUACG | 241 | 461-483 | CUGAGCGCGC | 424 |
| | CACGUAGUA | | | UGUGCGCGCU | | | ACACGUAGUA | |
| | CAA | | | CAG | | | CAC | |
| 56 | CGCGCACAC | 59 | 466-486 | UCGGUGUACU | 242 | 464-486 | AGCGCGCACA | 425 |
| | GUAGUACAC | | | ACGUGUGCGC | | | CGUAGUACAC | |
| | CGA | | | GCU | | | CGC | |
| 57 | GCGCACACG | 60 | 467-487 | UGCGGUGUAC | 243 | 465-487 | GCGCGCACAC | 426 |
| | UAGUACACC | | | UACGUGUGCG | | | GUAGUACACC | |
| | GCA | | | CGC | | | GCC | |
| 58 | CGCACACGU | 61 | 468-488 | UGGCGGUGUA | 244 | 466-488 | CGCGCACACG | 427 |
| | AGUACACCG | | | CUACGUGUGC | | | UAGUACACCG | |
| | CCA | | | GCG | | | CCU | |
| 59 | GCACACGUA | 62 | 469-489 | UAGGCGGUGU | 245 | 467-489 | GCGCACACGU | 428 |
| | GUACACCGC | | | ACUACGUGUG | | | AGUACACCGC | |
| | CUA | | | CGC | | | CUU | |
| 60 | CACACGUAG | 63 | 470-490 | UAAGGCGGUG | 246 | 468-490 | CGCACACGUA | 429 |
| | UACACCGCC | | | UACUACGUGU | | | GUACACCGCC | |
| | UUA | | | GCG | | | UUG | |
| 61 | ACACGUAGU | 64 | 471-491 | UCAAGGCGGU | 247 | 469-491 | GCACACGUAG | 430 |
| | ACACCGCCU | | | GUACUACGUG | | | UACACCGCCU | |
| | UGA | | | UGC | | | UGC | |
| 62 | ACGUAGUAC | 65 | 473-493 | UUGCAAGGCG | 248 | 471-493 | ACACGUAGUA | 431 |
| | ACCGCCUUG | | | GUGUACUACG | | | CACCGCCUUG | |
| | CAA | | | UGU | | | CAG | |
| 63 | CGUAGUACA | 66 | 474-494 | UCUGCAAGGC | 249 | 472-494 | CACGUAGUAC | 432 |
| | CCGCCUUGC | | | GGUGUACUAC | | | ACCGCCUUGC | |
| | AGA | | | GUG | | | AGC | |
| 64 | GUAGUACAC | 67 | 475-495 | UGCUGCAAGG | 250 | 473-495 | ACGUAGUACA | 433 |
| | CGCCUUGCA | | | CGGUGUACUA | | | CCGCCUUGCA | |
| | GCA | | | CGU | | | GCC | |
| 65 | GCCUGCCGG | 68 | 515-535 | UGCCUUCCUG | 251 | 513-535 | AGGCCUGCCG | 434 |
| | GUCAGGAAG | | | ACCCGGCAGG | | | GGUCAGGAAG | |
| | GCA | | | CCU | | | GCC | |
| 66 | CAGGAAGGC | 69 | 526-546 | UCGCUCUUUG | 252 | 524-546 | GUCAGGAAGG | 435 |
| | CACAAAGAG | | | UGGCCUUCCU | | | CCACAAAGAG | |
| | CGA | | | GAC | | | CGG | |
| 67 | GGAAGGCCA | 70 | 528-548 | UGCCGCUCUU | 253 | 526-548 | CAGGAAGGCC | 436 |
| | CAAAGAGCG | | | UGUGGCCUUC | | | ACAAAGAGCG | |
| | GCA | | | CUG | | | GCG | |
| 68 | GAAGGCCAC | 71 | 529-549 | UCGCCGCUCU | 254 | 527-549 | AGGAAGGCCA | 437 |
| | AAAGAGCGG | | | UUGUGGCCUU | | | CAAAGAGCGG | |
| | CGA | | | CCU | | | CGU | |
| 69 | AAGGCCACA | 72 | 530-550 | UACGCCGCUC | 255 | 528-550 | GGAAGGCCAC | 438 |
| | AAGAGCGGC | | | UUUGUGGCCU | | | AAAGAGCGGC | |
| | GUA | | | UCC | | | GUG | |
| 70 | AGGCCACAA | 73 | 531-551 | UCACGCCGCU | 256 | 529-551 | GAAGGCCACA | 439 |
| | AGAGCGGCG | | | CUUUGUGGCC | | | AAGAGCGGCG | |
| | UGA | | | UUC | | | UGA | |
| 71 | GGCCACAAA | 74 | 532-552 | UUCACGCCGC | 257 | 530-552 | AAGGCCACAA | 440 |
| | GAGCGGCGU | | | UCUUUGUGGC | | | AGAGCGGCGU | |
| | GAA | | | CUU | | | GAG | |
| 72 | CCACAAAGA | 75 | 534-554 | UGCUCACGCC | 258 | 532-554 | GGCCACAAAG | 441 |
| | GCGGCGUGA | | | GCUCUUUGUG | | | AGCGGCGUGA | |
| | GCA | | | GCC | | | GCA | |
| 73 | CACAAAGAG | 76 | 535-555 | UUGCUCACGC | 259 | 533-555 | GCCACAAAGA | 442 |
| | CGGCGUGAG | | | CGCUCUUUGU | | | GCGGCGUGAG | |
| | CAA | | | GGC | | | CAG | |
| 74 | AGCACCGCG | 77 | 557-577 | UCUGGCCGAC | 260 | 555-577 | GCAGCACCGC | 443 |
| | CCGUCGGCC | | | GGCGCGGUGC | | | GCCGUCGGCC | |
| | AGA | | | UGC | | | AGC | |
| 75 | CGCGCCGUC | 78 | 562-582 | UUGGCGCUGG | 261 | 560-582 | ACCGCGCCGU | 444 |
| | GGCCAGCGC | | | CCGACGGCGC | | | CGGCCAGCGC | |
| | CAA | | | GGU | | | CAG | |
| 76 | GCGCCGUCG | 79 | 563-583 | UCUGGCGCUG | 262 | 561-583 | CCGCGCCGUC | 445 |
| | GCCAGCGCC | | | GCCGACGGCG | | | GGCCAGCGCC | |
| | AGA | | | CGG | | | AGG | |
| 77 | CAGCACAAG | 80 | 586-606 | UUGGCGGCCA | 263 | 584-606 | UGCAGCACAA | 446 |
| | CGUGGCCGC | | | CGCUUGUGCU | | | GCGUGGCCGC | |
| | CAA | | | GCA | | | CAG | |
| 78 | AGCACAAGC | 81 | 587-607 | UCUGGCGGCC | 264 | 585-607 | GCAGCACAAG | 447 |
| | GUGGCCGCC | | | ACGCUUGUGC | | | CGUGGCCGCC | |
| | AGA | | | UGC | | | AGC | |
| 79 | GCACAAGCG | 82 | 588-608 | UGCUGGCGGC | 265 | 586-608 | CAGCACAAGC | 448 |
| | UGGCCGCCA | | | CACGCUUGUG | | | GUGGCCGCCA | |
| | GCA | | | CUG | | | GCG | |
| 80 | CAAGCGUGG | 83 | 591-611 | UACCGCUGGC | 266 | 589-611 | CACAAGCGUG | 449 |
| | CCGCCAGCG | | | GGCCACGCUU | | | GCCGCCAGCG | |
| | GUA | | | GUG | | | GUC | |
| 81 | AAGCGUGGC | 84 | 592-612 | UGACCGCUGG | 267 | 590-612 | ACAAGCGUGG | 450 |
| | CGCCAGCGG | | | CGGCCACGCU | | | CCGCCAGCGG | |
| | UCA | | | UGU | | | UCG | |
| 82 | AGCGUGGCC | 85 | 593-613 | UCGACCGCUG | 268 | 591-613 | CAAGCGUGGC | 451 |
| | GCCAGCGGU | | | GCGGCCACGC | | | CGCCAGCGGU | |
| | CGA | | | UUG | | | CGC | |
| 83 | GCGUGGCCG | 86 | 594-614 | UGCGACCGCU | 269 | 592-614 | AAGCGUGGCC | 452 |
| | CCAGCGGUC | | | GGCGGCCACG | | | GCCAGCGGUC | |
| | GCA | | | CUU | | | GCC | |
| 84 | GGCUGUGCC | 87 | 697-717 | UCGCGGGCCA | 270 | 695-717 | AAGGCUGUGC | 453 |
| | UGUGGCCCG | | | CAGGCACAGC | | | CUGUGGCCCG | |
| | CGA | | | CUU | | | CGA | |
| 85 | GCCUGUGGC | 88 | 703-723 | UAGACUUCGC | 271 | 701-723 | GUGCCUGUGG | 454 |
| | CCGCGAAGU | | | GGGCCACAGG | | | CCCGCGAAGU | |
| | CUA | | | CAC | | | CUU | |
| 86 | CUGUGGCCC | 89 | 705-725 | UGAAGACUUC | 272 | 703-725 | GCCUGUGGCC | 455 |
| | GCGAAGUCU | | | GCGGGCCACA | | | CGCGAAGUCU | |
| | UCA | | | GGC | | | UCC | |
| 87 | GCCCGCGAA | 90 | 710-730 | UAGCUGGAAG | 273 | 708-730 | UGGCCCGCGA | 456 |
| | GUCUUCCAG | | | ACUUCGCGGG | | | AGUCUUCCAG | |
| | CUA | | | CCA | | | CUC | |
| 88 | CCCGCGAAG | 91 | 711-731 | UGAGCUGGAA | 274 | 709-731 | GGCCCGCGAA | 457 |
| | UCUUCCAGC | | | GACUUCGCGG | | | GUCUUCCAGC | |
| | UCA | | | GCC | | | UCA | |
| 89 | CCGCGAAGU | 92 | 712-732 | UUGAGCUGGA | 275 | 710-732 | GCCCGCGAAG | 458 |
| | CUUCCAGCU | | | AGACUUCGCG | | | UCUUCCAGCU | |
| | CAA | | | GGC | | | CAG | |
| 90 | UCCAGCUCA | 93 | 723-743 | UCGAGACACU | 276 | 721-743 | CUUCCAGCUC | 459 |
| | GCAGUGUCU | | | GCUGAGCUGG | | | AGCAGUGUCU | |
| | CGA | | | AAG | | | CGU | |
| 91 | CAGCUCAGC | 94 | 725-745 | UAACGAGACA | 277 | 723-745 | UCCAGCUCAG | 460 |
| | AGUGUCUCG | | | CUGCUGAGCU | | | CAGUGUCUCG | |
| | UUA | | | GGA | | | UUC | |
| 92 | GCGCACCAG | 95 | 944-964 | UGACUUUAGC | 278 | 942-964 | CAGCGCACCA | 461 |
| | AAGCUAAAG | | | UUCUGGUGCG | | | GAAGCUAAAG | |
| | UCA | | | CUG | | | UCU | |
| 93 | CAGAAGCUA | 96 | 950-970 | UCAUCAAGAC | 279 | 948-970 | ACCAGAAGCU | 462 |
| | AAGUCUUGA | | | UUUAGCUUCU | | | AAAGUCUUGA | |
| | UGA | | | GGU | | | UGC | |
| 94 | AGAAGCUAA | 97 | 951-971 | UGCAUCAAGA | 280 | 949-971 | CCAGAAGCUA | 463 |
| | AGUCUUGAU | | | CUUUAGCUUC | | | AAGUCUUGAU | |
| | GCA | | | UGG | | | GCC | |
| 95 | GAAGCUAAA | 98 | 952-972 | UGGCAUCAAG | 281 | 950-972 | CAGAAGCUAA | 464 |
| | GUCUUGAUG | | | ACUUUAGCUU | | | AGUCUUGAUG | |
| | CCA | | | CUG | | | CCA | |
| 96 | AAGCUAAAG | 99 | 953-973 | UUGGCAUCAA | 282 | 951-973 | AGAAGCUAAA | 465 |
| | UCUUGAUGC | | | GACUUUAGCU | | | GUCUUGAUGC | |
| | CAA | | | UCU | | | CAU | |
| 97 | GCUAAAGUC | 100 | 955-975 | UGAUGGCAUC | 283 | 953-975 | AAGCUAAAGU | 466 |
| | UUGAUGCCA | | | AAGACUUUAG | | | CUUGAUGCCA | |
| | UCA | | | CUU | | | UCA | |
| 98 | GAUGCCAUC | 101 | 966-986 | UGGAUGUCCU | 284 | 964-986 | UUGAUGCCAU | 467 |
| | AAAGGACAU | | | UUGAUGGCAU | | | CAAAGGACAU | |
| | CCA | | | CAA | | | CCC | |
| 99 | AUGCCAUCA | 102 | 967-987 | UGGGAUGUCC | 285 | 965-987 | UGAUGCCAUC | 468 |
| | AAGGACAUC | | | UUUGAUGGCA | | | AAAGGACAUC | |
| | CCA | | | UCA | | | CCU | |
| 100 | GCCAUCAAA | 103 | 969-989 | UCAGGGAUGU | 286 | 967-989 | AUGCCAUCAA | 469 |
| | GGACAUCCC | | | CCUUUGAUGG | | | AGGACAUCCC | |
| | UGA | | | CAU | | | UGC | |
| 101 | CAUCUCUGU | 104 | 998-1018 | UUAGUGGACG | 287 | 996-1018 | CACAUCUCUG | 470 |
| | CACGUCCAC | | | UGACAGAGAU | | | UCACGUCCAC | |
| | UAA | | | GUG | | | UAA | |
| 102 | AUCUCUGUC | 105 | 999-1019 | UUUAGUGGAC | 288 | 997-1019 | ACAUCUCUGU | 471 |
| | ACGUCCACU | | | GUGACAGAGA | | | CACGUCCACU | |
| | AAA | | | UGU | | | AAU | |
| 103 | UCUCUGUCA | 106 | 1000-1020 | UAUUAGUGGA | 289 | 998-1020 | CAUCUCUGUC | 472 |
| | CGUCCACUA | | | CGUGACAGAG | | | ACGUCCACUA | |
| | AUA | | | AUG | | | AUC | |
| 104 | CUCUGUCAC | 107 | 1001-1021 | UGAUUAGUGG | 290 | 999-1021 | AUCUCUGUCA | 473 |
| | GUCCACUAA | | | ACGUGACAGA | | | CGUCCACUAA | |
| | UCA | | | GAU | | | UCG | |
| 105 | UCUGUCACG | 108 | 1002-1022 | UCGAUUAGUG | 291 | 1000-1022 | UCUCUGUCAC | 474 |
| | UCCACUAAU | | | GACGUGACAG | | | GUCCACUAAU | |
| | CGA | | | AGA | | | CGG | |
| 106 | CUGUCACGU | 109 | 1003-1023 | UCCGAUUAGU | 292 | 1001-1023 | CUCUGUCACG | 475 |
| | CCACUAAUC | | | GGACGUGACA | | | UCCACUAAUC | |
| | GGA | | | GAG | | | GGC | |
| 107 | UGUCACGUC | 110 | 1004-1024 | UGCCGAUUAG | 293 | 1002-1024 | UCUGUCACGU | 476 |
| | CACUAAUCG | | | UGGACGUGAC | | | CCACUAAUCG | |
| | GCA | | | AGA | | | GCA | |
| 108 | GUCACGUCC | 111 | 1005-1025 | UUGCCGAUUA | 294 | 1003-1025 | CUGUCACGUC | 477 |
| | ACUAAUCGG | | | GUGGACGUGA | | | CACUAAUCGG | |
| | CAA | | | CAG | | | CAA | |
| 109 | UCACGUCCA | 112 | 1006-1026 | UUUGCCGAUU | 295 | 1004-1026 | UGUCACGUCC | 478 |
| | CUAAUCGGC | | | AGUGGACGUG | | | ACUAAUCGGC | |
| | AAA | | | ACA | | | AAA | |
| 110 | CACGUCCAC | 113 | 1007-1027 | UUUUGCCGAU | 296 | 1005-1027 | GUCACGUCCA | 479 |
| | UAAUCGGCA | | | UAGUGGACGU | | | CUAAUCGGCA | |
| | AAA | | | GAC | | | AAA | |
| 111 | CGUCCACUA | 114 | 1009-1029 | UCUUUUGCCG | 297 | 1007-1029 | CACGUCCACU | 480 |
| | AUCGGCAAA | | | AUUAGUGGAC | | | AAUCGGCAAA | |
| | AGA | | | GUG | | | AGG | |
| 112 | GUCCACUAA | 115 | 1010-1030 | UCCUUUUGCC | 298 | 1008-1030 | ACGUCCACUA | 481 |
| | UCGGCAAAA | | | GAUUAGUGGA | | | AUCGGCAAAA | |
| | GGA | | | CGU | | | GGA | |
| 113 | UCCACUAAU | 116 | 1011-1031 | UUCCUUUUGC | 299 | 1009-1031 | CGUCCACUAA | 482 |
| | CGGCAAAAG | | | CGAUUAGUGG | | | UCGGCAAAAG | |
| | GAA | | | ACG | | | GAG | |
| 114 | CCACUAAUC | 117 | 1012-1032 | UCUCCUUUUG | 300 | 1010-1032 | GUCCACUAAU | 483 |
| | GGCAAAAGG | | | CCGAUUAGUG | | | CGGCAAAAGG | |
| | AGA | | | GAC | | | AGA | |
| 115 | CACUAAUCG | 118 | 1013-1033 | UUCUCCUUUU | 301 | 1011-1033 | UCCACUAAUC | 484 |
| | GCAAAAGGA | | | GCCGAUUAGU | | | GGCAAAAGGA | |
| | GAA | | | GGA | | | GAA | |
| 116 | ACUAAUCGG | 119 | 1014-1034 | UUUCUCCUUU | 302 | 1012-1034 | CCACUAAUCG | 485 |
| | CAAAAGGAG | | | UGCCGAUUAG | | | GCAAAAGGAG | |
| | AAA | | | UGG | | | AAA | |
| 117 | GAGAAGAUG | 120 | 1040-1060 | UCACACUUAG | 303 | 1038-1060 | GAGAGAAGAU | 486 |
| | ACCUAAGUG | | | GUCAUCUUCU | | | GACCUAAGUG | |
| | UGA | | | CUC | | | UGA | |
| 118 | UCCUCCGAC | 121 | 1110-1130 | UCUUCCUCCU | 304 | 1108-1130 | CCUCCUCCGA | 487 |
| | CAAGGAGGA | | | UGGUCGGAGG | | | CCAAGGAGGA | |
| | AGA | | | AGG | | | AGG | |
| 119 | CCAGGGAAG | 122 | 1176-1196 | UCCGGAGAGU | 305 | 1174-1196 | UUCCAGGGAA | 488 |
| | GAACUCUCC | | | UCCUUCCCUG | | | GGAACUCUCC | |
| | GGA | | | GAA | | | GGU | |
| 120 | CAGGGAAGG | 123 | 1177-1197 | UACCGGAGAG | 306 | 1175-1197 | UCCAGGGAAG | 489 |
| | AACUCUCCG | | | UUCCUUCCCU | | | GAACUCUCCG | |
| | GUA | | | GGA | | | GUC | |
| 121 | AGGAACUCU | 124 | 1183-1203 | UUGGUGGACC | 307 | 1181-1203 | GAAGGAACUC | 490 |
| | CCGGUCCAC | | | GGAGAGUUCC | | | UCCGGUCCAC | |
| | CAA | | | UUC | | | CAU | |
| 122 | GGAACUCUC | 125 | 1184-1204 | UAUGGUGGAC | 308 | 1182-1204 | AAGGAACUCU | 491 |
| | CGGUCCACC | | | CGGAGAGUUC | | | CCGGUCCACC | |
| | AUA | | | CUU | | | AUG | |
| 123 | ACUCUCCGG | 126 | 1187-1207 | UUCCAUGGUG | 309 | 1185-1207 | GAACUCUCCG | 492 |
| | UCCACCAUG | | | GACCGGAGAG | | | GUCCACCAUG | |
| | GAA | | | UUC | | | GAG | |
| 124 | CUCCGGUCC | 127 | 1190-1210 | UUACUCCAUG | 310 | 1188-1210 | CUCUCCGGUC | 493 |
| | ACCAUGGAG | | | GUGGACCGGA | | | CACCAUGGAG | |
| | UAA | | | GAG | | | UAC | |
| 125 | UGAACCCCA | 128 | 1221-1241 | UGAGUAAGUC | 311 | 1219-1241 | UCUGAACCCC | 494 |
| | GUGACUUAC | | | ACUGGGGUUC | | | AGUGACUUAC | |
| | UCA | | | AGA | | | UCA | |
| 126 | GUGACUUAC | 129 | 1230-1250 | UUACUGACCU | 312 | 1228-1250 | CAGUGACUUA | 495 |
| | UCAGGUCAG | | | GAGUAAGUCA | | | CUCAGGUCAG | |
| | UAA | | | CUG | | | UAU | |
| 127 | GACUUACUC | 130 | 1232-1252 | UGAUACUGAC | 313 | 1230-1252 | GUGACUUACU | 496 |
| | AGGUCAGUA | | | CUGAGUAAGU | | | CAGGUCAGUA | |
| | UCA | | | CAC | | | UCU | |
| 128 | CUUACUCAG | 131 | 1234-1254 | UUAGAUACUG | 314 | 1232-1254 | GACUUACUCA | 497 |
| | GUCAGUAUC | | | ACCUGAGUAA | | | GGUCAGUAUC | |
| | UAA | | | GUC | | | UAA | |
| 129 | CAGUAUCUA | 132 | 1245-1265 | UCGAGCUUAU | 315 | 1243-1265 | GUCAGUAUCU | 498 |
| | AUAUAAGCU | | | AUUAGAUACU | | | AAUAUAAGCU | |
| | CGA | | | GAC | | | CGG | |
| 130 | GUAUCUAAU | 133 | 1247-1267 | UUCCGAGCUU | 316 | 1245-1267 | CAGUAUCUAA | 499 |
| | AUAAGCUCG | | | AUAUUAGAUA | | | UAUAAGCUCG | |
| | GAA | | | CUG | | | GAG | |
| 131 | UAUCUAAUA | 134 | 1248-1268 | UCUCCGAGCU | 317 | 1246-1268 | AGUAUCUAAU | 500 |
| | UAAGCUCGG | | | UAUAUUAGAU | | | AUAAGCUCGG | |
| | AGA | | | ACU | | | AGU | |
| 132 | AUCUAAUAU | 135 | 1249-1269 | UACUCCGAGC | 318 | 1247-1269 | GUAUCUAAUA | 501 |
| | AAGCUCGGA | | | UUAUAUUAGA | | | UAAGCUCGGA | |
| | GUA | | | UAC | | | GUU | |
| 133 | UCUAAUAUA | 136 | 1250-1270 | UAACUCCGAG | 319 | 1248-1270 | UAUCUAAUAU | 502 |
| | AGCUCGGAG | | | CUUAUAUUAG | | | AAGCUCGGAG | |
| | UUA | | | AUA | | | UUU | |
| 134 | CUAAUAUAA | 137 | 1251-1271 | UAAACUCCGA | 320 | 1249-1271 | AUCUAAUAUA | 503 |
| | GCUCGGAGU | | | GCUUAUAUUA | | | AGCUCGGAGU | |
| | UUA | | | GAU | | | UUG | |
| 135 | UAAUAUAAG | 138 | 1252-1272 | UCAAACUCCG | 321 | 1250-1272 | UCUAAUAUAA | 504 |
| | CUCGGAGUU | | | AGCUUAUAUU | | | GCUCGGAGUU | |
| | UGA | | | AGA | | | UGG | |
| 136 | AUAUAAGCU | 139 | 1254-1274 | UUCCAAACUC | 322 | 1252-1274 | UAAUAUAAGC | 505 |
| | CGGAGUUUG | | | CGAGCUUAUA | | | UCGGAGUUUG | |
| | GAA | | | UUA | | | GAC | |
| 137 | UAUAAGCUC | 140 | 1255-1275 | UGUCCAAACU | 323 | 1253-1275 | AAUAUAAGCU | 506 |
| | GGAGUUUGG | | | CCGAGCUUAU | | | CGGAGUUUGG | |
| | ACA | | | AUU | | | ACG | |
| 138 | AUAAGCUCG | 141 | 1256-1276 | UCGUCCAAAC | 324 | 1254-1276 | AUAUAAGCUC | 507 |
| | GAGUUUGGA | | | UCCGAGCUUA | | | GGAGUUUGGA | |
| | CGA | | | UAU | | | CGG | |
| 139 | GCUCGGAGU | 142 | 1260-1280 | UCCUCCGUCC | 325 | 1258-1280 | AAGCUCGGAG | 508 |
| | UUGGACGGA | | | AAACUCCGAG | | | UUUGGACGGA | |
| | GGA | | | CUU | | | GGG | |
| 140 | GGAGUUUGG | 143 | 1264-1284 | UAGACCCUCC | 326 | 1262-1284 | UCGGAGUUUG | 509 |
| | ACGGAGGGU | | | GUCCAAACUC | | | GACGGAGGGU | |
| | CUA | | | CGA | | | CUG | |
| 141 | CACCCCAGC | 144 | 1299-1319 | UACGGAAAGG | 327 | 1297-1319 | ACCACCCCAG | 510 |
| | GACCUUUCC | | | UCGCUGGGGU | | | CGACCUUUCC | |
| | GUA | | | GGU | | | GUG | |
| 142 | GUCUGUGAU | 145 | 1319-1339 | UGUCCGCUUG | 328 | 1317-1339 | GUGUCUGUGA | 511 |
| | CACAAGCGG | | | UGAUCACAGA | | | UCACAAGCGG | |
| | ACA | | | CAC | | | ACC | |
| 143 | UCUGUGAUC | 146 | 1320-1340 | UGGUCCGCUU | 329 | 1318-1340 | UGUCUGUGAU | 512 |
| | ACAAGCGGA | | | GUGAUCACAG | | | CACAAGCGGA | |
| | CCA | | | ACA | | | CCA | |
| 144 | CUGUGAUCA | 147 | 1321-1341 | UUGGUCCGCU | 330 | 1319-1341 | GUCUGUGAUC | 513 |
| | CAAGCGGAC | | | UGUGAUCACA | | | ACAAGCGGAC | |
| | CAA | | | GAC | | | CAU | |
| 145 | UGUGAUCAC | 148 | 1322-1342 | UAUGGUCCGC | 331 | 1320-1342 | UCUGUGAUCA | 514 |
| | AAGCGGACC | | | UUGUGAUCAC | | | CAAGCGGACC | |
| | AUA | | | AGA | | | AUC | |
| 146 | GUGAUCACA | 149 | 1323-1343 | UGAUGGUCCG | 332 | 1321-1343 | CUGUGAUCAC | 515 |
| | AGCGGACCA | | | CUUGUGAUCA | | | AAGCGGACCA | |
| | UCA | | | CAG | | | UCC | |
| 147 | AGGAGCUGC | 150 | 1371-1391 | UUGCUUUGGC | 333 | 1369-1391 | CCAGGAGCUG | 516 |
| | UAGCCAAAG | | | UAGCAGCUCC | | | CUAGCCAAAG | |
| | CAA | | | UGG | | | CAU | |
| 148 | AGCUGCUAG | 151 | 1374-1394 | UCAAUGCUUU | 334 | 1372-1394 | GGAGCUGCUA | 517 |
| | CCAAAGCAU | | | GGCUAGCAGC | | | GCCAAAGCAU | |
| | UGA | | | UCC | | | UGG | |
| 149 | CUGCUAGCC | 152 | 1376-1396 | UUCCAAUGCU | 335 | 1374-1396 | AGCUGCUAGC | 518 |
| | AAAGCAUUG | | | UUGGCUAGCA | | | CAAAGCAUUG | |
| | GAA | | | GCU | | | GAG | |
| 150 | UGCUAGCCA | 153 | 1377-1397 | UCUCCAAUGC | 336 | 1375-1397 | GCUGCUAGCC | 519 |
| | AAGCAUUGG | | | UUUGGCUAGC | | | AAAGCAUUGG | |
| | AGA | | | AGC | | | AGA | |
| 151 | CUAGCCAAA | 154 | 1379-1399 | UGUCUCCAAU | 337 | 1377-1399 | UGCUAGCCAA | 520 |
| | GCAUUGGAG | | | GCUUUGGCUA | | | AGCAUUGGAG | |
| | ACA | | | GCA | | | ACC | |
| 152 | UAGCCAAAG | 155 | 1380-1400 | UGGUCUCCAA | 338 | 1378-1400 | GCUAGCCAAA | 521 |
| | CAUUGGAGA | | | UGCUUUGGCU | | | GCAUUGGAGA | |
| | CCA | | | AGC | | | CCC | |
| 153 | GGAGCUCCU | 156 | 1729-1749 | UAGGUCCAAC | 339 | 1727-1749 | AGGGAGCUCC | 522 |
| | UCGUUGGAC | | | GAAGGAGCUC | | | UUCGUUGGAC | |
| | CUA | | | CCU | | | CUC | |
| 154 | GAGCUCCUU | 157 | 1730-1750 | UGAGGUCCAA | 340 | 1728-1750 | GGGAGCUCCU | 523 |
| | CGUUGGACC | | | CGAAGGAGCU | | | UCGUUGGACC | |
| | UCA | | | CCC | | | UCC | |
| 155 | AGCUCCUUC | 158 | 1731-1751 | UGGAGGUCCA | 341 | 1729-1751 | GGAGCUCCUU | 524 |
| | GUUGGACCU | | | ACGAAGGAGC | | | CGUUGGACCU | |
| | CCA | | | UCC | | | CCA | |
| 156 | AGGCCUGGG | 159 | 1762-1782 | UGCAACAUAU | 342 | 1760-1782 | CAAGGCCUGG | 525 |
| | CCAUAUGUU | | | GGCCCAGGCC | | | GCCAUAUGUU | |
| | GCA | | | UUG | | | GCU | |
| 157 | GCCUGGGCC | 160 | 1764-1784 | UCAGCAACAU | 343 | 1762-1784 | AGGCCUGGGC | 526 |
| | AUAUGUUGC | | | AUGGCCCAGG | | | CAUAUGUUGC | |
| | UGA | | | CCU | | | UGG | |
| 158 | CUGGGCCAU | 161 | 1766-1786 | UCCCAGCAAC | 344 | 1764-1786 | GCCUGGGCCA | 527 |
| | AUGUUGCUG | | | AUAUGGCCCA | | | UAUGUUGCUG | |
| | GGA | | | GGC | | | GGA | |
| 159 | UGGGCCAUA | 162 | 1767-1787 | UUCCCAGCAA | 345 | 1765-1787 | CCUGGGCCAU | 528 |
| | UGUUGCUGG | | | CAUAUGGCCC | | | AUGUUGCUGG | |
| | GAA | | | AGG | | | GAA | |
| 160 | AUGUUGCUG | 163 | 1775-1795 | UGAGGAAAUU | 346 | 1773-1795 | AUAUGUUGCU | 529 |
| | GGAAUUUCC | | | CCCAGCAACA | | | GGGAAUUUCC | |
| | UCA | | | UAU | | | UCC | |
| 161 | CUGGGAAUU | 164 | 1781-1801 | UAGGGUGGAG | 347 | 1779-1801 | UGCUGGGAAU | 530 |
| | UCCUCCACC | | | GAAAUUCCCA | | | UUCCUCCACC | |
| | CUA | | | GCA | | | CUU | |
| 162 | UUCCUCCAC | 165 | 1789-1809 | UCAUGACGAA | 348 | 1787-1809 | AUUUCCUCCA | 531 |
| | CCUUCGUCA | | | GGGUGGAGGA | | | CCCUUCGUCA | |
| | UGA | | | AAU | | | UGC | |
| 163 | AAGGGCCGC | 166 | 1838-1858 | UUAGGAAUGG | 349 | 1836-1858 | AGAAGGGCCG | 532 |
| | CUCCAUUCC | | | AGGCGGCCCU | | | CCUCCAUUCC | |
| | UAA | | | UCU | | | UAC | |
| 164 | GGCCGCCUC | 167 | 1841-1861 | UUAGUAGGAA | 350 | 1839-1861 | AGGGCCGCCU | 533 |
| | CAUUCCUAC | | | UGGAGGCGGC | | | CCAUUCCUAC | |
| | UAA | | | CCU | | | UAA | |
| 165 | ACUGCAAAG | 168 | 1904-1924 | UGCUGUCAUA | 351 | 1902-1924 | CCACUGCAAA | 534 |
| | ACUAUGACA | | | GUCUUUGCAG | | | GACUAUGACA | |
| | GCA | | | UGG | | | GCA | |
| 166 | CAGCAUCAA | 169 | 1920-1940 | UGGUCCUGAA | 352 | 1918-1940 | GACAGCAUCA | 535 |
| | AUUUCAGGA | | | AUUUGAUGCU | | | AAUUUCAGGA | |
| | CCA | | | GUC | | | CCU | |
| 167 | UCAGGACCU | 170 | 1932-1952 | UGUACUGUCU | 353 | 1930-1952 | UUUCAGGACC | 536 |
| | GCAGACAGU | | | GCAGGUCCUG | | | UGCAGACAGU | |
| | ACA | | | AAA | | | ACA | |
| 168 | GCAGACAGU | 171 | 1941-1961 | UAUCUAGCCU | 354 | 1939-1961 | CUGCAGACAG | 537 |
| | ACAGGCUAG | | | GUACUGUCUG | | | UACAGGCUAG | |
| | AUA | | | CAG | | | AUA | |
| 169 | CAGACAGUA | 172 | 1942-1962 | UUAUCUAGCC | 355 | 1940-1962 | UGCAGACAGU | 538 |
| | CAGGCUAGA | | | UGUACUGUCU | | | ACAGGCUAGA | |
| | UAA | | | GCA | | | UAA | |
| 170 | AGACAGUAC | 173 | 1943-1963 | UUUAUCUAGC | 356 | 1941-1963 | GCAGACAGUA | 539 |
| | AGGCUAGAU | | | CUGUACUGUC | | | CAGGCUAGAU | |
| | AAA | | | UGC | | | AAC | |
| 171 | UCCCACUUG | 174 | 2087-2107 | UGCCUUUCAG | 357 | 2085-2107 | CCUCCCACUU | 540 |
| | CCCUGAAAG | | | GGCAAGUGGG | | | GCCCUGAAAG | |
| | GCA | | | AGG | | | GCC | |
| 172 | CUGCUGCUA | 175 | 2148-2168 | UGGGAUUAGA | 358 | 2146-2168 | GACUGCUGCU | 541 |
| | CAUCUAAUC | | | UGUAGCAGCA | | | ACAUCUAAUC | |
| | CCA | | | GUC | | | CCC | |
| 173 | UGCUGCUAC | 176 | 2149-2169 | UGGGGAUUAG | 359 | 2147-2169 | ACUGCUGCUA | 542 |
| | AUCUAAUCC | | | AUGUAGCAGC | | | CAUCUAAUCC | |
| | CCA | | | AGU | | | CCU | |
| 174 | GCUGCUACA | 177 | 2150-2170 | UAGGGGAUUA | 360 | 2148-2170 | CUGCUGCUAC | 543 |
| | UCUAAUCCC | | | GAUGUAGCAG | | | AUCUAAUCCC | |
| | CUA | | | CAG | | | CUA | |
| 175 | ACAUCUAAU | 178 | 2156-2176 | UAUUGGUAGG | 361 | 2154-2176 | CUACAUCUAA | 544 |
| | CCCCUACCA | | | GGAUUAGAUG | | | UCCCCUACCA | |
| | AUA | | | UAG | | | AUG | |
| 176 | AUCUAAUCC | 179 | 2158-2178 | UGCAUUGGUA | 362 | 2156-2178 | ACAUCUAAUC | 545 |
| | CCUACCAAU | | | GGGGAUUAGA | | | CCCUACCAAU | |
| | GCA | | | UGU | | | GCC | |
| 177 | CCCCUACCA | 180 | 2165-2185 | UACAGGAGGC | 363 | 2163-2185 | AUCCCCUACC | 546 |
| | AUGCCUCCU | | | AUUGGUAGGG | | | AAUGCCUCCU | |
| | GUA | | | GAU | | | GUC | |
| 178 | CCCUACCAA | 181 | 2166-2186 | UGACAGGAGG | 364 | 2164-2186 | UCCCCUACCA | 547 |
| | UGCCUCCUG | | | CAUUGGUAGG | | | AUGCCUCCUG | |
| | UCA | | | GGA | | | UCC | |
| 179 | ACCAAUGCC | 182 | 2170-2190 | UUAGGGACAG | 365 | 2168-2190 | CUACCAAUGC | 548 |
| | UCCUGUCCC | | | GAGGCAUUGG | | | CUCCUGUCCC | |
| | UAA | | | UAG | | | UAA | |
| 180 | CCAAUGCCU | 183 | 2171-2191 | UUUAGGGACA | 366 | 2169-2191 | UACCAAUGCC | 549 |
| | CCUGUCCCU | | | GGAGGCAUUG | | | UCCUGUCCCU | |
| | AAA | | | GUA | | | AAA | |
| 181 | AUGCCUCCU | 184 | 2174-2194 | UAGUUUAGGG | 367 | 2172-2194 | CAAUGCCUCC | 550 |
| | GUCCCUAAA | | | ACAGGAGGCA | | | UGUCCCUAAA | |
| | CUA | | | UUG | | | CUC | |
| 182 | UGCCUCCUG | 185 | 2175-2195 | UGAGUUUAGG | 368 | 2173-2195 | AAUGCCUCCU | 551 |
| | UCCCUAAAC | | | GACAGGAGGC | | | GUCCCUAAAC | |
| | UCA | | | AUU | | | UCC | |
| 183 | UACUGAUGA | 186 | 2202-2222 | UAGAGAGGGC | 369 | 2200-2222 | CAUACUGAUG | 552 |
| | CAGCCCUCU | | | UGUCAUCAGU | | | ACAGCCCUCU | |
| | CUA | | | AUG | | | CUG | |
| 184 | AGCUCAGCA | 553 | 726-746 | UGAACGAGAC | 602 | 724-746 | CCAGCUCAGC | 1132 |
| | GUGUCUCGU | | | ACUGCUGAGC | | | AGUGUCUCGU | |
| | UCA | | | UGG | | | UCC | |
| 185 | GCUCAGCAG | 554 | 727-747 | UGGAACGAGA | 603 | 725-747 | CAGCUCAGCA | 1133 |
| | UGUCUCGUU | | | CACUGCUGAG | | | GUGUCUCGUU | |
| | CCA | | | CUG | | | CCC | |
| 186 | GGACGGUAG | 555 | 752-772 | UAUGUCGGUC | 604 | 750-772 | GGGGACGGUA | 1134 |
| | CAGACCGAC | | | UGCUACCGUC | | | GCAGACCGAC | |
| | AUA | | | CCC | | | AUC | |
| 187 | ACGGUAGCA | 556 | 754-774 | UGGAUGUCGG | 605 | 752-774 | GGACGGUAGC | 1135 |
| | GACCGACAU | | | UCUGCUACCG | | | AGACCGACAU | |
| | CCA | | | UCC | | | CCU | |
| 188 | CGGUAGCAG | 557 | 755-775 | UAGGAUGUCG | 606 | 753-775 | GACGGUAGCA | 1136 |
| | ACCGACAUC | | | GUCUGCUACC | | | GACCGACAUC | |
| | CUA | | | GUC | | | CUU | |
| 189 | AGCAGACCG | 558 | 759-779 | UCAGAAGGAU | 607 | 757-779 | GUAGCAGACC | 1137 |
| | ACAUCCUUC | | | GUCGGUCUGC | | | GACAUCCUUC | |
| | UGA | | | UAC | | | UGG | |
| 190 | GCAGACCGA | 559 | 760-780 | UCCAGAAGGA | 608 | 758-780 | UAGCAGACCG | 1138 |
| | CAUCCUUCU | | | UGUCGGUCUG | | | ACAUCCUUCU | |
| | GGA | | | CUA | | | GGG | |
| 191 | UCCAUGCAA | 560 | 894-914 | UGUGAUGGAC | 609 | 892-914 | CUUCCAUGCA | 1139 |
| | CUGUCCAUC | | | AGUUGCAUGG | | | ACUGUCCAUC | |
| | ACA | | | AAG | | | ACG | |
| 192 | CCAUGCAAC | 561 | 895-915 | UCGUGAUGGA | 610 | 893-915 | UUCCAUGCAA | 1140 |
| | UGUCCAUCA | | | CAGUUGCAUG | | | CUGUCCAUCA | |
| | CGA | | | GAA | | | CGG | |
| 193 | CAUGCAACU | 562 | 896-916 | UCCGUGAUGG | 611 | 894-916 | UCCAUGCAAC | 1141 |
| | GUCCAUCAC | | | ACAGUUGCAU | | | UGUCCAUCAC | |
| | GGA | | | GGA | | | GGC | |
| 194 | AACUGUCCA | 563 | 901-921 | UUGCAGCCGU | 612 | 989-921 | GCAACUGUCC | 1142 |
| | UCACGGCUG | | | GAUGGACAGU | | | AUCACGGCUG | |
| | CAA | | | UGC | | | CAA | |
| 195 | ACUGUCCAU | 564 | 902-922 | UUUGCAGCCG | 613 | 900-922 | CAACUGUCCA | 1143 |
| | CACGGCUGC | | | UGAUGGACAG | | | UCACGGCUGC | |
| | AAA | | | UUG | | | AAC | |
| 196 | GUCCAUCAC | 565 | 905-925 | UCAGUUGCAG | 614 | 903-925 | CUGUCCAUCA | 1144 |
| | GGCUGCAAC | | | CCGUGAUGGA | | | CGGCUGCAAC | |
| | UGA | | | CAG | | | UGA | |
| 197 | AUCACGGCU | 566 | 909-929 | UAUUUCAGUU | 615 | 907-929 | CCAUCACGGC | 1145 |
| | GCAACUGAA | | | GCAGCCGUGA | | | UGCAACUGAA | |
| | AUA | | | UGG | | | AUC | |
| 198 | UGGGACACA | 567 | 935-955 | UUUCUGGUGC | 616 | 933-955 | GCUGGGACAC | 1146 |
| | GCGCACCAG | | | GCUGUGUCCC | | | AGCGCACCAG | |
| | AAA | | | AGC | | | AAG | |
| 199 | GACACAGCG | 568 | 938-958 | UAGCUUCUGG | 617 | 936-958 | GGGACACAGC | 1147 |
| | CACCAGAAG | | | UGCGCUGUGU | | | GCACCAGAAG | |
| | CUA | | | CCC | | | CUA | |
| 200 | ACAGCGCAC | 569 | 941-961 | UUUUAGCUUC | 618 | 939-961 | ACACAGCGCA | 1148 |
| | CAGAAGCUA | | | UGGUGCGCUG | | | CCAGAAGCUA | |
| | AAA | | | UGU | | | AAG | |
| 201 | GCCACAUUC | 570 | 1659-1677 | UGAGCCCGUA | 619 | 1659-1679 | AAGCCACAUU | 1149 |
| | UACGGGCUC | | | GAAUGUGGCU | | | CUACGGGCUC | |
| | A | | | U | | | U | |
| 202 | GAAAGUACU | 571 | 1715-1733 | UAGCUCCCUG | 620 | 1715-1735 | AAGAAAGUAC | 1150 |
| | CAGGGAGCU | | | AGUACUUUCU | | | UCAGGGAGCU | |
| | A | | | U | | | C | |
| 203 | GUCUAUACC | 572 | 2033-2051 | UUCAGGUAAG | 621 | 2033-2053 | AAGUCUAUAC | 1151 |
| | CUUACCUGA | | | GGUAUAGACU | | | CCUUACCUGA | |
| | A | | | U | | | A | |
| 204 | GCUGCUAGC | 573 | 1373-1391 | UAAUGCUUUG | 622 | 1373-1393 | GAGCUGCUAG | 1152 |
| | CAAAGCAUU | | | GCUAGCAGCU | | | CCAAAGCAUU | |
| | A | | | C | | | G | |
| 205 | GGAGUGGAG | 574 | 1534-1552 | UAUGACAGCA | 623 | 1534-1554 | AAGGAGUGGA | 1153 |
| | UGCUGUCAU | | | CUCCACUCCU | | | GUGCUGUCAU | |
| | A | | | U | | | A | |
| 206 | GGCCAAGAU | 575 | 2102-2120 | UGACAUCUUG | 624 | 2102-2122 | AAGGCCAAGA | 1154 |
| | CAAGAUGUC | | | AUCUUGGCCU | | | UCAAGAUGUC | |
| | A | | | U | | | C | |
| 207 | CUUGAGAUC | 576 | 2228-2246 | UAUGAAGACA | 625 | 2228-2248 | ACCUUGAGAU | 1155 |
| | UGUCUUCAU | | | GAUCUCAAGU | | | CUGUCUUCAU | |
| | A | | | U | | | ACC | |
| 208 | CUUGAGAUC | 576 | 2228-2246 | UAUGAAGACA | 626 | 2228-2248 | UUACCUUGAG | 1156 |
| | UGUCUUCAU | | | GAUCUCAAGG | | | AUCUGUCUUC | |
| | A | | | U | | | AUA | |
| 209 | CCUCAAACU | 577 | 2254-2272 | UGUUUUUGUU | 627 | 2254-2274 | CCCCUCAAAC | 1157 |
| | AACAAAAAC | | | AGUUUGAGGG | | | UAACAAAAAC | |
| | A | | | G | | | A | |
| 210 | CUCAAACUA | 578 | 2255-2273 | UUGUUUUUGU | 628 | 2255-2275 | CCCUCAAACU | 1158 |
| | ACAAAAACA | | | UAGUUUGAGG | | | AACAAAAACA | |
| | A | | | G | | | U | |
| 211 | CAAACUAAC | 579 | 2257-2275 | UAAUGUUUUU | 629 | 2257-2277 | CUCAAACUAA | 1159 |
| | AAAAACAUU | | | GUUAGUUUGA | | | CAAAAACAUU | |
| | A | | | G | | | U | |
| 212 | CUAACAAAA | 580 | 2261-2279 | UUGGAAAUGU | 630 | 2261-2281 | AACUAACAAA | 1160 |
| | ACAUUUCCA | | | UUUUGUUAGU | | | AACAUUUCCA | |
| | A | | | U | | | A | |
| 213 | CAAAAACAU | 581 | 2265-2283 | UUUAUUGGAA | 631 | 2265-2285 | AACAAAAACA | 1161 |
| | UUCCAAUAA | | | AUGUUUUUGU | | | UUUCCAAUAA | |
| | A | | | U | | | A | |
| 214 | CCAAUAAAA | 582 | 2276-2294 | UAUUUGAUAU | 632 | 2276-2296 | UUCCAAUAAA | 1162 |
| | AUAUCAAAU | | | UUUUAUUGGA | | | AAUAUCAAAU | |
| | A | | | A | | | A | |
| 215 | CAAUAAAAA | 583 | 2277-2295 | UUAUUUGAUA | 633 | 2277-2297 | UCCAAUAAAA | 1163 |
| | UAUCAAAUA | | | UUUUUAUUGG | | | AUAUCAAAUA | |
| | A | | | A | | | U | |
| 216 | CCAAAGCAU | 584 | 1381-1399 | UGGGUCUCCA | 634 | 1381-1401 | AGCCAAAGCA | 1164 |
| | UGGAGACCC | | | AUGCUUUGGU | | | UUGGAGACCC | |
| | A | | | U | | | U | |
| 217 | CAGGUCAGU | 585 | 1238-1256 | UAUAUUAGAU | 635 | 1238-1258 | CUCAGGUCAG | 1165 |
| | AUCUAAUAU | | | ACUGACCUGU | | | UAUCUAAUAU | |
| | A | | | U | | | A | |
| 218 | AUGUCAAAG | 586 | 1651-1669 | UAGAAUGUGG | 636 | 1651-1671 | GAAUGUCAAA | 1166 |
| | CCACAUUCU | | | CUUUGACAUU | | | GCCACAUUCU | |
| | A | | | C | | | A | |
| 219 | GCUCUACUC | 587 | 1673-1691 | UCAACUCAUA | 637 | 1673-1693 | GGGCUCUACU | 1167 |
| | UAUGAGUUG | | | GAGUAGAGCU | | | CUAUGAGUUG | |
| | A | | | U | | | U | |
| 220 | CUAUGAGUU | 588 | 1681-1699 | UGAAAGUCAC | 638 | 1681-1701 | CUCUAUGAGU | 1168 |
| | GUGACUUUC | | | AACUCAUAGA | | | UGUGACUUUC | |
| | A | | | G | | | A | |
| 221 | UAUGAGUUG | 589 | 1682-1700 | UUGAAAGUCA | 639 | 1682-1702 | UCUAUGAGUU | 1169 |
| | UGACUUUCA | | | CAACUCAUAG | | | GUGACUUUCA | |
| | A | | | A | | | A | |
| 222 | GGCCCAAAG | 590 | 1707-1725 | UGAGUACUUU | 640 | 1707-1727 | UUGGCCCAAA | 1170 |
| | AAAGUACUC | | | CUUUGGGCCU | | | GAAAGUACUC | |
| | A | | | U | | | A | |
| 223 | GCCAUAUGU | 591 | 1768-1786 | UUUCCCAGCA | 641 | 1768-1788 | GGGCCAUAUG | 1171 |
| | UGCUGGGAA | | | ACAUAUGGCU | | | UUGCUGGGAA | |
| | A | | | U | | | U | |
| 224 | CCCACUGCA | 592 | 1899-1979 | UCAUAGUCUU | 642 | 1899-1981 | GACCCACUGC | 1172 |
| | AAGACUAUG | | | UGCAGUGGGU | | | AAAGACUAUG | |
| | A | | | U | | | A | |
| 225 | GACUAUGAC | 593 | 1910-1928 | UUUUGAUGCU | 643 | 1910-1930 | AAGACUAUGA | 1173 |
| | AGCAUCAAA | | | GUCAUAGUCU | | | CAGCAUCAAA | |
| | A | | | U | | | U | |
| 226 | CUAUGACAG | 594 | 1912-1930 | UAAUUUGAUG | 644 | 1912-1932 | GACUAUGACA | 1174 |
| | CAUCAAAUU | | | CUGUCAUAGU | | | GCAUCAAAUU | |
| | A | | | C | | | U | |
| 227 | CUGAAGAAU | 595 | 2047-2065 | UAAGAACAGC | 645 | 2047-2067 | ACCUGAAGAA | 1175 |
| | GCUGUUCUU | | | AUUCUUCAGU | | | UGCUGUUCUU | |
| | A | | | U | | | U | |
| 228 | CUGAAGAAU | 595 | 2047-2065 | UAAGAACAGC | 646 | 2047-2067 | ACCUGAAGAA | 1176 |
| | GCUGUUCUU | | | AUUCUUCAGG | | | UGCUGUUCUU | |
| | A | | | U | | | U | |
| 229 | CCUGAAAGG | 596 | 2095-2113 | UUGAUCUUGG | 647 | 2095-2115 | GCCCUGAAAG | 1177 |
| | CCAAGAUCA | | | CCUUUCAGGU | | | GCCAAGAUCA | |
| | A | | | U | | | A | |
| 230 | CCUGAAAGG | 596 | 2095-2113 | UUGAUCUUGG | 648 | 2095-2115 | GCCCUGAAAG | 1178 |
| | CCAAGAUCA | | | CCUUUCAGGG | | | GCCAAGAUCA | |
| | A | | | C | | | A | |
| 231 | GACUGCUGC | 597 | 2144-2162 | UUUAGAUGUA | 649 | 2144-2164 | CUGACUGCUG | 1179 |
| | UACAUCUAA | | | GCAGCAGUCU | | | CUACAUCUAA | |
| | A | | | U | | | U | |
| 232 | AGGUCAGUA | 598 | 1239-1257 | UUAUAUUAGA | 650 | 1239-1259 | UCAGGUCAGU | 1180 |
| | UCUAAUAUA | | | UACUGACCUU | | | AUCUAAUAUA | |
| | A | | | U | | | A | |
| 233 | GGAAUUUCC | 599 | 1782-1800 | UAAGGGUGGA | 651 | 1782-1802 | UGGGAAUUUC | 1181 |
| | UCCACCCUU | | | GGAAAUUCCU | | | CUCCACCCUU | |
| | A | | | U | | | C | |
| 234 | GAUUCACCU | 600 | 1597-1615 | UACACGUCAA | 652 | 1597-1617 | CCGAUUCACC | 1182 |
| | UUGACGUGU | | | AGGUGAAUCU | | | UUUGACGUGU | |
| | A | | | U | | | A | |
| 235 | GAGGAUGAC | 601 | 1479-1497 | UCAGGCACGU | 653 | 1479-1499 | UGGAGGAUGA | 1183 |
| | ACGUGCCUG | | | GUCAUCCUCU | | | CACGUGCCUG | |
| | A | | | U | | | A | |
| 236 | CUCUAUGAG | 602 | 1679-1697 | UAAGUCACAA | 654 | 1679-1699 | UACUCUAUGA | 1184 |
| | UUGUGACUU | | | CUCAUAGAGU | | | GUUGUGACUU | |
| | A | | | A | | | U | |
| 237 | AGAGGAGGA | 603 | 1430-1448 | UGCAGUUCCA | 655 | 1430-1450 | CUAGAGGAGG | 1185 |
| | UGGAACUGC | | | UCCUCCUCUA | | | AUGGAACUGC | |
| | A | | | G | | | A | |
| 238 | GAUUCACCU | 600 | 1597-1615 | UACACGUCAA | 656 | 1597-1617 | CCGAUUCACC | 1186 |
| | UUGACGUGU | | | AGGUGAAUCG | | | UUUGACGUGU | |
| | A | | | G | | | A | |
| 239 | UCAGGUCAG | 601 | 1237-1255 | UUAUUAGAUA | 657 | 1237-1257 | ACUCAGGUCA | 1187 |
| | UAUCUAAUA | | | CUGACCUGAU | | | GUAUCUAAUA | |
| | A | | | U | | | U | |

The nucleotide sequence of exemplary modified versions of the dsRNA agents set forth in Table 2 are set forth in Table 3. More specifically, Table 3 sets forth the nucleotide sequence of exemplary sense strands, antisense strands, and dsRNA agent pairs of sense and antisense strands. It is to be understood that while the sense and antisense strands are set forth in pairs in Table 3, the disclosure encompasses dsRNA agents comprising any sense strand and any antisense set forth in Table 3 (e.g., that are at least partially complementary (e.g., as could be determined by a person of ordinary skill in the art)). It is to be understood that while the nucleotide sequence of the sense strands and antisense strands in Table 3 are set forth as modified (i.e., contain at least one modified nucleotide), the disclosure encompasses the sense and antisense sense strands set forth in Table 3 comprising other nucleotide modifications (e.g., as described herein) or that are unmodified.

TABLE 3
Modified Sense and Antisense Strand
Sequences of CIDEB dsRNA Agents.

| dsRNA | Sense | SEQ | | Antisense | SEQ | | mRNA | SEQ |
|---|---|---|---|---|---|---|---|---|
| Agent | Sequence | ID | Range in | Sequence | ID | Range in | Target | ID |
| ID | 5′ to 3′ | NO | NM_014430.4 | 5′ to 3′ | NO | NM_014430.4 | Sequence | NO |
| 240 | asgsagcaA | 658 | 63-83 | (vinu)sGfs | 893 | 61-83 | CCAGAGCAAG | 370 |
| | fgCfCfGfa | | | cuuGfcCfUf | | | CCGAAGGCAA | |
| | aggcaagca | | | ucggCfuUfg | | | GCA | |
| | | | | cucusgsg | | | | |
| 241 | asasgccgA | 659 | 68-88 | (vinu)sAfs | 894 | 66-88 | GCAAGCCGAA | 371 |
| | faGfGfCfa | | | ucgUfgCfUf | | | GGCAAGCACG | |
| | agcacgaua | | | ugccUfuCfg | | | AUG | |
| | | | | gcuusgsc | | | | |
| 242 | asgsccgaA | 660 | 69-89 | (vinu)sCfs | 895 | 67-89 | CAAGCCGAAG | 372 |
| | fgGfCfAfa | | | aucGfuGfCf | | | GCAAGCACGA | |
| | gcacgauga | | | uugcCfuUfc | | | UGG | |
| | | | | ggcususg | | | | |
| 243 | cscsgaagG | 661 | 71-91 | (vinu)sGfs | 896 | 69-91 | AGCCGAAGGC | 373 |
| | fcAfAfGfc | | | ccaUfcGfUf | | | AAGCACGAUG | |
| | acgauggca | | | gcuuGfcCfu | | | GCG | |
| | | | | ucggscsu | | | | |
| 244 | csgsaaggC | 662 | 72-92 | (vinu)sCfs | 897 | 70-92 | GCCGAAGGCA | 374 |
| | faAfGfCfa | | | gccAfuCfGf | | | AGCACGAUGG | |
| | cgauggcga | | | ugcuUfgCfc | | | CGC | |
| | | | | uucgsgsc | | | | |
| 245 | gsgscaagC | 663 | 76-96 | (vinu)sUfs | 898 | 74-96 | AAGGCAAGCA | 375 |
| | faCfGfAfu | | | gagCfgCfCf | | | CGAUGGCGCU | |
| | ggcgcucaa | | | aucgUfgCfu | | | CAC | |
| | | | | ugccsusu | | | | |
| 246 | gscsaagcA | 664 | 77-97 | (vinu)sGfs | 899 | 75-97 | AGGCAAGCAC | 376 |
| | fcGfAfUfg | | | ugaGfcGfCf | | | GAUGGCGCUC | |
| | gcgcucaca | | | caucGfuGfc | | | ACC | |
| | | | | uugcscsu | | | | |
| 247 | csasagcaC | 665 | 78-98 | (vinu)sGfs | 900 | 76-98 | GGCAAGCACG | 377 |
| | fgAfUfGfg | | | gugAfgCfGf | | | AUGGCGCUCA | |
| | cgcucacca | | | ccauCfgUfg | | | CCA | |
| | | | | cuugscsc | | | | |
| 248 | asasgcacG | 666 | 79-99 | (vinu)sUfs | 901 | 77-99 | GCAAGCACGA | 378 |
| | faUfGfGfc | | | gguGfaGfCf | | | UGGCGCUCAC | |
| | gcucaccaa | | | gccaUfcGfu | | | CAG | |
| | | | | gcuusgsc | | | | |
| 249 | ascsgaugG | 667 | 83-103 | (vinu)sCfs | 902 | 81-103 | GCACGAUGGC | 379 |
| | fcGfCfUfc | | | ggcUfgGfUf | | | GCUCACCAGC | |
| | accagccga | | | gagcGfcCfa | | | CGG | |
| | | | | ucgusgsc | | | | |
| 250 | cscsccgcA | 668 | 143-163 | (vinu)sAfs | 903 | 141-163 | CGCCCCGCAG | 380 |
| | fgCfCfGfu | | | cgcUfgGfCf | | | CCGUGCCAGC | |
| | gccagcgua | | | acggCfuGfc | | | GUC | |
| | | | | ggggscsg | | | | |
| 251 | gscsagccG | 669 | 147-167 | (vinu)sCfs | 904 | 145-167 | CCGCAGCCGU | 381 |
| | fuGfCfCfa | | | gugAfcGfCf | | | GCCAGCGUCA | |
| | gcgucacga | | | uggcAfcGfg | | | CGC | |
| | | | | cugcsgsg | | | | |
| 252 | csasgccgU | 670 | 148-168 | (vinu)sGfs | 905 | 146-168 | CGCAGCCGUG | 382 |
| | fgCfCfAfg | | | cguGfaCfGf | | | CCAGCGUCAC | |
| | cgucacgca | | | cuggCfaCfg | | | GCU | |
| | | | | gcugscsg | | | | |
| 253 | asgsccguG | 671 | 149-169 | (vinu)sAfs | 906 | 147-169 | GCAGCCGUGC | 383 |
| | fcCfAfGfc | | | gcgUfgAfCf | | | CAGCGUCACG | |
| | gucacgcua | | | gcugGfcAfc | | | CUG | |
| | | | | ggcusgsc | | | | |
| 254 | gscscgugC | 672 | 150-170 | (vinu)sCfs | 907 | 148-170 | CAGCCGUGCC | 384 |
| | fcAfGfCfg | | | agcGfuGfAf | | | AGCGUCACGC | |
| | ucacgcuga | | | cgcuGfgCfa | | | UGU | |
| | | | | cggcsusg | | | | |
| 255 | csgsugccA | 673 | 152-172 | (vinu)sUfs | 908 | 150-172 | GCCGUGCCAG | 385 |
| | fgCfGfUfc | | | acaGfcGfUf | | | CGUCACGCUG | |
| | acgcuguaa | | | gacgCfuGfg | | | UAG | |
| | | | | cacgsgsc | | | | |
| 256 | gscscagcG | 674 | 155-175 | (vinu)sUfs | 909 | 153-175 | GUGCCAGCGU | 386 |
| | fuCfAfCfg | | | gcuAfcAfGf | | | CACGCUGUAG | |
| | cuguagcaa | | | cgugAfcGfc | | | CAG | |
| | | | | uggcsasc | | | | |
| 257 | cscsagcgU | 675 | 156-176 | (vinu)sCfs | 910 | 154-176 | UGCCAGCGUC | 387 |
| | fcAfCfGfc | | | ugcUfaCfAf | | | ACGCUGUAGC | |
| | uguagcaga | | | gcguGfaCfg | | | AGC | |
| | | | | cuggscsa | | | | |
| 258 | csasgcguC | 676 | 157-177 | (vinu)sGfs | 911 | 155-177 | GCCAGCGUCA | 388 |
| | faCfGfCfu | | | cugCfuAfCf | | | CGCUGUAGCA | |
| | guagcagca | | | agcgUfgAfc | | | GCC | |
| | | | | gcugsgsc | | | | |
| 259 | gscsgucaC | 677 | 159-179 | (vinu)sCfs | 912 | 157-179 | CAGCGUCACG | 389 |
| | fgCfUfGfu | | | ggcUfgCfUf | | | CUGUAGCAGC | |
| | agcagccga | | | acagCfgUfg | | | CGA | |
| | | | | acgcsusg | | | | |
| 260 | csgsucacG | 678 | 160-180 | (vinu)sUfs | 913 | 158-180 | AGCGUCACGC | 390 |
| | fcUfGfUfa | | | cggCfuGfCf | | | UGUAGCAGCC | |
| | gcagccgaa | | | uacaGfcGfu | | | GAG | |
| | | | | gacgscsu | | | | |
| 261 | gsuscacgC | 679 | 161-181 | (vinu)sCfs | 914 | 159-181 | GCGUCACGCU | 391 |
| | fuGfUfAfg | | | ucgGfcUfGf | | | GUAGCAGCCG | |
| | cagccgaga | | | cuacAfgCfg | | | AGC | |
| | | | | ugacsgsc | | | | |
| 262 | csascgcuG | 680 | 163-183 | (vinu)sUfs | 915 | 161-183 | GUCACGCUGU | 392 |
| | fuAfGfCfa | | | gcuCfgGfCf | | | AGCAGCCGAG | |
| | gccgagcaa | | | ugcuAfcAfg | | | CAU | |
| | | | | cgugsasc | | | | |
| 263 | ascsgcugU | 681 | 164-184 | (vinu)sAfs | 916 | 162-184 | UCACGCUGUA | 393 |
| | faGfCfAfg | | | ugcUfcGfGf | | | GCAGCCGAGC | |
| | ccgagcaua | | | cugcUfaCfa | | | AUC | |
| | | | | gcgusgsa | | | | |
| 264 | gscsuguaG | 682 | 166-186 | (vinu)sUfs | 917 | 164-186 | ACGCUGUAGC | 394 |
| | fcAfGfCfc | | | gauGfcUfCf | | | AGCCGAGCAU | |
| | gagcaucaa | | | ggcuGfcUfa | | | CAG | |
| | | | | cagcsgsu | | | | |
| 265 | asgsccgaG | 683 | 174-194 | (vinu)sUfs | 918 | 172-194 | GCAGCCGAGC | 395 |
| | fcAfUfCfa | | | uucGfgGfCf | | | AUCAGCCCGA | |
| | gcccgaaaa | | | ugauGfcUfc | | | AAG | |
| | | | | ggcusgsc | | | | |
| 266 | csgsagcaU | 684 | 177-197 | (vinu)sUfs | 919 | 175-197 | GCCGAGCAUC | 396 |
| | fcAfGfCfc | | | ccuUfuCfGf | | | AGCCCGAAAG | |
| | cgaaaggaa | | | ggcuGfaUfg | | | GAA | |
| | | | | cucgsgsc | | | | |
| 267 | csasgcccG | 685 | 184-204 | (vinu)sUfs | 920 | 182-204 | AUCAGCCCGA | 397 |
| | faAfAfGfg | | | cguGfcUfUf | | | AAGGAAGCAC | |
| | aagcacgaa | | | ccuuUfcGfg | | | GAA | |
| | | | | gcugsasu | | | | |
| 268 | asgscccgA | 686 | 185-205 | (vinu)sUfs | 921 | 183-205 | UCAGCCCGAA | 398 |
| | faAfGfGfa | | | ucgUfgCfUf | | | AGGAAGCACG | |
| | agcacgaaa | | | uccuUfuCfg | | | AAA | |
| | | | | ggcusgsa | | | | |
| 269 | gscsccgaA | 687 | 186-206 | (vinu)sUfs | 922 | 184-206 | CAGCCCGAAA | 399 |
| | faGfGfAfa | | | uucGfuGfCf | | | GGAAGCACGA | |
| | gcacgaaaa | | | uuccUfuUfc | | | AAG | |
| | | | | gggcsusg | | | | |
| 270 | csgsgcggC | 688 | 234-254 | (vinu)sGfs | 923 | 232-254 | GGCGGCGGCG | 400 |
| | fgUfGfGfa | | | ucgCfcGfGf | | | UGGACCGGCG | |
| | ccggcgaca | | | uccaCfgCfc | | | ACG | |
| | | | | gccgscsc | | | | |
| 271 | gsgscggcG | 689 | 235-255 | (vinu)sCfs | 924 | 233-255 | GCGGCGGCGU | 401 |
| | fuGfGfAfc | | | gucGfcCfGf | | | GGACCGGCGA | |
| | cggcgacga | | | guccAfcGfc | | | CGG | |
| | | | | cgccsgsc | | | | |
| 272 | gscsggcgU | 690 | 236-256 | (vinu)sCfs | 925 | 234-256 | CGGCGGCGUG | 402 |
| | fgGfAfCfc | | | cguCfgCfCf | | | GACCGGCGAC | |
| | ggcgacgga | | | ggucCfaCfg | | | GGG | |
| | | | | ccgcscsg | | | | |
| 273 | gsusggacc | 691 | 241-261 | (vinu)sGfs | 926 | 239-261 | GCGUGGACCG | 403 |
| | fgGfCfGfa | | | ccaCfcCfGf | | | GCGACGGGUG | |
| | cggguggca | | | ucgcCfgGfu | | | GCA | |
| | | | | ccacsgsc | | | | |
| 274 | usgsgaccG | 692 | 242-262 | (vinu)sUfs | 927 | 240-262 | CGUGGACCGG | 404 |
| | fgCfGfAfc | | | gccAfcCfCf | | | CGACGGGUGG | |
| | ggguggcaa | | | gucgCfcGfg | | | CAC | |
| | | | | uccascsg | | | | |
| 275 | gsgsaccgG | 693 | 243-263 | (vinu)sGfs | 928 | 241-263 | GUGGACCGGC | 405 |
| | fcGfAfCfg | | | ugcCfaCfCf | | | GACGGGUGGC | |
| | gguggcaca | | | cgucGfcCfg | | | ACA | |
| | | | | guccsasc | | | | |
| 276 | gsasccggC | 694 | 244-264 | (vinu)sUfs | 929 | 242-264 | UGGACCGGCG | 406 |
| | fgAfCfGfg | | | gugCfcAfCf | | | ACGGGUGGCA | |
| | guggcacaa | | | ccguCfgCfc | | | CAG | |
| | | | | ggucscsa | | | | |
| 277 | csgsgcgaC | 695 | 247-267 | (vinu)sAfs | 930 | 245-267 | ACCGGCGACG | 407 |
| | fgGfGfUfg | | | gcuGfuGfCf | | | GGUGGCACAG | |
| | gcacagcua | | | caccCfgUfc | | | CUG | |
| | | | | gccgsgsu | | | | |
| 278 | gscsgacgG | 696 | 249-269 | (vinu)sCfs | 931 | 247-269 | CGGCGACGGG | 408 |
| | fgUfGfGfc | | | cagCfuGfUf | | | UGGCACAGCU | |
| | acagcugga | | | gccaCfcCfg | | | GGC | |
| | | | | ucgcscsg | | | | |
| 279 | gsgsuggcA | 697 | 255-275 | (vinu)sCfs | 932 | 253-275 | CGGGUGGCAC | 409 |
| | fcAfGfCfu | | | guaUfgCfCf | | | AGCUGGCAUA | |
| | ggcauacga | | | agcuGfuGfc | | | CGC | |
| | | | | caccscsg | | | | |
| 280 | gsusggcaC | 698 | 256-276 | (vinu)sGfs | 933 | 254-276 | GGGUGGCACA | 410 |
| | faGfCfUfg | | | cguAfuGfCf | | | GCUGGCAUAC | |
| | gcauacgca | | | cagcUfgUfg | | | GCG | |
| | | | | ccacscsc | | | | |
| 281 | usgsgcacA | 699 | 257-277 | (vinu)sCfs | 934 | 255-277 | GGUGGCACAG | 411 |
| | fgCfUfGfg | | | gcgUfaUfGf | | | CUGGCAUACG | |
| | cauacgcga | | | ccagCfuGfu | | | CGG | |
| | | | | gccascsc | | | | |
| 282 | cscsuccaC | 700 | 280-300 | (vinu)sUfs | 935 | 278-300 | UCCCUCCACA | 412 |
| | faGfGfUfg | | | cuaCfcGfCf | | | GGUGGCGGUA | |
| | gcgguagaa | | | caccUfgUfg | | | GAC | |
| | | | | gaggsgsa | | | | |
| 283 | uscscacaG | 701 | 282-302 | (vinu)sCfs | 936 | 280-302 | CCUCCACAGG | 413 |
| | fgUfGfGfc | | | gucUfaCfCf | | | UGGCGGUAGA | |
| | gguagacga | | | gccaCfcUfg | | | CGG | |
| | | | | uggasgsg | | | | |
| 284 | csascaggU | 702 | 284-304 | (vinu)sGfs | 937 | 282-304 | UCCACAGGUG | 414 |
| | fgGfCfGfg | | | ccgUfcUfAf | | | GCGGUAGACG | |
| | uagacggca | | | ccgcCfaCfc | | | GCG | |
| | | | | ugugsgsa | | | | |
| 285 | gsusggcgG | 703 | 289-309 | (vinu)sCfs | 938 | 287-309 | AGGUGGCGGU | 415 |
| | fuAfGfAfc | | | ggcCfgCfCf | | | AGACGGCGGC | |
| | ggcggccga | | | gucuAfcCfg | | | CGG | |
| | | | | ccacscsu | | | | |
| 286 | csgsgccgG | 704 | 303-323 | (vinu)sGfs | 939 | 301-323 | GGCGGCCGGG | 416 |
| | fgAfCfGfg | | | uugCfuCfGf | | | ACGGCGAGCA | |
| | cgagcaaca | | | ccguCfcCfg | | | ACA | |
| | | | | gccgscsc | | | | |
| 287 | gsgsccggG | 705 | 304-324 | (vinu)sUfs | 940 | 302-324 | GCGGCCGGGA | 417 |
| | faCfGfGfc | | | guuGfcUfCf | | | CGGCGAGCAA | |
| | gagcaacaa | | | gccgUfcCfc | | | CAG | |
| | | | | ggccsgsc | | | | |
| 288 | csusggcgU | 706 | 449-469 | (vinu)sGfs | 941 | 447-469 | CGCUGGCGUA | 418 |
| | faCfAfUfg | | | cgcUfcAfGf | | | CAUGCUGAGC | |
| | cugagcgca | | | caugUfaCfg | | | GCG | |
| | | | | ccagscsg | | | | |
| 289 | usgsgcguA | 707 | 450-470 | (vinu)sCfs | 942 | 448-470 | GCUGGCGUAC | 419 |
| | fcAfUfGfc | | | gcgCfuCfAf | | | AUGCUGAGCG | |
| | ugagcgcga | | | gcauGfuAfc | | | CGC | |
| | | | | gccasgsc | | | | |
| 290 | gsgscguaC | 708 | 451-471 | (vinu)sGfs | 943 | 449-471 | CUGGCGUACA | 420 |
| | faUfGfCfu | | | cgcGfcUfCf | | | UGCUGAGCGC | |
| | gagcgcgca | | | agcaUfgUfa | | | GCA | |
| | | | | cgccsasg | | | | |
| 291 | gscsguacA | 709 | 452-472 | (vinu)sUfs | 944 | 450-472 | UGGCGUACAU | 421 |
| | fuGfCfUfg | | | gcgCfgCfUf | | | GCUGAGCGCG | |
| | agcgcgcaa | | | cagcAfuGfu | | | CAC | |
| | | | | acgcscsa | | | | |
| 292 | gscsugagC | 710 | 460-480 | (vinu)sAfs | 945 | 458-480 | AUGCUGAGCG | 422 |
| | fgCfGfCfa | | | cuaCfgUfGf | | | CGCACACGUA | |
| | cacguagua | | | ugcgCfgCfu | | | GUA | |
| | | | | cagcsasu | | | | |
| 293 | csusgagcG | 711 | 461-481 | (vinu)sUfs | 946 | 459-481 | UGCUGAGCGC | 423 |
| | fcGfCfAfc | | | acuAfcGfUf | | | GCACACGUAG | |
| | acguaguaa | | | gugcGfcGfc | | | UAC | |
| | | | | ucagscsa | | | | |
| 294 | gsasgcgcG | 712 | 463-483 | (vinu)sUfs | 947 | 461-483 | CUGAGCGCGC | 424 |
| | fcAfCfAfc | | | guaCfuAfCf | | | ACACGUAGUA | |
| | guaguacaa | | | guguGfcGfc | | | CAC | |
| | | | | gcucsasg | | | | |
| 295 | csgscgcaC | 713 | 466-486 | (vinu)sCfs | 948 | 464-486 | AGCGCGCACA | 425 |
| | faCfGfUfa | | | gguGfuAfCf | | | CGUAGUACAC | |
| | guacaccga | | | uacgUfgUfg | | | CGC | |
| | | | | cgcgscsu | | | | |
| 296 | gscsgcacA | 714 | 467-487 | (vinu)sGfs | 949 | 465-487 | GCGCGCACAC | 426 |
| | fcGfUfAfg | | | cggUfgUfAf | | | GUAGUACACC | |
| | uacaccgca | | | cuacGfuGfu | | | GCC | |
| | | | | gcgcsgsc | | | | |
| 297 | csgscacaC | 715 | 468-488 | (vinu)sGfs | 950 | 466-488 | CGCGCACACG | 427 |
| | fgUfAfGfu | | | gcgGfuGfUf | | | UAGUACACCG | |
| | acaccgcca | | | acuaCfgUfg | | | CCU | |
| | | | | ugcgscsg | | | | |
| 298 | gscsacacG | 716 | 469-489 | (vinu)sAfs | 951 | 467-489 | GCGCACACGU | 428 |
| | fuAfGfUfa | | | ggcGfgUfGf | | | AGUACACCGC | |
| | caccgccua | | | uacuAfcGfu | | | CUU | |
| | | | | gugcsgsc | | | | |
| 299 | csascacgU | 717 | 470-490 | (vinu)sAfs | 952 | 468-490 | CGCACACGUA | 429 |
| | faGfUfAfc | | | aggCfgGfUf | | | GUACACCGCC | |
| | accgccuua | | | guacUfaCfg | | | UUG | |
| | | | | ugugscsg | | | | |
| 300 | ascsacguA | 718 | 471-491 | (vinu)sCfs | 953 | 469-491 | GCACACGUAG | 430 |
| | fgUfAfCfa | | | aagGfcGfGf | | | UACACCGCCU | |
| | ccgccuuga | | | uguaCfuAfc | | | UGC | |
| | | | | gugusgsc | | | | |
| 301 | ascsguagU | 719 | 473-493 | (vinu)sUfs | 954 | 471-493 | ACACGUAGUA | 431 |
| | faCfAfCfc | | | gcaAfgGfCf | | | CACCGCCUUG | |
| | gccuugcaa | | | ggugUfaCfu | | | CAG | |
| | | | | acgusgsu | | | | |
| 302 | csgsuaguA | 720 | 474-494 | (vinu)sCfs | 955 | 472-494 | CACGUAGUAC | 432 |
| | fcAfCfCfg | | | ugcAfaGfGf | | | ACCGCCUUGC | |
| | ccuugcaga | | | cgguGfuAfc | | | AGC | |
| | | | | uacgsusg | | | | |
| 303 | gsusaguaC | 721 | 475-495 | (vinu)sGfs | 956 | 473-495 | ACGUAGUACA | 433 |
| | faCfCfGfc | | | cugCfaAfGf | | | CCGCCUUGCA | |
| | cuugcagca | | | gcggUfgUfa | | | GCC | |
| | | | | cuacsgsu | | | | |
| 304 | gscscugcC | 722 | 515-535 | (vinu)sGfs | 957 | 513-535 | AGGCCUGCCG | 434 |
| | fgGfGfUfc | | | ccuUfcCfUf | | | GGUCAGGAAG | |
| | aggaaggca | | | gaccCfgGfc | | | GCC | |
| | | | | aggcscsu | | | | |
| 305 | csasggaaG | 723 | 526-546 | (vinu)sCfs | 958 | 524-546 | GUCAGGAAGG | 435 |
| | fgCfCfAfc | | | gcuCfuUfUf | | | CCACAAAGAG | |
| | aaagagcga | | | guggCfcUfu | | | CGG | |
| | | | | ccugsasc | | | | |
| 306 | gsgsaaggC | 724 | 528-548 | (vinu)sGfs | 959 | 526-548 | CAGGAAGGCC | 436 |
| | fcAfCfAfa | | | ccgCfuCfUf | | | ACAAAGAGCG | |
| | agagcggca | | | uuguGfgCfc | | | GCG | |
| | | | | uuccsusg | | | | |
| 307 | gsasaggcC | 725 | 529-549 | (vinu)sCfs | 960 | 527-549 | AGGAAGGCCA | 437 |
| | faCfAfAfa | | | gccGfcUfCf | | | CAAAGAGCGG | |
| | gagcggcga | | | uuugUfgGfc | | | CGU | |
| | | | | cuucscsu | | | | |
| 308 | asasggccA | 726 | 530-550 | (vinu)sAfs | 961 | 528-550 | GGAAGGCCAC | 438 |
| | fcAfAfAfg | | | cgcCfgCfUf | | | AAAGAGCGGC | |
| | agcggcgua | | | cuuuGfuGfg | | | GUG | |
| | | | | ccuuscsc | | | | |
| 309 | asgsgccaC | 727 | 531-551 | (vinu)sCfs | 962 | 529-551 | GAAGGCCACA | 439 |
| | faAfAfGfa | | | acgCfcGfCf | | | AAGAGCGGCG | |
| | gcggcguga | | | ucuuUfgUfg | | | UGA | |
| | | | | gccususc | | | | |
| 310 | gsgsccacA | 728 | 532-552 | (vinu)sUfs | 963 | 530-552 | AAGGCCACAA | 440 |
| | faAfGfAfg | | | cacGfcCfGf | | | AGAGCGGCGU | |
| | cggcgugaa | | | cucuUfuGfu | | | GAG | |
| | | | | ggccsusu | | | | |
| 311 | cscsacaaA | 729 | 534-554 | (vinu)sGfs | 964 | 532-554 | GGCCACAAAG | 441 |
| | fgAfGfCfg | | | cucAfcGfCf | | | AGCGGCGUGA | |
| | gcgugagca | | | cgcuCfuUfu | | | GCA | |
| | | | | guggscsc | | | | |
| 312 | csascaaaG | 730 | 535-555 | (vinu)sUfs | 965 | 533-555 | GCCACAAAGA | 442 |
| | faGfCfGfg | | | gcuCfaCfGf | | | GCGGCGUGAG | |
| | cgugagcaa | | | ccgcUfcUfu | | | CAG | |
| | | | | ugugsgsc | | | | |
| 313 | asgscaccG | 731 | 557-577 | (vinu)sCfs | 966 | 555-577 | GCAGCACCGC | 443 |
| | fcGfCfCfg | | | uggCfcGfAf | | | GCCGUCGGCC | |
| | ucggccaga | | | cggcGfcGfg | | | AGC | |
| | | | | ugcusgsc | | | | |
| 314 | csgscgccG | 732 | 562-582 | (vinu)sUfs | 967 | 560-582 | ACCGCGCCGU | 444 |
| | fuCfGfGfc | | | ggcGfcUfGf | | | CGGCCAGCGC | |
| | cagcgccaa | | | gccgAfcGfg | | | CAG | |
| | | | | cgcgsgsu | | | | |
| 315 | gscsgccgU | 733 | 563-583 | (vinu)sCfs | 968 | 561-583 | CCGCGCCGUC | 445 |
| | fcGfGfCfc | | | uggCfgCfUf | | | GGCCAGCGCC | |
| | agcgccaga | | | ggccGfaCfg | | | AGG | |
| | | | | gcgcsgsg | | | | |
| 316 | csasgcacA | 734 | 586-606 | (vinu)sUfs | 969 | 584-606 | UGCAGCACAA | 446 |
| | faGfCfGfu | | | ggcGfgCfCf | | | GCGUGGCCGC | |
| | ggccgccaa | | | acgcUfuGfu | | | CAG | |
| | | | | gcugscsa | | | | |
| 317 | asgscacaA | 735 | 587-607 | (vinu)sCfs | 970 | 585-607 | GCAGCACAAG | 447 |
| | fgCfGfUfg | | | uggCfgGfCf | | | CGUGGCCGCC | |
| | gccgccaga | | | cacgCfuUfg | | | AGC | |
| | | | | ugcusgsc | | | | |
| 318 | gscsacaaG | 736 | 588-608 | (vinu)sGfs | 971 | 586-608 | CAGCACAAGC | 448 |
| | fcGfUfGfg | | | cugGfcGfGf | | | GUGGCCGCCA | |
| | ccgccagca | | | ccacGfcUfu | | | GCG | |
| | | | | gugcsusg | | | | |
| 319 | csasagcgU | 737 | 591-611 | (vinu)sAfs | 972 | 589-611 | CACAAGCGUG | 449 |
| | fgGfCfCfg | | | ccgCfuGfGf | | | GCCGCCAGCG | |
| | ccagcggua | | | cggcCfaCfg | | | GUC | |
| | | | | cuugsusg | | | | |
| 320 | asasgcguG | 738 | 592-612 | (vinu)sGfs | 973 | 590-612 | ACAAGCGUGG | 450 |
| | fgCfCfGfc | | | accGfcUfGf | | | CCGCCAGCGG | |
| | cagcgguca | | | gcggCfcAfc | | | UCG | |
| | | | | gcuusgsu | | | | |
| 321 | asgscgugG | 739 | 593-613 | (vinu)sCfs | 974 | 591-613 | CAAGCGUGGC | 451 |
| | fcCfGfCfc | | | gacCfgCfUf | | | CGCCAGCGGU | |
| | agcggucga | | | ggcgGfcCfa | | | CGC | |
| | | | | cgcususg | | | | |
| 322 | gscsguggC | 740 | 594-614 | (vinu)sGfs | 975 | 592-614 | AAGCGUGGCC | 452 |
| | fcGfCfCfa | | | cgaCfcGfCf | | | GCCAGCGGUC | |
| | gcggucgca | | | uggcGfgCfc | | | GCC | |
| | | | | acgcsusu | | | | |
| 323 | gsgscuguG | 741 | 697-717 | (vinu)sCfs | 976 | 695-717 | AAGGCUGUGC | 453 |
| | fcCfUfGfu | | | gcgGfgCfCf | | | CUGUGGCCCG | |
| | ggcccgcga | | | acagGfcAfc | | | CGA | |
| | | | | agccsusu | | | | |
| 324 | gscscuguG | 742 | 703-723 | (vinu)sAfs | 977 | 701-723 | GUGCCUGUGG | 454 |
| | fgCfCfCfg | | | gacUfuCfGf | | | CCCGCGAAGU | |
| | cgaagucua | | | cgggCfcAfc | | | CUU | |
| | | | | aggcsasc | | | | |
| 325 | csusguggC | 743 | 705-725 | (vinu)sGfs | 978 | 703-725 | GCCUGUGGCC | 455 |
| | fcCfGfCfg | | | aagAfcUfUf | | | CGCGAAGUCU | |
| | aagucuuca | | | cgcgGfgCfc | | | UCC | |
| | | | | acagsgsc | | | | |
| 326 | gscsccgcG | 744 | 710-730 | (vinu)sAfs | 979 | 708-730 | UGGCCCGCGA | 456 |
| | faAfGfUfc | | | gcuGfgAfAf | | | AGUCUUCCAG | |
| | uuccagcua | | | gacuUfcGfc | | | CUC | |
| | | | | gggcscsa | | | | |
| 327 | cscscgcgA | 745 | 711-731 | (vinu)sGfs | 980 | 709-731 | GGCCCGCGAA | 457 |
| | faGfUfCfu | | | agcUfgGfAf | | | GUCUUCCAGC | |
| | uccagcuca | | | agacUfuCfg | | | UCA | |
| | | | | cgggscsc | | | | |
| 328 | cscsgcgaA | 746 | 712-732 | (vinu)sUfs | 981 | 710-732 | GCCCGCGAAG | 458 |
| | fgUfCfUfu | | | gagCfuGfGf | | | UCUUCCAGCU | |
| | ccagcucaa | | | aagaCfuUfc | | | CAG | |
| | | | | gcggsgsc | | | | |
| 329 | uscscagcU | 747 | 723-743 | (vinu)sCfs | 982 | 721-743 | CUUCCAGCUC | 459 |
| | fcAfGfCfa | | | gagAfcAfCf | | | AGCAGUGUCU | |
| | gugucucga | | | ugcuGfaGfc | | | CGU | |
| | | | | uggasasg | | | | |
| 330 | csasgcucA | 748 | 725-745 | (vinu)sAfs | 983 | 723-745 | UCCAGCUCAG | 460 |
| | fgCfAfGfu | | | acgAfgAfCf | | | CAGUGUCUCG | |
| | gucucguua | | | acugCfuGfa | | | UUC | |
| | | | | gcugsgsa | | | | |
| 331 | gscsgcacC | 749 | 944-964 | (vinu)sGfs | 984 | 942-964 | CAGCGCACCA | 461 |
| | faGfAfAfg | | | acuUfuAfGf | | | GAAGCUAAAG | |
| | cuaaaguca | | | cuucUfgGfu | | | UCU | |
| | | | | gcgcsusg | | | | |
| 332 | csasgaagC | 750 | 950-970 | (vinu)sCfs | 985 | 948-970 | ACCAGAAGCU | 462 |
| | fuAfAfAfg | | | aucAfaGfAf | | | AAAGUCUUGA | |
| | ucuugauga | | | cuuuAfgCfu | | | UGC | |
| | | | | ucugsgsu | | | | |
| 333 | asgsaagcU | 751 | 951-971 | (vinu)sGfs | 986 | 949-971 | CCAGAAGCUA | 463 |
| | faAfAfGfu | | | cauCfaAfGf | | | AAGUCUUGAU | |
| | cuugaugca | | | acuuUfaGfc | | | GCC | |
| | | | | uucusgsg | | | | |
| 334 | gsasagcuA | 752 | 952-972 | (vinu)sGfs | 987 | 950-972 | CAGAAGCUAA | 464 |
| | faAfGfUfc | | | gcaUfcAfAf | | | AGUCUUGAUG | |
| | uugaugcca | | | gacuUfuAfg | | | CCA | |
| | | | | cuucsusg | | | | |
| 335 | asasgcuaA | 753 | 953-973 | (vinu)sUfs | 988 | 951-973 | AGAAGCUAAA | 465 |
| | faGfUfCfu | | | ggcAfuCfAf | | | GUCUUGAUGC | |
| | ugaugccaa | | | agacUfuUfa | | | CAU | |
| | | | | gcuuscsu | | | | |
| 336 | gscsuaaaG | 754 | 955-975 | (vinu)sGfs | 989 | 953-975 | AAGCUAAAGU | 466 |
| | fuCfUfUfg | | | augGfcAfUf | | | CUUGAUGCCA | |
| | augccauca | | | caagAfcUfu | | | UCA | |
| | | | | uagcsusu | | | | |
| 337 | gsasugccA | 755 | 966-986 | (vinu)sGfs | 990 | 964-986 | UUGAUGCCAU | 467 |
| | fuCfAfAfa | | | gauGfuCfCf | | | CAAAGGACAU | |
| | ggacaucca | | | uuugAfuGfg | | | CCC | |
| | | | | caucsasa | | | | |
| 338 | asusgccaU | 756 | 967-987 | (vinu)sGfs | 991 | 965-987 | UGAUGCCAUC | 468 |
| | fcAfAfAfg | | | ggaUfgUfCf | | | AAAGGACAUC | |
| | gacauccca | | | cuuuGfaUfg | | | CCU | |
| | | | | gcauscsa | | | | |
| 339 | gscscaucA | 757 | 969-989 | (vinu)sCfs | 992 | 967-989 | AUGCCAUCAA | 469 |
| | faAfGfGfa | | | aggGfaUfGf | | | AGGACAUCCC | |
| | caucccuga | | | uccuUfuGfa | | | UGC | |
| | | | | uggcsasu | | | | |
| 340 | csasucucU | 758 | 998-1018 | (vinu)sUfs | 993 | 996-1018 | CACAUCUCUG | 470 |
| | fgUfCfAfc | | | aguGfgAfCf | | | UCACGUCCAC | |
| | guccacuaa | | | gugaCfaGfa | | | UAA | |
| | | | | gaugsusg | | | | |
| 341 | asuscucuG | 759 | 999-1019 | (vinu)sUfs | 994 | 997-1019 | ACAUCUCUGU | 471 |
| | fuCfAfCfg | | | uagUfgGfAf | | | CACGUCCACU | |
| | uccacuaaa | | | cgugAfcAfg | | | AAU | |
| | | | | agausgsu | | | | |
| 342 | uscsucugU | 760 | 1000- | (vinu)sAfs | 995 | 998-1020 | CAUCUCUGUC | 472 |
| | fcAfCfGfu | | 1020 | uuaGfuGfGf | | | ACGUCCACUA | |
| | ccacuaaua | | | acguGfaCfa | | | AUC | |
| | | | | gagasusg | | | | |
| 343 | csuscuguC | 761 | 1001-1021 | (vinu)sGfs | 996 | 999-1021 | AUCUCUGUCA | 473 |
| | faCfGfUfc | | | auuAfgUfGf | | | CGUCCACUAA | |
| | cacuaauca | | | gacgUfgAfc | | | UCG | |
| | | | | agagsasu | | | | |
| 344 | uscsugucA | 762 | 1002-1022 | (vinu)sCfs | 997 | 1000-1022 | UCUCUGUCAC | 474 |
| | fcGfUfCfc | | | gauUfaGfUf | | | GUCCACUAAU | |
| | acuaaucga | | | ggacGfuGfa | | | CGG | |
| | | | | cagasgsa | | | | |
| 345 | csusgucaC | 763 | 1003-1023 | (vinu)sCfs | 998 | 1001-1023 | CUCUGUCACG | 475 |
| | fgUfCfCfa | | | cgaUfuAfGf | | | UCCACUAAUC | |
| | cuaaucgga | | | uggaCfgUfg | | | GGC | |
| | | | | acagsasg | | | | |
| 346 | usgsucacG | 764 | 1004-1024 | (vinu)sGfs | 999 | 1002-1024 | UCUGUCACGU | 476 |
| | fuCfCfAfc | | | ccgAfuUfAf | | | CCACUAAUCG | |
| | uaaucggca | | | guggAfcGfu | | | GCA | |
| | | | | gacasgsa | | | | |
| 347 | gsuscacgU | 765 | 1005-1025 | (vinu)sUfs | 1000 | 1003-1025 | CUGUCACGUC | 477 |
| | fcCfAfCfu | | | gccGfaUfUf | | | CACUAAUCGG | |
| | aaucggcaa | | | agugGfaCfg | | | CAA | |
| | | | | ugacsasg | | | | |
| 348 | uscsacguC | 766 | 1006-1026 | (vinu)sUfs | 1001 | 1004-1026 | UGUCACGUCC | 478 |
| | fcAfCfUfa | | | ugcCfgAfUf | | | ACUAAUCGGC | |
| | aucggcaaa | | | uaguGfgAfc | | | AAA | |
| | | | | gugascsa | | | | |
| 349 | csascgucc | 767 | 1007-1027 | (vinu)sUfs | 1002 | 1005-1027 | GUCACGUCCA | 479 |
| | faCfUfAfa | | | uugCfcGfAf | | | CUAAUCGGCA | |
| | ucggcaaaa | | | uuagUfgGfa | | | AAA | |
| | | | | cgugsasc | | | | |
| 350 | csgsuccaC | 768 | 1009-1029 | (vinu)sCfs | 1003 | 1007-1029 | CACGUCCACU | 480 |
| | fuAfAfUfc | | | uuuUfgCfCf | | | AAUCGGCAAA | |
| | ggcaaaaga | | | gauuAfgUfg | | | AGG | |
| | | | | gacgsusg | | | | |
| 351 | gsusccacU | 769 | 1010-1030 | (vinu)sCfs | 1004 | 1008-1030 | ACGUCCACUA | 481 |
| | faAfUfCfg | | | cuuUfuGfCf | | | AUCGGCAAAA | |
| | gcaaaagga | | | cgauUfaGfu | | | GGA | |
| | | | | ggacsgsu | | | | |
| 352 | uscscacuA | 770 | 1011-1031 | (vinu)sUfs | 1005 | 1009-1031 | CGUCCACUAA | 482 |
| | faUfCfGfg | | | ccuUfuUfGf | | | UCGGCAAAAG | |
| | caaaaggaa | | | ccgaUfuAfg | | | GAG | |
| | | | | uggascsg | | | | |
| 353 | cscsacuaA | 771 | 1012-1032 | (vinu)sCfs | 1006 | 1010-1032 | GUCCACUAAU | 483 |
| | fuCfGfGfc | | | uccUfuUfUf | | | CGGCAAAAGG | |
| | aaaaggaga | | | gccgAfuUfa | | | AGA | |
| | | | | guggsasc | | | | |
| 354 | csascuaaU | 772 | 1013-1033 | (vinu)sUfs | 1007 | 1011-1033 | UCCACUAAUC | 484 |
| | fcGfGfCfa | | | cucCfuUfUf | | | GGCAAAAGGA | |
| | aaaggagaa | | | ugccGfaUfu | | | GAA | |
| | | | | agugsgsa | | | | |
| 355 | ascsuaauC | 773 | 1014-1034 | (vinu)sUfs | 1008 | 1012-1034 | CCACUAAUCG | 485 |
| | fgGfCfAfa | | | ucuCfcUfUf | | | GCAAAAGGAG | |
| | aaggagaaa | | | uugcCfgAfu | | | AAA | |
| | | | | uagusgsg | | | | |
| 356 | gsasgaagA | 774 | 1040-1060 | (vinu)sCfs | 1009 | 1038-1060 | GAGAGAAGAU | 486 |
| | fuGfAfCfc | | | acaCfuUfAf | | | GACCUAAGUG | |
| | uaaguguga | | | ggucAfuCfu | | | UGA | |
| | | | | ucucsusc | | | | |
| 357 | uscscuccG | 775 | 1110-1130 | (vinu)sCfs | 1010 | 1108-1130 | CCUCCUCCGA | 487 |
| | faCfCfAfa | | | uucCfuCfCf | | | CCAAGGAGGA | |
| | ggaggaaga | | | uuggUfcGfg | | | AGG | |
| | | | | aggasgsg | | | | |
| 358 | cscsagggA | 776 | 1176-1196 | (vinu)sCfs | 1011 | 1174-1196 | UUCCAGGGAA | 488 |
| | faGfGfAfa | | | cggAfgAfGf | | | GGAACUCUCC | |
| | cucuccgga | | | uuccUfuCfc | | | GGU | |
| | | | | cuggsasa | | | | |
| 359 | csasgggaA | 777 | 1177-1197 | (vinu)sAfs | 1012 | 1175-1197 | UCCAGGGAAG | 489 |
| | fgGfAfAfc | | | ccgGfaGfAf | | | GAACUCUCCG | |
| | ucuccggua | | | guucCfuUfc | | | GUC | |
| | | | | ccugsgsa | | | | |
| 360 | asgsgaacU | 778 | 1183-1203 | (vinu)sUfs | 1013 | 1181-1203 | GAAGGAACUC | 490 |
| | fcUfCfCfg | | | gguGfgAfCf | | | UCCGGUCCAC | |
| | guccaccaa | | | cggaGfaGfu | | | CAU | |
| | | | | uccususc | | | | |
| 361 | gsgsaacuC | 779 | 1184-1204 | (vinu)sAfs | 1014 | 1182-1204 | AAGGAACUCU | 491 |
| | fuCfCfGfg | | | uggUfgGfAf | | | CCGGUCCACC | |
| | uccaccaua | | | ccggAfgAfg | | | AUG | |
| | | | | uuccsusu | | | | |
| 362 | ascsucucC | 780 | 1187-1207 | (vinu)sUfs | 1015 | 1185-1207 | GAACUCUCCG | 492 |
| | fgGfUfCfc | | | ccaUfgGfUf | | | GUCCACCAUG | |
| | accauggaa | | | ggacCfgGfa | | | GAG | |
| | | | | gagususc | | | | |
| 363 | csusccggU | 781 | 1190-1210 | (vinu)sUfs | 1016 | 1188-1210 | CUCUCCGGUC | 493 |
| | fcCfAfCfc | | | acuCfcAfUf | | | CACCAUGGAG | |
| | auggaguaa | | | ggugGfaCfc | | | UAC | |
| | | | | ggagsasg | | | | |
| 364 | usgsaaccC | 782 | 1221-1241 | (vinu)sGfs | 1017 | 1219-1241 | UCUGAACCCC | 494 |
| | fcAfGfUfg | | | aguAfaGfUf | | | AGUGACUUAC | |
| | acuuacuca | | | cacuGfgGfg | | | UCA | |
| | | | | uucasgsa | | | | |
| 365 | gsusgacuU | 783 | 1230-1250 | (vinu)sUfs | 1018 | 1228-1250 | CAGUGACUUA | 495 |
| | faCfUfCfa | | | acuGfaCfCf | | | CUCAGGUCAG | |
| | ggucaguaa | | | ugagUfaAfg | | | UAU | |
| | | | | ucacsusg | | | | |
| 366 | gsascuuaC | 784 | 1232-1252 | (vinu)sGfs | 1019 | 1230-1252 | GUGACUUACU | 496 |
| | fuCfAfGfg | | | auaCfuGfAf | | | CAGGUCAGUA | |
| | ucaguauca | | | ccugAfgUfa | | | UCU | |
| | | | | agucsasc | | | | |
| 367 | csusuacuC | 785 | 1234-1254 | (vinu)sUfs | 1020 | 1232-1254 | GACUUACUCA | 497 |
| | faGfGfUfc | | | agaUfaCfUf | | | GGUCAGUAUC | |
| | aguaucuaa | | | gaccUfgAfg | | | UAA | |
| | | | | uaagsusc | | | | |
| 368 | csasguauC | 786 | 1245-1265 | (vinu)sCfs | 1021 | 1243-1265 | GUCAGUAUCU | 498 |
| | fuAfAfUfa | | | gagCfuUfAf | | | AAUAUAAGCU | |
| | uaagcucga | | | uauuAfgAfu | | | CGG | |
| | | | | acugsasc | | | | |
| 369 | gsusaucuA | 787 | 1247-1267 | (vinu)sUfs | 1022 | 1245-1267 | CAGUAUCUAA | 499 |
| | faUfAfUfa | | | ccgAfgCfUf | | | UAUAAGCUCG | |
| | agcucggaa | | | uauaUfuAfg | | | GAG | |
| | | | | auacsusg | | | | |
| 370 | usasucuaA | 788 | 1248-1268 | (vinu)sCfs | 1023 | 1246-1268 | AGUAUCUAAU | 500 |
| | fuAfUfAfa | | | uccGfaGfCf | | | AUAAGCUCGG | |
| | gcucggaga | | | uuauAfuUfa | | | AGU | |
| | | | | gauascsu | | | | |
| 371 | asuscuaaU | 789 | 1249-1269 | (vinu)sAfs | 1024 | 1247-1269 | GUAUCUAAUA | 501 |
| | faUfAfAfg | | | cucCfgAfGf | | | UAAGCUCGGA | |
| | cucggagua | | | cuuaUfaUfu | | | GUU | |
| | | | | agausasc | | | | |
| 372 | uscsuaauA | 790 | 1250-1270 | (vinu)sAfs | 1025 | 1248-1270 | UAUCUAAUAU | 502 |
| | fuAfAfGfc | | | acuCfcGfAf | | | AAGCUCGGAG | |
| | ucggaguua | | | gcuuAfuAfu | | | UUU | |
| | | | | uagasusa | | | | |
| 373 | csusaauaU | 791 | 1251-1271 | (vinu)sAfs | 1026 | 1249-1271 | AUCUAAUAUA | 503 |
| | faAfGfCfu | | | aacUfcCfGf | | | AGCUCGGAGU | |
| | cggaguuua | | | agcuUfaUfa | | | UUG | |
| | | | | uuagsasu | | | | |
| 374 | usasauauA | 792 | 1252-1272 | (vinu)sCfs | 1027 | 1250-1272 | UCUAAUAUAA | 504 |
| | faGfCfUfc | | | aaaCfuCfCf | | | GCUCGGAGUU | |
| | ggaguuuga | | | gagcUfuAfu | | | UGG | |
| | | | | auuasgsa | | | | |
| 375 | asusauaaG | 793 | 1254-1274 | (vinu)sUfs | 1028 | 1252-1274 | UAAUAUAAGC | 505 |
| | fcUfCfGfg | | | ccaAfaCfUf | | | UCGGAGUUUG | |
| | aguuuggaa | | | ccgaGfcUfu | | | GAC | |
| | | | | auaususa | | | | |
| 376 | usasuaagC | 794 | 1255-1275 | (vinu)sGfs | 1029 | 1253-1275 | AAUAUAAGCU | 506 |
| | fuCfGfGfa | | | uccAfaAfCf | | | CGGAGUUUGG | |
| | guuuggaca | | | uccgAfgCfu | | | ACG | |
| | | | | uauasusu | | | | |
| 377 | asusaagcU | 795 | 1256-1276 | (vinu)sCfs | 1030 | 1254-1276 | AUAUAAGCUC | 507 |
| | fcGfGfAfg | | | gucCfaAfAf | | | GGAGUUUGGA | |
| | uuuggacga | | | cuccGfaGfc | | | CGG | |
| | | | | uuausasu | | | | |
| 378 | gscsucggA | 796 | 1260-1280 | (vinu)sCfs | 1031 | 1258-1280 | AAGCUCGGAG | 508 |
| | fgUfUfUfg | | | cucCfgUfCf | | | UUUGGACGGA | |
| | gacggagga | | | caaaCfuCfc | | | GGG | |
| | | | | gagcsusu | | | | |
| 379 | gsgsaguuU | 797 | 1264-1284 | (vinu)sAfs | 1032 | 1262-1284 | UCGGAGUUUG | 509 |
| | fgGfAfCfg | | | gacCfcUfCf | | | GACGGAGGGU | |
| | gagggucua | | | cgucCfaAfa | | | CUG | |
| | | | | cuccsgsa | | | | |
| 380 | csasccccA | 798 | 1299-1319 | (vinu)sAfs | 1033 | 1297-1319 | ACCACCCCAG | 510 |
| | fgCfGfAfc | | | cggAfaAfGf | | | CGACCUUUCC | |
| | cuuuccgua | | | gucgCfuGfg | | | GUG | |
| | | | | ggugsgsu | | | | |
| 381 | gsuscuguG | 799 | 1319-1339 | (vinu)sGfs | 1034 | 1317-1339 | GUGUCUGUGA | 511 |
| | faUfCfAfc | | | uccGfcUfUf | | | UCACAAGCGG | |
| | aagcggaca | | | gugaUfcAfc | | | ACC | |
| | | | | agacsasc | | | | |
| 382 | uscsugugA | 800 | 1320-1340 | (vinu)sGfs | 1035 | 1318-1340 | UGUCUGUGAU | 512 |
| | fuCfAfCfa | | | gucCfgCfUf | | | CACAAGCGGA | |
| | agcggacca | | | ugugAfuCfa | | | CCA | |
| | | | | cagascsa | | | | |
| 383 | csusgugaU | 801 | 1321-1341 | (vinu)sUfs | 1036 | 1319-1341 | GUCUGUGAUC | 513 |
| | fcAfCfAfa | | | gguCfcGfCf | | | ACAAGCGGAC | |
| | gcggaccaa | | | uuguGfaUfc | | | CAU | |
| | | | | acagsasc | | | | |
| 384 | usgsugauC | 802 | 1322-1342 | (vinu)sAfs | 1037 | 1320-1342 | UCUGUGAUCA | 514 |
| | faCfAfAfg | | | uggUfcCfGf | | | CAAGCGGACC | |
| | cggaccaua | | | cuugUfgAfu | | | AUC | |
| | | | | cacasgsa | | | | |
| 385 | gsusgaucA | 803 | 1323-1343 | (vinu)sGfs | 1038 | 1321-1343 | CUGUGAUCAC | 515 |
| | fcAfAfGfc | | | augGfuCfCf | | | AAGCGGACCA | |
| | ggaccauca | | | gcuuGfuGfa | | | UCC | |
| | | | | ucacsasg | | | | |
| 386 | asgsgagcU | 804 | 1371-1391 | (vinu)sUfs | 1039 | 1369-1391 | CCAGGAGCUG | 516 |
| | fgCfUfAfg | | | gcuUfuGfGf | | | CUAGCCAAAG | |
| | ccaaagcaa | | | cuagCfaGfc | | | CAU | |
| | | | | uccusgsg | | | | |
| 387 | asgscugcU | 805 | 1374-1394 | (vinu)sCfs | 1040 | 1372-1394 | GGAGCUGCUA | 517 |
| | faGfCfCfa | | | aauGfcUfUf | | | GCCAAAGCAU | |
| | aagcauuga | | | uggcUfaGfc | | | UGG | |
| | | | | agcuscsc | | | | |
| 388 | csusgcuaG | 806 | 1376-1396 | (vinu)sUfs | 1041 | 1374-1396 | AGCUGCUAGC | 518 |
| | fcCfAfAfa | | | ccaAfuGfCf | | | CAAAGCAUUG | |
| | gcauuggaa | | | uuugGfcUfa | | | GAG | |
| | | | | gcagscsu | | | | |
| 389 | usgscuagC | 807 | 1377-1397 | (vinu)sCfs | 1042 | 1375-1397 | GCUGCUAGCC | 519 |
| | fcAfAfAfg | | | uccAfaUfGf | | | AAAGCAUUGG | |
| | cauuggaga | | | cuuuGfgCfu | | | AGA | |
| | | | | agcasgsc | | | | |
| 390 | csusagccA | 808 | 1379-1399 | (vinu)sGfs | 1043 | 1377-1399 | UGCUAGCCAA | 520 |
| | faAfGfCfa | | | ucuCfcAfAf | | | AGCAUUGGAG | |
| | uuggagaca | | | ugcuUfuGfg | | | ACC | |
| | | | | cuagscsa | | | | |
| 391 | usasgccaA | 809 | 1380-1400 | (vinu)sGfs | 1044 | 1378-1400 | GCUAGCCAAA | 521 |
| | faGfCfAfu | | | gucUfcCfAf | | | GCAUUGGAGA | |
| | uggagacca | | | augcUfuUfg | | | CCC | |
| | | | | gcuasgsc | | | | |
| 392 | gsgsagcuC | 810 | 1729-1749 | (vinu)sAfs | 1045 | 1727-1749 | AGGGAGCUCC | 522 |
| | fcUfUfCfg | | | gguCfcAfAf | | | UUCGUUGGAC | |
| | uuggaccua | | | cgaaGfgAfg | | | CUC | |
| | | | | cuccscsu | | | | |
| 393 | gsasgcucc | 811 | 1730-1750 | (vinu)sGfs | 1046 | 1728-1750 | GGGAGCUCCU | 523 |
| | fuUfCfGfu | | | aggUfcCfAf | | | UCGUUGGACC | |
| | uggaccuca | | | acgaAfgGfa | | | UCC | |
| | | | | gcucscsc | | | | |
| 394 | asgscuccU | 812 | 1731-1751 | (vinu)sGfs | 1047 | 1729-1751 | GGAGCUCCUU | 524 |
| | fuCfGfUfu | | | gagGfuCfCf | | | CGUUGGACCU | |
| | ggaccucca | | | aacgAfaGfg | | | CCA | |
| | | | | agcuscsc | | | | |
| 395 | asgsgccuG | 813 | 1762-1782 | (vinu)sGfs | 1048 | 1760-1782 | CAAGGCCUGG | 525 |
| | fgGfCfCfa | | | caaCfaUfAf | | | GCCAUAUGUU | |
| | uauguugca | | | uggcCfcAfg | | | GCU | |
| | | | | gccususg | | | | |
| 396 | gscscuggG | 814 | 1764-1784 | (vinu)sCfs | 1049 | 1762-1784 | AGGCCUGGGC | 526 |
| | fcCfAfUfa | | | agcAfaCfAf | | | CAUAUGUUGC | |
| | uguugcuga | | | uaugGfcCfc | | | UGG | |
| | | | | aggcscsu | | | | |
| 397 | csusgggcC | 815 | 1766-1786 | (vinu)sCfs | 1050 | 1764-1786 | GCCUGGGCCA | 527 |
| | faUfAfUfg | | | ccaGfcAfAf | | | UAUGUUGCUG | |
| | uugcuggga | | | cauaUfgGfc | | | GGA | |
| | | | | ccagsgsc | | | | |
| 398 | usgsggccA | 816 | 1767-1787 | (vinu)sUfs | 1051 | 1765-1787 | CCUGGGCCAU | 528 |
| | fuAfUfGfu | | | cccAfgCfAf | | | AUGUUGCUGG | |
| | ugcugggaa | | | acauAfuGfg | | | GAA | |
| | | | | cccasgsg | | | | |
| 399 | asusguugC | 817 | 1775-1795 | (vinu)sGfs | 1052 | 1773-1795 | AUAUGUUGCU | 529 |
| | fuGfGfGfa | | | aggAfaAfUf | | | GGGAAUUUCC | |
| | auuuccuca | | | ucccAfgCfa | | | UCC | |
| | | | | acausasu | | | | |
| 400 | csusgggaA | 818 | 1781-1801 | (vinu)sAfs | 1053 | 1779-1801 | UGCUGGGAAU | 530 |
| | fuUfUfCfc | | | gggUfgGfAf | | | UUCCUCCACC | |
| | uccacccua | | | ggaaAfuUfc | | | CUU | |
| | | | | ccagscsa | | | | |
| 401 | ususccucC | 819 | 1789-1809 | (vinu)sCfs | 1054 | 1787-1809 | AUUUCCUCCA | 531 |
| | faCfCfCfu | | | augAfcGfAf | | | CCCUUCGUCA | |
| | ucgucauga | | | agggUfgGfa | | | UGC | |
| | | | | ggaasasu | | | | |
| 402 | asasgggcC | 820 | 1838-1858 | (vinu)sUfs | 1055 | 1836-1858 | AGAAGGGCCG | 532 |
| | fgCfCfUfc | | | aggAfaUfGf | | | CCUCCAUUCC | |
| | cauuccuaa | | | gaggCfgGfc | | | UAC | |
| | | | | ccuuscsu | | | | |
| 403 | gsgsccgcC | 821 | 1841-1861 | (vinu)sUfs | 1056 | 1839-1861 | AGGGCCGCCU | 533 |
| | fuCfCfAfu | | | aguAfgGfAf | | | CCAUUCCUAC | |
| | uccuacuaa | | | auggAfgGfc | | | UAA | |
| | | | | ggccscsu | | | | |
| 404 | ascsugcaA | 822 | 1904-1924 | (vinu)sGfs | 1057 | 1902-1924 | CCACUGCAAA | 534 |
| | faGfAfCfu | | | cugUfcAfUf | | | GACUAUGACA | |
| | augacagca | | | agucUfuUfg | | | GCA | |
| | | | | cagusgsg | | | | |
| 405 | csasgcauC | 823 | 1920-1940 | (vinu)sGfs | 1058 | 1918-1940 | GACAGCAUCA | 535 |
| | faAfAfUfu | | | gucCfuGfAf | | | AAUUUCAGGA | |
| | ucaggacca | | | aauuUfgAfu | | | CCU | |
| | | | | gcugsusc | | | | |
| 406 | uscsaggaC | 824 | 1932-1952 | (vinu)sGfs | 1059 | 1930-1952 | UUUCAGGACC | 536 |
| | fcUfGfCfa | | | uacUfgUfCf | | | UGCAGACAGU | |
| | gacaguaca | | | ugcaGfgUfc | | | ACA | |
| | | | | cugasasa | | | | |
| 407 | gscsagacA | 825 | 1941-1961 | (vinu)sAfs | 1060 | 1939-1961 | CUGCAGACAG | 537 |
| | fgUfAfCfa | | | ucuAfgCfCf | | | UACAGGCUAG | |
| | ggcuagaua | | | uguaCfuGfu | | | AUA | |
| | | | | cugcsasg | | | | |
| 408 | csasgacaG | 826 | 1942-1962 | (vinu)sUfs | 1061 | 1940-1962 | UGCAGACAGU | 538 |
| | fuAfCfAfg | | | aucUfaGfCf | | | ACAGGCUAGA | |
| | gcuagauaa | | | cuguAfcUfg | | | UAA | |
| | | | | ucugscsa | | | | |
| 409 | asgsacagU | 827 | 1943-1963 | (vinu)sUfs | 1062 | 1941-1963 | GCAGACAGUA | 539 |
| | faCfAfGfg | | | uauCfuAfGf | | | CAGGCUAGAU | |
| | cuagauaaa | | | ccugUfaCfu | | | AAC | |
| | | | | gucusgsc | | | | |
| 410 | uscsccacU | 828 | 2087-2107 | (vinu)sGfs | 1063 | 2085-2107 | CCUCCCACUU | 540 |
| | fuGfCfCfc | | | ccuUfuCfAf | | | GCCCUGAAAG | |
| | ugaaaggca | | | gggcAfaGfu | | | GCC | |
| | | | | gggasgsg | | | | |
| 411 | csusgcugC | 829 | 2148-2168 | (vinu)sGfs | 1064 | 2146-2168 | GACUGCUGCU | 541 |
| | fuAfCfAfu | | | ggaUfuAfGf | | | ACAUCUAAUC | |
| | cuaauccca | | | auguAfgCfa | | | CCC | |
| | | | | gcagsusc | | | | |
| 412 | usgscugcU | 830 | 2149-2169 | (vinu)sGfs | 1065 | 2147-2169 | ACUGCUGCUA | 542 |
| | faCfAfUfc | | | gggAfuUfAf | | | CAUCUAAUCC | |
| | uaaucccca | | | gaugUfaGfc | | | CCU | |
| | | | | agcasgsu | | | | |
| 413 | gscsugcuA | 831 | 2150-2170 | (vinu)sAfs | 1066 | 2148-2170 | CUGCUGCUAC | 543 |
| | fcAfUfCfu | | | gggGfaUfUf | | | AUCUAAUCCC | |
| | aauccccua | | | agauGfuAfg | | | CUA | |
| | | | | cagcsasg | | | | |
| 414 | ascsaucuA | 832 | 2156-2176 | (vinu)sAfs | 1067 | 2154-2176 | CUACAUCUAA | 544 |
| | faUfCfCfc | | | uugGfuAfGf | | | UCCCCUACCA | |
| | cuaccaaua | | | gggaUfuAfg | | | AUG | |
| | | | | augusasg | | | | |
| 415 | asuscuaaU | 833 | 2158-2178 | (vinu)sGfs | 1068 | 2156-2178 | ACAUCUAAUC | 545 |
| | fcCfCfCfu | | | cauUfgGfUf | | | CCCUACCAAU | |
| | accaaugca | | | agggGfaUfu | | | GCC | |
| | | | | agausgsu | | | | |
| 416 | cscsccuaC | 834 | 2165-2185 | (vinu)sAfs | 1069 | 2163-2185 | AUCCCCUACC | 546 |
| | fcAfAfUfg | | | cagGfaGfGf | | | AAUGCCUCCU | |
| | ccuccugua | | | cauuGfgUfa | | | GUC | |
| | | | | ggggsasu | | | | |
| 417 | cscscuacC | 835 | 2166-2186 | (vinu)sGfs | 1070 | 2164-2186 | UCCCCUACCA | 547 |
| | faAfUfGfc | | | acaGfgAfGf | | | AUGCCUCCUG | |
| | cuccuguca | | | gcauUfgGfu | | | UCC | |
| | | | | agggsgsa | | | | |
| 418 | ascscaauG | 836 | 2170-2190 | (vinu)sUfs | 1071 | 2168-2190 | CUACCAAUGC | 548 |
| | fcCfUfCfc | | | aggGfaCfAf | | | CUCCUGUCCC | |
| | ugucccuaa | | | ggagGfcAfu | | | UAA | |
| | | | | uggusasg | | | | |
| 419 | cscsaaugC | 837 | 2171-2191 | (vinu)sUfs | 1072 | 2169-2191 | UACCAAUGCC | 549 |
| | fcUfCfCfu | | | uagGfgAfCf | | | UCCUGUCCCU | |
| | gucccuaaa | | | aggaGfgCfa | | | AAA | |
| | | | | uuggsusa | | | | |
| 420 | asusgccuC | 838 | 2174-2194 | (vinu)sAfs | 1073 | 2172-2194 | CAAUGCCUCC | 550 |
| | fcUfGfUfc | | | guuUfaGfGf | | | UGUCCCUAAA | |
| | ccuaaacua | | | gacaGfgAfg | | | CUC | |
| | | | | gcaususg | | | | |
| 421 | usgsccucC | 839 | 2175-2195 | (vinu)sGfs | 1074 | 2173-2195 | AAUGCCUCCU | 551 |
| | fuGfUfCfc | | | aguUfuAfGf | | | GUCCCUAAAC | |
| | cuaaacuca | | | ggacAfgGfa | | | UCC | |
| | | | | ggcasusu | | | | |
| 422 | usascugaU | 840 | 2202-2222 | (vinu)sAfs | 1075 | 2200-2222 | CAUACUGAUG | 552 |
| | fgAfCfAfg | | | gagAfgGfGf | | | ACAGCCCUCU | |
| | cccucucua | | | cuguCfaUfc | | | CUG | |
| | | | | aguasusg | | | | |
| 423 | asgscucaG | 841 | 726-746 | (vinu)sGfs | 1076 | 724-746 | CCAGCUCAGC | 1132 |
| | fcAfGfUfg | | | aacGfaGfAf | | | AGUGUCUCGU | |
| | ucucguuca | | | cacuGfcUfg | | | UCC | |
| | | | | agcusgsg | | | | |
| 424 | gscsucagC | 842 | 727-747 | (vinu)sGfs | 1077 | 725-747 | CAGCUCAGCA | 1133 |
| | faGfUfGfu | | | gaaCfgAfGf | | | GUGUCUCGUU | |
| | cucguucca | | | acacUfgCfu | | | CCC | |
| | | | | gagcsusg | | | | |
| 425 | gsgsacggU | 843 | 752-772 | (vinu)sAfs | 1078 | 750-772 | GGGGACGGUA | 1134 |
| | faGfCfAfg | | | uguCfgGfUf | | | GCAGACCGAC | |
| | accgacaua | | | cugcUfaCfc | | | AUC | |
| | | | | guccscsc | | | | |
| 426 | ascsgguaG | 844 | 754-774 | (vinu)sGfs | 1079 | 752-774 | GGACGGUAGC | 1135 |
| | fcAfGfAfc | | | gauGfuCfGf | | | AGACCGACAU | |
| | cgacaucca | | | gucuGfcUfa | | | CCU | |
| | | | | ccguscsc | | | | |
| 427 | csgsguagC | 845 | 755-775 | (vinu)sAfs | 1080 | 753-775 | GACGGUAGCA | 1136 |
| | faGfAfCfc | | | ggaUfgUfCf | | | GACCGACAUC | |
| | gacauccua | | | ggucUfgCfu | | | CUU | |
| | | | | accgsusc | | | | |
| 428 | asgscagaC | 846 | 759-779 | (vinu)sCfs | 1081 | 757-779 | GUAGCAGACC | 1137 |
| | fcGfAfCfa | | | agaAfgGfAf | | | GACAUCCUUC | |
| | uccuucuga | | | ugucGfgUfc | | | UGG | |
| | | | | ugcusasc | | | | |
| 429 | gscsagacC | 847 | 760-780 | (vinu)sCfs | 1082 | 758-780 | UAGCAGACCG | 1138 |
| | fgAfCfAfu | | | cagAfaGfGf | | | ACAUCCUUCU | |
| | ccuucugga | | | auguCfgGfu | | | GGG | |
| | | | | cugcsusa | | | | |
| 430 | uscscaugC | 848 | 894-914 | (vinu)sGfs | 1083 | 892-914 | CUUCCAUGCA | 1139 |
| | faAfCfUfg | | | ugaUfgGfAf | | | ACUGUCCAUC | |
| | uccaucaca | | | caguUfgCfa | | | ACG | |
| | | | | uggasasg | | | | |
| 431 | cscsaugcA | 849 | 895-915 | (vinu)sCfs | 1084 | 893-915 | UUCCAUGCAA | 1140 |
| | faCfUfGfu | | | gugAfuGfGf | | | CUGUCCAUCA | |
| | ccaucacga | | | acagUfuGfc | | | CGG | |
| | | | | auggsasa | | | | |
| 432 | csasugcaA | 850 | 896-916 | (vinu)sCfs | 1085 | 894-916 | UCCAUGCAAC | 1141 |
| | fcUfGfUfc | | | cguGfaUfGf | | | UGUCCAUCAC | |
| | caucacgga | | | gacaGfuUfg | | | GGC | |
| | | | | caugsgsa | | | | |
| 433 | asascuguC | 851 | 901-921 | (vinu)sUfs | 1086 | 989-921 | GCAACUGUCC | 1142 |
| | fcAfUfCfa | | | gcaGfcCfGf | | | AUCACGGCUG | |
| | cggcugcaa | | | ugauGfgAfc | | | CAA | |
| | | | | aguusgsc | | | | |
| 434 | ascsugucC | 852 | 902-922 | (vinu)sUfs | 1087 | 900-922 | CAACUGUCCA | 1143 |
| | faUfCfAfc | | | ugcAfgCfCf | | | UCACGGCUGC | |
| | ggcugcaaa | | | gugaUfgGfa | | | AAC | |
| | | | | cagususg | | | | |
| 435 | gsusccauC | 853 | 905-925 | (vinu)sCfs | 1088 | 903-925 | CUGUCCAUCA | 1144 |
| | faCfGfGfc | | | aguUfgCfAf | | | CGGCUGCAAC | |
| | ugcaacuga | | | gccgUfgAfu | | | UGA | |
| | | | | ggacsasg | | | | |
| 436 | asuscacgG | 854 | 909-929 | (vinu)sAfs | 1089 | 907-929 | CCAUCACGGC | 1145 |
| | fcUfGfCfa | | | uuuCfaGfUf | | | UGCAACUGAA | |
| | acugaaaua | | | ugcaGfcCfg | | | AUC | |
| | | | | ugausgsg | | | | |
| 437 | usgsggacA | 855 | 935-955 | (vinu)sUfs | 1090 | 933-955 | GCUGGGACAC | 1146 |
| | fcAfGfCfg | | | ucuGfgUfGf | | | AGCGCACCAG | |
| | caccagaaa | | | cgcuGfuGfu | | | AAG | |
| | | | | cccasgsc | | | | |
| 438 | gsascacaG | 856 | 938-958 | (vinu)sAfs | 1091 | 936-958 | GGGACACAGC | 1147 |
| | fcGfCfAfc | | | gcuUfcUfGf | | | GCACCAGAAG | |
| | cagaagcua | | | gugcGfcUfg | | | CUA | |
| | | | | ugucscsc | | | | |
| 439 | ascsagcgC | 857 | 941-961 | (vinu)sUfs | 1092 | 939-961 | ACACAGCGCA | 1148 |
| | faCfCfAfg | | | uuaGfcUfUf | | | CCAGAAGCUA | |
| | aagcuaaaa | | | cuggUfgCfg | | | AAG | |
| | | | | cugusgsu | | | | |
| 440 | gscscaCfa | 858 | 1659-1677 | (vinu)sGfs | 1093 | 1659-1679 | AAGCCACAUU | 1149 |
| | UfUfCfuac | | | agcCfcGfUf | | | CUACGGGCUC | |
| | gggcuca | | | agaaUfgUfg | | | U | |
| | | | | gcsusu | | | | |
| 441 | gsasaaGfu | 859 | 1715-1733 | (vinu)sAfs | 1094 | 1715-1735 | AAGAAAGUAC | 1150 |
| | AfCfUfcag | | | gcuCfcCfUf | | | UCAGGGAGCU | |
| | ggagcua | | | gaguAfcUfu | | | C | |
| | | | | ucsusu | | | | |
| 442 | gsuscuAfu | 860 | 2033-2051 | (vinu)sUfs | 1095 | 2033-2053 | AAGUCUAUAC | 1151 |
| | AfCfCfcuu | | | cagGfuAfAf | | | CCUUACCUGA | |
| | accugaa | | | ggguAfuAfg | | | A | |
| | | | | acsusu | | | | |
| 443 | gscsugCfu | 861 | 1373-1391 | (vinu)sAfs | 1096 | 1373-1393 | GAGCUGCUAG | 1152 |
| | AfGfCfcaa | | | augCfuUfUf | | | CCAAAGCAUU | |
| | agcauua | | | ggcuAfgCfa | | | G | |
| | | | | gcsusc | | | | |
| 444 | gsgsagUfg | 862 | 1534-1552 | (vinu)sAfs | 1097 | 1534-1554 | AAGGAGUGGA | 1153 |
| | GfAfGfugc | | | ugaCfaGfCf | | | GUGCUGUCAU | |
| | ugucaua | | | acucCfaCfu | | | A | |
| | | | | ccsusu | | | | |
| 445 | gsgsccAfa | 863 | 2102-2120 | (vinu)sGfs | 1098 | 2102-2122 | AAGGCCAAGA | 1154 |
| | GfAfUfcaa | | | acaUfcUfUf | | | UCAAGAUGUC | |
| | gauguca | | | gaucUfuGfg | | | C | |
| | | | | ccsusu | | | | |
| 446 | csusugAfg | 864 | 2228-2246 | (vinu)sAfs | 1099 | 2228-2248 | ACCUUGAGAU | 1155 |
| | AfUfCfugu | | | ugaAfgAfCf | | | CUGUCUUCAU | |
| | cuucaua | | | agauCfuCfa | | | ACC | |
| | | | | agsusu | | | | |
| 447 | csusugAfg | 864 | 2228-2246 | (vinu)sAfs | 1100 | 2228-2248 | UUACCUUGAG | 1156 |
| | AfUfCfugu | | | ugaAfgAfCf | | | AUCUGUCUUC | |
| | cuucaua | | | agauCfuCfa | | | AUA | |
| | | | | agsgsu | | | | |
| 448 | cscsucAfa | 865 | 2254-2272 | (vinu)sGfs | 1101 | 2254-2274 | CCCCUCAAAC | 1157 |
| | AfCfUfaac | | | uuuUfuGfUf | | | UAACAAAAAC | |
| | aaaaaca | | | uaguUfuGfa | | | A | |
| | | | | ggsgsg | | | | |
| 449 | csuscaAfa | 866 | 2255-2273 | (vinu)sUfs | 1102 | 2255-2275 | CCCUCAAACU | 1158 |
| | CfUfAfaca | | | guuUfuUfGf | | | AACAAAAACA | |
| | aaaacaa | | | uuagUfuUfg | | | U | |
| | | | | agsgsg | | | | |
| 450 | csasaaCfu | 867 | 2257-2275 | (vinu)sAfs | 1103 | 2257-2277 | CUCAAACUAA | 1159 |
| | AfAfCfaaa | | | augUfuUfUf | | | CAAAAACAUU | |
| | aacauua | | | uguuAfgUfu | | | U | |
| | | | | ugsasg | | | | |
| 451 | csusaaCfa | 868 | 2261-2279 | (vinu)sUfs | 1104 | 2261-2281 | AACUAACAAA | 1160 |
| | AfAfAfaca | | | ggaAfaUfGf | | | AACAUUUCCA | |
| | uuuccaa | | | uuuuUfgUfu | | | A | |
| | | | | agsusu | | | | |
| 452 | csasaaAfa | 869 | 2265-2283 | (vinu)sUfs | 1105 | 2265-2285 | AACAAAAACA | 1161 |
| | CfAfUfuuc | | | uauUfgGfAf | | | UUUCCAAUAA | |
| | caauaaa | | | aaugUfuUfu | | | A | |
| | | | | ugsusu | | | | |
| 453 | cscsaaUfa | 870 | 2276-2294 | (vinu)sAfs | 1106 | 2276-2296 | UUCCAAUAAA | 1162 |
| | AfAfAfaua | | | uuuGfaUfAf | | | AAUAUCAAAU | |
| | ucaaaua | | | uuuuUfaUfu | | | A | |
| | | | | ggsasa | | | | |
| 454 | csasauAfa | 871 | 2277-2295 | (vinu)sUfs | 1107 | 2277-2297 | UCCAAUAAAA | 1163 |
| | AfAfAfuau | | | auuUfgAfUf | | | AUAUCAAAUA | |
| | caaauaa | | | auuuUfuAfu | | | U | |
| | | | | ugsgsa | | | | |
| 455 | cscsaaAfg | 872 | 1381-1399 | (vinu)sGfs | 1108 | 1381-1401 | AGCCAAAGCA | 1164 |
| | CfAfUfugg | | | gguCfuCfCf | | | UUGGAGACCC | |
| | agaccca | | | aaugCfuUfu | | | U | |
| | | | | ggsusu | | | | |
| 456 | csasggUfc | 873 | 1238-1256 | (vinu)sAfs | 1109 | 1238-1258 | CUCAGGUCAG | 1165 |
| | AfGfUfauc | | | uauUfaGfAf | | | UAUCUAAUAU | |
| | uaauaua | | | uacuGfaCfc | | | A | |
| | | | | ugsusu | | | | |
| 457 | asusguCfa | 874 | 1651-1669 | (vinu)sAfs | 1110 | 1651-1671 | GAAUGUCAAA | 1166 |
| | AfAfGfcca | | | gaaUfgUfGf | | | GCCACAUUCU | |
| | cauucua | | | gcuuUfgAfc | | | A | |
| | | | | aususc | | | | |
| 458 | gscsucUfa | 875 | 1673-1691 | (vinu)sCfs | 1111 | 1673-1693 | GGGCUCUACU | 1167 |
| | CfUfCfuau | | | aacUfcAfUf | | | CUAUGAGUUG | |
| | gaguuga | | | agagUfaGfa | | | U | |
| | | | | gcsusu | | | | |
| 459 | csusauGfa | 876 | 1681-1699 | (vinu)sGfs | 1112 | 1681-1701 | CUCUAUGAGU | 1168 |
| | GfUfUfgug | | | aaaGfuCfAf | | | UGUGACUUUC | |
| | acuuuca | | | caacUfcAfu | | | A | |
| | | | | agsasg | | | | |
| 460 | usasugAfg | 877 | 1682-1700 | (vinu)sUfs | 1113 | 1682-1702 | UCUAUGAGUU | 1169 |
| | UfUfGfuga | | | gaaAfgUfCf | | | GUGACUUUCA | |
| | cuuucaa | | | acaaCfuCfa | | | A | |
| | | | | uasgsa | | | | |
| 461 | gsgsccCfa | 878 | 1707-1725 | (vinu)sGfs | 1114 | 1707-1727 | UUGGCCCAAA | 1170 |
| | AfAfGfaaa | | | aguAfcUfUf | | | GAAAGUACUC | |
| | guacuca | | | ucuuUfgGfg | | | A | |
| | | | | ccsusu | | | | |
| 462 | gscscaUfa | 879 | 1768-1786 | (vinu)sUfs | 1115 | 1768-1788 | GGGCCAUAUG | 1171 |
| | UfGfUfugc | | | uccCfaGfCf | | | UUGCUGGGAA | |
| | ugggaaa | | | aacaUfaUfg | | | U | |
| | | | | gcsusu | | | | |
| 463 | cscscaCfu | 880 | 1899-1979 | (vinu)sCfs | 1116 | 1899-1981 | GACCCACUGC | 1172 |
| | GfCfAfaag | | | auaGfuCfUf | | | AAAGACUAUG | |
| | acuauga | | | uugcAfgUfg | | | A | |
| | | | | ggsusu | | | | |
| 464 | gsascuAfu | 881 | 1910-1928 | (vinu)sUfs | 1117 | 1910-1930 | AAGACUAUGA | 1173 |
| | GfAfCfagc | | | uugAfuGfCf | | | CAGCAUCAAA | |
| | aucaaaa | | | ugucAfuAfg | | | U | |
| | | | | ucsusu | | | | |
| 465 | csusauGfa | 882 | 1912-1930 | (vinu)sAfs | 1118 | 1912-1932 | GACUAUGACA | 1174 |
| | CfAfGfcau | | | auuUfgAfUf | | | GCAUCAAAUU | |
| | caaauua | | | gcugUfcAfu | | | U | |
| | | | | agsusc | | | | |
| 466 | csusgaAfg | 883 | 2047-2065 | (vinu)sAfs | 1119 | 2047-2067 | ACCUGAAGAA | 1175 |
| | AfAfUfgcu | | | agaAfcAfGf | | | UGCUGUUCUU | |
| | guucuua | | | cauuCfuUfc | | | U | |
| | | | | agsusu | | | | |
| 467 | csusgaAfg | 883 | 2047-2065 | (vinu)sAfs | 1120 | 2047-2067 | ACCUGAAGAA | 1176 |
| | AfAfUfgcu | | | agaAfcAfGf | | | UGCUGUUCUU | |
| | guucuua | | | cauuCfuUfc | | | U | |
| | | | | agsgsu | | | | |
| 468 | cscsugAfa | 884 | 2095-2113 | (vinu)sUfs | 1121 | 2095-2115 | GCCCUGAAAG | 1177 |
| | AfGfGfcca | | | gauCfuUfGf | | | GCCAAGAUCA | |
| | agaucaa | | | gccuUfuCfa | | | A | |
| | | | | ggsusu | | | | |
| 469 | cscsugAfa | 884 | 2095-2113 | (vinu)sUfs | 1122 | 2095-2115 | GCCCUGAAAG | 1178 |
| | AfGfGfcca | | | gauCfuUfGf | | | GCCAAGAUCA | |
| | agaucaa | | | gccuUfuCfa | | | A | |
| | | | | ggsgsc | | | | |
| 470 | gsascuGfc | 885 | 2144-2162 | (vinu)sUfs | 1123 | 2144-2164 | CUGACUGCUG | 1179 |
| | UfGfCfuac | | | uagAfuGfUf | | | CUACAUCUAA | |
| | aucuaaa | | | agcaGfcAfg | | | U | |
| | | | | ucsusu | | | | |
| 471 | asgsguCfa | 886 | 1239-1257 | (vinu)sUfs | 1124 | 1239-1259 | UCAGGUCAGU | 1180 |
| | GfUfAfucu | | | auaUfuAfGf | | | AUCUAAUAUA | |
| | aauauaa | | | auacUfgAfc | | | A | |
| | | | | cususu | | | | |
| 472 | gsgsaaUfu | 887 | 1782-1800 | (vinu)sAfs | 1125 | 1782-1802 | UGGGAAUUUC | 1181 |
| | UfCfCfucc | | | aggGfuGfGf | | | CUCCACCCUU | |
| | acccuua | | | aggaAfaUfu | | | C | |
| | | | | ccsusu | | | | |
| 473 | gsasuuCfa | 888 | 1597-1615 | (vinu)sAfs | 1126 | 1597-1617 | CCGAUUCACC | 1182 |
| | CfCfUfuug | | | cacGfuCfAf | | | UUUGACGUGU | |
| | acgugua | | | aaggUfgAfa | | | A | |
| | | | | ucsusu | | | | |
| 474 | gsasggAfu | 889 | 1479-1497 | (vinu)sCfs | 1127 | 1479-1499 | UGGAGGAUGA | 1183 |
| | GfAfCfacg | | | aggCfaCfGf | | | CACGUGCCUG | |
| | ugccuga | | | ugucAfuCfc | | | A | |
| | | | | ucsusu | | | | |
| 475 | csuscuAfu | 890 | 1679-1697 | (vinu)sAfs | 1128 | 1679-1699 | UACUCUAUGA | 1184 |
| | GfAfGfuug | | | aguCfaCfAf | | | GUUGUGACUU | |
| | ugacuua | | | acucAfuAfg | | | U | |
| | | | | agsusa | | | | |
| 476 | asgsagGfa | 891 | 1430-1448 | (vinu)sGfs | 1129 | 1430-1450 | CUAGAGGAGG | 1185 |
| | GfGfAfugg | | | cagUfuCfCf | | | AUGGAACUGC | |
| | aacugca | | | auccUfcCfu | | | A | |
| | | | | cusasg | | | | |
| 477 | gsasuuCfa | 888 | 1597-1615 | (vinu)sAfs | 1130 | 1597-1617 | CCGAUUCACC | 1186 |
| | CfCfUfuug | | | cacGfuCfAf | | | UUUGACGUGU | |
| | acgugua | | | aaggUfgAfa | | | A | |
| | | | | ucsgsg | | | | |
| 478 | uscsagGfu | 892 | 1237-1255 | (vinu)sUfs | 1131 | 1237-1257 | ACUCAGGUCA | 1187 |
| | CfAfGfuau | | | auuAfgAfUf | | | GUAUCUAAUA | |
| | cuaauaa | | | acugAfcCfu | | | U | |
| | | | | gasusu | | | | |
| 479 | csasguauC | 786 | 1245-1265 | (vinu)sCfs | 1188 | 1243-1265 | GUCAGUAUCU | 498 |
| | fuAfAfUfa | | | gagCfuuaua | | | AAUAUAAGCU | |
| | uaagcucga | | | uuAfgAfuac | | | CGG | |
| | | | | ugsasc | | | | |
| 480 | usasauauA | 792 | 1252-1272 | (vinu)sCfs | 1189 | 1250-1272 | UCUAAUAUAA | 504 |
| | faGfCfUfc | | | aaaCfuccga | | | GCUCGGAGUU | |
| | ggaguuuga | | | gcUfuAfuau | | | UGG | |
| | | | | uasgsa | | | | |
| 481 | csasguauC | 786 | 1245-1265 | (vinu)sCfs | 1190 | 1243-1265 | GUCAGUAUCU | 498 |
| | fuAfAfUfa | | | gagCfuuaua | | | AAUAUAAGCU | |
| | uaagcucga | | | uuAfgauacu | | | CGG | |
| | | | | gsasc | | | | |
| 482 | csasgaCfa | 1192 | 1942-1962 | (vinu)sUfs | 1191 | 1940-1962 | UGCAGACAGU | 538 |
| | guAfCfAfg | | | aucUfagccu | | | ACAGGCUAGA | |
| | gcuagauaa | | | guAfcugucu | | | UAA | |
| | | | | gscsa | | | | |

The nucleotide sequence presented in Table 3, utilize the following abbreviations set forth in Table 4.

TABLE 4
Abbreviations of Nucleotide Modifications.

| Abbreviation | Nucleotide/Linkage |
|---|---|
| (vin) | vinyl-phosphonate |
| (vinu) | 5′-vinyl-phosphonate-2′-O-methyluridine |
| a | 2′-O-methyladenosine |
| c | 2′-O-methylcytidine |
| g | 2′-O-methylguanosine |
| u | 2′-O-methyluridine |
| Af | 2′-fluoroadenosine |
| Cf | 2′-fluorocytidine |
| Gf | 2′-fluoroguanosine |
| Uf | 2′-fluorouridine |
| S | Phosphorothioate linkage (between the two bases) |

Various salts, mixed salts and free acid forms of the dsRNA agents are also provided herein. In some embodiments, the dsRNA agent is in a free acid form. In some embodiments, the dsRNA agent is in a salt form. In some embodiments, the dsRNA agent is in a sodium salt form. In some embodiments, wherein the dsRNA agent is in the sodium salt form, sodium ions are present in the composition comprising the dsRNA agent as counterions for substantially all of the phosphodiester or phosphorothioate groups present in the dsRNA agent. In some embodiments, wherein the dsRNA agent is in the sodium salt form, sodium ions are present in the agent as counterions for all of the phosphodiester or phosphorothioate groups present in the dsRNA agent.

### 4.3 Modified RNAi Agents

In some embodiments, the agent (or any component thereof (e.g., any nucleic acid molecule thereof)) (e.g., described herein, e.g., an antisense strand, a sense strand, a dsRNA agent, RNAi agent, etc.) comprises one or more modified nucleotide(s) (as defined herein). The modified agent may have one or more different (e.g., improved) properties relative to a corresponding unmodified agent. For example, the modified agent may exhibit decreased immunostimulatory activity (e.g., when administered to a subject), increased stability (e.g., in vivo, in a cell, when administered to a subject), and/or increased inhibition of expression of a target nucleic acid molecule (e.g., a target mRNA (e.g., a CIDEB mRNA)), or any combination thereof.

### 4.3.1 Nature of Nucleotide Modifications

Nucleotide modifications can include modification to any one of more of the nucleoside and/or the internucleoside linkage. Nucleoside modifications include modification to the sugar (e.g., ribose) moiety and/or the nucleobase. In some embodiments, the modified agent (or component thereof) (e.g., antisense strand, sense strand, dsRNA agent, etc.) comprises one or more nucleotides comprising a modified sugar moiety. In some embodiments, the modified agent (or component thereof) (e.g., antisense strand, sense strand, dsRNA agent, etc.) comprises one or more nucleotides comprising a modified nucleobase. In some embodiments, the modified agent (or component thereof) (e.g., antisense strand, sense strand, dsRNA agent, etc.) comprises one or more nucleotides comprising a modified internucleoside linkage. In some embodiments, the modified agent (or component thereof) (e.g., antisense strand, sense strand, dsRNA agent, etc.) comprises one or more nucleotides comprising one, two, or three of a modified sugar moiety, a modified nucleobase, and/or a modified internucleoside linkage.

Exemplary nucleotide modifications are described below and also known in the art, see, e.g., WO2021257782, WO2013075035, WO2022246251, and WO2022271573, the entire contents of each of which is incorporated by reference herein for all purposes. Exemplary modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### 4.3.1.1 Modified Nucleosides

In some embodiments, the modified agent (or any component thereof) (e.g., antisense strand, sense strand, dsRNA agent, etc.) comprises one or more nucleotide comprising a modified nucleoside. As discussed above, nucleoside modifications can include modification of the sugar (e.g., ribose) moiety and/or modification of the nucleobase.

### (i) Sugar Modifications

In some embodiments, the modified agent (or any component thereof) (e.g., antisense strand, sense strand, dsRNA agent, etc.) comprises one or more nucleotides comprising a modified sugar (e.g., ribose) moiety.

The modified sugar (e.g., ribose) moiety can comprise, for example, a substituent at any one or more position of the sugar (e.g., ribose), including e.g., positions 2′, 4′, and/or 5′. In some embodiments, the modified sugar (e.g., ribose) comprises a substituent at 2′ position of the sugar (e.g., ribose). In some embodiments, the modified sugar (e.g., ribose) comprises a substituent at 5′ position of the sugar (e.g., ribose). In some embodiments, the modified sugar (e.g., ribose) comprises a substituent at 5′ position of the sugar (e.g., ribose).

In some embodiments, the agent (or any component thereof) comprises any one or more of the following substituents (e.g., at any position of the sugar (e.g., ribose) (e.g., at position 2′)): a group for improving the stability of the agent, a group for improving the pharmacokinetic properties of the agent, or a group for improving the pharmacodynamic properties of the agent, an RNA cleaving group, a reporter group, an intercalator, or other substituents having similar properties.

Exemplary substituents include, for example, but are not limited to, substitution (e.g., at any position of the sugar (e.g., ribose) (e.g., at position 2′)) with any one of the following: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Additional exemplary substitutions (e.g., at any position of the sugar (e.g., ribose) (e.g., at position 2′)) include, for example, but are not limited to, substitution with any one of the following: O[(CH₂)ₙO]m, CH₃, O(CH₂)ₙOCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to about 10.

In some embodiments, the modified sugar (e.g., ribose) comprises any one of the following modifications: 2′-O-methyl (2′-OMe), 2′O-methoxyethyl (2′-O-MOE), 2′deoxy-2′-fluoro (2′-F), 2′-arabino-fluoro (2′-Ara-F), 2′-O-benzyl, 2′-O-methyl-4-pyridine (2-O-methyl-4-pyridine (2′-O—CH2Py(4)).

In some embodiments, the agent (or any component thereof) comprises any of the following substituents at the 2′-position of the sugar (e.g., ribose): C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, or a substituted silyl. In some embodiments, the agent (or any component thereof) comprises a 2′-methoxyethoxy (2′-O—CH₂CH₂OCH₃, also known as 2′-O-(2-methoxyethyl) or 2′-MOE) (see, e.g., Martin et al., Helv. Chim. Acta, 1995, 78:486-504, the entire contents of which is incorporated by reference herein for all purposes) (i.e., an alkoxy-alkoxy group). In some embodiments, the agent (or any component thereof) comprises a 2′-dimethylaminooxyethoxy, i.e., a O(CH₂)₂ON(CH₃)₂ group, also known as 2′-DMAOE; a 2′-dimethylaminoethoxyethoxy (also known in the art as 2′-O-dimethylaminoethoxyethyl or 2′-DMAEOE), i.e., 2′-O—CH₂—O—CH₂—N(CH₃) 2; a 5′-Me-2′-F nucleotide, a 5′-Me-2′-OMe nucleotide, a 5′-Me-2′-deoxynucleotide, (both R and S isomers in these three families); a 2′-alkoxyalkyl; and 2′-NMA (N-methylacetamide).

Exemplary US patents that describe the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; the entire contents of each of the foregoing are hereby incorporated herein by reference for all purposes. Exemplary sugar modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (a) Non-Bicyclic Sugar Modifications

In some embodiments, the modified sugar (e.g., ribose) moiety comprises a non-bicyclic modified sugar (e.g., ribose) moiety. In some embodiments, the modified sugar (e.g., ribose) moiety comprises a furanosyl ring comprising one or more substituent groups none of which bridges two atoms of the furanosyl ring to form a bicyclic structure. In some embodiments one or more non-bridging substituent of a non-bicyclic modified sugar moiety is branched. Such non bridging substituents may be at any position of the furanosyl, including but not limited to substituents at the 2′, 4′, and/or 5′ positions.

In some embodiments, non-bicyclic modified sugar moiety comprises a substituent group at the 2′-position of the sugar (e.g., ribose). Examples of 2′-substituent groups suitable for non-bicyclic modified sugar moieties include but are not limited to: 2′-O-methyl (2′-OMe), 2′O-methoxyethyl (2′-O-MOE), 2′deoxy-2′-fluoro (2′-F), 2′-arabino-fluoro (2′-Ara-F), 2′-O-benzyl, 2′-O-methyl-4-pyridine (2-O-methyl-4-pyridine (2′-O—CH₂Py (4)), and 2′-O—N-alkyl acetamide (e.g., 2′-O—N-methyl acetamide (“NMA”), 2′-O—N-dimethyl acetamide, 2′-O—N-ethyl acetamide, and 2′-O—N-propyl acetamide). For example, see, e.g., U.S. Pat. No. 6,147,200, Prakash et al., 2003, Org. Lett., 5, 403-6, the entire contents of which is incorporated by reference herein for all purposes.

In some embodiments, the 2′-substituent group is a halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH2)2SCH3,0(CH2)2ON(Rm)(Rn) or OCH2C(═O)—N(Rm)(Rn), where each Rₘ and Rₙ is, independently, H, an amino protecting group, or substituted or unsubstituted C₁-C₁₀ alkyl, or a 2′-substituent group described in any one of the following: Cook et al., U.S. Pat. No. 6,531,584; Cook et al., U.S. Pat. No. 5,859,221; and Cook et al., U.S. Pat. No. 6,005,087, the entire contents of which are incorporated herein by reference for all purposes. In some embodiments, these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In some embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In some embodiments, a 2′-substituted non-bicyclic modified nucleoside comprises a sugar moiety comprising a non-bridging 2′-substituent group selected from: F, OCF, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In some embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

In some embodiments, non-bicyclic modified sugar moiety comprises a substituent group at the 3′-position of the sugar (e.g., ribose). Examples of substituent groups suitable for the 3′-position of modified sugar moieties include but are not limited to alkoxy (e.g., methoxy), alkyl (e.g., methyl, ethyl).

In some embodiments, non-bicyclic modified sugar moiety comprises a substituent group at the 4′-position of the sugar (e.g., ribose). Examples of 4′-substituent groups suitable for non-bicyclic modified sugar moieties include but are not limited to alkoxy (e.g., methoxy), alkyl, and those described in Manoharan et al., WO 2015/106128.

In some embodiments, non-bicyclic modified sugar moiety comprises a substituent group at the 5′-position of the sugar (e.g., ribose). Examples of substituent groups suitable for the 5′-position of modified sugar moieties include, but are not limited to, vinyl (e.g., 5′-vinyl), alkoxy (e.g., methoxy (e.g., 5′-methoxy)), and alkyl (e.g., methyl (R or S) (e.g., 5′-methyl (R or S)), ethyl).

In some embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836, the entire contents of each of which is incorporated herein by reference for all purposes.

In some embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, the entire contents of which are incorporated by reference herein for all purposes.

In some embodiments, the sugar (e.g., ribose) modification comprises an unlocked nucleotide (UNA). UNA is unlocked acyclic nucleic acid, wherein any of the bonds of the sugar has been removed, forming an unlocked sugar (e.g., ribose) residue. For example, in some embodiments, the bonds between C1′-C4′ have been removed (i.e., the covalent carbon-oxygen-carbon bond between the C1′ and C4′ carbons). In some embodiments, the C2′-C3′ bond (i.e., the covalent carbon-carbon bond between the C2′ and C3′ carbons) of the sugar (e.g., ribose) have been removed. See, e.g., Nuc. Acids Symp. Series, 52, 133-134 (2008) and Fluiter et al., Mol. Biosyst., 2009, 10, 1039, the entire contents of which are incorporated herein by reference. UNAs and methods of making are known in the art. See, e.g., U.S. Pat. No. 8,314,227; and US2013/0096289; US2013/0011922; and US2011/0313020, the entire contents of each of which are hereby incorporated herein by reference.

### (b) Bicyclic Sugar Modifications

In some embodiments, the modified sugar (e.g., ribose) moiety comprises a substituent that bridges two atoms of the furanosyl ring to form a second ring, resulting in a bicyclic sugar (e.g., ribose) moiety. In some embodiments, the bicyclic sugar (e.g., ribose) moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-₂′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2QQ9, 74, 118-134), 4′-CH₂—C(═CH₂)-2′ and analogs thereof (see, e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672). The entire contents of all of the foregoing references is incorporated by reference herein for all purposes.

In some embodiments, such 4′ to 2′ bridges independently comprise from 1 to 4 linked groups independently selected from: —[C(Rₐ)(R_{b})]n-, —[C(Rₐ)(R_{b})]n-O—, —C(Rₐ)═C(R_{b})—, —C(Rₐ)═N—, —C(═NRₐ)—, —C(═O)—, —C(═S)—, —O—, —Si(Rₐ)₂—, —S(═O)X—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(=0)2-J1), or sulfoxyl (S(═O)-J1); and each J1 and J2 is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25 (22), 4429-4443, Alback et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007,129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et. al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727. The entire contents of all of the foregoing references is incorporated by reference herein for all purposes.

In some embodiments, the modified sugar (e.g., ribose) comprises a constrained ethyl nucleotide comprising a 4′-CH(CH₃)—O-2′ bridge. In some embodiments, the constrained ethyl nucleotide is in the S conformation (S-cEt). In some embodiments, the modified sugar (e.g., ribose) comprises a conformationally restricted nucleotide (CRN). CRNs are nucleotide analogs with a linker connecting the C2′ and C4′ carbons of ribose or the C3 and —C5′ carbons of ribose. Representative publications that teach the preparation of certain of the above include, but are not limited to, US2013/0190383; and WO2013/036868, the entire contents of each of which are hereby incorporated herein by reference.

In some embodiments, bicyclic sugar moieties and nucleosides incorporating such bicyclic sugar moieties are further defined by isomeric configuration. For example, an LNA nucleoside (described herein) may be in the α-L configuration or in the β-D configuration. Herein, general descriptions of bicyclic nucleosides include both isomeric configurations. Any of the foregoing bicyclic nucleosides can be prepared having one or more stereochemical sugar configurations including for example α-L-ribofuranose and β-D-ribofuranose (see, e.g., WO 99/14226, the entire contents of which are incorporated herein by reference for all purposes).

Additional representative U.S. patents and U.S. Patent Publications that teach the preparation of bicyclic nucleosides (e.g., locked nucleic acid) include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

### (ii) Nucleobase Modifications

In some embodiments, the modified agent (or any component thereof) (e.g., antisense strand, sense strand, dsRNA agent, etc.) comprises one or more nucleotides comprising a modified nucleobase.

As used herein, “unmodified” nucleobases refer to the purine bases adenine (A) and guanine (G), and the pyrimidine bases thymine (T), cytosine (C), and uracil (U). Modified nucleobases include other synthetic and natural nucleobases.

Modified nucleobases include, but are not limited to, 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 5-methylcytosine, 2-aminopropyladenine, 5-hydroxymethyl cytosine, xanthine, hypoxanthine, deoxythimidine (dT), 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-Nbenzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminocthoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808; The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443; the entire contents of each of which is incorporated herein by reference for all purposes.

In some embodiments, the modified nucleobase comprises a pseudouridine, 2′thiouridine (s2U), N6′-methyladenosine, 5′methylcytidine (m⁵C), 5′fluoro-2′deoxyuridine, N-ethylpiperidine 7-EAA triazole modified adenine, N-ethylpiperidine 6′triazole modified adenine, 6-phenylpyrrolo-cytosine (PhpC), 2′,4′-difluorotoluyl ribonucleoside (rF), or 5′nitroindole. In some embodiments, the modified nucleobase comprises a 5-substituted pyrimidine; 6-azapyrimidine; or N-2, N-6 and 0-6 substituted purines (including 2-aminopropyladenine, 5-propynyluracil and 5-propynylcytosine). 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents an published applications that teach the preparation of certain of the above noted modified nucleobases as well as other modified nucleobases include, but are not limited to, U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; 7,495,088; 5,130,302; 5,134,066; 5,175,273; 5,367,066; 5,432,272; 5,434,257; 5,457,187; 5,459,255; 5,484,908; 5,502,177; 5,525,711; 5,552,540; U.S. Pat. Nos. 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,166,199; and 6,005,096, the entire contents of each of which is hereby incorporated herein by reference for all purposes. Exemplary nucleobase modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### 4.3.1.2 Internucleoside Linkage Modifications

In some embodiments, the modified agent (or any component thereof) (e.g., antisense strand, sense strand, dsRNA agent, etc.) comprises one or more modified internucleoside linkage. Modified internucleoside linkages, compared to naturally occurring phosphate linkages, can be used to alter, typically increase, nuclease resistance of an agent (e.g., described herein).

The naturally occurring internucleoside linkage of RNA and DNA is a 3′ to 5′ phosphodiester linkage. In some embodiments, the modified internucleoside linkage contains a normal 3′-5′ linkage. In some embodiments, the modified internucleoside linkage contains a 2′-5′ linkage. In some embodiments, the modified internucleoside linkage has an inverted polarity wherein the adjacent pairs of nucleoside units are linked e.g., 3′-5′ to 5′-3′ or 2′-5′ to 5′-2′.

The two main classes of modified internucleoside linking can be defined by the presence or absence of a phosphorous atom.

Exemplary internucleoside linkage modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (i) Modified Phosphorous Containing Internucleoside Linkages

In some embodiments, the modified internucleoside linkage comprises a phosphorous atom. Representative modified phosphorus-containing internucleoside linkages include but are not limited to phosphorothioates (PS (Rp isomer or Sp isomer)) (e.g., 5′phosphorothioate), phosphotriesters, phosphoramidates (e.g., 3′-amino phosphoramidate and aminoalkylphosphoramidates), chiral phosphorothioates, phosphorodithioates (PS2), aminoalkylphosphotriesters, methyl and other alkyl phosphonates (e.g., methylphosphonate (MP), 3′-alkylene phosphonates), methpxypropyl-phosphonates (MOP), vinyl-phosphonate, 5′-(E)-vinylphosphonates, 5′methyl phosphonates, (S)-5′C-methyl with phosphates, phosphinates, thionophosphoramidates, thionoalkylphosphonates, thionoalkylphosphotriesters, boranophosphates, and peptide nucleic acids (PNAs).

In some embodiments, the modified internucleotide linkage comprises a vinyl-phosphonate. In some embodiments, the modified internucleotide linkage comprises a vinyl-phosphonate-2′-O-methyl (e.g., a vinyl-phosphonate-2′-O-methyluridine).

Methods of preparing polynucleotides containing one or more modified phosphorus-containing internucleoside linkage are known in the art. See, e.g., U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference for all purposes. Exemplary modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (ii) Modified Non-Phosphorous Containing Internucleoside Linkages

In some embodiments, the modified internucleoside linkage does not contain a phosphorous atom. Modified internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative non-phosphorous containing internucleoside linking groups include but are not limited to methylenemethylimino (—CH₂—N(CH₃)—O—CH₂—), thiodiester, thionocarbamate (—O—C(═O) (NH)—S—); siloxane (—O—SiH₂—O—); and N,N′-dimethylhydrazine (—CH₂—N(CH₃)—N(CH₃)—).

Methods of preparing polynucleotides comprising modified internucleoside linkages do not contain a phosphorous atom are known in the art. See, e.g., U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### 4.3.1.3 Additional Exemplary Nucleotide Modifications

In some embodiments, the modified agent comprises one or more RNA mimetic in which both the sugar and the internucleoside linkage of the nucleotide units are replaced with novel groups. The nucleobase units are maintained for hybridization with an appropriate nucleic acid target (e.g., a target mRNA). In some embodiments, the RNA mimetic is a peptide nucleic acid (PNA). In PNAs, the ribose moiety of the RNA nucleotide is replaced with an amide containing moiety (e.g., an aminoethylglycine). The nucleobases are retained and are bound directly or indirectly to aza nitrogen atoms of the amide. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the agents described herein are described in, for example, in Nielsen et al., Science, 1991, 254, 1497-1500, the entire contents of which is incorporated by reference herein for all purposes.

Potentially stabilizing modifications to the terminal ends of the agents (e.g., described herein) can also be incorporated to agents described herein. For example, N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT (idT) and others. Such modifications are known in the art. See, e.g., WO2011/005861, the entire contents of which is incorporated herein by reference.

### 4.3.2 Extent of Modified Nucleotides

In some embodiments, at least 5%, 10%, 15%, 20%, 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent) are modified. In some embodiments, about 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent, etc.) are modified. In some embodiments, 100% of the nucleotides of the agent (or any component (e.g., nucleic acid molecule) thereof) (e.g., described herein, e.g., an antisense strand, a sense strand, a dsRNA agent, RNAi agent, etc.) are modified. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the nucleotides of the agent (or any component (e.g., nucleic acid molecule) thereof) (e.g., described herein, e.g., an antisense strand, a sense strand, a dsRNA agent, RNAi agent, etc.) are modified. In some embodiments, substantially all of the nucleotides of the agent (or any component (e.g., nucleic acid molecule) thereof) (e.g., described herein, e.g., an antisense strand, a sense strand, a dsRNA agent, RNAi agent, etc.) are modified. In specific embodiments, all of the nucleotides of the agent (or any component (e.g., nucleic acid molecule) thereof) (e.g., described herein, e.g., an antisense strand, a sense strand, a dsRNA agent, RNAi agent, etc.) are modified.

In some embodiments, at least 25%, 30%, 35%, 40%, 45%, 50%, 55%, 60%, 65%, 70%, 75%, 80%, 85%, 90%, 95%, or 100% of the nucleotides of the sense and/or antisense strand are modified. For example, at least 50% of the nucleotides of the sense strand and/or antisense strand may be modified. For example, at least 55% of the nucleotides of the sense strand and/or antisense strand may be modified. For example, at least 60% of the nucleotides of the sense strand and/or antisense strand may be modified. For example, at least 65% of the nucleotides of the sense strand and/or antisense strand may be modified. For example, at least 70% of the nucleotides of the sense strand and/or antisense strand may be modified. For example, at least 75% of the nucleotides of the sense strand and/or antisense strand may be modified. For example, at least 80% of the nucleotides of the sense strand and/or antisense strand may be modified. For example, at least 85% of the nucleotides of the sense strand and/or antisense strand may be modified. For example, at least 90% of the nucleotides of the sense strand and/or antisense strand may be modified. For example, at least 90% of the nucleotides of the sense strand and/or antisense strand may be modified. For example, at least 95% of the nucleotides of the sense strand and/or antisense strand may be modified. In some embodiments, substantially all (or all) of the nucleotides in the sense strand and/or antisense strand are modified.

In some embodiments, substantially all (or all) of the nucleotides in the sense strand are modified. In some embodiments, substantially all (or all) of the nucleotides in the antisense strand are modified. In some embodiments, substantially all (or all) of the nucleotides in the sense strand and antisense strand are modified.

In some embodiments, at least one of the modified nucleotides comprises a modified sugar (e.g., ribose moiety). In some embodiments, at least one of the modified nucleotides comprises a modified nucleobase. In some embodiments, the sense strand comprises at least one modified internucleoside linkage and/or the antisense strand comprises at least one modified internucleoside linkage.

In some embodiments, not more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 of the nucleotides of the of the agent (or any component (e.g., nucleic acid molecule) thereof) (e.g., described herein, e.g., an antisense strand, a sense strand, a dsRNA agent, RNAi agent, etc.) are unmodified. In some embodiments, not more than 10, 9, 8, 7, 6, 5, 4, 3, 2, or 1 of the nucleotides of the agent (or any component (e.g., nucleic acid molecule) thereof) (e.g., described herein, e.g., an antisense strand, a sense strand, a dsRNA agent, RNAi agent, etc.) are unmodified. In some embodiments, not more than 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent, etc.) are unmodified.

In some embodiments, at least 5%, 10%, 15%, 20%, 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent) are unmodified. In some embodiments, about 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent, etc.) are unmodified. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the nucleotides of the agent (or any component (e.g., nucleic acid molecule) thereof) (e.g., described herein, e.g., an antisense strand, a sense strand, a dsRNA agent, RNAi agent, etc.) are unmodified. In some embodiments, substantially all of the nucleotides of the agent (or any component (e.g., nucleic acid molecule) thereof) (e.g., described herein, e.g., an antisense strand, a sense strand, a dsRNA agent, RNAi agent, etc.) are unmodified. In some embodiments, all of the nucleotides of the agent (or any component (e.g., nucleic acid molecule) thereof) (e.g., described herein, e.g., an antisense strand, a sense strand, a dsRNA agent, RNAi agent, etc.) are unmodified.

In some embodiments, not more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 of the nucleotides of the of the agent (or any component (e.g., nucleic acid molecule) thereof) (e.g., described herein, e.g., an antisense strand, a sense strand, a dsRNA agent, RNAi agent, etc.) are modified. In some embodiments, not more than 10, 9, 8, 7, 6, 5, 4, 3, 2, or 1 of the nucleotides of the agent (or any component (e.g., nucleic acid molecule) thereof) (e.g., described herein, e.g., an antisense strand, a sense strand, a dsRNA agent, RNAi agent, etc.) are modified. In some embodiments, not more than 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, RNAi agent, etc.) are modified.

In some embodiments, the RNAi agent (e.g., antisense strand, sense strand, dsRNA agent (e.g., described herein)) comprises one or more non-naturally internucleoside linkage. In some embodiments, at least 25%, 30%, 35%, 40%, 45%, 50%, 55%, 60%, 65%, 70%, 75%, 80%, 85%, 90%, 95%, or 100% of the internucleoside linkages of the RNAi agent (e.g., antisense strand, sense strand, dsRNA agent (e.g., described herein)) are non-naturally occurring. In some embodiments, at least 25%, 30%, 35%, 40%, 45%, 50%, 55%, 60%, 65%, 70%, 75%, 80%, 85%, 90%, 95%, or 100% of the or internucleoside linkages of the RNAi agent (e.g., antisense strand, sense strand, dsRNA agent (e.g., described herein)) are chemically modified.

### 4.4 Conjugates

In some embodiments, the agent (e.g., described herein) (e.g., RNAi agent, dsRNA agent, antisense strand, sense strand) comprises a heterologous moiety (e.g., operably connected to the agent). Therefore, further provided herein are conjugates comprising an agent (e.g., described herein) (e.g., RNAi agent, dsRNA agent, antisense strand, sense strand) and a heterologous moiety (e.g., operably connected to the agent). It is clear from the disclosure, but for the sake of clarity, the conjugate an comprise a modified agent (e.g., described herein, see, e.g., § 4.3).

In some embodiments, the heterologous moiety modifies one or more property of the agent (e.g., described herein) (e.g., RNAi agent, dsRNA agent, antisense strand, sense strand) to which it is conjugated. Exemplary properties include, but are not limited to, pharmacodynamics, pharmacokinetics, stability, absorption, activity, tissue distribution, cellular distribution, cellular uptake, charge, half-life, clearance, and binding affinity to a target nucleic acid molecule (e.g., a target mRNA).

In some embodiments, the heterologous moiety enhances the distribution and/or uptake (e.g., into a cell, e.g., into a cell in a subject) of the agent (e.g., described herein) (e.g., RNAi agent, dsRNA agent, antisense strand, sense strand) (e.g., as compared to an agent that lacks the heterologous moiety). In some embodiments, the heterologous moiety alters (e.g., extends) the lifetime (e.g., in vivo) of the agent (e.g., described herein) (e.g., RNAi agent, dsRNA agent, antisense strand, sense strand) (e.g., as compared to an agent that lacks the heterologous moiety). In some embodiments, the heterologous moiety provides an enhanced affinity for a selected target, e.g., a selected molecule, cell type, compartment (e.g., cell type, tissue, organ or region of the body) (e.g., as compared to an agent that lacks the heterologous moiety).

In some embodiments, the heterologous moiety enhances the activity (e.g., in a cell, e.g., in a cell in a subject) of the agent (e.g., described herein) (e.g., RNAi agent, dsRNA agent, antisense strand, sense strand) (e.g., as compared to an agent that lacks the heterologous moiety). Activity can include, e.g., degradation of a target mRNA (e.g., a CIDEB mRNA), inhibition of expression of a target gene (e.g., a CIDEB gene), and/or reduction in the expression of a target gene (e.g., a CIDEB gene).

In some embodiments, the heterologous moiety imparts a new property on the agent (e.g., described herein) (e.g., RNAi agent, dsRNA agent, antisense strand, sense strand) to which it is conjugated. For example, fluorophores or reporter groups that enable detection of the agent (e.g., described herein) (e.g., RNAi agent, dsRNA agent, antisense strand, sense strand).

It is to be understood the heterologous moieties can impart multiple (e.g., any combination of the foregoing) properties of the agent (e.g., described herein) (e.g., RNAi agent, dsRNA agent, antisense strand, sense strand).

In some embodiments, wherein the agent is a dsRNA agent comprising a double stranded region, the heterologous moiety does not take part in, does not alter, and/or does not interfere with, the creation of a double strand region.

### 4.4.1 Heterologous Moieties

Heterologous moieties, include for example, but are not limited to, carbohydrates, peptides, proteins (e.g., antibodies or functional fragments or variants thereof; ligands (e.g., of a target receptor)), lipids, polymers, small molecules, intercalators, reporter molecules, polyamines, polyamides, vitamin moictics, polyethylene glycols, thioethers, polyethers, cholesterols, thiocholesterols, cholic acid moieties, folate, lipophilic groups, phospholipids, biotin, phenazine, phenanthridine, anthraquinone, adamantane, acridine, fluoresceins, rhodamines, coumarins, fluorophores, and dyes.

In some embodiments, the heterologous moiety is a carbohydrate, peptide, protein (e.g., antibody or functional fragment or variant thereof, e.g., ligand (e.g., of a target receptor)), lipid, polymer, small molecule, or any combination thereof. In some embodiments, the heterologous moiety comprises an active drug substance. In some embodiments, the heterologous moiety does not contain an active drug substance.

Exemplary heterologous moieties (e.g., targeting moieties), further include but are not limited, to carbohydrate moities (e.g., GalNAc and GalNAc derivatives (See, e.g., U.S. Pat. No. 8,106,022 and WO2019055633)); lipid moieties such as a cholesterol moiety (see, e.g., Letsinger et al., Proc. Natl. Acid. Sci. USA, 1989, 86:6553-6556); cholic acid (see, e.g., Manoharan et al., Biorg. Med. Chem. Let., 1994, 4:1053-1060), a thioether, e.g., beryl-S-tritylthiol (see, e.g., Manoharan et al., Ann. N.Y. Acad. Sci., 1992, 660:306-309; Manoharan et al., Biorg. Med. Chem. Let., 1993, 3:2765-2770); thiocholesterols (see, e.g., Oberhauser et al., Nucl. Acids Res., 1992, 20:533-538); aliphatic chains (e.g., dodecandiol or undecyl residues) (see, e.g., Saison-Behmoaras et al., EMBO J, 1991, 10:1111-1118; Kabanov et al., FEBS Lett., 1990, 259:327-330; Svinarchuk et al., Biochimie, 1993, 75:49-54), phospholipids (e.g., dihexadecyl-rac-glycerol or triethyl-ammonium 1,2-di-O-hexadecyl-rac-glycero-3-phosphonate) (see, e.g., Manoharan et al., Tetrahedron Lett., 1995, 36:3651-3654; Shea et al., Nucl. Acids Res., 1990, 18:3777-3783); polyamine or polyethylene glycol chains (see, e.g., Manoharan et al., Nucleosides & Nucleotides, 1995, 14:969-973); adamantane acetic acids (see, e.g., Manoharan et al., Tetrahedron Lett., 1995, 36:3651-3654); palmityl moieties (see, e.g., Mishra et al., Biochim. Biophys. Acta, 1995, 1264:229-237); and octadecylamine or hexylamino-carbonyloxycholesterol moiety (see, e.g., Crooke et al., J. Pharmacol. Exp. Ther., 1996, 277:923-937). The entire contents of each of the foregoing references is incorporated herein by reference for all purposes. Additional carbohydrate heterologous moieties (and linkers) suitable for use in conjugates described herein include those described in PCT Publication Nos. WO 2014/179620 and WO 2014/179627, the entire contents of each of which are incorporated herein by reference for all purposes.

### 4.4.1.1 Targeting Moieties

In some embodiments, the heterologous moiety is a targeting moiety. In some embodiments, the targeting moiety enhances distribution of the agent (e.g., described herein) (e.g., RNAi agent, dsRNA agent, antisense strand, sense strand) to a target cell (or population of cells), tissue, and/or organ (e.g., as compared to an agent that lacks the targeting moiety). In some embodiments, the targeting moiety enhances the uptake of the agent (e.g., described herein) (e.g., RNAi agent, dsRNA agent, antisense strand, sense strand) into a target cell (or population of cells) (e.g., as compared to an agent that lacks the targeting moiety). In some embodiments, the targeting moiety provides enhanced affinity for a selected target, e.g., molecule, cell, cell type, compartment, e.g., a cellular or organ compartment, tissue, organ or region of the body, (e.g., as compared to an agent that lacks the targeting moiety).

In some embodiments, the targeting moiety specifically binds to a target molecule (e.g., protein, carbohydrate, lipid, etc.) expressed on the surface of a target cell, tissue, and/or organ. In some embodiments, the target molecule is a protein, carbohydrate, or lipid. In some embodiments, the target molecule is a receptor.

### (i) Hepatocyte Targeting Moieties

In some embodiments, the targeting moiety specifically binds to a target molecule (e.g., protein, carbohydrate, lipid, etc.) expressed by hepatocytes (e.g., on the surface of the surface of hepatocytes). In some embodiments, the targeting moiety specifically binds to a target molecule protein (e.g., receptor) expressed on the surface of hepatocytes.

In some embodiments, the targeting moiety comprises a carbohydrate. Exemplary carbohydrate targeting moieties are described in WO2019055633, the entire contents of which is incorporated by reference herein for all purposes. In some embodiments, the carbohydrate is a monosaccharide. In some embodiments, the carbohydrate is a polysaccharide.

In some embodiments, the targeting moiety comprises at least one (e.g., at least 2, 3, 4, or more) N-acetylgalactosamine (GalNAc) or GalNAc derivative. In some embodiments, the targeting moiety comprise a plurality (e.g., 2, 3, 4, 5, or 6) of GalNAc moieties and/or GalNAc derivatives. In some embodiments, the targeting moiety comprise a plurality (e.g., 2, 3, 4, 5, or 6) of GalNAc moieties and/or GalNAc derivatives each independently attached to a plurality of nucleotides of the agent (e.g., described herein) (e.g., RNAi agent, dsRNA agent, antisense strand, sense strand) through a plurality of linkers (e.g., monovalent linkers). In some embodiments, the GalNAc targeting moiety serves to target the agent to hepatocytes through specific binding to the asialoglycoprotein receptor.

Exemplary GalNAc conjugates, which comprise one or more GalNAc and/or derivative thereof, are known the art. See, e.g., in U.S. Pat. No. 8,106,022, the entire contents of which is hereby incorporated herein by reference for all purposes. Additional exemplary GalNAc targeting moieties are described below.

In some embodiments, the targeting moiety (e.g., GalNAc targeting moiety) comprises any one of the following formulas:

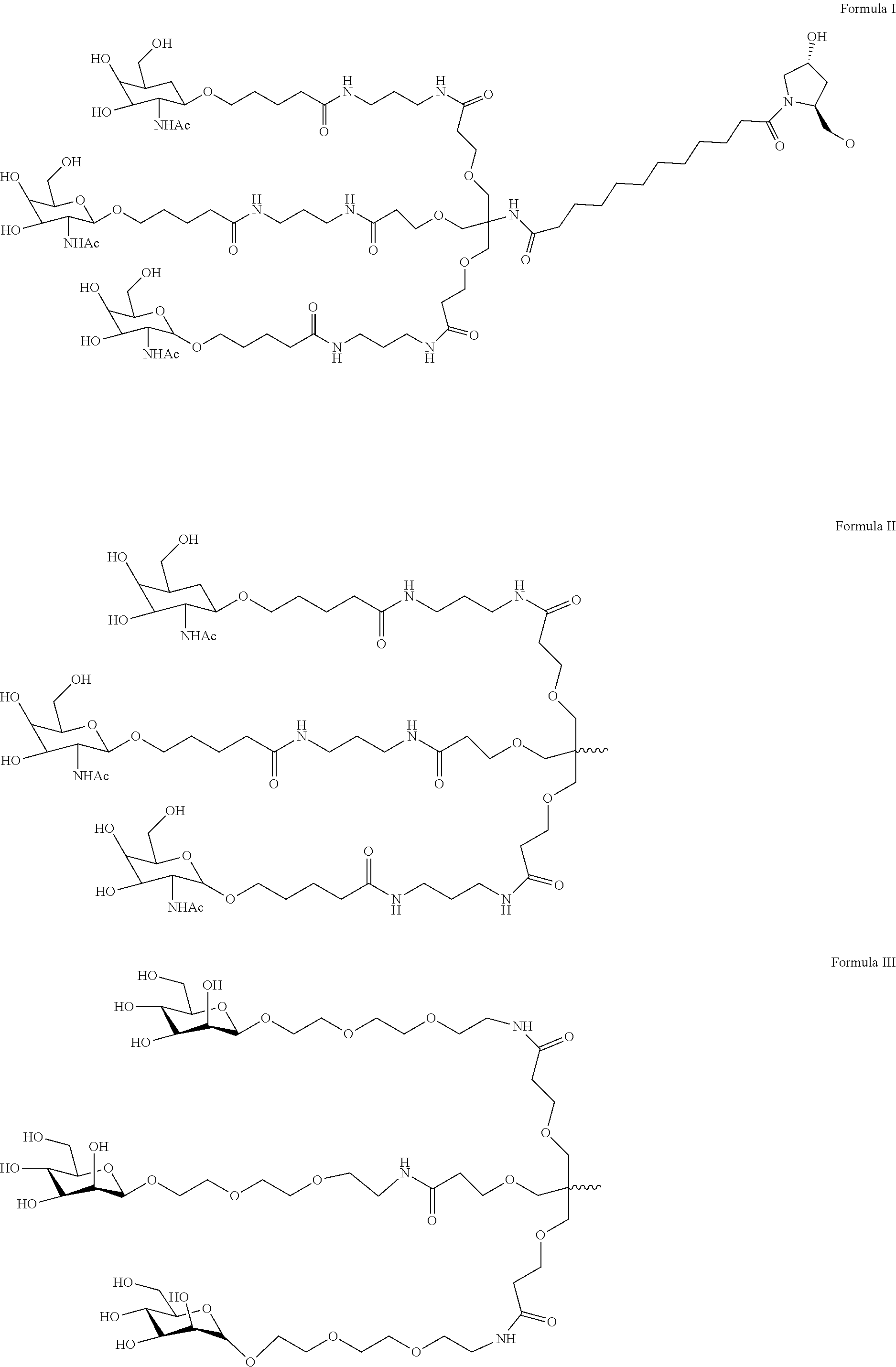

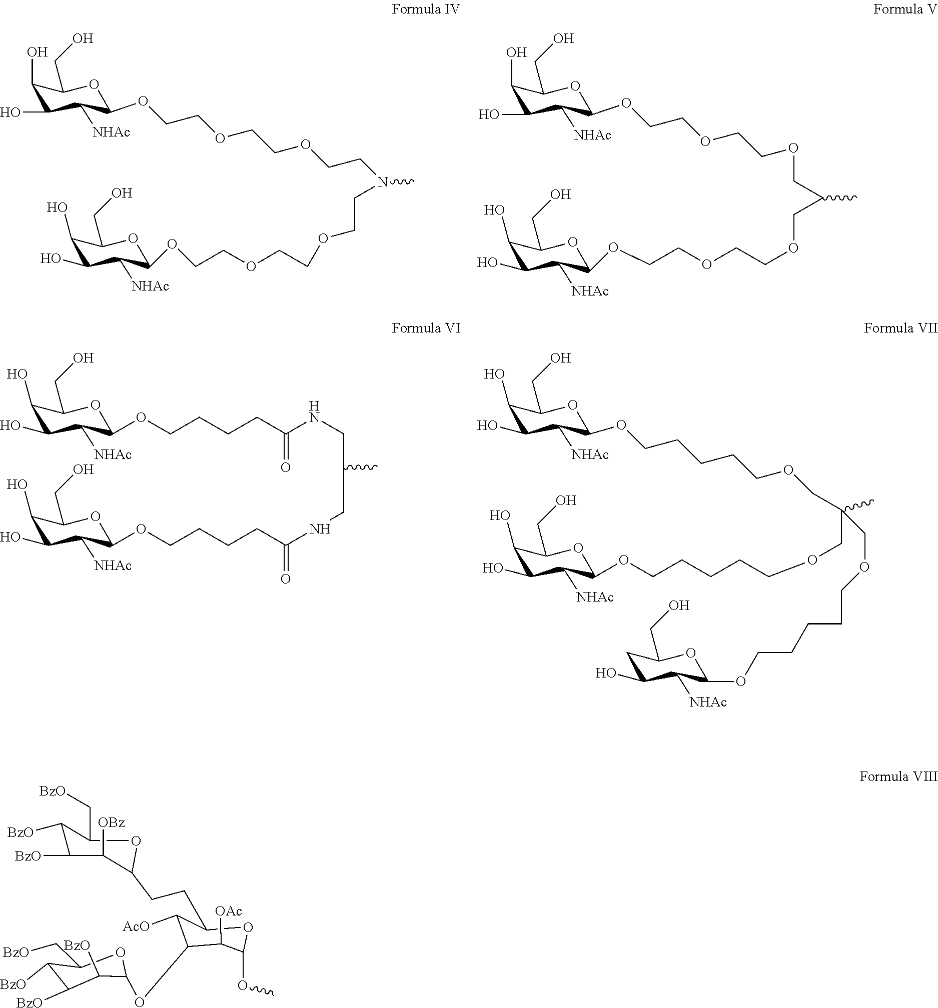

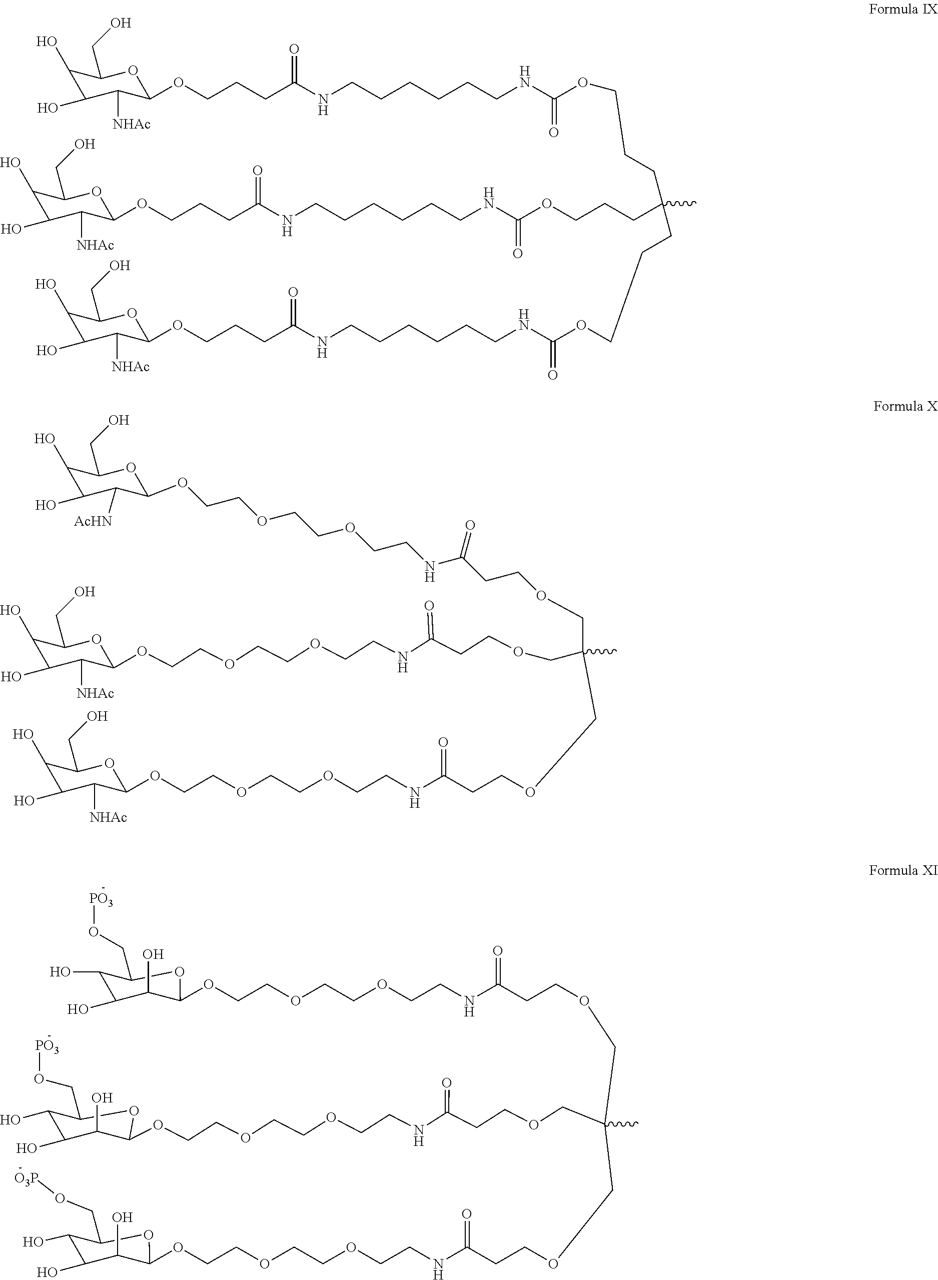

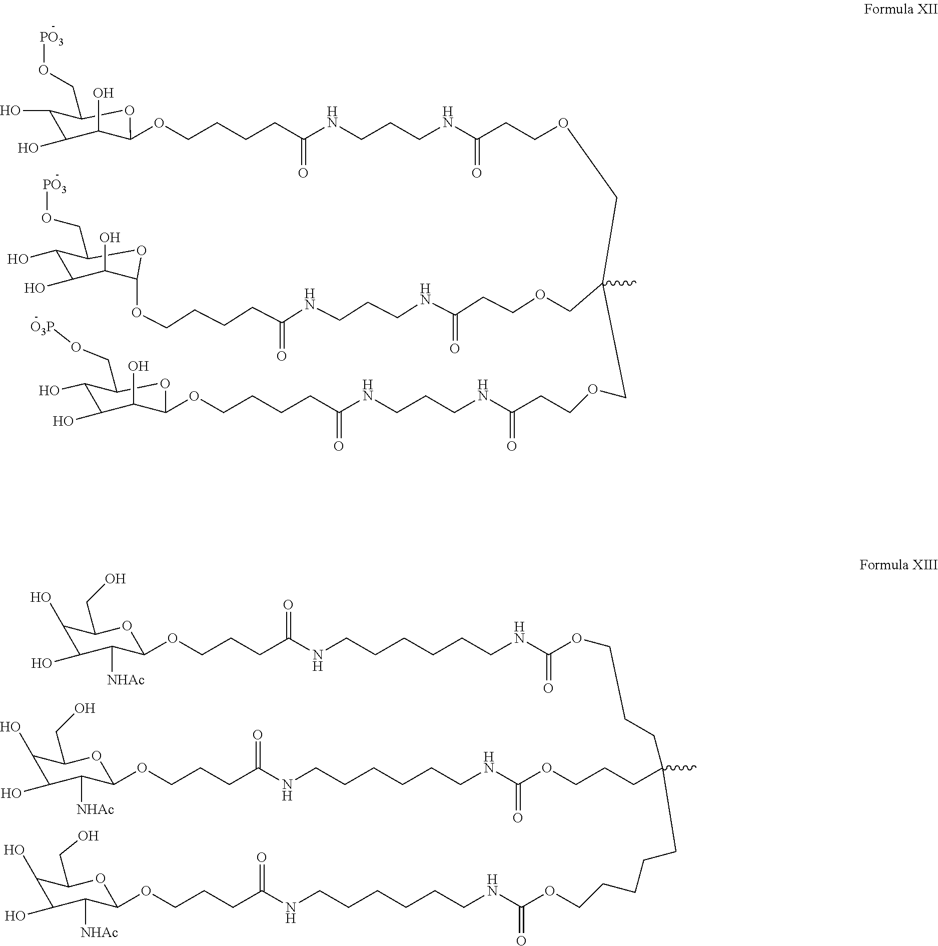

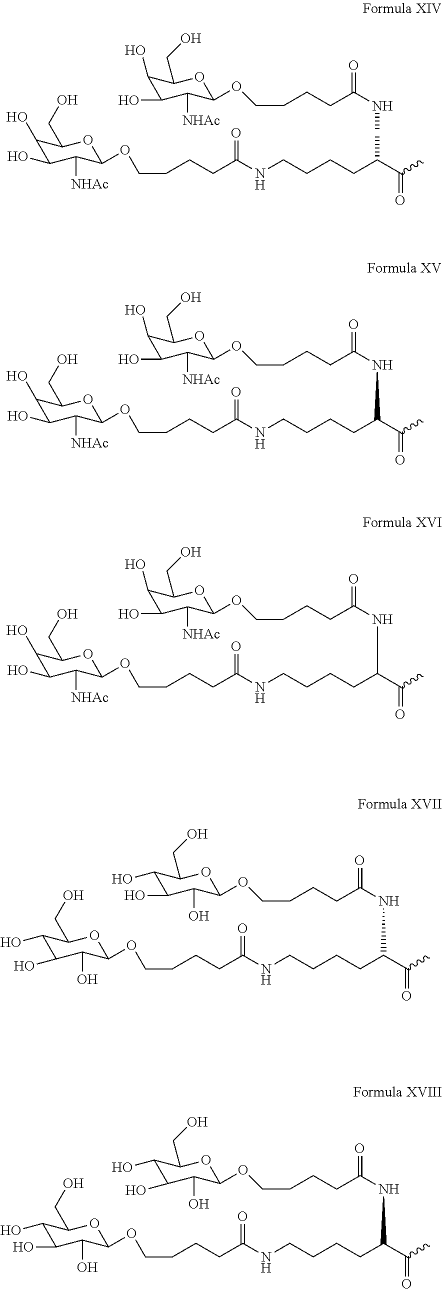

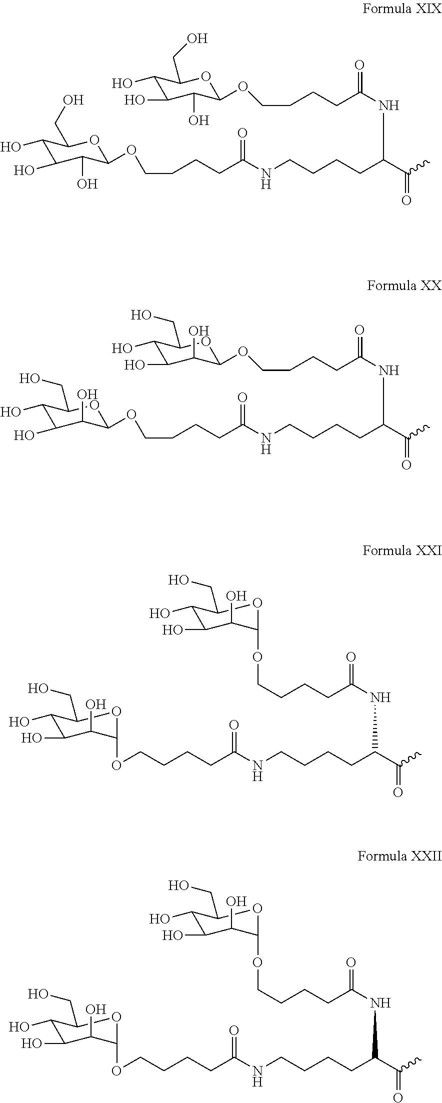

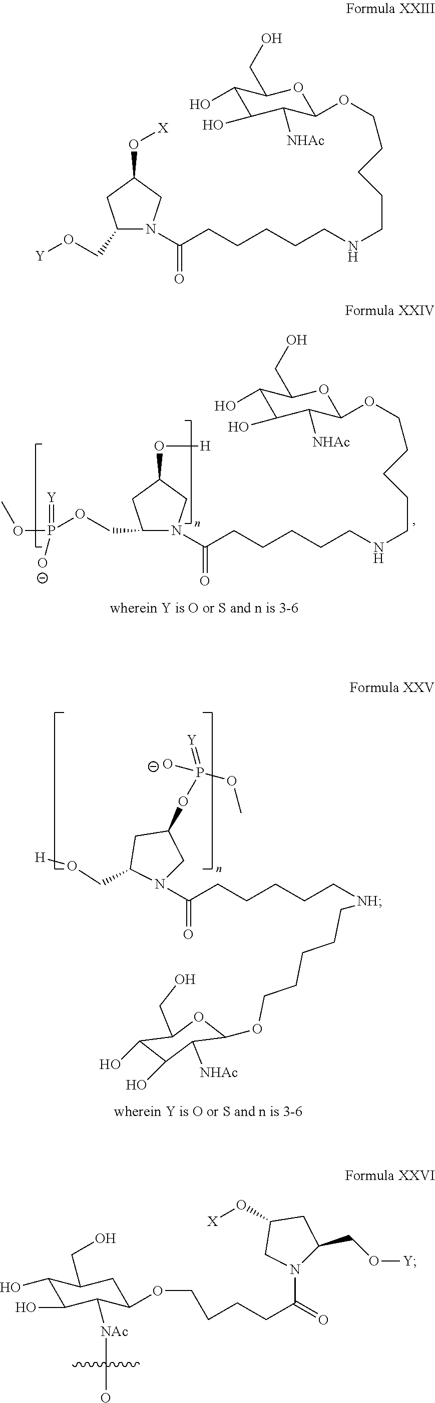

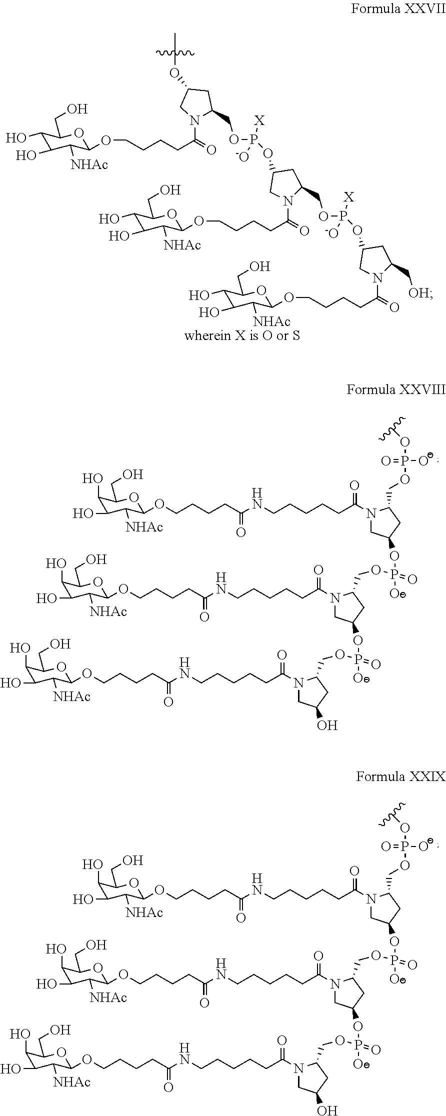

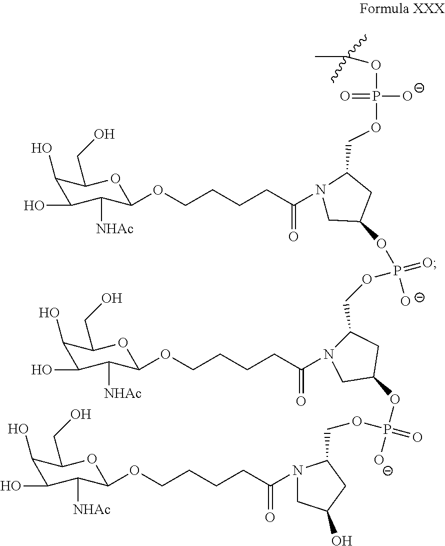 [SMILES: *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCOC3OC(CO)C(O)C(O)C3NC(C)=O)CN2C(=O)CCCCOC2OC(CO)C(O)C(O)C2NC(C)=O)CN1C(=O)CCCCOC1OC(CO)C(O)C(O)C1NC(C)=O]

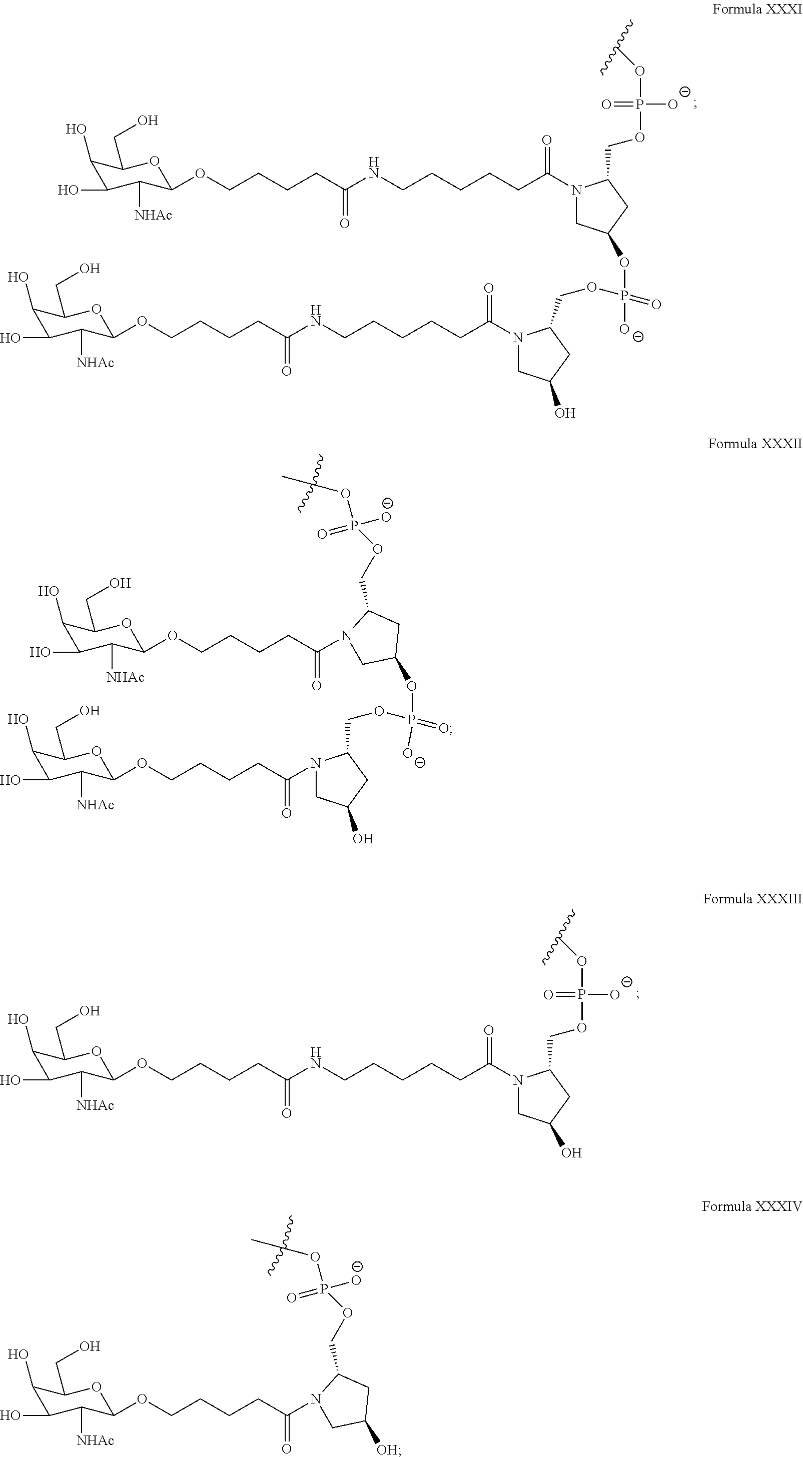 [SMILES: *OP(=O)([O-])OC[C@@H]1C[C@@H](OP(=O)([O-])OC[C@@H]2C[C@@H](O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](O)CN2C(=O)CCCCOC2OC(CO)C(O)C(O)C2NC(C)=O)CN1C(=O)CCCCOC1OC(CO)C(O)C(O)C1NC(C)=O ; *OP(=O)([O-])OC[C@@H]1C[C@@H](O)CN1C(=O)CCCCCNC(=O)CCCCOC1OC(CO)C(O)C(O)C1NC(C)=O ; *OP(=O)([O-])OC[C@@H]1C[C@@H](O)CN1C(=O)CCCCOC1OC(CO)C(O)C(O)C1NC(C)=O]

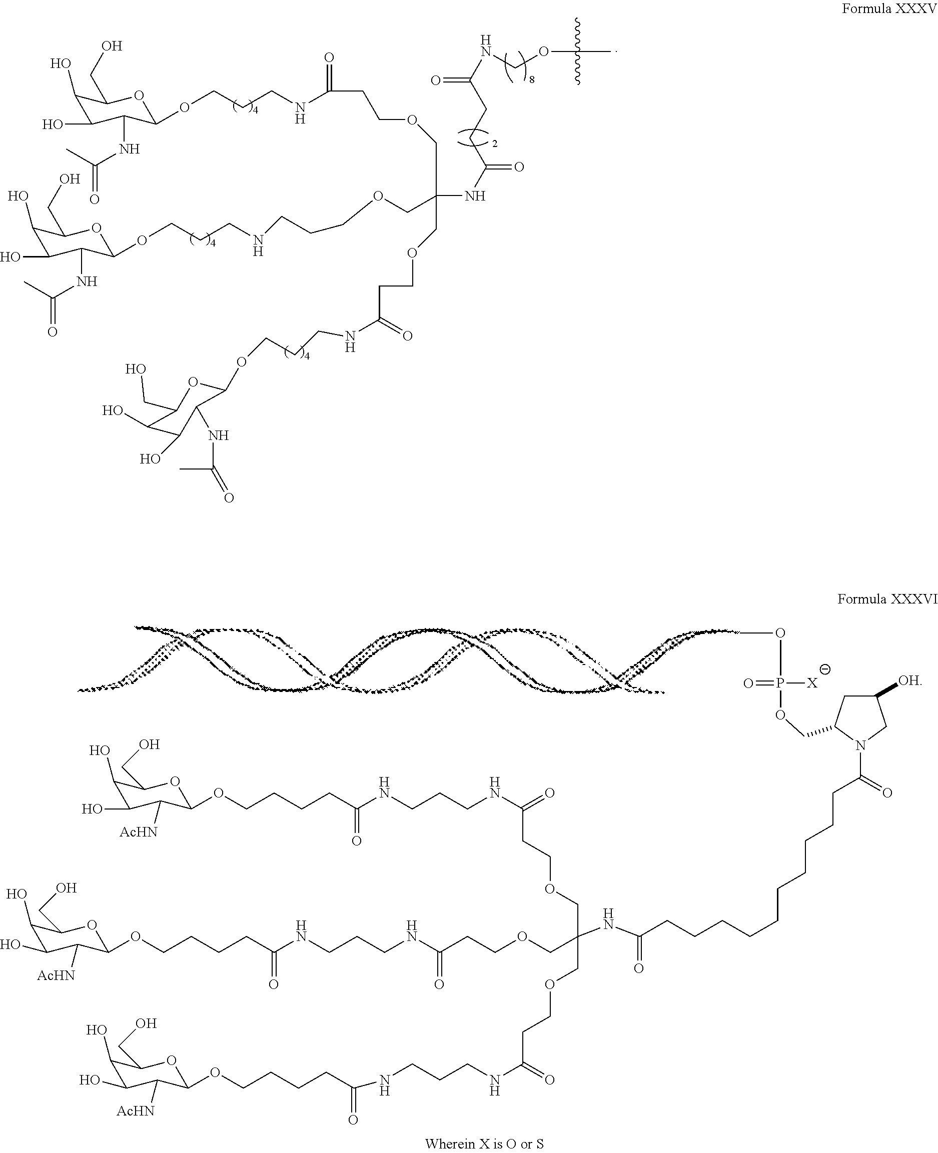

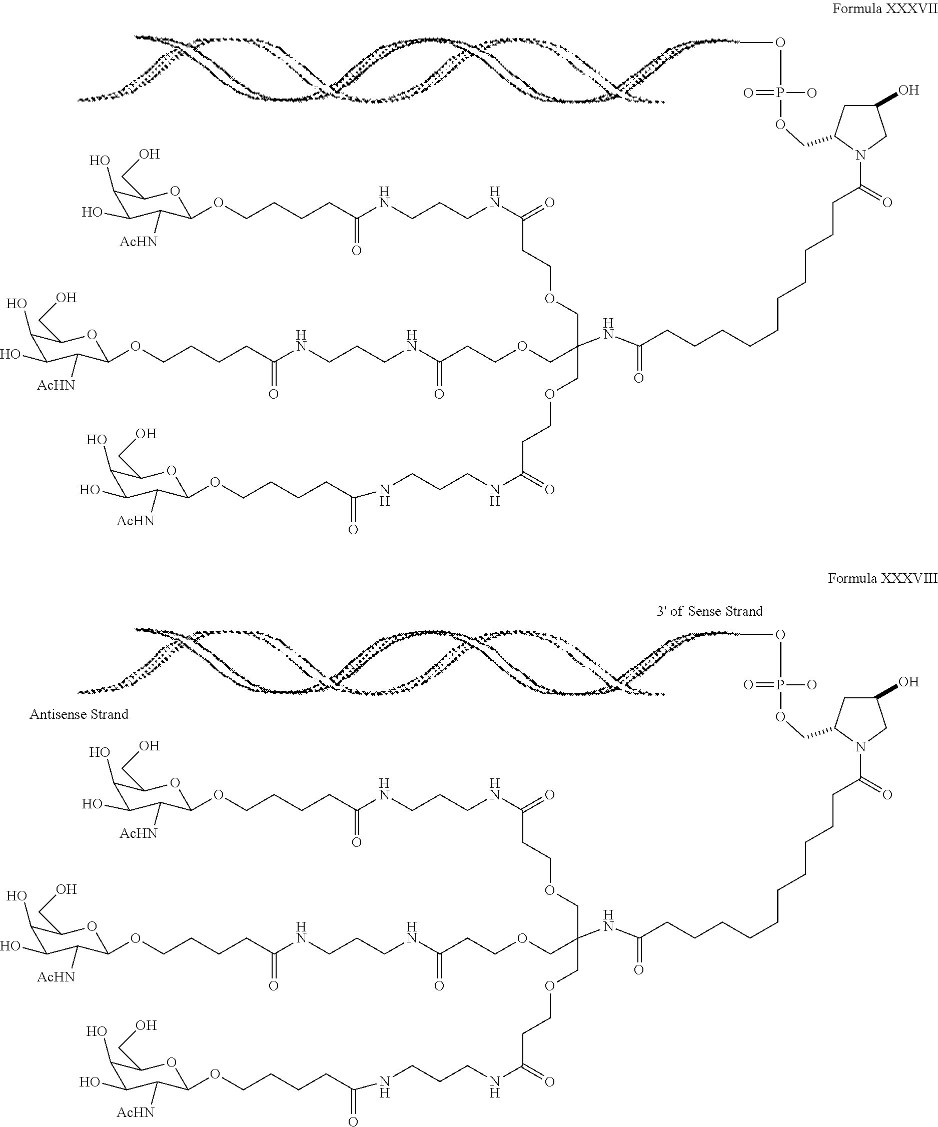 [SMILES: COP(=O)(O)OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O ; COP(=O)(O)OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

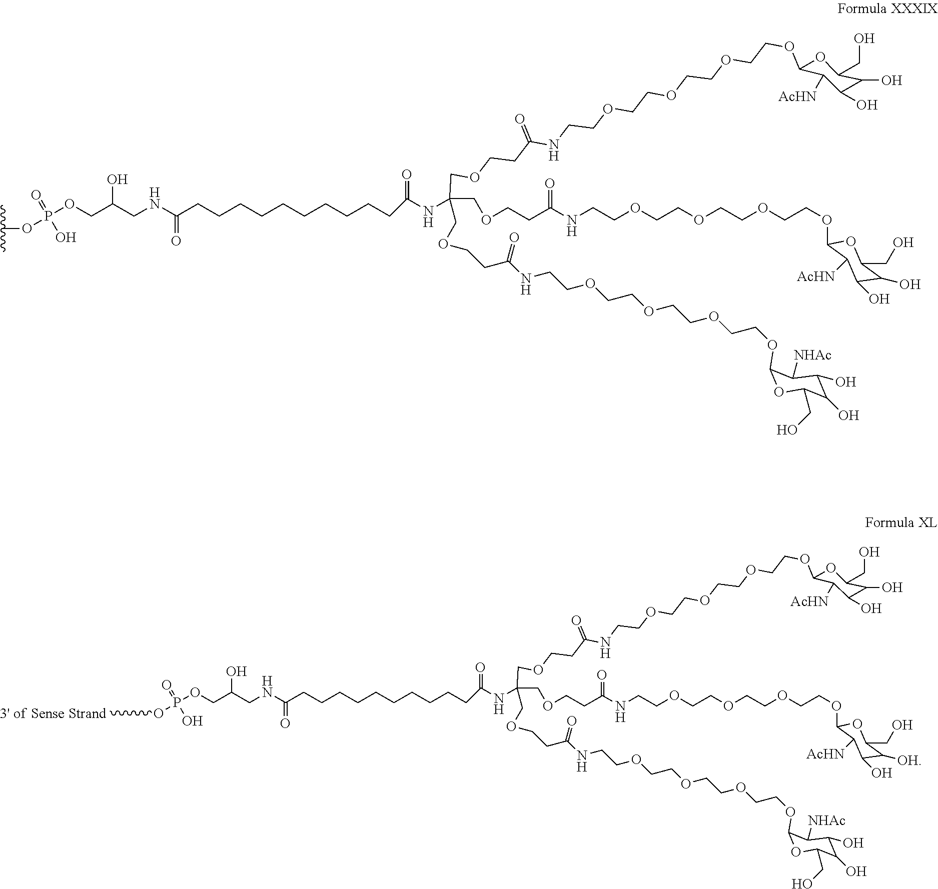

In one embodiment, the targeting moiety comprises the GalNAc targeting moiety and linker set forth below in any one of Formulas I.

In one embodiment, the targeting moiety comprises the GalNAc targeting moiety set forth in Formula I. In one embodiment, the targeting moiety comprises the GalNAc targeting moiety and linker set forth below in Formula II.

In specific embodiments, the targeting moiety comprises the GalNAc targeting moiety set forth in Formula XXXVI. In specific embodiments, the targeting moiety comprises the GalNAc targeting moiety set forth in Formula XXXVII. In specific embodiments, the targeting moiety comprises the GalNAc targeting moiety set forth in Formula XXXVIII.

In specific embodiments, the targeting moiety comprises the GalNAc targeting moiety set forth in Formula XXXIX. In specific embodiments, the targeting moiety comprises the GalNAc targeting moiety set forth in Formula XL.

In specific preferred embodiments, the targeting moiety comprises the GalNAc targeting moiety set forth in Formula XXXIX, wherein the dsRNA agent is operably connected to the targeting moiety via the 3′ end of the sense strand. In specific embodiments, the targeting moiety comprises the GalNAc targeting moiety set forth in Formula XXXIX, wherein the dsRNA agent is operably connected to the targeting moiety via the 5′ end of the sense strand.

In some embodiments, the targeting moiety comprises the GalNAc targeting moiety set forth in Formula XXXIX, wherein the dsRNA agent is operably connected to the targeting moiety via the 3′ end of the antisense strand. In some embodiments, the targeting moiety comprises the GalNAc targeting moiety set forth in Formula XXXIX, wherein the dsRNA agent is operably connected to the targeting moiety via the 5′ end of the antisense strand.

In one embodiment, the targeting moiety comprises N-[tris(GalNAc)-amido-dodecanoyl)]-4-hydroxyprolinol [Hyp-(GalNAc-alkyl)3]. In one embodiment, the targeting moiety comprises (2S,4R)-1-[29-[[2-(acetylamino)-2-deoxy-β-D-galactopyranosyl]oxy]-14,14-bis[[3-[[3-[[5-[[2-(acetylamino)-2-deoxy-β-D-galactopyranosyl]oxy]-1-oxopentyl]amino]propyl]amino]-3-oxopropoxy]methyl]-1,12,19,25-tetraoxo-16-oxa-13,20,24-triazanonacos-1-yl]-4-hydroxy-2-hydroxymethylpyrrolidine.

### 4.4.2 Linkers

In some embodiments, the agent (e.g., described herein) (e.g., RNAi agent, dsRNA agent, antisense strand, sense strand) is directly attached to the heterologous moiety (e.g., targeting moiety) (e.g., directly attached through a single chemical bond). In some embodiments, the agent (e.g., described herein) (e.g., RNAi agent, dsRNA agent, antisense strand, sense strand) is indirectly attached to the heterologous moiety (e.g., targeting moiety). In some embodiments, the agent (e.g., described herein) (e.g., RNAi agent, dsRNA agent, antisense strand, sense strand) is indirectly attached to the heterologous moiety via a linker.

Suitable linkers for use in the conjugates described herein are known in the art and can be evaluated by a person of ordinary skill in the art using standard methods. Exemplary linkers and components thereof for use in the conjugates described herein are also described below.

Linkers typically comprise a direct bond or an atom such as oxygen or sulfur, a unit such as NR8, C(O), C(O)NH, SO, SO2, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkynyl, alkynylheteroarylalkenyl, alkylheterocyclylalkyl, alkylhererocyclylalkynyl, alkylheterocyclylalkenyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

In some embodiments, the linker comprises ethylene glycol (e.g., triethylene glycol), nucleosides, or amino acid units. In some embodiments, the linker comprises one or more groups selected from alkyl, amino, oxo, amide, disulfide, polyethylene glycol, ether, thioether, and hydroxylamino. In some embodiments, the linker comprises groups selected from alkyl, amino, oxo, amide and ether groups. In some embodiments, the linker comprises groups selected from alkyl and amide groups. In some embodiments, the linker comprises groups selected from alkyl and other groups. In some embodiments, the linker comprises at least one phosphorus moiety. In some embodiments, the linker comprises at least one phosphate group. In some embodiments, the linker comprises at least one neutral linking group. Exemplary linkers include but are not limited to triethylene glycol (TEG), pyrrolidine, 8-amino-3,6-dioxaoctanoic acid (ADO), succinimidyl 4-(N-maleimidomethyl) cyclohexane-1-carboxylate (SMCC), 6-aminohexanoic acid (AHEX or AHA). Additional exemplary linkers include but are not limited to substituted or unsubstituted Ci-Cw alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In some embodiments, the linker is bifunctional. In general, a bifunctional linker comprises at least two functional groups. One of the functional groups is selected to react with a particular site on an agent (e.g., described herein) and the other is selected to react with a heterologous moiety (e.g., described herein). Examples of functional groups used in a bifunctional linkers include but are not limited to electrophiles for reacting with nucleophilic groups and nucleophiles for reacting with electrophilic groups. In some embodiments, bifunctional linking moieties comprise one or more groups selected from amino, hydroxyl, carboxylic acid, thiol, alkyl, alkenyl, and alkynyl.

In some embodiments, the linker is a monovalent linker, a bivalent linker, a trivalent linker, or a tetravalent linker. In some embodiments, the linker comprises or consists of the linker set forth above in Formula I.

In specific embodiments, the linker comprises triethylene glycol (TEG). In specific embodiments, the linker consists of triethylene glycol (TEG). In specific embodiments, the linker is triethylene glycol (TEG).

### 4.4.2.1 Cleavable Linkers

In some embodiments, the linker is non-cleavable. In some embodiments, the linker is cleavable. Cleavable linkers contain at least one (or a plurality of) cleavable bonds that are susceptible to one or more cleavage agent. Exemplary classes of cleavable linkers include, but are not limited to, redox cleavable linkers, phosphate based cleavable linkers, acid cleavable linkers, ester-based cleavable linkers, and peptide-based cleavable linkers. In certain embodiments, a cleavable bond is selected from among: an amide, an ester, an ether, one or both esters of a phosphodiester, a phosphate ester, a carbamate, or a disulfide.

Cleavable linkers may be advantageous when a stable conjugate is desired under a first set of conditions but under a second set of conditions it is advantageous to release the agent (e.g., described herein) from the heterologous moiety (e.g., described herein). For example, in some embodiments, it may be desirable to have a sufficiently stable conjugate outside of a cell (e.g., within a subject (e.g., within the blood or serum of a subject)), and upon entry into a cell (e.g., a target cell (e.g., a target cell within a subject)) have the linker cleaved to release the agent (e.g., described herein) from the heterologous moiety (e.g., described herein). In some embodiments, the linker is not cleaved (or is cleaved at a lower rate) under a first condition relative to under a second condition. In some embodiments, the first condition is within the blood (e.g., of a subject) (or in an in vitro system sufficient to mimic the conditions of the blood within a subject) and the second condition is with a cell (e.g., a cell within a subject) (or in an in vitro system sufficient to mimic the conditions of a cell within a subject).

The suitability of a cleavable linker can be assessed by standard methods known in the art. In general, the suitability of a cleavable linker can be evaluated by testing the ability of a cleavage agent (or condition) to cleave the candidate linker (e.g., the cleavage bond(s)). In some embodiments, it may be desirable to further test the ability of the linker to resist cleavage under a certain condition (e.g., within the blood or serum of subject, when in contact with a non-target cell, tissue, organ).

In some embodiments, the linker is a redox cleavable linker that is cleaved upon reduction or oxidation. An example of a reductively cleavable linker is a disulphide (—S—S—) containing linker. Redox cleavable linkers can be evaluated using methods analogous to those described above.

In some embodiments, the linker is a phosphate-based cleavable linker. A phosphate-based cleavable linker is cleaved by agents that degrade or hydrolyze the phosphate group. For example, in cells, enzymes such as phosphatases are capable of cleaving phosphate groups. Examples of phosphate-based linkers include those comprising any of the following —O—P(O)(ORk)-O—, —OP(S)(ORk)-O—, —O—P(S)(SRk)-O—, —S—P(O)(ORk)-O—, —O—P(O)(ORk)-S—, —S—P(O)(ORk)-S—, —OP(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Exemplary embodiments include are —OP(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, or —O—P(S)(H)—S—. Phosphate based cleavable linker can be evaluated using methods analogous to those described above.

In some embodiments, the linker is an acid cleavable linker. An acid cleavable linker is cleaved under acidic conditions. For example, in some embodiments the acid cleavable linker can be cleaved in an acidic environment with a pH of about 6.5 or less (e.g., about 6.0, 5.5, 5.0, or less). In some embodiments the acid cleavable linker can be cleaved by enzymes that can act as a general acid. In a cell (e.g., within a subject), specific low pH organelles, such as endosomes and lysosomes can provide a cleaving environment for acid cleavable linkers. Examples of acid cleavable linkers include but are not limited to hydrazones, esters, and esters of amino acids. Acid cleavable groups can have the general formula —C═NN—, C(O) O, or —OC(O). Acid cleavable linkers can be evaluated using methods analogous to those described above.

In some embodiments, the linker is an ester-based cleavable linker. An ester-based cleavable linker is cleaved by enzymes such as esterases and amidases in cells. Examples of ester-based cleavable include, but are not limited to, esters of alkylene, alkenylene and alkynylene groups. The cleavable bonds of ester cleavable linkers have the general formula —C(O)O— or —OC(O)—. Ester-based cleavable linkers can be evaluated using methods analogous to those described above.

In some embodiments, the linker is a peptide-based cleavable linker. A peptide-based cleavable linker is cleaved by enzymes such as peptidases and proteases (e.g., present in cells (e.g., cells within a subject)). Peptide-based cleavable linkers comprise peptide bonds formed between amino acids to yield polypeptides (e.g., dipeptides, tripeptides, etc.). As known in the art, peptide bonds. The peptide bonds (i.e., the amide bond) of the peptide linker is generally the site of cleavage. Peptide-based cleavable linkers can be evaluated using methods analogous to those described above.

### 4.4.3 Orientation

The heterologous moiety may be attached at any suitable position to the agent (e.g., described herein) (e.g., RNAi agent, dsRNA agent, antisense strand, sense strand).

In some embodiments, the heterologous moiety is conjugated to the 5′ end of the agent (e.g., described herein) (e.g., RNAi agent, dsRNA agent, antisense strand, sense strand). In some embodiments, the heterologous moiety is conjugated to the 3′ end of the agent (e.g., described herein) (e.g., RNAi agent, dsRNA agent, antisense strand, sense strand). In some embodiments, a first heterologous moiety is conjugated to the 5′ end of the agent (e.g., described herein) (e.g., RNAi agent, dsRNA agent, antisense strand, sense strand) and a second heterologous moiety is conjugated to the 3′ end of the agent (e.g., described herein) (e.g., RNAi agent, dsRNA agent, antisense strand, sense strand). The first and second heterologous moieties can be the same or different. In some embodiments, the heterologous moiety is conjugated to an internal site of the agent (e.g., described herein) (e.g., RNAi agent, dsRNA agent, antisense strand, sense strand).

In some embodiments, the agent (e.g., RNAi agent, dsRNA agent) comprises an antisense strand. In some embodiments, the heterologous moiety is conjugated to the 5′ end of the antisense strand. In some embodiments, the heterologous moiety is conjugated to the 3′ end of the antisense strand. In some embodiments, a first heterologous moiety is conjugated to the 5′ end of the antisense strand and a second heterologous moiety is conjugated to the 3′ end of the antisense strand. The first and second heterologous moieties can be the same or different. In some embodiments, the heterologous moiety is conjugated to an internal site of the antisense strand.

In some embodiments, the agent (e.g., RNAi agent, dsRNA agent) comprises a sense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand.

The heterologous moiety may be attached to the 3′ end of the sense and/or antisense strand. The heterologous moiety may be attached to the 5′ end of the sense and/or antisense strand. The heterologous moiety may be attached to at an internal site of the sense and/or antisense strand. The heterologous moiety may be attached to the 3′ end of the sense and antisense strand. The heterologous moiety may be attached to the 5′ end of the sense and antisense strand. The heterologous moiety may be attached to at an internal site of the sense and antisense strand.

In some embodiments, the agent (e.g., RNAi agent) comprises a dsRNA agent comprising a sense strand and an antisense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand.

In some embodiments, a first heterologous moiety is conjugated to the 5′ end of the sense strand and a second heterologous moiety is conjugated to the 5′ end of the antisense strand. In some embodiments, a first heterologous moiety is conjugated to the 3′ end of the sense strand and a second heterologous moiety is conjugated to the 3′ end of the antisense strand. In some embodiments, a first heterologous moiety is conjugated to the 5′ end of the sense strand and a second heterologous moiety is conjugated to the 3′ end of the antisense strand. In some embodiments, a first heterologous moiety is conjugated to the 3′ end of the sense strand and a second heterologous moiety is conjugated to the 5′ end of the antisense strand. The first and second heterologous moieties can be the same or different.

In some embodiments, a first heterologous moiety is conjugated to an internal site of the sense strand and a second heterologous moiety is conjugated to the 5′ end of the antisense strand. In some embodiments, a first heterologous moiety is conjugated to an internal site of the sense strand and a second heterologous moiety is conjugated to the 3′ end of the antisense strand. In some embodiments, a first heterologous moiety is conjugated to an internal site of the antisense strand and a second heterologous moiety is conjugated to the 3′ end of the antisense strand. In some embodiments, a first heterologous moiety is conjugated to an internal site of the antisense strand and a second heterologous moiety is conjugated to the 5′ end of the antisense strand. The first and second heterologous moieties can be the same or different.

### 4.4.4 Exemplary Conjugates

The structure of exemplary conjugates comprising a GalNAc targeting moiety and a linker via a linker for conjugation to an agent (e.g., RNAi agent, dsRNA agent, antisense strand, sense strand) described herein is provided below.

For example, in some embodiments, the agent (e.g., RNAi agent, dsRNA agent, antisense strand, sense strand) is conjugated to a GalNAc targeting moiety through a linker e.g., as shown in the following schematic, wherein X is O or S (and further described in § 4.4.2).

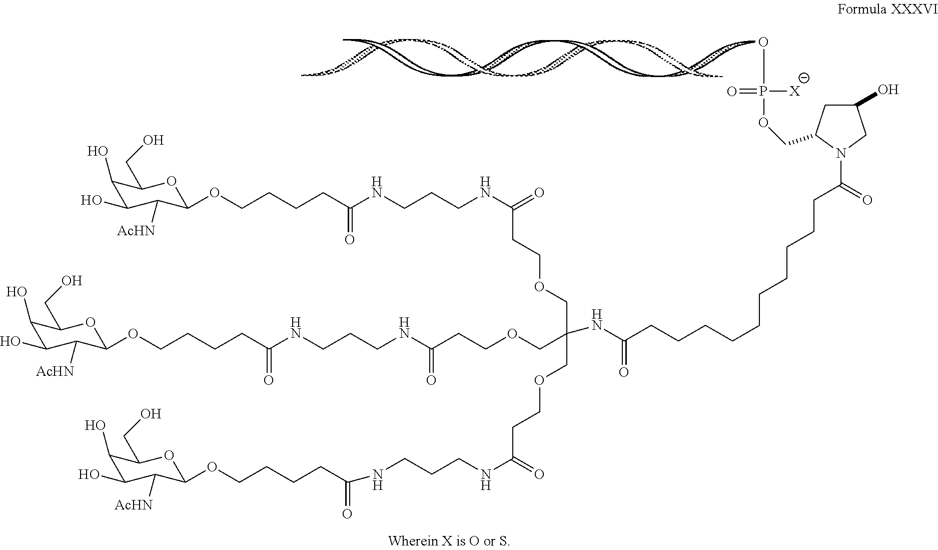

In some embodiments, the agent (e.g., RNAi agent, dsRNA agent, antisense strand, sense strand) is conjugated to the GalNAc targeting moiety as shown in the schematic below:

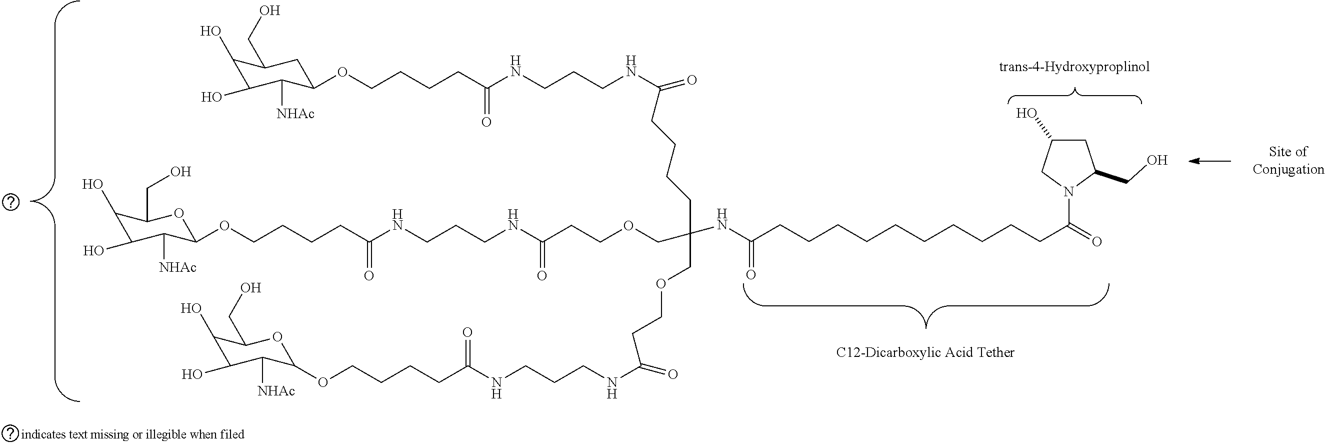

In some embodiments, the GalNAc targeting moiety and linker comprises that set forth below in Formula XXXVI wherein one of X or Y is a polynucleotide, the other is a hydrogen.

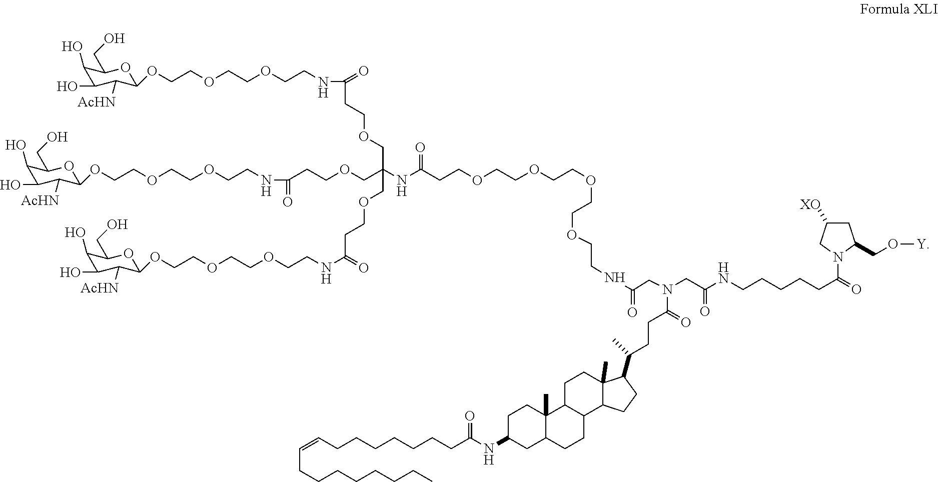

In some embodiments, the GalNAc targeting moiety and linker comprises that set forth in any one of the following formulas, wherein in any of the following formulas wherein one of X or Y is a polynucleotide, the other is a hydrogen:

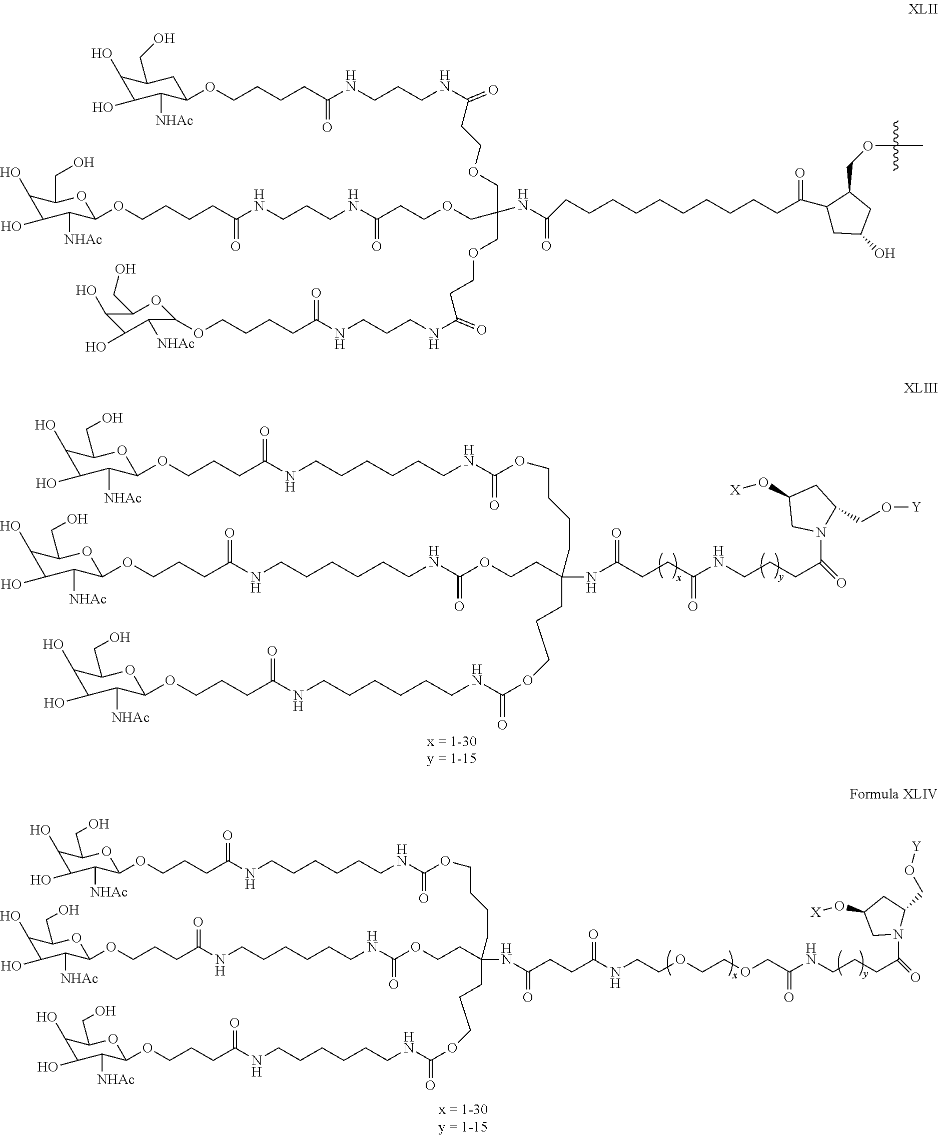

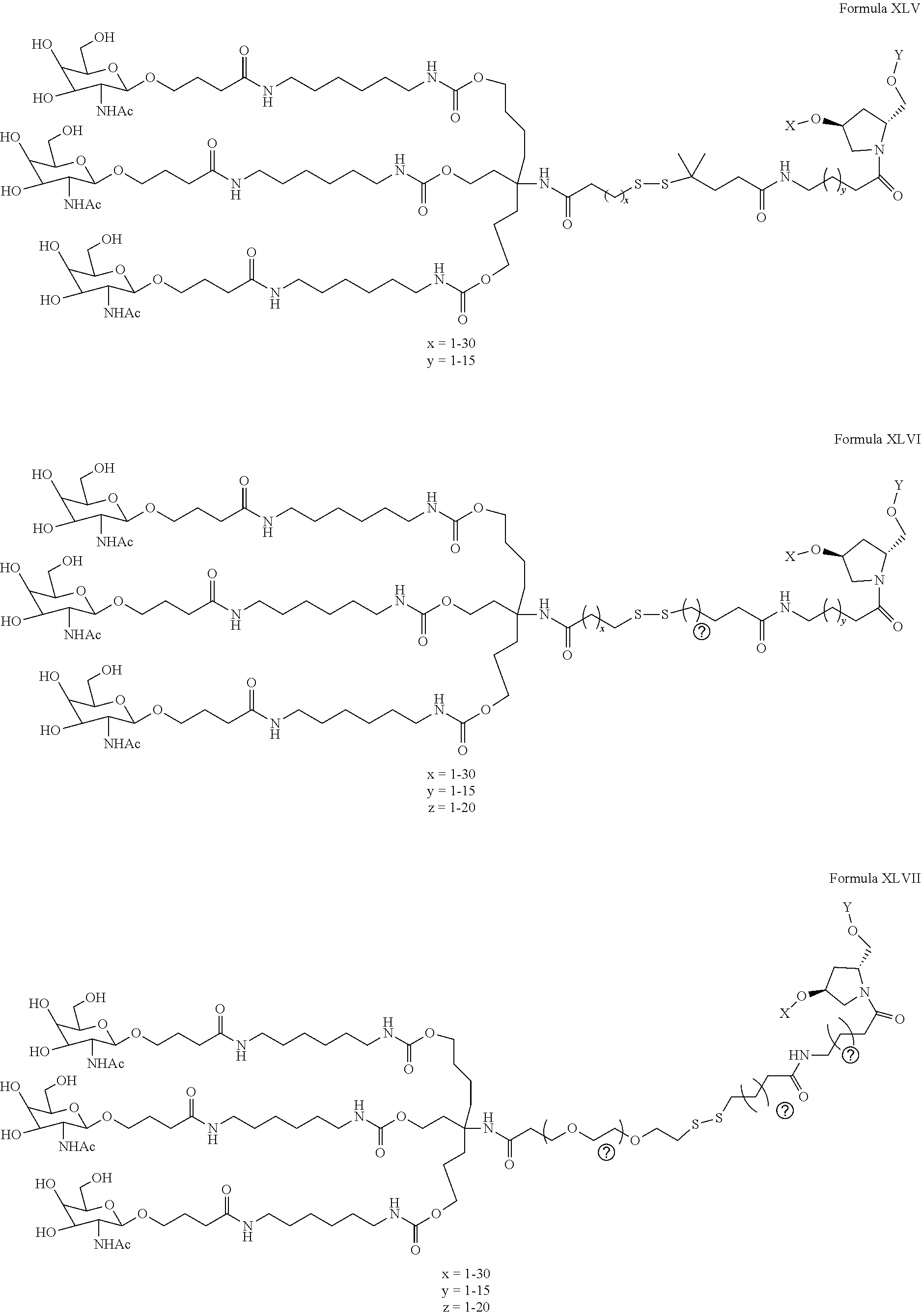

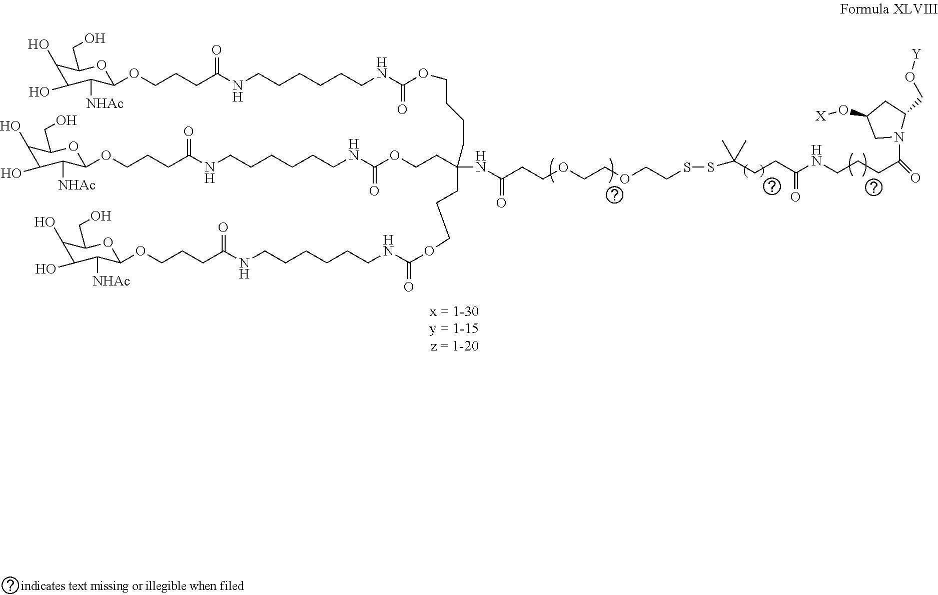

In specific embodiments, the targeting moiety comprises the GalNAc targeting moiety and linker set forth below in Formula I:

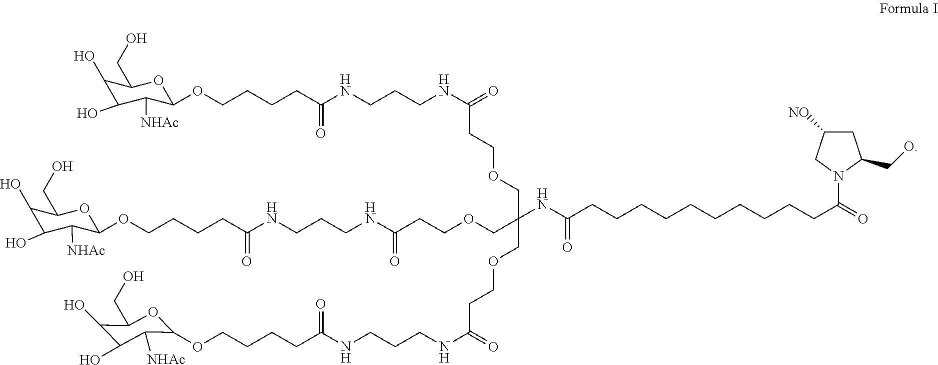

In specific embodiments, the targeting moiety comprises the GalNAc targeting moiety and linker set forth below in Formula XXXIX.

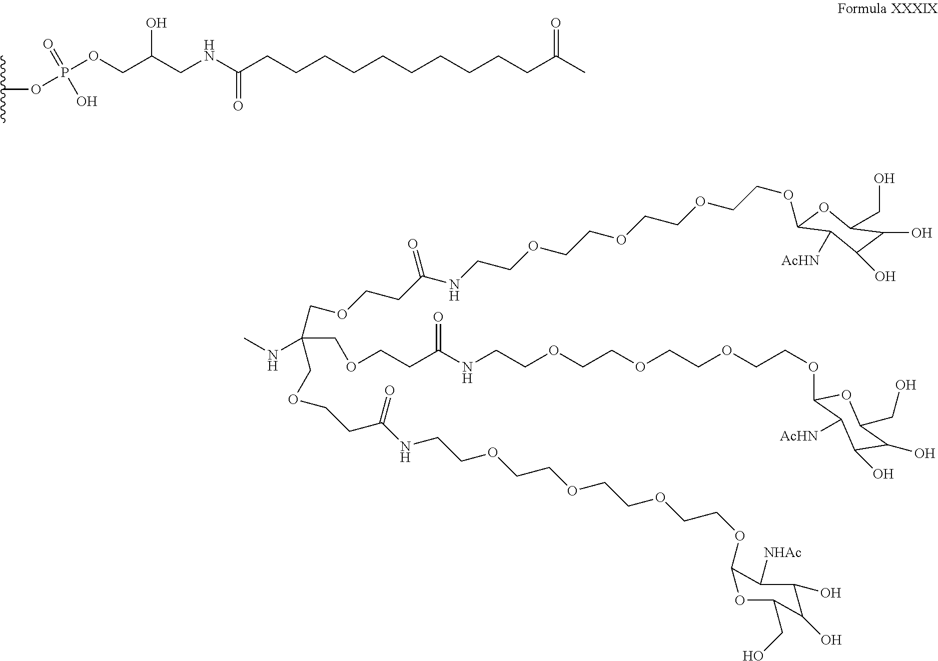

In specific preferred embodiments, the targeting moiety comprises the GalNAc targeting moiety and linker set forth below in Formula XXXIX, wherein the dsRNA agent is operably connected to the targeting moiety via the 3′ end of the sense strand. In specific embodiments, the targeting moiety comprises the GalNAc targeting moiety and linker set forth below in Formula XXXIX, wherein the dsRNA agent is operably connected to the targeting moiety via the 5′ end of the sense strand.

In some embodiments, the targeting moiety comprises the GalNAc targeting moiety and linker set forth below in Formula XXXIX, wherein the dsRNA agent is operably connected to the targeting moiety via the 3′ end of the antisense strand. In some embodiments, the targeting moiety comprises the GalNAc targeting moiety and linker set forth below in Formula XXXIX, wherein the dsRNA agent is operably connected to the targeting moiety via the 5′ end of the antisense strand.

In specific embodiments, the targeting moiety comprises the GalNAc targeting moiety and linker set forth below in Formula XL.

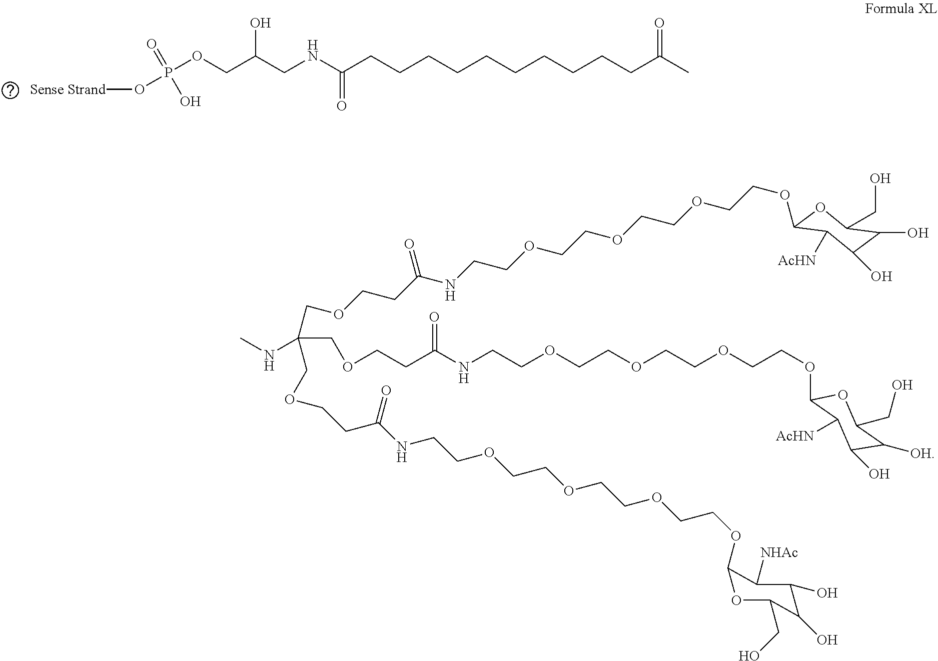

In specific preferred embodiments, the targeting moiety comprises the GalNAc targeting moiety and linker set forth below in Formula XL, wherein the dsRNA agent is operably connected to the targeting moiety via the 3′ end of the sense strand.

Further exemplary select conjugates are provided in Table 11 below.

TABLE 11
Exemplary dsRNA Agent Conjugates.

| | Corresponding | Corresponding | | | | |
|---|---|---|---|---|---|---|
| | Unconjugated | Unconjugated | | | | |
| | Unmodified | Modified | | | | |
| dsRNA | dsRNA | dsRNA | Sense | SEQ | Antisense | SEQ |
| Agent | Agent ID | Agent ID | Sequence | ID | Sequence | ID |
| ID | (Table 2) | (Table 3) | 5′ to 3′ | NO | 5′ to 3′ | NO |
| 483 | 129 | 479 | csasguauCfuAf | 1193 | (vinu)sCfsgag | 1188 |
| | | | AfUfauaagcucg | | CfuuauauuAfgA | |
| | | | a (GalNAc-TEG) | | fuacugsasc | |
| 484 | 129 | 481 | csasguauCfuAf | 1193 | (vinu)sCfsgag | 1190 |
| | | | AfUfauaagcucg | | CfuuauauuAfga | |
| | | | a (GalNAc-TEG) | | uacugsasc | |
| 485 | 172 | 482 | csasgaCfaguAf | 1194 | (vinu)sUfsauc | 1191 |
| | | | CfAfggcuagaua | | UfagccuguAfcu | |
| | | | a (GalNAc-TEG) | | gucugscsa | |
| 486 | 135 | 480 | usasauauAfaGf | 1195 | (vinu)sCfsaaa | 1189 |
| | | | CfUfcggaguuug | | CfuccgagcUfuA | |
| | | | a (GalNAc-TEG) | | fuauuasgsa | |
| 487 | 173 | 409 | asgsacagUfaCf | 1196 | (vinu)sUfsuau | 1062 |
| | | | AfGfgcuagauaa | | CfuAfGfccugUf | |
| | | | a (GalNAc-TEG) | | aCfugucusgsc | |

The nucleotide modifications set forth in Table 10, utilize the following abbreviations set forth in Table 4 above. As recited above, “(GalNAc-TEG)” recited in Table 10 indicates the conjugation of the GalNAc-TEG (see, e.g., Formula XXXIX above) to the 3′ end of the sense strand of each dsRNA agent set forth in Table 10. The corresponding base modified dsRNA agent (set forth in Table 3) as well as the corresponding base unmodified dsRNA agent (set forth in Table 2) for each of the GalNAc-dsRNA agents 483-487 is also set forth in Table 11.

In specific embodiments, the conjugate comprises the sense strand and the antisense strand of a dsRNA agent conjugate set forth in Table 11. In specific embodiments, the conjugate comprises the sense strand and the antisense strand of any one of dsRNA agent conjugates 483-487 set forth in Table 11. In specific embodiments, the conjugate comprises the sense strand and the antisense strand of dsRNA agent conjugates 483. In specific embodiments, the conjugate comprises the sense strand and the antisense strand of dsRNA agent conjugates 484. In specific embodiments, the conjugate comprises the sense strand and the antisense strand of dsRNA agent conjugates 485. In specific embodiments, the conjugate comprises the sense strand and the antisense strand of dsRNA agent conjugates 486. In specific embodiments, the conjugate comprises the sense strand and the antisense strand of dsRNA agent conjugates 487.

In specific embodiments, the conjugate is substantially similar to any one of dsRNA agent conjugates 483-487 set forth in Table 11. In specific embodiments, the conjugate is substantially similar to dsRNA agent conjugate 483. In specific embodiments, the conjugate is substantially similar to dsRNA agent conjugate 484. In specific embodiments, the conjugate is substantially similar to dsRNA agent conjugate 485. In specific embodiments, the conjugate is substantially similar to dsRNA agent conjugate 486. In specific embodiments, the conjugate is substantially similar to dsRNA agent conjugate 487.

In specific embodiments, the conjugate is any one of dsRNA agent conjugates 483-487 set forth in Table 11. In specific embodiments, the conjugate is dsRNA agent conjugate 483. In specific embodiments, the conjugate is dsRNA agent conjugate 484. In specific embodiments, the conjugate is dsRNA agent conjugate 485. In specific embodiments, the conjugate dsRNA agent conjugate 486. In specific embodiments, the conjugate is dsRNA agent conjugate 487.

In specific embodiments, the dsRNA agent is a conjugate comprising a sense strand and an antisense stand, wherein the sense strand comprises the sequence set forth in SEQ ID NO: 1193; and the antisense strand comprises the sequence set forth in SEQ ID NO: 1188. In specific embodiments, the dsRNA agent is a conjugate comprising a sense strand and an antisense stand, wherein the sense strand comprises the sequence set forth in SEQ ID NO: 1193; and the antisense strand comprises the sequence set forth in SEQ ID NO: 1190. In specific embodiments, the dsRNA agent is a conjugate comprising a sense strand and an antisense stand, wherein the sense strand comprises the sequence set forth in SEQ ID NO: 1194; and the antisense strand comprises the sequence set forth in SEQ ID NO: 1191. In specific embodiments, the dsRNA agent is a conjugate comprising a sense strand and an antisense stand, wherein the sense strand comprises the sequence set forth in SEQ ID NO: 1195; and the antisense strand comprises the sequence set forth in SEQ ID NO: 1189. In specific embodiments, the dsRNA agent is a conjugate comprising a sense strand and an antisense stand, wherein the sense strand comprises the sequence set forth in SEQ ID NO: 1196; and the antisense strand comprises the sequence set forth in SEQ ID NO: 1062.

In specific embodiments, the dsRNA agent is a conjugate comprising a sense strand and an antisense stand, wherein the sense strand consists of the sequence set forth in SEQ ID NO: 1193; and the antisense strand consists of the sequence set forth in SEQ ID NO: 1188. In specific embodiments, the dsRNA agent is a conjugate comprising a sense strand and an antisense stand, wherein the sense strand consists of the sequence set forth in SEQ ID NO: 1193; and the antisense strand consists of the sequence set forth in SEQ ID NO: 1190. In specific embodiments, the dsRNA agent is a conjugate comprising a sense strand and an antisense stand, wherein the sense strand consists of the sequence set forth in SEQ ID NO: 1194; and the antisense strand consists of the sequence set forth in SEQ ID NO: 1191. In specific embodiments, the dsRNA agent is a conjugate comprising a sense strand and an antisense stand, wherein the sense strand consists of the sequence set forth in SEQ ID NO: 1195; and the antisense strand consists of the sequence set forth in SEQ ID NO: 1189. In specific embodiments, the dsRNA agent is a conjugate comprising a sense strand and an antisense stand, wherein the sense strand consists of the sequence set forth in SEQ ID NO: 1196; and the antisense strand consists of the sequence set forth in SEQ ID NO: 1062.

In specific embodiments, the dsRNA agent is a conjugate comprising the sense strand set forth in SEQ ID NO: 1193; and the antisense strand set forth in SEQ ID NO: 1188. In specific embodiments, the dsRNA agent is a conjugate comprising the sense strand set forth in SEQ ID NO: 1193; and the antisense strand set forth in SEQ ID NO: 1190. In specific embodiments, the dsRNA agent is a conjugate comprising the sense strand set forth in SEQ ID NO: 1194; and the antisense strand set forth in SEQ ID NO: 1191. In specific embodiments, the dsRNA agent is a conjugate comprising the sense strand set forth in SEQ ID NO: 1195; and the antisense strand set forth in SEQ ID NO: 1189. In specific embodiments, the dsRNA agent is a conjugate comprising the sense strand set forth in SEQ ID NO: 1196; and the antisense strand set forth in SEQ ID NO: 1062.

### 4.5 Activity of RNAi Agents & Conjugates Thereof

In some embodiments, the agent (e.g., RNAi agent, e.g., dsRNA agent) inhibits expression of a target gene (e.g., CIDEB). In some embodiments, the agent (e.g., RNAi agent, e.g., dsRNA agent) inhibits expression of a target gene (e.g., CIDEB) in a cell. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.). In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 30%, 40%, 50%, 55%, 60%, 65%, 70%, 75%, 80%, 85%, 90%, 95%, 96%, 97%, 98%, 99%, or 100%. In some embodiments, the agent (e.g., RNAi agent, e.g., dsRNA agent) mediates degradation of a target mRNA (e.g., a CIDEB (e.g., hCIDEB) mRNA). In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 50%, 55%, 60%, 65%, 70%, 75%, 80%, 85%, 90%, 95%, 96%, 97%, 98%, 99%, or 100%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 75%, 80%, 85%, 90%, 95%, 96%, 97%, 98%, 99%, or 100%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 90%, 95%, 96%, 97%, 98%, 99%, or 100%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 50%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 75%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 80%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 90%. In some embodiments, the agent (e.g., RNAi agent, e.g., dsRNA agent) inhibits expression of a target gene (e.g., CIDEB) in a cell in a subject (e.g., a mammalian subject, e.g., a primate, human, non-human primate, mouse, rat, etc.) by at least about 95%.

Any one or more of the above activities can be evaluated in vitro, ex vivo, or in vivo. Any one or more of the above activities can be evaluated by standard methods known in the art. For example, by PCR (e.g., qPCR), branched DNA assays, or by a protein-based methods (such as immunofluorescence analysis (using, e.g., western blotting or flow cytometric techniques). In some embodiments, inhibition of gene (e.g., CIDEB (e.g., hCIDEB)) expression is determined by qPCR.

### 4.6 Methods of Making RNAi Agents & Conjugates Thereof

An agent (e.g., described herein) (e.g., RNAi agent, dsRNA agent, antisense strand, sense strand) can be synthesized by standard methods known in the art (e.g., chemical synthesis (e.g., solid phase synthesis)). See, e.g., “Current protocols in nucleic acid chemistry,” Beaucage, S. L. et al. (Edrs.), John Wiley & Sons, Inc., New York, N.Y., USA, and See, e.g., Dong Y, Siegwart D J, Anderson D G. Strategies, design, and chemistry in siRNA delivery systems. Adv Drug Deliv Rev. 2019 April; 144:133-147. doi: 10.1016/j.addr.2019.05.004. Epub 2019 May 15. PMID: 31102606; PMCID: PMC6745264, the entire contents of each of which is incorporated by reference herein for all purposes. As such, further provided herein are methods of making an agent described herein) (e.g., RNAi agent, dsRNA agent, antisense strand, sense strand).

For example, single stranded nucleic acid molecules (e.g., described herein) (e.g., antisense strands, sense strands) can be prepared using solution-phase or solid-phase organic synthesis or both. dsRNA agents (e.g., described herein) can be prepared using a two-step procedure, wherein the individual strands of the dsRNA agent are prepared separately and subsequently annealed. The individual strands of the dsRNA agent can be prepared using solution-phase or solid-phase organic synthesis or both. Regardless of the method of synthesis, the agents (e.g., described herein) (e.g., dsRNA agents described herein) can be prepared in a solution (e.g., an aqueous or organic solution) that is appropriate for formulation. For example, the dsRNA agent can be precipitated and redissolved in pure double-distilled water, and lyophilized. The lyophilized dsRNA agent can be resuspended in a solution appropriate for the intended formulation process.

Likewise, conjugates (e.g., described herein) can be synthesized utilizing standard methods known in the art. See, e.g., Dong Y, Siegwart D J, Anderson D G. Strategies, design, and chemistry in siRNA delivery systems. Adv Drug Deliv Rev. 2019 April; 144:133-147. doi: 10.1016/j.addr.2019.05.004. Epub 2019 May 15. PMID: 31102606; PMCID: PMC6745264, the entire contents of which is incorporated herein by reference for all purposes. A person of ordinary skill in the art can determine the appropriate conjugation method based on e.g., the heterologous moiety and the agent to be conjugated. For example, standard conjugation methods include, e.g., parallel synthesis methods and linear synthesis methods.

### 4.7 Vectors

In some embodiments, one or more of the agents described herein (e.g., RNAi agents, double stranded RNA (dsRNA) agents, sense strands, antisense strands) (see, e.g., §§ 4.2, 4.3) are contained in a vector (e.g., a non-viral vector (e.g., a plasmid), a viral vector). Thus, in one aspect, also provided herein are vectors (e.g., non-viral vectors (e.g., plasmids) viral vectors) comprising one or more agent described herein (e.g., RNAi agent, double stranded RNA (dsRNA) agent, sense strand, antisense strand) (see, e.g., §§ 4.2, 4.3). Such vectors can be easily manipulated by methods well known to the ordinary person of skill in the art. The vector used can be any vector that is suitable for cloning nucleic acid molecules that can be used for transcription of the nucleic acid molecule of interest (e.g., an agent described herein (e.g., RNAi agent, double stranded RNA (dsRNA) agent, sense strand, antisense strand) (see, e.g., §§ 4.2, 4.3).

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, anellovectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an agent described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

In some embodiments, the vector is a plasmid. A person of ordinary skill in the art is aware of suitable plasmids for expression of the DNA of interest. For example, Suitable plasmid DNA may be generated to allow efficient production of the encoded RNA in cell lines, e.g., in insect cell lines, for example using vectors as described in WO2009150222A2 and as defined in PCT claims 1 to 33, the disclosure relating to claims 1 to 33 of WO2009150222A2 the entire contents of which is incorporated by reference herein for all purposes.

### 4.8 Carriers

In some embodiments, one or more of the agents described herein (e.g., RNAi agents, double stranded RNA (dsRNA) agents, sense strands, antisense strands (or a conjugate comprising the same)) or a vector comprising any of the foregoing is formulated within one or more carrier.

Therefore, further provided herein are carriers comprising any one or more of the agents described herein (e.g., RNAi agents, double stranded RNA (dsRNA) agents, sense strands, antisense strands (or a conjugate comprising the same)) or a vector comprising any of the foregoing.

Any of the foregoing (e.g., one or more of the agents described herein (e.g., RNAi agents, double stranded RNA (dsRNA) agents, sense strands, antisense strands (or a conjugate comprising the same)) or a vector comprising any of the foregoing) can be encapsulated within a carrier, chemically conjugated to a carrier, associated with the carrier. In this context, the term “associated” refers to the essentially stable combination of an agent described herein (or a conjugate comprising the same) (or a vector comprising the same) with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of an agent described herein (or a conjugate comprising the same) (or a vector comprising the same) into a carrier (e.g., a lipid-based carrier, e.g., an LNP, liposome, lipoplex, and/or nanoliposome) wherein the agent described herein (or the conjugate comprising the same) (or the vector comprising the same) is entirely contained within the interior space of the carrier (e.g., the lipid-based carrier, e.g., the LNP, liposome, lipoplex, and/or nanoliposome).

Exemplary carriers includes, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 4.8.1.

### 4.8.1 Lipid Based Carriers/Lipid Nanoformulations

In some embodiments, an agent described herein (e.g., RNAi agent, double stranded RNA (dsRNA) agent, sense strand, antisense strand (or a conjugate comprising the same)) (or a vector comprising any of the foregoing) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, an agent described herein (e.g., RNAi agent, double stranded RNA (dsRNA) agent, sense strand, antisense strand (or a conjugate comprising the same)) (or a vector comprising any of the foregoing) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., RNAi agent, double stranded RNA (dsRNA) agent, sense strand, antisense strand (or a conjugate comprising the same)) (or a vector comprising any of the foregoing) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., RNAi agent, double stranded RNA (dsRNA) agent, sense strand, antisense strand (or a conjugate comprising the same)) (or a vector comprising any of the foregoing) is encapsulated in LNPs (e.g., as described herein).

The agents (e.g., RNAi agents, double stranded RNA (dsRNA) agents, sense strands, antisense strands (or a conjugate comprising the same)) (or a vector comprising any of the foregoing) described herein may be completely or partially located in the interior space of the LNPs, liposomes, lipoplexes, and/or nanoliposomes, within the lipid layer/membrane, or associated with the exterior surface of the lipid layer/membrane. One purpose of incorporating an agent described herein (e.g., RNAi agent, double stranded RNA (dsRNA) agent, sense strand, antisense strand (or a conjugate comprising the same)) (or a vector comprising any of the foregoing) into LNPs, liposomes, lipoplexes, and/or nanoliposomes is to protect the agent from an environment which may contain enzymes or chemicals or conditions that degrade the agent from molecules or conditions that cause the rapid excretion of the agent. Moreover, incorporating an agent described herein (e.g., RNAi agent, double stranded RNA (dsRNA) agent, sense strand, antisense strand (or a conjugate comprising the same)) (or a vector comprising any of the foregoing) into LNPs, liposomes, lipoplexes, and/or nanoliposomes may promote the uptake of the agent, and hence, may enhance the therapeutic effect of the agent (e.g., RNAi agent, double stranded RNA (dsRNA) agent, sense strand, antisense strand (or a conjugate comprising the same)) (or a vector comprising any of the foregoing). Accordingly, incorporating an agent described herein (e.g., RNAi agent, double stranded RNA (dsRNA) agent, sense strand, antisense strand (or a conjugate comprising the same)) (or a vector comprising any of the foregoing), into LNPs, liposomes, lipoplexes, and/or nanoliposomes may be particularly suitable for a pharmaceutical composition described herein, e.g., for intramuscular and/or intradermal administration.

In some embodiments, an agent described herein (e.g., RNAi agent, double stranded RNA (dsRNA) agent, sense strand, antisense strand (or a conjugate comprising the same)) (or a vector comprising any of the foregoing) is formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more agent described herein (e.g., RNAi agent, double stranded RNA (dsRNA) agent, sense strand, antisense strand (or a conjugate comprising the same)) (or a vector comprising any of the foregoing), or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more agent described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 4.8.1.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Diolcoyl-3-Dimethylammonium-propane (DODAP), 1,2-Diolcoylcarbamyl-3-Dimethylammonium-propane (DOCDAP), 1,2-Dilincoyl-3-Dimethylammonium-propane (DLINDAP), 3-Dimethylamino-2-(Cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis, cis-9′,12′-octadecadienoxy) propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

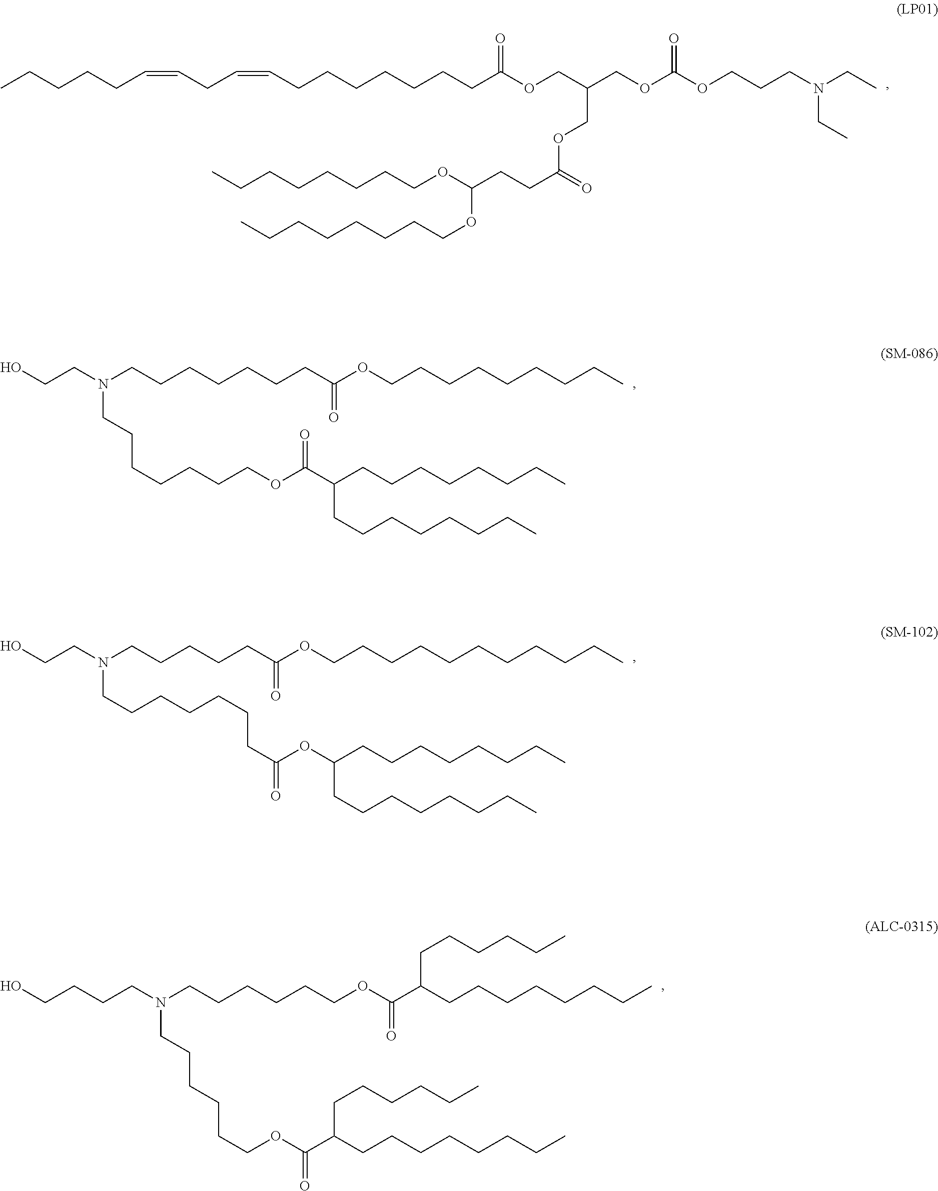 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCOC(=O)C(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

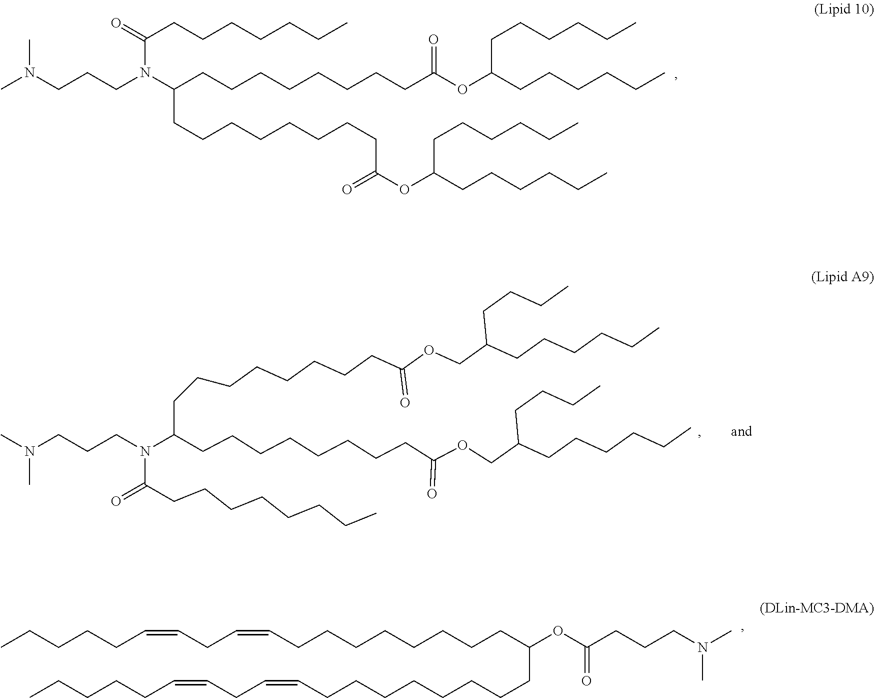

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or 1-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-O13 or 503-O13 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 4.8.1.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or olcoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyristoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US 2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 4.8.1.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, the entire contents of each of which are incorporated by reference herein for all purposes. Additional exemplary sterols include phytosterols, including those described in Eygeris et al. (2020), Nano Lett. 2020; 20(6): 4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 4.8.1.4 Polymers and Polyethylene Glycol (PEG)-Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy (Polyethylene glycol)-(mPEG 350 PE); 1,2-Diacyl-sn-Glycero-3-Phosphoethanolamine-N-[Methoxy (Polyethylene glycol)-550] (mPEG 550 PE); 1,2-Diacyl-sn-Glycero-3-Phosphoethanolamine-N-[Methoxy (Polyethylene glycol)-750] (mPEG 750 PE); 1,2-Diacyl-sn-Glycero-3-Phosphoethanolamine-N-[Methoxy (Polyethylene glycol)-1000] (mPEG 1000 PE); 1,2-Diacyl-sn-Glycero-3-Phosphoethanolamine-N-[Methoxy (Polyethylene glycol)-2000] (mPEG 2000 PE); 1,2-Diacyl-sn-Glycero-3-Phosphoethanolamine-N-[Methoxy (Polyethylene glycol)-3000] (mPEG 3000 PE); 1,2-Diacyl-sn-Glycero-3-Phosphoethanolamine-N-[Methoxy (Polyethylene glycol)-(mPEG 5000 PE); N-Acyl-Sphingosine-1-[Succinyl(Methoxy Polyethylene Glycol) 750] (mPEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

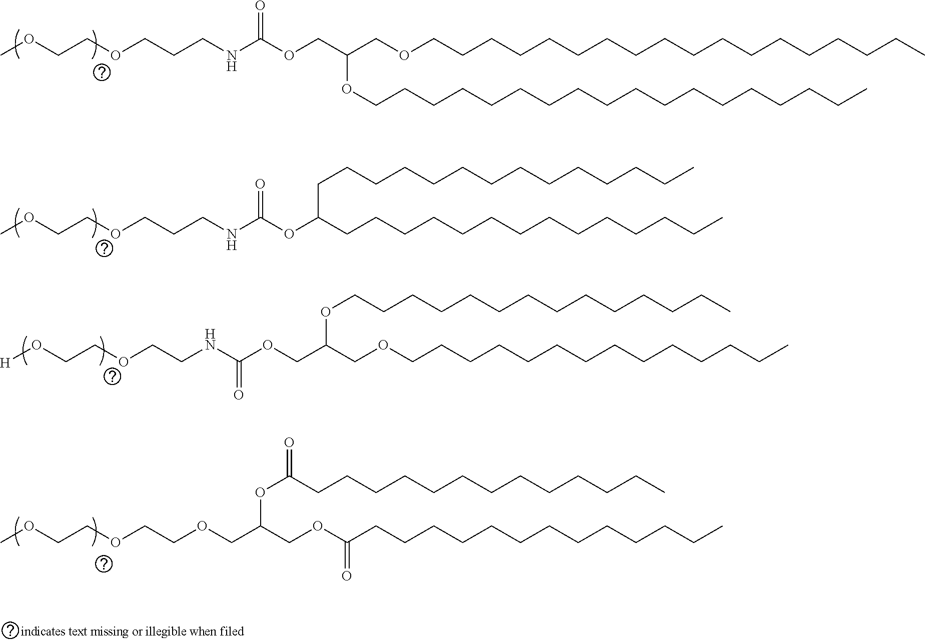

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), the entire contents of each of which are incorporated by reference herein for all purposes.

### 4.8.1.5 Percentages of Lipid Nanoformulation Components

In some embodiments, the lipid-based carrier (or lipid nanoformulation) comprises one of more of the agents described herein (e.g., RNAi agents, double stranded RNA (dsRNA) agents, sense strands, antisense strands (or a conjugate comprising the same)) (or a vector comprising any of the foregoing), optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises an agent (e.g., an agent described herein (e.g., RNAi agent, double stranded RNA (dsRNA) agent, sense strand, antisense strand (or a conjugate comprising the same)) (or a vector comprising any of the foregoing))). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises an agent described herein (e.g., RNAi agent, double stranded RNA (dsRNA) agent, sense strand, antisense strand (or a conjugate comprising the same)) (or a vector comprising any of the foregoing) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the agent may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises an agent described herein (e.g., RNAi agent, double stranded RNA (dsRNA) agent, sense strand, antisense strand (or a conjugate comprising the same)) (or a vector comprising any of the foregoing) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the agent may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises an agent described herein (e.g., RNAi agent, double stranded RNA (dsRNA) agent, sense strand, antisense strand (or a conjugate comprising the same)) (or a vector comprising any of the foregoing) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the agent may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises an agent described herein (e.g., RNAi agent, double stranded RNA (dsRNA) agent, sense strand, antisense strand (or a conjugate comprising the same)) (or a vector comprising any of the foregoing) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the agent may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises an agent described herein (e.g., RNAi agent, double stranded RNA (dsRNA) agent, sense strand, antisense strand (or a conjugate comprising the same)) (or a vector comprising any of the foregoing) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the agent may be at least 70%.

In some embodiments, the lipid-based carrier (or lipid nanoformulation) comprises (i) an agent described herein (e.g., RNAi agent, double stranded RNA (dsRNA) agent, sense strand, antisense strand (or a conjugate comprising the same)) (or a vector comprising any of the foregoing); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) an agent described herein (e.g., RNAi agent, double stranded RNA (dsRNA) agent, sense strand, antisense strand (or a conjugate comprising the same)) (or a vector comprising any of the foregoing); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the agent (e.g., an encapsulated agent such as an agent described herein (e.g., RNAi agent, double stranded RNA (dsRNA) agent, sense strand, antisense strand (or a conjugate comprising the same)) (or a vector comprising any of the foregoing) can be varied as desired. For example, the total lipid components to the agent (mass or weight) ratio can be from about 10:1 to about 30:1. In some embodiments, the total lipid components to the agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the agent can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of an agent (e.g., an agent described herein), describes the amount of agent that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, close to 100%). The encapsulation efficiency may be measured, for example, by comparing the amount of agent in a solution containing the liposome or LNP before and after breaking up the liposome or LNP with one or more organic solvents or detergents. An anion exchange resin may be used to measure the amount of free agent in a solution. Fluorescence may be used to measure the amount of free agent in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 4.9 Host Cells

In one aspect, provided herein are host cells comprising any one more agent described herein (e.g., an RNAi agent, a dsRNA agent, an antisense strand, a sense strand) (see, e.g., §§ 4.2, 0); a vector described herein (see, e.g., § 4.6); a conjugate described herein (see, e.g., § 4.4); a carrier described herein (see, e.g., § 4.8); or any combination thereof. In some embodiments, the host cell comprises an RNAi agent described herein, a dsRNA agent described herein, an antisense strand described herein, and/or a sense strand described herein. In some embodiments, the host cell is in vitro, ex vivo, or in vivo.

### 4.10 Pharmaceutical Compositions

In one aspect, provided herein are pharmaceutical compositions comprising any one more agent described herein (e.g., an RNAi agent, a dsRNA agent, an antisense strand, a sense strand) (see, e.g., §§ 4.2, 4.3); a vector described herein (see, e.g., § 4.6); a conjugate described herein (see, e.g., § 4.4); a carrier described herein (see, e.g., § 4.8); and/or a host cell described herein (see, e.g., § 4.9); or any combination thereof; and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing any one more agent described herein (e.g., an RNAi agent, a dsRNA agent, an antisense strand, a sense strand) (see, e.g., §§ 4.2, 4.3); a vector described herein (see, e.g., § 4.6); a conjugate described herein (see, e.g., § 4.4); a carrier described herein (see, e.g., § 4.8); and/or a host cell described herein (see, e.g., § 4.9); and formulating it into a pharmaceutically acceptable composition by the addition of one or more pharmaceutically acceptable excipient.

Also provided herein are pharmaceutical compositions comprising any one more agent described herein (e.g., an RNAi agent, a dsRNA agent, an antisense strand, a sense strand); vector described herein; conjugate described herein; carrier described herein; and/or cell described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal.

In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 4.11 Methods of Use

Provided herein are various methods of utilizing any one more agent described herein (e.g., an RNAi agent, a dsRNA agent, an antisense strand, a sense strand) (see, e.g., §§ 4.2, 4.3); a vector described herein (see, e.g., § 4.6); a conjugate described herein (see, e.g., § 4.4); a carrier described herein (see, e.g., § 4.8); a host cell described herein (see, e.g., § 4.9); and/or a pharmaceutical composition described herein (see, e.g., § 4.10); or any combination thereof.

In some aspects, the methods described herein comprise administering one or more of the foregoing to a subject. Exemplary subjects include mammals, e.g., humans, non-human mammals, e.g., non-human primates. In some embodiments, the subject is a human.

The dosage of any of the foregoing, to be administered to a subject in accordance with any of the methods described herein can be determined in accordance with standard techniques known to those of ordinary skill in the art, including the route of administration, the age and weight of the subject.

In some embodiments, the agent described herein (e.g., an RNAi agent, a dsRNA agent, an antisense strand, a sense strand) (see, e.g., §§ 4.2, 4.3) or a conjugate thereof (see, e.g., § 4.4) is administered to a subject at a dose of from about 1-10 mg/kg, 2-10 mg·kg, 3-10 mg/kg, 4-10 mg/kg, 5-10 mg/kg, 6-10 mg/kg, 7-10 mg/kg, 8-10 mg/kg 9-10 mg/kg, 1-5 mg/kg, 1-4 mg·kg, 1-3 mg/kg, 1-2 mg/kg, 1-2.5 mg/kg, 2-5 mg·kg, 2-4 mg/kg, 2-3 mg/kg, or 2-2.5 mg/kg. In some embodiments, the agent described herein (e.g., an RNAi agent, a dsRNA agent, an antisense strand, a sense strand) (see, e.g., §§ 4.2, 4.3) or a conjugate thereof (see, e.g., § 4.4) is administered to a subject at a dose of about 1 mg/kg, 2 mg/kg, 2.5 mg/kg, 3 mg·kg, 4 mg/kg, 5 mg/kg, 6 mg/kg, 7 mg/kg, 8 mg/kg, 9 mg/kg, or 10 mg/kg. In some embodiments, the agent described herein (e.g., an RNAi agent, a dsRNA agent, an antisense strand, a sense strand) (see, e.g., §§ 4.2, 4.3) or a conjugate thereof (see, e.g., § 4.4) is administered to a subject at least once per month, once per month, at least twice per month, twice per month, at least once a week, or once per week.

### 4.11.1 Methods of Delivery

Provided herein are, inter alia, various methods of delivering any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein; or any combination thereof to e.g., a cell, subject, a cell within a subject.

In one aspect, provided herein are methods of delivering any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein; or any combination thereof to a cell, the method comprising introducing into a cell any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein into the cell, to thereby deliver the agent (e.g., RNAi agent, dsRNA agent, antisense strand, sense strand), conjugate, vector, carrier, or pharmaceutical composition into the cell. In some embodiments, the agent (e.g., RNAi agent, dsRNA agent, antisense strand, sense strand), conjugate, vector, carrier, or pharmaceutical composition is introduced in an amount and for a time sufficient to deliver the agent (e.g., RNAi agent, dsRNA agent, antisense strand, sense strand), conjugate, vector, carrier, or pharmaceutical composition into the cell. In some embodiments, the cell is in vitro, ex vivo, or in vivo. In some embodiments, the cell is in vitro or ex vivo. In some embodiments, the cell is in vitro. In some embodiments, the cell is ex vivo. In some embodiments, the cell is in vivo.

In one aspect, provided herein are methods of delivering any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein; or any combination thereof to a subject, the method comprising administering to a subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein into the cell, to thereby deliver the agent (e.g., RNAi agent, dsRNA agent, antisense strand, sense strand), conjugate, vector, carrier, or pharmaceutical composition to the subject. In some embodiments, the agent (e.g., RNAi agent, dsRNA agent, antisense strand, sense strand), conjugate, vector, carrier, or pharmaceutical composition is administered in an amount and for a time sufficient to deliver the agent (e.g., RNAi agent, dsRNA agent, antisense strand, sense strand), conjugate, vector, carrier, or pharmaceutical composition to the subject.

In one aspect, provided herein are methods of delivering any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein; or any combination thereof to a cell within a subject, the method comprising administering to a cell within a subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein into the cell, to thereby deliver the agent (e.g., RNAi agent, dsRNA agent, antisense strand, sense strand), conjugate, vector, carrier, or pharmaceutical composition to the cell within the subject. In some embodiments, the agent (e.g., RNAi agent, dsRNA agent, antisense strand, sense strand), conjugate, vector, carrier, or pharmaceutical composition is administered in an amount and for a time sufficient to deliver the agent (e.g., RNAi agent, dsRNA agent, antisense strand, sense strand), conjugate, vector, carrier, or pharmaceutical composition to the cell within the subject.

### 4.11.2 Methods of Reducing or Inhibiting CIDEB Expression

In one aspect, provided herein are methods of reducing or inhibiting expression of CIDEB (e.g., hCIDEB) in a cell, the method comprising introducing into the cell any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein, to thereby reduce or inhibit expression of CIDEB (e.g., hCIDEB) in the cell. In some embodiments, the agent (e.g., RNAi agent, dsRNA agent, antisense strand, sense strand), conjugate, vector, carrier, or pharmaceutical composition is introduced in an amount and for a time sufficient to reduce or inhibit expression of CIDEB (e.g., hCIDEB) in the cell. In some embodiments, the cell is in vitro, ex vivo, or in vivo. In some embodiments, the cell is in vitro or ex vivo. In some embodiments, the cell is in vitro. In some embodiments, the cell is ex vivo. In some embodiments, the cell is in vivo.

In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the level of CIDEB (e.g., hCIDEB) expression in the cell is reduced by at least about 50% (e.g., as compared to suitable control) (e.g., as measured by an assay known in the art or described herein, e.g., qPCR). In some embodiments, the level of CIDEB (e.g., hCIDEB) expression in the cell is reduced by at least about 75% (e.g., as compared to suitable control) (e.g., as measured by an assay known in the art or described herein, e.g., qPCR). In some embodiments, the level of CIDEB (e.g., hCIDEB) expression in the cell is reduced by at least about 80% (e.g., as compared to suitable control) (e.g., as measured by an assay known in the art or described herein, e.g., qPCR). In some embodiments, the level of CIDEB (e.g., hCIDEB) expression in the cell is reduced by at least about 90% (e.g., as compared to suitable control) (e.g., as measured by an assay known in the art or described herein, e.g., qPCR). In some embodiments, the level of CIDEB (e.g., hCIDEB) expression in the cell is reduced by at least about 95% (e.g., as compared to suitable control) (e.g., as measured by an assay known in the art or described herein, e.g., qPCR).

In one aspect, provided herein are methods of reducing or inhibiting expression of CIDEB (e.g., hCIDEB) in a cell in a subject, the method comprising administering to a subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein, to thereby reduce or inhibit expression of CIDEB (e.g., hCIDEB) in the cell in the subject. In some embodiments, the agent (e.g., RNAi agent, dsRNA agent, antisense strand, sense strand), conjugate, vector, carrier, or pharmaceutical composition is administered to the subject in an amount and for a time sufficient to reduce or inhibit expression of CIDEB (e.g., hCIDEB) in the cell in the subject. In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR).

In one aspect, provided herein are dsRNA agents, conjugates, vectors, carriers, and pharmaceutical compositions for use in a method of reducing or inhibiting expression of CIDEB (e.g., hCIDEB) in a cell in a subject. In some embodiments, the agent (e.g., RNAi agent, dsRNA agent, antisense strand, sense strand), conjugate, vector, carrier, or pharmaceutical composition is administered to the subject in an amount and for a time sufficient to reduce or inhibit expression of CIDEB (e.g., hCIDEB) in the cell in the subject. In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR).

In one aspect, provided herein are dsRNA agents, conjugates, vectors, carriers, and pharmaceutical compositions for use in a method of reducing or inhibiting expression of CIDEB (e.g., hCIDEB) in a cell in a subject, the method comprising administering to the subject a dsRNA agent described herein, a conjugate described herein, a vector described herein, a carrier described herein, or a pharmaceutical composition described herein, to thereby reduce or inhibit expression of CIDEB (e.g., hCIDEB) in the cell in the subject. In some embodiments, the agent (e.g., RNAi agent, dsRNA agent, antisense strand, sense strand), conjugate, vector, carrier, or pharmaceutical composition is administered to the subject in an amount and for a time sufficient to reduce or inhibit expression of CIDEB (e.g., hCIDEB) in the cell in the subject. In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR).

In one aspect, provided herein are uses of dsRNA agents, conjugates, vectors, carriers, and pharmaceutical compositions in the manufacture of a medicament for the reduction or inhibition of CIDEB (e.g., hCIDEB) expression in a cell in a subject. In some embodiments, the agent (e.g., RNAi agent, dsRNA agent, antisense strand, sense strand), conjugate, vector, carrier, or pharmaceutical composition is administered to the subject in an amount and for a time sufficient to reduce or inhibit expression of CIDEB (e.g., hCIDEB) in the cell in the subject. In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR).

In one aspect, provided herein are uses of dsRNA agents, conjugates, vectors, carriers, and pharmaceutical compositions in the manufacture of a medicament for use in a method of reducing or inhibiting expression of CIDEB (e.g., hCIDEB) in a cell in a subject, the method comprising administering to the subject a dsRNA agent described herein, a conjugate described herein, a vector described herein, a carrier described herein, or a pharmaceutical composition described herein, to thereby reduce or inhibit expression of CIDEB (e.g., hCIDEB) in the cell in the subject. In some embodiments, the agent (e.g., RNAi agent, dsRNA agent, antisense strand, sense strand), conjugate, vector, carrier, or pharmaceutical composition is administered to the subject in an amount and for a time sufficient to reduce or inhibit expression of CIDEB (e.g., hCIDEB) in the cell in the subject. In some embodiments, the level of CIDEB (e.g., hCIDEB) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR).

### 4.11.3 Methods of Treating, Ameliorating, or Preventing a CIDEB Associated Disease

In one aspect, provided herein are methods of treating, ameliorating, or preventing a disease in a subject, the method comprising administering to the subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein, to thereby treat, ameliorate, or prevent a disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the disease in the subject.

In one aspect, provided herein are dsRNA agents, conjugates, vectors, carriers, and pharmaceutical compositions for use in a method of treating, ameliorating, or preventing a CIDEB (e.g., hCIDEB) associated disease in a subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the CIDEB (e.g., hCIDEB) associated disease in the subject.

In one aspect, provided herein are dsRNA agents, conjugates, vectors, carriers, and pharmaceutical compositions for use in a method of treating, ameliorating, or preventing a CIDEB (e.g., hCIDEB) associated disease in a subject, the method comprising administering to the subject a dsRNA agent described herein, a conjugate described herein, a vector described herein, a carrier described herein, or a pharmaceutical composition described herein, to thereby treat, ameliorate, or prevent the CIDEB (e.g., hCIDEB) associated disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the CIDEB (e.g., hCIDEB) associated disease in the subject.

In one aspect, provided herein are uses of dsRNA agents, conjugates, vectors, carriers, and pharmaceutical compositions in the manufacture of a medicament for the treatment, amelioration, or prevention of a CIDEB (e.g., hCIDEB) associated disease in a subject. In some embodiments, the agent (e.g., RNAi agent, dsRNA agent, antisense strand, sense strand), conjugate, vector, carrier, or pharmaceutical composition is administered to the sub eject in an amount and for a time sufficient to treat, ameliorate, or prevent the CIDEB (e.g., hCIDEB) associated disease in the subject.

In one aspect, provided herein are uses of dsRNA agents, conjugates, vectors, carriers, and pharmaceutical compositions in the manufacture of a medicament for use in a method of treating, ameliorating, or preventing a CIDEB (e.g., hCIDEB) associated disease in a subject, the method comprising administering to the subject a dsRNA agent described herein, a conjugate described herein, a vector described herein, a carrier described herein, or a pharmaceutical composition described herein, to thereby treat, ameliorate, or prevent the CIDEB (e.g., hCIDEB) associated disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the CIDEB (e.g., hCIDEB) associated disease in the subject.

In one aspect, provided herein are dsRNA agents, conjugates, vectors, carriers, and pharmaceutical compositions for use in treating a disease in a subject, preferably a CIDEB (e.g., hCIDEB) associated disease, more preferably a liver disease.

In one aspect, provided herein are methods of treating, ameliorating, or preventing a CIDEB (e.g., hCIDEB) associated disease in a subject, the method comprising administering to the subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein, to thereby treat, ameliorate, or prevent the CIDEB (e.g., hCIDEB) associated disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the CIDEB (e.g., hCIDEB) associated disease in the subject.

The following applicable to any of the foregoing methods, in specific embodiments, the treating, ameliorating, or preventing of the CIDEB associated disease is mediated (at least in part) by reducing or inhibiting expression of CIDEB (e.g., hCIDEB) in the subject (e.g., a population of cells within the subject).

The following applicable to any of the foregoing methods, in some embodiments, the CIDEB associated disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the CIDEB associated disease is fatty liver disease, liver inflammation, NASH or NAFLD. In specific embodiments, the CIDEB associated disease is obesity-induced metabolic syndrome, insulin insensitivity, obesity, type-2 diabetes, AFLD, or cirrhosis. In specific embodiments, the CIDEB associated disease is MASLD, MASH, MAFLD, MetALD, SLD, or cryptogenic SLD. See, e.g., Rinella M. et al., A multisociety Delphi consensus statement on new fatty liver disease nomenclature, Hepatology 78(6):p 1966-1986 December 2023, the entire contents of which is incorporated herein by reference for all purposes. In specific embodiments, the CIDEB associated disease is the liver fibrosis, liver inflammation, cirrhosis, liver failure, or jaundice. In specific embodiments, the disease is obesity or type-2 diabetes. In specific embodiments, the CIDEB associated disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the CIDEB associated disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the CIDEB associated disease is atherosclerosis, coronary artery disease, myocardial infarction, or heart failure.

In specific embodiments, the CIDEB associated disease is fatty liver disease, steatotic liver disease, liver inflammation, NASH, NAFLD, MASLD, MASH, MAFLD, MetALD, SLD, or cryptogenic SLD.

### 4.11.4 Methods of Treating, Ameliorating, or Preventing a Liver Disease

In one aspect, provided herein are methods of treating, ameliorate, or preventing a liver disease in a subject, the method comprising administering to the subject any one more agent described herein (e.g., an RNAi agent, a dsRNA agent, an antisense strand, a sense strand); a vector described herein; a conjugate described herein; a carrier described herein; a host cell described herein; and/or a pharmaceutical composition described herein, to thereby treat, ameliorate, or prevent the liver disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the liver disease in the subject.

In one aspect, provided herein are dsRNA agents, conjugates, vectors, carriers, and pharmaceutical compositions for use in a method of treating, ameliorating, or preventing a liver disease in a subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the liver disease in the subject.

In one aspect, provided herein are dsRNA agents, conjugates, vectors, carriers, and pharmaceutical compositions for use in a method of treating, ameliorating, or preventing a liver disease in a subject, the method comprising administering to the subject a dsRNA agent described herein, a conjugate described herein, a vector described herein, a carrier described herein, or a pharmaceutical composition described herein, to thereby treat, ameliorate, or prevent the liver disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the liver disease in the subject.

In one aspect, provided herein are uses of dsRNA agents, conjugates, vectors, carriers, and pharmaceutical compositions in the manufacture of a medicament for the treatment, amelioration, or prevention of a liver disease in a subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the liver disease in the subject.

In one aspect, provided herein are uses of dsRNA agents, conjugates, vectors, carriers, and pharmaceutical compositions in the manufacture of a medicament for use in a method of treating, ameliorating, or preventing a liver disease in a subject, the method comprising administering to the subject a dsRNA agent described herein, a conjugate described herein, a vector described herein, a carrier described herein, or a pharmaceutical composition described herein, to thereby treat, ameliorate, or prevent the liver disease in the subject. In some embodiments, the agent (e.g., RNAi agent, dsRNA agent, antisense strand, sense strand), conjugate, vector, carrier, or pharmaceutical composition is administered to the subject in an amount and for a time sufficient to treat, ameliorate, or prevent the liver disease in the subject.

The following applicable to any of the foregoing methods, in specific embodiments, the treating, ameliorating, or preventing of the liver disease is mediated (at least in part) by reducing or inhibiting expression of CIDEB (e.g., hCIDEB) in the subject (e.g., a population of cells within the subject).

The following applicable to any of the foregoing aspects, in some embodiments, the liver disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the liver disease is fatty liver disease, liver inflammation, NASH or NAFLD. In specific embodiments, the liver disease is obesity-induced metabolic syndrome, insulin insensitivity, obesity, type-2 diabetes, AFLD, or cirrhosis. In specific embodiments, the liver disease is MASLD, MASH, MAFLD, MetALD, SLD, or cryptogenic SLD. See, e.g., Rinella M. et al., A multisociety Delphi consensus statement on new fatty liver disease nomenclature, Hepatology 78(6): p 1966-1986 December 2023, the entire contents of which is incorporated herein by reference for all purposes. In specific embodiments, the liver disease is the liver fibrosis, liver inflammation, cirrhosis, liver failure, or jaundice. In specific embodiments, the disease is obesity or type-2 diabetes. In specific embodiments, the liver disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the liver disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the liver disease is atherosclerosis, coronary artery disease, myocardial infarction, or heart failure.

In specific embodiments, the liver disease is fatty liver disease, steatotic liver disease, liver inflammation, NASH, NAFLD, MASLD, MASH, MAFLD, MetALD, SLD, or cryptogenic SLD.

4.11.5 Methods of Diagnosing and/or Prognosticating a Liver Disease

In one aspect, provided herein are methods of diagnosing a liver disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the somatic mutation indicates that the subject has a liver disease.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of diagnosing and/or prognosticating a liver disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the somatic mutation indicates that the subject has a liver disease, is at risk of developing a liver disease, is at risk of developing a more severe form of a liver disease, and/or is at risk of developing a disease associated with a liver disease.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the method further comprises (c) administering to the subject an agent (e.g., inhibitory nucleic acid molecule (e.g., an RNA agent (e.g., a dsRNA agent described herein)) that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of prognosticating a liver disease in a subject, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the somatic mutation indicates that the subject is at risk of developing a liver disease, is at risk of developing a more severe form of a liver disease, and/or is at risk of developing a disease associated with a liver disease.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the method further comprises (c) administering to the subject an agent (e.g., inhibitory nucleic acid molecule (e.g., an RNA agent (e.g., a dsRNA agent described herein)) that inhibits expression of CIDEB if one or more somatic CIDEB mutation is detected in the DNA, RNA, or protein. In some embodiments, the agent comprises a nucleic acid molecule, small molecule, protein, peptide. In some preferred embodiments, the agent is a nucleic acid molecule. In some preferred embodiments, the agent is an inhibitory nucleic acid molecule. In some preferred embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

The following applicable to any of the foregoing aspects, in some embodiments, the liver disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the liver disease is fatty liver disease, liver inflammation, NASH or NAFLD. In specific embodiments, the liver disease is obesity-induced metabolic syndrome, insulin insensitivity, obesity, type-2 diabetes, AFLD, or cirrhosis. In specific embodiments, the liver disease is MASLD, MASH, MAFLD, MetALD, SLD, or cryptogenic SLD. See, e.g., Rinella M. et al., A multisociety Delphi consensus statement on new fatty liver disease nomenclature, Hepatology 78 (6): p 1966-1986 December 2023, the entire contents of which is incorporated herein by reference for all purposes. In specific embodiments, the liver disease is the liver fibrosis, liver inflammation, cirrhosis, liver failure, or jaundice. In specific embodiments, the disease is obesity or type-2 diabetes. In specific embodiments, the liver disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the liver disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the liver disease is atherosclerosis, coronary artery disease, myocardial infarction, or heart failure.

In specific embodiments, the liver disease is fatty liver disease, steatotic liver disease, liver inflammation, NASH, NAFLD, MASLD, MASH, MAFLD, MetALD, SLD, or cryptogenic SLD.

In specific embodiments, the liver disease is a CIDEB associated disease.

In some embodiments, the CIDEB associated disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the CIDEB associated disease is fatty liver disease, liver inflammation, NASH or NAFLD. In specific embodiments, the CIDEB associated disease is obesity-induced metabolic syndrome, insulin insensitivity, obesity, type-2 diabetes, AFLD, or cirrhosis. In specific embodiments, the CIDEB associated disease is MASLD, MASH, MAFLD, MetALD, SLD, or cryptogenic SLD. See, e.g., Rinella M. et al., A multisociety Delphi consensus statement on new fatty liver disease nomenclature, Hepatology 78(6):p 1966-1986 December 2023, the entire contents of which is incorporated herein by reference for all purposes. In specific embodiments, the CIDEB associated disease is the liver fibrosis, liver inflammation, cirrhosis, liver failure, or jaundice. In specific embodiments, the disease is obesity or type-2 diabetes. In specific embodiments, the CIDEB associated disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the CIDEB associated disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the CIDEB associated disease is atherosclerosis, coronary artery disease, myocardial infarction, or heart failure.

In specific embodiments, the CIDEB associated disease is fatty liver disease, steatotic liver disease, liver inflammation, NASH, NAFLD, MASLD, MASH, MAFLD, MetALD, SLD, or cryptogenic SLD.

4.11.6 Methods of Screening, Identifying, and Selecting a Subject for Treatment with a CIDEB Inhibitory Agent

In one aspect, provided herein are methods of screening a subject for administration of an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the subject is selected for administration of an agent (e.g., inhibitory nucleic acid molecule (e.g., an RNA agent (e.g., a dsRNA agent described herein)) that inhibits expression of CIDEB if the somatic mutation is present.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the agent comprises a nucleic acid molecule, small molecule, protein, peptide. In some preferred embodiments, the agent is a nucleic acid molecule. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of selecting a subject for administration of an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the subject is selected for administration of an agent (e.g., inhibitory nucleic acid molecule (e.g., an RNA agent (e.g., a dsRNA agent described herein)) that inhibits expression of CIDEB if the somatic mutation is present.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the agent comprises a nucleic acid molecule, small molecule, protein, peptide. In some preferred embodiments, the agent is a nucleic acid molecule. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of identifying a subject for administration of an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the presence of the somatic mutation indicates that the subject is identified for administration of an agent (e.g., inhibitory nucleic acid molecule (e.g., an RNA agent (e.g., a dsRNA agent described herein)) that inhibits expression of CIDEB.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the agent comprises a nucleic acid molecule, small molecule, protein, peptide. In some preferred embodiments, the agent is a nucleic acid molecule. In some preferred embodiments, the agent is an inhibitory nucleic acid molecule. In some preferred embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of identifying a subject having a disease who is likely to respond to treatment with an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, wherein the subject is identified as a subject likely to respond to treatment with an agent (e.g., inhibitory nucleic acid molecule (e.g., an RNA agent (e.g., a dsRNA agent described herein)) that inhibits expression of CIDEB if the somatic mutation is present.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the agent comprises a nucleic acid molecule, small molecule, protein, peptide. In some preferred embodiments, the agent is a nucleic acid molecule. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (c) withholding administration of an inhibitory nucleic acid molecule that inhibits expression of CIDEB to the subject if one or more somatic CIDEB mutation is not detected in the DNA, RNA, or protein. In some embodiments, the method further comprises (c) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

In one aspect, provided herein are methods of selecting a therapy for a subject having a disease who is likely to respond to treatment with an inhibitory nucleic acid molecule that inhibits expression of CIDEB, the method comprising (a) isolating and purifying DNA, RNA, or protein, or having DNA, RNA, or protein isolated and purified, from a sample obtained from the subject; (b) detecting, or having detected, the presence or absence of one or more somatic CIDEB mutation in the DNA, RNA, or protein, and (c) selecting a therapy for a subject comprising an agent (e.g., inhibitory nucleic acid molecule (e.g., an RNA agent (e.g., a dsRNA agent described herein)) that inhibits expression of CIDEB if the somatic mutation is present.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the agent comprises a nucleic acid molecule, small molecule, protein, peptide. In some preferred embodiments, the agent is a nucleic acid molecule. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (d) administering to the subject an inhibitory nucleic acid molecule that inhibits expression of CIDEB if one or more somatic CIDEB mutation is detected in the DNA, RNA, or protein. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In some embodiments, the method further comprises (d) withholding administration of an inhibitory nucleic acid molecule that inhibits expression of CIDEB to the subject if one or more somatic CIDEB mutation is not detected in the DNA, RNA, or protein. In some embodiments, the method further comprises (d) administering an alternative therapeutic agent for treatment of the disease that is not an inhibitory nucleic acid molecule that inhibits expression of CIDEB to the subject if a somatic CIDEB mutation in the sample is not detected. In some embodiments, the different therapeutic agent is a standard of care agent for the disease.

In some embodiments, the subject is undergoing or has undergone treatment with a different therapeutic agent for the disease that is not an inhibitory nucleic acid molecule that inhibits expression of CIDEB and/or function of CIDEB. In some embodiments, the treatment with the different therapeutic agent is discontinued if a somatic CIDEB mutation in the sample is detected and/or the inhibitory nucleic acid molecule that inhibits expression of CIDEB is administered to the subject.

The following applicable to any of the foregoing aspects, in some embodiments, the disease is a liver disease. In some embodiments, the liver disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the liver disease is fatty liver disease, liver inflammation, NASH or NAFLD. In specific embodiments, the liver disease is obesity-induced metabolic syndrome, insulin insensitivity, obesity, type-2 diabetes, AFLD, or cirrhosis. In specific embodiments, the liver disease is MASLD, MASH, MAFLD, MetALD, SLD, or cryptogenic SLD. See, e.g., Rinella M. et al., A multisociety Delphi consensus statement on new fatty liver disease nomenclature, Hepatology 78 (6): p 1966-1986 December 2023, the entire contents of which is incorporated herein by reference for all purposes. In specific embodiments, the liver disease is the liver fibrosis, liver inflammation, cirrhosis, liver failure, or jaundice. In specific embodiments, the disease is obesity or type-2 diabetes. In specific embodiments, the liver disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the liver disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the liver disease is atherosclerosis, coronary artery disease, myocardial infarction, or heart failure.

In specific embodiments, the liver disease is fatty liver disease, steatotic liver disease, liver inflammation, NASH, NAFLD, MASLD, MASH, MAFLD, MetALD, SLD, or cryptogenic SLD.

In specific embodiments, the disease is a CIDEB associated disease.

In some embodiments, the CIDEB associated disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the CIDEB associated disease is fatty liver disease, liver inflammation, NASH or NAFLD. In specific embodiments, the CIDEB associated disease is obesity-induced metabolic syndrome, insulin insensitivity, obesity, type-2 diabetes, AFLD, or cirrhosis. In specific embodiments, the CIDEB associated disease is MASLD, MASH, MAFLD, MetALD, SLD, or cryptogenic SLD. See, e.g., Rinella M. et al., A multisociety Delphi consensus statement on new fatty liver disease nomenclature, Hepatology 78 (6): p 1966-1986 December 2023, the entire contents of which is incorporated herein by reference for all purposes. In specific embodiments, the CIDEB associated disease is the liver fibrosis, liver inflammation, cirrhosis, liver failure, or jaundice. In specific embodiments, the disease is obesity or type-2 diabetes. In specific embodiments, the CIDEB associated disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the CIDEB associated disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the CIDEB associated disease is atherosclerosis, coronary artery disease, myocardial infarction, or heart failure.

In specific embodiments, the CIDEB associated disease is fatty liver disease, steatotic liver disease, liver inflammation, NASH, NAFLD, MASLD, MASH, MAFLD, MetALD, SLD, or cryptogenic SLD.

### 4.11.7 In Vitro Methods of Screening Samples for Somatic CIDEB Mutations

In one aspect, provided herein are methods of screening a sample from a subject for the presence or absence of one or more somatic CIDEB mutation, the method comprising (a) isolating and purifying DNA, RNA, or protein from a sample obtained from the subject; (b) detecting the presence or absence of one or more somatic CIDEB mutation in the DNA, RNA, or protein.

In some embodiments, (a) comprises isolating and purifying DNA, or having DNA isolated and purified, from a sample obtained from the subject; and (b) comprises detecting, or having detected, the presence or absence of one or more somatic CIDEB mutation in the DNA. In some embodiments, (a) comprises isolating and purifying RNA, or having RNA isolated and purified, from a sample obtained from the subject; and (b) comprises detecting, or having detected, the presence or absence of one or more somatic CIDEB mutation in the RNA. In some embodiments, (a) comprises isolating and purifying protein, or having protein isolated and purified, from a sample obtained from the subject; and (b) comprises detecting, or having detected, the presence or absence of one or more somatic CIDEB mutation in the protein.

In some embodiments, the method further comprises (c) administering to the subject an inhibitory nucleic acid molecule that inhibits expression of CIDEB if one or more somatic CIDEB mutation is detected in the DNA, RNA, or protein. In some embodiments, the agent comprises a nucleic acid molecule, small molecule, protein, peptide. In some preferred embodiments, the agent is a nucleic acid molecule. In some embodiments, the inhibitory nucleic acid molecule comprises one or more RNA agent described herein (e.g., an RNAi agent, a dsRNA agent, an antisense strand, a sense strand, an siRNA agent, an antisense oligonucleotide) (or a vector comprising or encoding the RNA agent described herein, e.g., a vector described herein; a conjugate comprising the RNA agent described herein, e.g., a conjugate described herein; a carrier comprising the RNA agent described herein (or a vector or conjugate comprising the same), e.g., a carrier described herein; and/or a pharmaceutical composition comprising the RNA agent described herein (or a vector, conjugate, or carrier comprising the same), e.g., a pharmaceutical composition described herein). In some embodiments, the RNA agent is a dsRNA agent described herein comprising a sense strand and an antisense strand.

In one aspect, provided herein are methods of characterizing a DNA molecule in a sample from a subject having a disease, the method comprising (a) isolating and purifying DNA (or having DNA isolated and purified) in a sample from the subject; (b) analyzing the DNA (e.g., sequencing at least a portion of the CIDEB gene in the DNA); and (c) determining the presence or absence of a somatic CIDEB mutation in the DNA molecule.

In one aspect, provided herein are methods of characterizing a disease in a subject, the method comprising (a) isolating and purifying DNA (or having DNA isolated and purified) in a sample from the subject; (b) detecting the presence or absence of a somatic CIDEB mutation in the DNA; and (c) characterizing the liver disease as somatic CIDEB mutation positive or somatic CIDEB mutation negative based on the detection.

The following applicable to any of the foregoing aspects, in some embodiments, the subject is suspected of having, has been diagnosed with, or has a liver disease. In some embodiments, the liver disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the liver disease is fatty liver disease, liver inflammation, NASH or NAFLD. In specific embodiments, the liver disease is obesity-induced metabolic syndrome, insulin insensitivity, obesity, type-2 diabetes, AFLD, or cirrhosis. In specific embodiments, the liver disease is MASLD, MASH, MAFLD, MetALD, SLD, or cryptogenic SLD. See, e.g., Rinella M. et al., A multisociety Delphi consensus statement on new fatty liver disease nomenclature, Hepatology 78 (6): p 1966-1986 December 2023, the entire contents of which is incorporated herein by reference for all purposes. In specific embodiments, the liver disease is the liver fibrosis, liver inflammation, cirrhosis, liver failure, or jaundice. In specific embodiments, the disease is obesity or type-2 diabetes. In specific embodiments, the liver disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the liver disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the liver disease is atherosclerosis, coronary artery disease, myocardial infarction, or heart failure.

In specific embodiments, the liver disease is fatty liver disease, steatotic liver disease, liver inflammation, NASH, NAFLD, MASLD, MASH, MAFLD, MetALD, SLD, or cryptogenic SLD.

In specific embodiments, the liver disease is a CIDEB associated disease.

In some embodiments, the CIDEB associated disease is fatty liver disease, liver inflammation, nonalcoholic steatohepatitis (NASH), nonalcoholic fatty liver disease (NAFLD), obesity-induced metabolic syndrome, insulin insensitivity, obesity, type-2 diabetes, alcoholic fatty liver disease (AFLD), cirrhosis, metabolic dysfunction associated steatotic liver disease (MASLD), metabolic-associated steatohepatitis (MASH), metabolic dysfunction associated fatty liver disease (MAFLD), metabolic dysfunction and alcohol associated steatotic liver disease (MetALD), steatotic liver disease (SLD), cryptogenic SLD, liver fibrosis, liver failure, jaundice, a liver infection (e.g., hepatitis C infection, hepatitis B infection), or a cardiovascular disease (e.g., atherosclerosis, coronary artery disease, myocardial infarction, or heart failure).

In specific embodiments, the CIDEB associated disease is fatty liver disease, liver inflammation, NASH or NAFLD. In specific embodiments, the CIDEB associated disease is obesity-induced metabolic syndrome, insulin insensitivity, obesity, type-2 diabetes, AFLD, or cirrhosis. In specific embodiments, the CIDEB associated disease is MASLD, MASH, MAFLD, MetALD, SLD, or cryptogenic SLD. See, e.g., Rinella M. et al., A multisociety Delphi consensus statement on new fatty liver disease nomenclature, Hepatology 78 (6): p 1966-1986 December 2023, the entire contents of which is incorporated herein by reference for all purposes. In specific embodiments, the CIDEB associated disease is the liver fibrosis, liver inflammation, cirrhosis, liver failure, or jaundice. In specific embodiments, the disease is obesity or type-2 diabetes. In specific embodiments, the CIDEB associated disease is a liver infection (e.g., hepatitis C infection, hepatitis B infection). In specific embodiments, the liver infection is a hepatitis infection. In specific embodiments, the hepatitis infection is a hepatitis C infection or hepatitis B infection. In specific embodiments, the CIDEB associated disease is a cardiovascular disease (e.g., atherosclerosis, coronary artery disease, myocardial infarction, or heart failure). In specific embodiments, the cardiovascular disease is atherosclerosis, coronary artery disease, myocardial infarction, or heart failure. In specific embodiments, the CIDEB associated disease is atherosclerosis, coronary artery disease, myocardial infarction, or heart failure.

In specific embodiments, the CIDEB associated disease is fatty liver disease, steatotic liver disease, liver inflammation, NASH, NAFLD, MASLD, MASH, MAFLD, MetALD, SLD, or cryptogenic SLD.

### 4.12 Kits

In a one aspect, provided herein are kits comprising any one or more agent described herein (e.g., an RNAi agent, a dsRNA agent, an antisense strand, a sense strand) (see, e.g., §§ 4.2, 4.3); a vector described herein (see, e.g., § 4.6); a conjugate described herein (see, e.g., § 4.4); a carrier described herein (see, e.g., § 4.8); a host cell described herein (see, e.g., § 4.9); and/or a pharmaceutical composition described herein (see, e.g., § 4.10); or any combination thereof. In addition, the kit may comprise a liquid vehicle for solubilizing or diluting, and/or technical instructions. The technical instructions of the kit may contain information about administration and dosage and subject groups.

In some embodiments, the agent (e.g., described herein) (e.g., RNAi agent, dsRNA agent, antisense strand, sense strand), vector (e.g., described herein), conjugate (e.g., described herein), carrier (e.g., described herein), host cell (e.g., described herein), and/or pharmaceutical composition (e.g., described herein) is provided in a separate part of the kit. In some embodiments, the agent (e.g., described herein) (e.g., RNAi agent, dsRNA agent, antisense strand, sense strand), vector (e.g., described herein), conjugate (e.g., described herein), carrier (e.g., described herein), host cell (e.g., described herein), and/or pharmaceutical composition (e.g., described herein) is optionally lyophilized, spray-dried, or spray-freeze dried. The kit may further contain as a part a vehicle (e.g., buffer solution) for solubilizing the dried or lyophilized any agent (e.g., described herein) (e.g., RNAi agent, dsRNA agent, antisense strand, sense strand), vector (e.g., described herein), conjugate (e.g., described herein), carrier (e.g., described herein), host cell (e.g., described herein), and/or pharmaceutical composition (e.g., described herein).

In some embodiments, the kit comprises a single dose container. In some embodiments, the kit comprises a multi-dose container. In some embodiments, the kit comprises an administration device (e.g., an injector for intradermal injection or a syringe for intramuscular injection).

Any of the kits described herein may be used in any of the methods described herein (see, e.g., § 4.11).

### 5. EXAMPLES

TABLE OF CONTENTS

| | |
|---|---|
| 5.1 | Example 1. CIDEB selection in Nonalcoholic Steatohepatitis. |
| 5.2 | Example 2. Design of CIDEB Targeting RNAi Agents. |
| 5.3 | Example 3. Synthesis of CIDEB Targeting RNAi Agents. |
| 5.4 | Example 4. In Vitro dsRNA Agent Mediated Knockdown |
| | of hCIDEB in Hep3B Cells. |
| 5.5 | Example 5. In Vitro dsRNA Agent Mediated Knockdown |
| | of hCIDEB in Primary Human Hepatocytes. |
| 5.6 | Example 6. In Vitro dsRNA Agent Mediated Knockdown |
| | of hCIDEB in Primary Human Hepatocytes. |
| 5.7 | Example 7. In Vitro dsRNA Agent Mediated Knockdown |
| | of hCIDEB in Primary Cyno Hepatocytes. |
| 5.8 | Example 8. In Vitro GalNAc-dsRNA Agent Mediated |
| | Knockdown of hCIDEB in Hep3B cells. |
| 5.9 | Example 9. In Vitro GalNAc-dsRNA Agent Mediated |
| | Knockdown of hCIDEB in Primary Human Hepatocytes. |
| 5.1 | Example 1. CIDEB selection in Nonalcoholic Steatohepatitis. |

The selection of CIDEB in the context of nonalcoholic steatohepatitis (NASH) was evaluated. Briefly, liver samples from 46 subjects with NASH and 20 subjects without liver disease were sequenced using Nanoseq at a mean sequencing depth of 549 Dx per sample in NASH and 1218 Dx per sample in normal liver. Results of interest in the primary sample (defined as a global q-value<0.1) were restricted hypothesis tested in the control analysis. In the primary sample, CIDEB had an enrichment ratio of 11.6 and a global q-value=0.077; there was no indication of selection in the normal liver samples. Taken together, these findings indicate that CIDEB is positively selected for in NASH, meaning that cell clones with mutations in the CIDEB gene proliferate under NASH disease conditions.

### 5.2 Example 2. Design of CIDEB Targeting RNAi Agents

dsRNA agents targeting hCIDEB (NCBI Ref.: NM_014430.4) were designed. The nucleotide sequence of the unmodified CIDEB sense and antisense strands of each of the dsRNA agents are set forth in Table 2; and the nucleotide sequence of the exemplary modified CIDEB sense and antisense strands of each of the dsRNA agents are set forth in Table 3.

### 5.3 Example 3. Synthesis of CIDEB Targeting RNAi Agents

The dsRNA agents set forth in Table 2 and Table 3 were synthesized using standard methods known in the art and described herein. Briefly, the dsRNA agents were synthesized using a Mermade 192 synthesizer (BioAutomation) on controlled pore glass (500-1000 A) solid supports loaded with a first nucleotide of interest. Upon completion of the solid phase synthesis, solid-supported polynucleotides were treated with Methylamine (40% aqueous) at room temperature in 96 well plates for approximately 2 hours to afford cleavage from the solid support and subsequent removal of relevant protecting groups. Polynucleotides were precipitated by the addition of 1 mL of 9:1 acetontrile:ethanol or 1:1 ethanol:isopropanol. The plates were then centrifuged at 4° C. for 45 minutes and the supernatant removed. The polynucleotide pellet was resuspended in 20 mM NaOAc and subsequently desalted using a HiTrap size exclusion column (5 mL, GE Healthcare) on an Agilent LC system equipped with an autosampler, UV detector, conductivity meter, and fraction collector. Desalted samples were collected in 96 well plates and then analyzed by LC-MS and UV spectrometry to confirm identity and quantify the amount of material, respectively.

Duplexing of single strands was performed on a Tecan liquid handling robot. Sense and antisense single strands were combined in an equimolar ratio to a final concentration of 10 μM in 1×PBS in 96 well plates, the plate was sealed, incubated at 100° C. for 10 minutes, and was subsequently allowed to return slowly to room temperature over a period of 2-3 hours. The concentration and identity of each duplex was confirmed and is then subsequently utilized for screening assays.

### 5.4 Example 4. In Vitro dsRNA Agent Mediated Knockdown of hCIDEB in Hep3B Cells

Each of dsRNA Agents 240-478 set forth in Table 3 above were evaluated for their ability to knockdown hCIDEB expression in vitro in Hep3B Cells.

Briefly, Hep3B cells (ATCC) were seeded at 15,000 cells per well in a standard 96 well plate. The cells were transfected with the indicated hCIDEB targeting dsRNA agent (either 0.3 nM or 20 nM) using Lipofectamine RNAiMax (0.3 μl/well) (Thermo Fisher Scientific) according to the manufacturer's instructions and incubated for 24 hours. The level of hCIDEB was assessed utilizing a standard branched DNA (bDNA) assay (Quantigene 2.0 (Thermo Fisher Scientific)) according to the manufacturer's instructions. The expression of hCIDEB was normalized to the expression of human GAPDH. Each treatment group was run in quadruplicate, the mean and standard deviation calculated.

Table 5 shows the percent of hCIDEB mRNA remaining after treatment with the indicated dsRNA agent (normalized to GAPDH and relative to control treated cells set to 100%).

TABLE 5
Range % hCIDEB mRNA Remaining (Normalized to GAPDH).

| dsRNA | CIDEB/GAPDH | |
|---|---|---|
| Agent | 20 nM | 0.3 nM |
| 240 | 80-95 | 75-85 |
| 241 | 70-90 | 65-85 |
| 242 | 60-80 | 55-80 |
| 243 | 70-100 | 70-90 |
| 244 | 85-115 | 70-95 |
| 245 | 95-120 | 90-105 |
| 246 | 65-100 | 70-90 |
| 247 | 65-95 | 70-95 |
| 248 | 70-95 | 65-80 |
| 249 | 75-90 | 75-90 |
| 250 | 75-95 | 70-85 |
| 251 | 80-95 | 60-95 |
| 252 | 70-95 | 60-85 |
| 253 | 70-105 | 50-95 |
| 254 | 75-110 | 60-95 |
| 255 | 95-145 | 70-100 |
| 256 | 60-95 | 55-85 |
| 257 | 70-105 | 60-100 |
| 258 | 30-45 | 35-45 |
| 259 | 85-130 | 75-105 |
| 260 | 65-100 | 70-80 |
| 261 | 85-100 | 60-85 |
| 262 | 70-110 | 55-80 |
| 263 | 85-110 | 70-90 |
| 264 | 30-40 | 35-45 |
| 265 | 80-95 | 50-65 |
| 266 | 70-95 | 50-65 |
| 267 | 55-85 | 50-135 |
| 268 | 60-95 | 55-65 |
| 269 | 70-110 | 70-90 |
| 270 | 70-105 | 60-90 |
| 271 | 60-110 | 55-85 |
| 272 | 65-105 | 60-95 |
| 273 | 70-105 | 60-95 |
| 274 | 60-90 | 55-90 |
| 275 | 65-90 | 45-95 |
| 276 | 60-90 | 55-95 |
| 277 | 65-105 | 70-105 |
| 278 | 65-110 | 65-110 |
| 279 | 70-115 | 70-100 |
| 280 | 80-110 | 70-90 |
| 281 | 70-100 | 60-75 |
| 282 | 60-95 | 55-70 |
| 283 | 65-105 | 50-65 |
| 284 | 65-105 | 55-70 |
| 285 | 65-110 | 60-70 |
| 286 | 55-75 | 55-65 |
| 287 | 30-55 | 40-50 |
| 288 | 70-115 | 65-75 |
| 289 | 65-115 | 70-85 |
| 290 | 90-110 | 70-95 |
| 291 | 70-120 | 60-90 |
| 292 | 70-120 | 65-90 |
| 293 | 70-105 | 55-95 |
| 294 | 90-130 | 60-105 |
| 295 | 70-110 | 60-100 |
| 296 | 80-125 | 65-95 |
| 297 | 55-80 | 65-90 |
| 298 | 45-75 | 60-85 |
| 299 | 75-130 | 70-110 |
| 300 | 65-85 | 60-75 |
| 301 | 45-85 | 60-80 |
| 302 | 35-80 | 55-75 |
| 303 | 30-55 | 40-55 |
| 304 | 50-85 | 55-80 |
| 305 | 45-80 | 50-75 |
| 306 | 50-90 | 60-75 |
| 307 | 50-85 | 60-85 |
| 308 | 50-80 | 50-70 |
| 309 | 55-75 | 65-80 |
| 310 | 60-90 | 70-85 |
| 311 | 55-80 | 70-110 |
| 312 | 50-75 | 70-95 |
| 313 | 45-75 | 75-105 |
| 314 | 45-65 | 75-100 |
| 315 | 40-75 | 80-100 |
| 316 | 45-75 | 70-115 |
| 317 | 45-70 | 65-100 |
| 318 | 45-70 | 65-95 |
| 319 | 60-90 | 65-90 |
| 320 | 60-75 | 70-85 |
| 321 | 60-80 | 75-90 |
| 322 | 50-80 | 80-100 |
| 323 | 55-90 | 85-115 |
| 324 | 55-85 | 85-115 |
| 325 | 50-90 | 80-105 |
| 326 | 80-95 | 85-105 |
| 327 | 75-115 | 85-95 |
| 328 | 125-140 | 105-115 |
| 329 | 70-100 | 75-100 |
| 330 | 65-90 | 85-100 |
| 331 | 105-130 | 100-115 |
| 332 | 105-150 | 100-110 |
| 333 | 60-75 | 70-90 |
| 334 | 70-80 | 85-95 |
| 335 | 60-90 | 75-95 |
| 336 | 85-135 | 80-95 |
| 337 | 55-75 | 65-80 |
| 338 | 55-80 | 70-90 |
| 339 | 60-85 | 80-95 |
| 340 | 65-90 | 75-90 |
| 341 | 60-95 | 75-85 |
| 342 | 85-105 | 80-110 |
| 343 | 50-80 | 55-95 |
| 344 | 55-100 | 60-80 |
| 345 | 55-100 | 65-75 |
| 346 | 50-95 | 60-75 |
| 347 | 50-100 | 65-80 |
| 348 | 55-90 | 65-80 |
| 349 | 80-105 | 80-100 |
| 350 | 65-115 | 70-90 |
| 351 | 55-105 | 65-95 |
| 352 | 70-105 | 75-85 |
| 353 | 70-105 | 90-105 |
| 354 | 55-90 | 70-95 |
| 355 | 70-120 | 90-105 |
| 356 | 50-80 | 65-75 |
| 357 | 50-90 | 70-80 |
| 358 | 5-15 | 10-15 |
| 359 | 10-20 | 10-15 |
| 360 | 15-30 | 25-35 |
| 361 | 10-20 | 15-20 |
| 362 | 35-60 | 40-55 |
| 363 | 5-15 | 10-20 |
| 364 | 5-15 | 10-20 |
| 365 | 5-15 | 10-15 |
| 366 | 5-10 | 5-15 |
| 367 | 0-10 | 5-10 |
| 368 | 0-15 | 5-10 |
| 369 | 5-10 | 5-15 |
| 370 | 15-30 | 20-30 |
| 371 | 20-25 | 20-30 |
| 372 | 10-20 | 10-20 |
| 373 | 10-15 | 5-15 |
| 374 | 5-10 | 5-10 |
| 375 | 5-20 | 10-15 |
| 376 | 5-10 | 5-15 |
| 377 | 15-35 | 25-35 |
| 378 | 30-40 | 45-60 |
| 379 | 55-70 | 50-75 |
| 380 | 5-20 | 15-25 |
| 381 | 10-20 | 10-20 |
| 382 | 45-60 | 35-50 |
| 383 | 35-50 | 50-60 |
| 384 | 25-35 | 25-35 |
| 385 | 35-50 | 35-45 |
| 386 | 30-45 | 20-25 |
| 387 | 5-10 | 10-15 |
| 388 | 5-10 | 10-15 |
| 389 | 15-20 | 10-20 |
| 390 | 10-15 | 10-20 |
| 391 | 5-15 | 10-15 |
| 392 | 60-65 | 65-80 |
| 393 | 30-60 | 25-85 |
| 394 | 15-20 | 20-30 |
| 395 | 45-70 | 55-75 |
| 396 | 30-45 | 50-70 |
| 397 | 10-15 | 15-20 |
| 398 | 10-20 | 15-25 |
| 399 | 10-20 | 15-30 |
| 400 | 10-20 | 15-25 |
| 401 | 10-20 | 10-20 |
| 402 | 45-60 | 40-60 |
| 403 | 5-15 | 15-20 |
| 404 | 5-10 | 5-15 |
| 405 | 10-20 | 10-20 |
| 406 | 10-20 | 10-15 |
| 407 | 10-20 | 10-20 |
| 408 | 10-15 | 10-15 |
| 409 | 10-15 | 5-15 |
| 410 | 10-25 | 25-35 |
| 411 | 5-15 | 10-20 |
| 412 | 5-15 | 10-15 |
| 413 | 10-20 | 15-25 |
| 414 | 5-15 | 10-15 |
| 415 | 10-20 | 15-25 |
| 416 | 10-15 | 20-30 |
| 417 | 10-20 | 20-35 |
| 418 | 10-15 | 15-25 |
| 419 | 10-15 | 15-30 |
| 420 | 45-65 | 40-70 |
| 421 | 10-20 | 15-20 |
| 422 | 5-15 | 10-25 |
| 423 | 40-55 | 65-70 |
| 424 | 55-80 | 85-105 |
| 425 | 65-95 | 100-115 |
| 426 | 50-80 | 85-105 |
| 427 | 55-85 | 90-115 |
| 428 | 55-80 | 85-100 |
| 429 | 60-90 | 85-100 |
| 430 | 65-90 | 80-95 |
| 431 | 70-90 | 80-90 |
| 432 | 60-90 | 65-85 |
| 433 | 45-70 | 60-70 |
| 434 | 30-60 | 50-55 |
| 435 | 35-65 | 50-65 |
| 436 | 60-105 | 80-90 |
| 437 | 55-85 | 75-90 |
| 438 | 55-90 | 75-80 |
| 439 | 80-125 | 85-100 |
| 440 | 15-25 | 20-25 |
| 441 | 10-15 | 15-20 |
| 442 | 5-15 | 5-15 |
| 443 | 5-10 | 5-10 |
| 444 | 10-20 | 15-20 |
| 445 | 40-70 | 65-80 |
| 446 | 5-10 | 5-10 |
| 447 | 5-10 | 5-10 |
| 448 | 15-30 | 15-25 |
| 449 | 15-25 | 15-20 |
| 450 | 15-25 | 10-15 |
| 451 | 10-15 | 5-15 |
| 452 | 10-20 | 10-15 |
| 453 | 15-35 | 10-20 |
| 454 | 5-15 | 10-20 |
| 455 | 15-25 | 15-20 |
| 456 | 5-20 | 10-20 |
| 457 | 10-20 | 15-20 |
| 458 | 10-20 | 10-20 |
| 459 | 10-25 | 15-20 |
| 460 | 10-20 | 10-15 |
| 461 | 5-20 | 10-20 |
| 462 | 5-10 | 5-15 |
| 463 | 0-10 | 5-10 |
| 464 | 0-10 | 5-10 |
| 465 | 0-10 | 5-10 |
| 466 | 10-15 | 15-20 |
| 467 | 5-15 | 10-15 |
| 468 | 35-55 | 50-60 |
| 469 | 35-50 | 30-40 |
| 470 | 5-10 | 5-15 |
| 471 | 5-10 | 5-15 |
| 472 | 5-15 | 10-20 |
| 473 | 5-15 | 20-25 |
| 474 | 5-10 | 10-20 |
| 475 | 5-10 | 10-20 |
| 476 | 0-10 | 10-15 |
| 477 | 10-20 | 15-25 |
| 478 | 5-10 | 10-15 |

### 5.5 Example 5. In Vitro dsRNA Agent Mediated Knockdown of hCIDEB in Primary Human Hepatocytes

Each of dsRNA Agents 366, 367, 388, 404, 411, 442, 443, 446, 447, 462, 463, 464, 465, 467, 470, 471, 474, 475, 476, and 478 set forth in Table 3 above were evaluated for their ability to knockdown hCIDEB expression in vitro in primary human hepatocytes.

Briefly, primary human hepatocytes were seeded at 15,000 cells per well in a standard 96 well plate. The cells were transfected with the indicated hCIDEB targeting dsRNA agent (20 nM, 0.3 nM) using Lipofectamine RNAiMax (0.3 μl/well) (Thermo Fisher Scientific) according to the manufacturer's instructions and incubated for 24 hours. The level of hCIDEB was assessed utilizing a standard bDNA assay (Quantigene 2.0 (Thermo Fisher Scientific)) according to manufacturer's instructions. The expression of hCIDEB was normalized to the expression of human GAPDH. Each treatment group was run in quadruplicate.

Table 6 shows the calculated IC50 (nM), IC80 (nM), and the remaining hCIDEB mRNA percent (normalized to GAPDH) (mean of quadruplicates).

TABLE 6
IC50, IC80, and Max % Inhibition of hCIDEB mRNA
(Normalized to GAPDH).

| dsRNA | | | % Maximum |
|---|---|---|---|
| Agent | IC50 [nM] | IC80 [nM] | Inhibition |
| 366 | >0.001 ≤ 0.01 | >0.01 ≤ 0.1 | >80 ≤ 90 |
| 367 | >0.0001 ≤ 0.001 | >0.001 ≤ 0.01 | >90 ≤ 100 |
| 388 | >0.01 ≤ 0.1 | >0.01 ≤ 0.1 | >90 ≤ 100 |
| 404 | >0.001 ≤ 0.01 | >0.01 ≤ 0.1 | >80 ≤ 90 |
| 411 | >0.01 ≤ 0.1 | >0.1 ≤ 1 | >90 ≤ 100 |
| 442 | >0.01 ≤ 0.1 | >0.01 ≤ 0.1 | >90 ≤ 100 |
| 443 | >0.001 ≤ 0.01 | >0.01 ≤ 0.1 | >90 ≤ 100 |
| 446 | >0.001 ≤ 0.01 | >0.01 ≤ 0.1 | >90 ≤ 100 |
| 447 | >0.001 ≤ 0.01 | >0.01 ≤ 0.1 | >90 ≤ 100 |
| 462 | >0.001 ≤ 0.01 | >0.01 ≤ 0.1 | >90 ≤ 100 |
| 463 | >0.001 ≤ 0.01 | >0.01 ≤ 0.1 | >90 ≤ 100 |
| 464 | >0.001 ≤ 0.01 | >0.01 ≤ 0.1 | >80 ≤ 90 |
| 465 | >0.001 ≤ 0.01 | >0.01 ≤ 0.1 | >90 ≤ 100 |
| 467 | >0.001 ≤ 0.01 | >0.01 ≤ 0.1 | >80 ≤ 90 |
| 470 | >0.001 ≤ 0.01 | >0.01 ≤ 0.1 | >90 ≤ 100 |
| 471 | >0.0001 ≤ 0.001 | >0.001 ≤ 0.01 | >90 ≤ 100 |
| 474 | >0.001 ≤ 0.01 | >0.01 ≤ 0.1 | >80 ≤ 90 |
| 475 | >0.001 ≤ 0.01 | >0.01 ≤ 0.1 | >90 ≤ 100 |
| 476 | >0.001 ≤ 0.01 | >0.001 ≤ 0.01 | >90 ≤ 100 |
| 478 | >0.001 ≤ 0.01 | >0.01 ≤ 0.1 | >90 ≤ 100 |

### 5.6 Example 6. In Vitro dsRNA Agent Mediated Knockdown of hCIDEB in Primary Human Hepatocytes

Each of dsRNA Agents 368, 369, 373, 374, 375, 376, 380, 443, 387, 388, 390, 391, 474, 473, 477, 457, 397, 472, 403, 405, 406, 408, 409, 411, 414, 415, or 417 set forth in Table 3 above were evaluated for their ability to knockdown hCIDEB expression in vitro in primary human hepatocytes.

Briefly, primary human hepatocytes were seeded at 80,000 cells per well in a standard 96 well plate. The cells were transfected with the indicated hCIDEB targeting dsRNA agent (20 nM, 4 nM, 0.8 nM, 0.16 nM, 0.032 nM, 0.0064 nM, 0.00128 nM, 0.000256 nM, 0.0000512 nM, or 0.00001024 nM) using Lipofectamine RNAiMax (0.3 μl/well) (Thermo Fisher Scientific) according to the manufacturer's instructions and incubated for 24 hours. The level of hCIDEB was assessed utilizing a standard bDNA assay (Quantigene 2.0 (Thermo Fisher Scientific)) according to manufacturer's instructions. The expression of hCIDEB was normalized to the expression of human GAPDH. Each treatment group was run in quadruplicate.

Table 7 shows the calculated IC50 (nM), IC80 (nM), and the remaining hCIDEB mRNA percent (normalized to GAPDH) (mean of quadruplicates).

TABLE 7
IC50, IC80, and Max % Inhibition of hCIDEB mRNA
(Normalized to GAPDH).

| | | | % Maximum |
|---|---|---|---|
| DsRNA Agent | IC50 [nM] | IC80 [nM] | Inhibition |
| 368 | >0.001 ≤ 0.01 | >0.01 ≤ 0.1 | >80 ≤ 90 |
| 369 | >0.01 ≤ 0.1 | >0.1 ≤ 1 | >80 ≤ 90 |
| 373 | >0.01 ≤ 0.1 | >0.1 ≤ 1 | >80 ≤ 90 |
| 374 | >0.01 ≤ 0.1 | >0.1 ≤ 1 | >80 ≤ 90 |
| 375 | >0.01 ≤ 0.1 | N/A | >70 ≤ 80 |
| 376 | >0.01 ≤ 0.1 | >0.1 ≤ 1 | >80 ≤ 90 |
| 380 | >0.1 ≤ 1 | N/A | >60 ≤ 70 |
| 443 | >0.01 ≤ 0.1 | >0.1 ≤ 1 | >80 ≤ 90 |
| 387 | >0.01 ≤ 0.1 | >0.1 ≤ 1 | >80 ≤ 90 |
| 388 | >0.01 ≤ 0.1 | >1 ≤ 5 | >80 ≤ 90 |
| 390 | >0.01 ≤ 0.1 | N/A | >70 ≤ 80 |
| 391 | >0.01 ≤ 0.1 | N/A | >80 ≤ 90 |
| 474 | >0.1 ≤ 1 | N/A | >70 ≤ 80 |
| 473 | >0.01 ≤ 0.1 | N/A | >70 ≤ 80 |
| 477 | >0.01 ≤ 0.1 | N/A | >70 ≤ 80 |
| 457 | >0.01 ≤ 0.1 | >1 ≤ 5 | >80 ≤ 90 |
| 397 | >0.1 ≤ 1 | N/A | >70 ≤ 80 |
| 472 | >0.01 ≤ 0.1 | >0.1 ≤ 1 | >80 ≤ 90 |
| 403 | >0.1 ≤ 1 | N/A | >70 ≤ 80 |
| 405 | >0.01 ≤ 0.1 | >0.1 ≤ 1 | >80 ≤ 90 |
| 406 | >0.01 ≤ 0.1 | N/A | >70 ≤ 80 |
| 408 | >0.001 ≤ 0.01 | >0.1 ≤ 1 | >80 ≤ 90 |
| 409 | >0.001 ≤ 0.01 | >0.1 ≤ 1 | >80 ≤ 90 |
| 411 | >0.01 ≤ 0.1 | N/A | >70 ≤ 80 |
| 414 | >0.01 ≤ 0.1 | >0.1 ≤ 1 | >80 ≤ 90 |
| 415 | >0.1 ≤ 1 | N/A | >70 ≤ 80 |
| 417 | >0.1 ≤ 1 | N/A | >70 ≤ 80 |

### 5.7 Example 7. In Vitro dsRNA Agent Mediated Knockdown of hCIDEB in Primary Cyno Hepatocytes

Each of dsRNA Agents 368, 369, 373, 374, 375, 376, 387, 388, 390, 391, 405, 406, 408, 409, 411, 414, 443, 457, 472 or 477 set forth in Table 3 above were evaluated for their ability to knockdown hCIDEB expression in vitro in primary cynomolgus macaque (cyno) hepatocytes.

Briefly, primary cyno hepatocytes were seeded at 60,000 cells per well in a standard 96 well plate. The cells were transfected with the indicated hCIDEB targeting dsRNA agent (20 nM, 0.1 nM) using Lipofectamine RNAiMax (0.3 μl/well) (Thermo Fisher Scientific) according to the manufacturer's instructions and incubated for 24 hours. The level of cyno CIDEB was assessed utilizing a standard bDNA assay (Quantigene 2.0 (Thermo Fisher Scientific)) according to manufacturer's instructions. The expression of cyno CIDEB was normalized to the expression of cyno GAPDH. Each treatment group was run in quadruplicate.

Table 8 shows the percent of cyno CIDEB mRNA remaining after treatment with the indicated dsRNA agent (normalized to GAPDH and relative to control treated cells set to 100%).

TABLE 8
Range % cyno CIDEB mRNA Remaining (Normalized to GAPDH).

| dsRNA | CIDEB/GAPDH | |
|---|---|---|
| Agent | 20 nM | 0.1 nM |
| 368 | 10-15 | 15-20 |
| 369 | 10-15 | 35-40 |
| 373 | 10-15 | 20-25 |
| 374 | 10-15 | 20-25 |
| 375 | 20-25 | 60-70 |
| 376 | 10-15 | 30-40 |
| 387 | 10-15 | 30-40 |
| 388 | 15-20 | 50-60 |
| 390 | 20-25 | 60-70 |
| 391 | 15-20 | 30-40 |
| 405 | 20-25 | 40-50 |
| 406 | 20-25 | 30-40 |
| 408 | 10-15 | 15-20 |
| 409 | 15-20 | 20-25 |
| 411 | 20-25 | 70-80 |
| 414 | 50-60 | 80-90 |
| 443 | 10-15 | 25-30 |
| 457 | 15-20 | 30-40 |
| 472 | 10-15 | 40-50 |
| 477 | 50-60 | 70-80 |

### 5.8 Example 8. In Vitro GalNAc-dsRNA Agent Mediated Knockdown of hCIDEB in Hep3B Cells

Each of GalNAc-dsRNA Agents 483, 484, 485, 486, and 487 set forth in Table 11 above were evaluated for their ability to knockdown hCIDEB expression in vitro in Hep3B cells. Each base modified siRNA was synthesized as a GalNAc conjugate, introduced as the triantennary GalNAc-TEG cluster at the 3′-end of each of the sense strand.

The corresponding base modified dsRNA agent (set forth in Table 3) as well as the corresponding base unmodified dsRNA agent (set forth in Table 2) for each of the GalNAc-dsRNA agents 483-487 is also set forth in Table 11.

Briefly, Hep3B cells (ATCC) were seeded at 15,000 cells per well in a standard 96 well plate. The cells were transfected with the indicated hCIDEB targeting dsRNA agent (either 0.3 nM or 20 nM) using Lipofectamine RNAiMax (0.3 μl/well) (Thermo Fisher Scientific) according to the manufacturer's instructions and incubated for 24 hours. The level of hCIDEB was assessed utilizing a standard branched DNA (bDNA) assay (Quantigene 2.0 (Thermo Fisher Scientific)) according to the manufacturer's instructions. The expression of hCIDEB was normalized to the expression of human GAPDH. Each treatment group was run in quadruplicate, the mean and standard deviation calculated.

Table 9 shows the percent of hCIDEB mRNA remaining after treatment with the indicated GalNAc-dsRNA Agent (normalized to GAPDH and relative to control treated cells set to 100%).

TABLE 9
Range % hCIDEB mRNA Remaining (Normalized to GAPDH).

| | CIDEB/GAPDH | |
|---|---|---|
| dsRNA Agent | 20 nM | 0.3 nM |
| 487 | 10-15 | 5-10 |
| 483 | 5-10 | 5-10 |
| 486 | 5-10 | 5-10 |
| 484 | 5-10 | 5-10 |
| 485 | 5-10 | 10-15 |

### 5.9 Example 9. In Vitro GalNAc-dsRNA Agent Mediated Knockdown of hCIDEB in Primary Human Hepatocytes

Each of GalNAc-dsRNA Agents 483, 484, 485, 486, and 487 set forth in Table 11 above were evaluated for their ability to knockdown hCIDEB expression in vitro. Each base modified siRNA was synthesized as GalNAc conjugates, introduced as the triantennary GalNAc-TEG cluster at the 3′-end of each of the sense strands.

The corresponding base modified dsRNA agent (set forth in Table 3) as well as the corresponding base unmodified dsRNA agent (set forth in Table 2) for each of the GalNAc-dsRNA agents 483-487 is set forth in Table 11.

Briefly, primary human hepatocytes were seeded at 90,000 cells per well in a standard 96 well plate. The cells were treated with the indicated hCIDEB targeting GalNAc-dsRNA agent by uptake (5000 nM, 1250 nM, 313 nM, 78.1 nM, 19.5 nM, 4.88 nM, 1.22 nM, 0.305 nM, 0.0763 nM, or 0.0191 nM) and incubated for 48 hours. The level of hCIDEB was assessed utilizing a standard bDNA assay (Quantigene 2.0 (Thermo Fisher Scientific)) according to manufacturer's instructions. The expression of hCIDEB was normalized to the expression of human GAPDH. Each treatment group was run in quadruplicate.

Table 10 shows the calculated IC50 (nM), IC80 (nM), and the remaining hCIDEB mRNA percent (normalized to GAPDH) (mean of quadruplicates).

TABLE 10
IC50, IC80, and Max % Inhibition of hCIDEB mRNA
(Normalized to GAPDH).

| DsRNA | | | % Maximum |
|---|---|---|---|
| Agent | IC50 [nM] | IC80 [nM] | Inhibition |
| 487 | >1 ≤ 5 | >20 ≤ 30 | >80 ≤ 90 |
| 483 | >1 ≤ 5 | >10 ≤ 20 | >80 ≤ 90 |
| 486 | >1 ≤ 5 | >40 ≤ 50 | >80 ≤ 90 |
| 484 | >1 ≤ 5 | >50 ≤ 60 | >80 ≤ 90 |
| 485 | >1 ≤ 5 | >30 ≤ 40 | >80 ≤ 90 |

The invention is not to be limited in scope by the specific embodiments described herein. Indeed, various modifications of the invention in addition to those described will become apparent to those skilled in the art from the foregoing description and accompanying FIGURES. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A double stranded ribonucleic acid (dsRNA) agent for inhibiting expression of cell death inducing DNA fragmentation factor alpha like effector (CIDEB),
wherein the dsRNA agent comprises a sense strand and an antisense strand forming a double stranded region,
wherein the nucleotide sequence of the antisense strand differs by no more than 5 nucleotides from the nucleotide sequence of any one of the antisense strands set forth in Table 2 or Table 3 or set forth in any one of SEQ ID NOS: 187-369, 602-657, 893-1131, or 1188-1191.

2. The dsRNA agent of claim 1, wherein the nucleotide sequence of the sense strand differs by no more than 5 nucleotides from the nucleotide sequence of the corresponding sense strand set forth in Table 2 or Table 3 or the corresponding sense strand set forth in one of SEQ ID NOS: 4-186, 553-601, 658-892, or 1192.

3.-4. (canceled)

5. The dsRNA agent of claim 1, wherein
(a) the nucleotide sequence of the antisense strand differs by no more than 5 nucleotides from the nucleotide sequence set forth in SEQ ID NO: 355; and the nucleotide sequence of the sense strand differs by no more than 5 nucleotides from the nucleotide sequence set forth in SEQ ID NO: 172;
(b) the nucleotide sequence of the antisense strand differs by no more than 5 nucleotides from the nucleotide sequence set forth in SEQ ID NO: 315; and the nucleotide sequence of the sense strand differs by no more than 5 nucleotides from the nucleotide sequence set forth in SEQ ID NO: 132;
(c) the nucleotide sequence of the antisense strand differs by no more than 5 nucleotides from the nucleotide sequence set forth in SEQ ID NO: 356; and the nucleotide sequence of the sense strand differs by no more than 5 nucleotides from the nucleotide sequence set forth in SEQ ID NO: 173; or
(d) the nucleotide sequence of the antisense strand differs by no more than 5 nucleotides from the nucleotide sequence set forth in SEQ ID NO: 321; and the nucleotide sequence of the sense strand differs by no more than 5 nucleotides from the nucleotide sequence set forth in SEQ ID NO: 138.

6.-10. (canceled)

11. A dsRNA agent for inhibiting expression of CIDEB,
wherein the dsRNA agent comprises a sense strand and an antisense strand forming a double stranded region, and
wherein the nucleotide sequence of the antisense strand differs by no more than 5 nucleotides from the nucleotide sequence of any one of the antisense strands of any one of dsRNA agents 1-482 set forth in Table 2 or Table 3; and
the nucleotide sequence of the sense strand differs by no more than 5 nucleotides from the nucleotide sequence of the sense strand of the corresponding dsRNA agent set forth in Table 2 or Table 3.

12. (canceled)

13. The dsRNA agent of claim 11, wherein
(a) the nucleotide sequence of the antisense strand differs by no more than 5 nucleotides from the nucleotide sequence of the antisense strand of dsRNA agent 129; and the nucleotide sequence of the sense strand differs by no more than 5 nucleotides from the nucleotide sequence of dsRNA agent 129;
(b) the nucleotide sequence of the antisense strand differs by no more than 5 nucleotides from the nucleotide sequence of the antisense strand of dsRNA agent 135; and the nucleotide sequence of the sense strand differs by no more than 5 nucleotides from the nucleotide sequence of dsRNA agent 135;
(c) the nucleotide sequence of the antisense strand differs by no more than 5 nucleotides from the nucleotide sequence of the antisense strand of dsRNA agent 169; and the nucleotide sequence of the sense strand differs by no more than 5 nucleotides from the nucleotide sequence of dsRNA agent 169; or
(d) the nucleotide sequence of the antisense strand differs by no more than 5 nucleotides from the nucleotide sequence of the antisense strand of dsRNA agent 170; and the nucleotide sequence of the sense strand differs by no more than 5 nucleotides from the nucleotide sequence of dsRNA agent 170.

14.-18. (canceled)

19. A dsRNA agent for inhibiting expression of CIDEB,
wherein the dsRNA agent comprises a sense strand and an antisense strand forming a double stranded region, and
wherein the sense strand comprises at least 15 (e.g., 16, 17, 18, 19, 20, 21) contiguous nucleotides of and differing by no more than 5 (e.g., 0, 1, 2, 3, 4, or 5 (e.g., 3 (e.g., 0, 1, 2, or 3))) nucleotides from nucleotides 50-90, 60-90, 68-106, 81-103, 138-190, 169-206, 232-254, 230-300, 280-312, 298-327, 444-498, 510-538, 521-558, 560-617, 692-778, 720-750, 740-780, 750-790, 760-780, 880-920, 890-940, 890-950, 920-970, 930-980, 961-1037, 1035-1060, 1105-1133, 1171-1213, 1216-1287, 1220-1270, 1230-1280, 1240-1290, 1250-1300, 1240-1270, 1240-1280, 1235-1270, 1245-1265, 1247-1267, 1252-1272, 1294-1346, 1366-1400, 1370-1410, 1360-1400, 1470-1510, 1410-1460, 1520-1560, 1580-1620, 1640-1680, 1640-1690, 1670-1710, 1700-1740,1700-1745, 1724-1753, 1750-1790, 1757-1812, 1770-1810, 1833-1864, 1880-1920, 1900-1940, 1900-1927, 1915-1966, 1920-1970, 1930-1970, 1930-1965, 1940-1970, 1940-1965, 1937-1957, 1942-1962, 1938-1958, 1943-1963, 2010-2060, 2020-2060, 2030-2070, 2082-2110, 2080-2120, 2090-2130, 2130-2170, 2143-2198, 2197-2225, 2210-2250, 2215-2260, 2240-2280, 2250-2280, 2250-2290, or 2260-2290 of SEQ ID NO: 1;
wherein the nucleotide sequence of the antisense strand comprises at least 15 contiguous nucleotides of and differing by no more than 5 nucleotides from the nucleotide sequence of the corresponding complementary nucleotide sequence of SEQ ID NO: 2.

20.-24. (canceled)

25. A dsRNA agent for inhibiting expression of CIDEB,
wherein the dsRNA agent comprises a sense strand and an antisense strand forming a double stranded region,
wherein the nucleotide sequence of the antisense strand comprises at least 15 contiguous nucleotides of and differing by no more than 5 nucleotides from the nucleotide sequence of any one of the antisense strands set forth in Table 2 or Table 3 or set forth in any one of SEQ ID NOS: 187-369, 602-657, 893-1131, or 1188-1191.

26.-70. (canceled)

71. A conjugate comprising the dsRNA agent of claim 1 and a heterologous moiety.

72.-95. (canceled)

96. A vector encoding the antisense strand, the sense strand, or both the antisense and sense strand of claim 1.

97. A carrier comprising the dsRNA agent of claim 1.

98.-99. (canceled)

100. A cell (or population of cells) comprising the dsRNA agent of claim 1.

101. A pharmaceutical composition comprising the dsRNA agent of claim 1, and a pharmaceutically acceptable excipient.

102. A kit comprising the dsRNA agent of claim 1.

103. A method of delivering a dsRNA agent to a cell, the method comprising introducing into a cell the dsRNA agent of claim 1, to thereby deliver the dsRNA agent into the cell.

104.-106. (canceled)

107. A method of reducing or inhibiting expression of CIDEB in a cell, the method comprising delivering into the cell the dsRNA agent of claim 1, to thereby reduce or inhibit expression of CIDEB in the cell.

108. (canceled)

109. A method of treating, ameliorating, or preventing a CIDEB associated disease in a subject, the method comprising administering to the subject the dsRNA agent of claim 1, to thereby treat, ameliorate, or prevent the CIDEB associated disease in the subject.

110.-116. (canceled)

117. A method of diagnosing a CIDEB associated disease in a subject, the method comprising
(a) isolating and purifying DNA, RNA, or protein, or having DNA, RNA, or protein isolated and purified, from a sample obtained from the subject;
(b) detecting, or having detected, the presence or absence of one or more somatic CIDEB mutation in the DNA, RNA, or protein,
wherein the presence of the one or more somatic mutation indicates that the subject has a CIDEB associated disease.

118.-126. (canceled)

127. A method of selecting a subject for administration of an inhibitory nucleic acid molecule that inhibits expression of CIDEB, the method comprising
(a) isolating and purifying DNA, RNA, or protein, or having DNA, RNA, or protein isolated and purified, from a sample obtained from the subject;
(b) detecting, or having detected, the presence or absence of one or more somatic CIDEB mutation in the DNA, RNA, or protein,
wherein the subject is selected for administration of the inhibitory nucleic acid molecule if the one or more somatic mutation is present.

128.-130. (canceled)

131. A method of treating, ameliorating, or preventing a CIDEB associated disease in a subject, the method comprising
(a) isolating and purifying DNA, RNA, or protein, or having DNA, RNA, or protein isolated and purified, from a sample obtained from the subject;
(b) detecting, or having detected, the presence or absence of one or more somatic CIDEB mutation in the DNA, RNA, or protein, and
(c) administering to the subject an inhibitory nucleic acid that inhibits expression of CIDEB if the one or more CIDEB somatic mutation is detected in the DNA, RNA, or protein.

132.-141. (canceled)

142. An in vitro method of screening a sample from a subject for one or more somatic CIDEB mutation, the method comprising
(a) isolating and purifying DNA, RNA, or protein from a sample obtained from the subject; and
(b) detecting the presence or absence of one or more somatic CIDEB mutation in the DNA, RNA, or protein.

143.-152. (canceled)
